SEQUENCE LISTING (423 sequences)

SEQ ID NO: 1 - protein, 33 aa, Arabidopsis thaliana
MFCSIQCVARHLFPLHVREIKKATRAIKKGKTL

SEQ ID NO: 2 - protein, 24 aa, Arabidopsis thaliana
MRRQTSVPFACKRDKESDKSHKER

SEQ ID NO: 3 - protein, 37 aa, Arabidopsis thaliana
MVMFFLDLDKNPRFDLLKGLKWNLFSSHISPSLPPSL

SEQ ID NO: 4 - protein, 11 aa, Arabidopsis thaliana
MKDNFPLLLRL

SEQ ID NO: 5 - protein, 4 aa, Arabidopsis thaliana
MSDD

SEQ ID NO: 6 - protein, 16 aa, Arabidopsis thaliana
MIYINKYGSISAVEDD

SEQ ID NO: 7 - protein, 4 aa, Arabidopsis thaliana
MSQR

SEQ ID NO: 8 - protein, 16 aa, Arabidopsis lyrata
MTCPLLSLSFLLSKYI

SEQ ID NO: 9 - protein, 16 aa, Arabidopsis thaliana
MTWPLLSLSFLLSKYV

SEQ ID NO: 10 - protein, 18 aa, Capsella rubella
MTCTLSALSLSLNMFRVN

SEQ ID NO: 11 - protein, 5 aa, Arabidopsis thaliana
MFYLS

SEQ ID NO: 12 - protein, 37 aa, Arabidopsis thaliana
MVNTSSFFISSFILPLVLSESNCLLFRTIYKFSMVLY

SEQ ID NO: 13 - protein, 24 aa, Arabidopsis thaliana
MFCLLIPIFSFVFSPNRHLRLQEQ

SEQ ID NO: 14 - protein, 5 aa, Arabidopsis thaliana
MFSPQ

SEQ ID NO: 15 - protein, 23 aa, Arabidopsis thaliana
MKYIHILILFKSRSTYKLSTNHI

SEQ ID NO: 16 - protein, 10 aa, Arabidopsis thaliana
MKIPLFLPKL

SEQ ID NO: 17 - protein, 27 aa, Arabidopsis thaliana
MVSGQEDSWLKLSSLCFLFLSLLDSLI

SEQ ID NO: 18 - protein, 34 aa, Arabidopsis thaliana
MFLLIFLRLIMICVCSSTDFLRSVNYFCLFIYDL

SEQ ID NO: 19 - protein, 9 aa, Arabidopsis thaliana
MSTTQEHRS

SEQ ID NO: 20 - protein, 25 aa, Arabidopsis thaliana
MILKCWSSRFLRVSPYQNAHSLSLG

SEQ ID NO: 21 - protein, 13 aa, Arabidopsis lyrata
MPLAVIRQGIVWP

SEQ ID NO: 22 - protein, 24 aa, Arabidopsis lyrata
MPSWHDMVLLPYVKHTHANTRHIT

SEQ ID NO: 23 - protein, 8 aa, Arabidopsis lyrata
MTWFFCLT

SEQ ID NO: 24 - protein, 37 aa, Arabidopsis thaliana
MPSWHGMVLLPYVKHTHASTHTHTHNIYGCACELVFH

SEQ ID NO: 25 - protein, 22 aa, Arabidopsis thaliana
MAWYGSFALRKTHSRQHTHTHT

SEQ ID NO: 26 - protein, 8 aa, Arabidopsis thaliana
MVWFFCLT

SEQ ID NO: 27 - protein, 7 aa, Brassica rapa
MMIILWK

SEQ ID NO: 28 - protein, 24 aa, Brassica rapa
MLWAKLVSFSTLHSLVFLLSPSFA

SEQ ID NO: 29 - protein, 29 aa, Brassica rapa
MPSWHGIVILPFVKHTHANIHYSYSCVCI

SEQ ID NO: 30 - protein, 34 aa, Carica papaya
MIACHPYLPFPLFLSLTFYSIFFSPSPPSPSLPL

SEQ ID NO: 31 - protein, 53 aa, Carica papaya
MPSLLAFSPFPFSNILLNLLLPLPPFPLSAIITIIKPLSLSLPLSLSLSGFSV

SEQ ID NO: 32 - protein, 15 aa, Capsella rubella
MELKGLRTWQLLDKV

SEQ ID NO: 33 - protein, 14 aa, Capsella rubella
MPSWHGMACFYCLT

SEQ ID NO: 34 - protein, 24 aa, Capsella rubella
MAWHGMFLLPYVKHTHANTYSLYM

SEQ ID NO: 35 - protein, 35 aa, Gossypium raimondii
MMRSRILKFQYRFGMGIGGRKQLKNQLCQIQGRIS

SEQ ID NO: 36 - protein, 10 aa, Gossypium raimondii
MSNSRSYQLK

SEQ ID NO: 37 - protein, 17 aa, Gossypium raimondii
MNEDLEISTRKRTPQLC

SEQ ID NO: 38 - protein, 12 aa, Medicago truncatula
MPKFDIFFYIFV

SEQ ID NO: 39 - protein, 83 aa, Medicago truncatula
MSYISLSPKLLPINTKPFPWLVQFNFYFSSNTKCNKLHFLGEKLLVGEAGHVQILFLIHSLIMHINIFCTCSPSPTRLPHPSL

SEQ ID NO: 40 - protein, 20 aa, Oryza sativa
MQTHSNTPQSTYSLSLSLSE

SEQ ID NO: 41 - protein, 15 aa, Oryza sativa
MCVCDINMHSMLMLL

SEQ ID NO: 42 - protein, 25 aa, Arabidopsis lyrata
MRIKLFQLRGMLSGSRILYIYTCVC

SEQ ID NO: 43 - protein, 18 aa, Arabidopsis thaliana
MRVKLFQLRGMLSGSRIL

SEQ ID NO: 44 - protein, 36 aa, Brassica carinata
MRMKLFQLRGMLSGSRILYIHKYVYMLIQVFDHICI

SEQ ID NO: 45 - protein, 27 aa, Brassica juncea
MRMKLFQLRGMLSGSRILYIHKYVYIC

SEQ ID NO: 46 - protein, 36 aa, Brassica napus
MRMKLFQLRGMLSGSRILYIHKYVYMIIQVFDHICI

SEQ ID NO: 47 - protein, 36 aa, Brassica oleracea; identical to SEQ ID NO: 44

SEQ ID NO: 48 - protein, 27 aa, Brassica rapa
MRMKLFQLRGMLSGSRILYIHKYVYIC

SEQ ID NO: 49 - protein, 19 aa, Arabidopsis thaliana
MLDLFRSNNRIEPSDFRFD

SEQ ID NO: 50 - protein, 4 aa, Arabidopsis thaliana
MRDR

SEQ ID NO: 51 - protein, 10 aa, Arabidopsis thaliana
MNRKISLSLS

SEQ ID NO: 52 - protein, 8 aa, Arabidopsis thaliana
MMGCFVGF

SEQ ID NO: 53 - protein, 8 aa, Arabidopsis thaliana
MQEETYEG

SEQ ID NO: 54 - protein, 59 aa, Arabidopsis thaliana
MPHTNLKDLFIFSPNVFFSFAIYLHNSWNKNYIHKRENFHNTSFALIFFFSSIMSINYG

SEQ ID NO: 55 - protein, 39 aa, Arabidopsis thaliana
MFFFRLLFISTILGTKTTFTNERIFTTPLLLSFFFFRPL

SEQ ID NO: 56 - protein, 6 aa, Arabidopsis thaliana
MRHKES

SEQ ID NO: 57 - protein, 34 aa, Arabidopsis thaliana
MNLLKKERQRRRQRSIGSHCIASLVLKDGYMKKI

SEQ ID NO: 58 - protein, 9 aa, Arabidopsis thaliana
MVLSGKLTF

SEQ ID NO: 59 - protein, 20 aa, Medicago truncatula
MLLHRLSKFCKIERDIVYIS

SEQ ID NO: 60 - protein, 5 aa, Medicago truncatula
MKIEE

SEQ ID NO: 61 - protein, 34 aa, Zea mays
MHLPSTPSRPPPQHTSLSFLGKEMTKGTTTACFG

SEQ ID NO: 62 - protein, 10 aa, Arabidopsis thaliana
MLSLSHFHIC

SEQ ID NO: 63 - protein, 23 aa, Medicago truncatula
MMVFGKPKKAMLVRFNPKTDLHV

SEQ ID NO: 64 - protein, 10 aa, Medicago truncatula
MASAAKVYMA

SEQ ID NO: 65 - protein, 6 aa, Arabidopsis thaliana
MASKIW

SEQ ID NO: 66 - protein, 10 aa, Arabidopsis thaliana
MVRFQLSIRD

SEQ ID NO: 67 - protein, 12 aa, Arabidopsis thaliana
MCTYYYLINKYF

SEQ ID NO: 68 - protein, 11 aa, Arabidopsis thaliana
MFPAKWCRLES

SEQ ID NO: 69 - protein, 22 aa, Arabidopsis thaliana
MGSLSLFKSQLEILMLLLSLSK

SEQ ID NO: 70 - protein, 20 aa, Arabidopsis thaliana
MSVYIHVPISLNCFSPKSSC

SEQ ID NO: 71 - protein, 11 aa, Arabidopsis thaliana
MGVPNFRPRNR

SEQ ID NO: 72 - protein, 23 aa, Arabidopsis cebennensis
MRSRVSFFFFKIMLFRLLGYRSM

SEQ ID NO: 73 - protein, 44 aa, Arabidopsis cebennensis
MHTYIHTISNISSIFFCSKRSFSPFTYIRIIVVIDPFRIALTFR

SEQ ID NO: 74 - protein, 22 aa, Arabidopsis halleri
MRSRVSLFLSFSSNFAAYSPRS

SEQ ID NO: 75 - protein, 48 aa, Arabidospis lyrata
MHTYIPSSSFPISNISSVFFCYKRSFSPYTYIRIIVVIDPFRIALTFR

SEQ ID NO: 76 - protein, 50 aa, Arabidopsis thaliana
MNIHTYHHLLFPSLVFHQSSDVPNALSLHIHTYEYIIVVIDPFRITLAFR

SEQ ID NO: 77 - protein, 19 aa, Arabidopsis thaliana
MFQTLYLFIYIHTNILLLS

SEQ ID NO: 78 - protein, 45 aa, Brassica rapa
MFKLYFSAILSTQYMHTYHHRIALIFLSILYPSTNYLMSPILNPT

SEQ ID NO: 79 - protein, 20 aa, Carica papaya
MKIKLGFSLIKIIILLDKNS

SEQ ID NO: 80 - protein, 21 aa, Capsella rubella
MHPHTYIHIPSSSFLISSFCL

SEQ ID NO: 81 - protein, 45 aa, Eucalyptus grandis
MKHIQRWRYGETSGRQGDWKRLEIKVHSNPSLKVKKNTNNFSSSL

SEQ ID NO: 82 - protein, 28 aa, Gossypium raimondii
MIHFNLSQWRAICMANFHLTYSFLFGVL

SEQ ID NO: 83 - protein, 21 aa, Medicago truncatula
MHVYLELFMVIKGLGFLLLVK

SEQ ID NO: 84 - protein, 23 aa, Oryza sativa
MEMIQRPCLILKFFFKLSTLYIP

SEQ ID NO: 85 - protein, 38 aa, Physcomitrella patens
MFHRRRSSVLLPPFGQTQPNPRCLPDLRFPSCFTPCTA

SEQ ID NO: 86 - protein, 42 aa, Thellungiella halophila
MTICKVSKACFYAGKIENSRLIKKIGIPKREGAPFSPIRENQ

SEQ ID NO: 87 - protein, 37 aa, Thellungiella halophila
MEIQIKKKNLYIMNTQKLPNLYIYIYKYVFIKLMVVE

SEQ ID NO: 88 - protein, 43 aa, Arabidopsis thaliana
MVPQINLWSSRVILKIRIDSSTHREEDHCIQNHKHGLSFIFSF

SEQ ID NO: 89 - protein, 16 aa, Arabidopsis thaliana
MSLQFYERVSFKNTVK

SEQ ID NO: 90 - protein, 12 aa, Arabidopsis thaliana
MTEQEEESQMST

SEQ ID NO: 91 - protein, 39 aa, Arabidopsis thaliana
MYLQYIDNVISIYSNNRRVGRMFSRVPLSTSLEIQFFIK

SEQ ID NO: 92 - protein, 12 aa, Arabidopsis thaliana
MSKEIFFSPGFE

SEQ ID NO: 93 - protein, 44 aa, Arabidopsis thaliana
MRTHEQSTAITTLRHCYSSRFMCSQVTPAELFLYRPCFINAVAR

SEQ ID NO: 94 - protein, 5 aa, Arabidopsis thaliana
MKRNM

SEQ ID NO: 95 - protein, 5 aa, Arabidopsis thaliana
MQCEI

SEQ ID NO: 96 - protein, 4 aa, Arabidopsis thaliana
MFCA

SEQ ID NO: 97 - protein, 6 aa, Arabidopsis thaliana
MVMAHH

SEQ ID NO: 98 - protein, 54 aa, Arabidopsis thaliana
MMKPRWNCSLYGITEWTNNQNQKSKRKGRRKTQIWRIGDRLDTVECITLMLSAY

SEQ ID NO: 99 - protein, 10 aa, Arabidopsis thaliana
MLLIIVELVL

SEQ ID NO: 100 - protein, 14 aa, Arabidopsis thaliana
MLCFNFRCVRRFAE

SEQ ID NO: 101 - protein, 36 aa, Arabidopsis thaliana
MYTYQLDNSFSWFLCTRFCLYRYFLFNFRCFRRFSE

SEQ ID NO: 102 - protein, 88 aa, Drosophila melanogaster
MWREVCAQKSQTKRRNFITGNQRRNKTKANRKAETKQQKVYEFFVQARERCKTRKKHEKKTLKKTKKIQNRYRAVSENEWGKGFPSHI

SEQ ID NO: 103 - protein, 36 aa, Drosophila melanogaster
MRTKKSNKKAQFYYGQPTTKQNKSQPKSRNKAAKSL

SEQ ID NO: 104 - protein, 71 aa, Drosophila melanogaster
MEPGFVFVLFPTHLSTQHTQREKSILVMGLNLQSAKQSDKQNSKERKKNTQINSQRIPYRQGGQCSKVLSP

SEQ ID NO: 105 - DNA, 92 bp, Arabidopsis thaliana
  source 1..92: mol_type = unassigned DNA
atgttctgttcaattcaatgcgtcgccagacatctgttccctttgcatgtaagagagataaagaaagcgacaagagccataaagaaaggtaa

SEQ ID NO: 106 - DNA, 75 bp, Arabidopsis thaliana
  source 1..75: mol_type = unassigned DNA
atgcgtcgccagacatctgttccctttgcatgtaagagagataaagaaagcgacaagagccataaagaaaggtaa

SEQ ID NO: 107 - DNA, 114 bp, Arabidopsis thaliana
  source 1..114: mol_type = unassigned DNA
atggttatgttttttctcgatttagacaaaaaccctagatttgatcttctaaagggtctcaaatggaatctcttctcttctcatatctctccctctctccctccctctctttga

SEQ ID NO: 108 - DNA, 36 bp, Arabidopsis thaliana
  source 1..36: mol_type = unassigned DNA
atgaaggacaactttcctcttctccttcggttataa

SEQ ID NO: 109 - DNA, 15 bp, Arabidopsis thaliana
  source 1..15: mol_type = unassigned DNA
atgagtgatgactga

SEQ ID NO: 110 - DNA, 51 bp, Arabidopsis thaliana
  source 1..51: mol_type = unassigned DNA
atgatatatataaataaatatgggtcgatatcggctgtggaggacgactag

SEQ ID NO: 111 - DNA, 15 bp, Arabidopsis thaliana
  source 1..15: mol_type = unassigned DNA
atgagccaaagataa

SEQ ID NO: 112 - DNA, 51 bp, Arabidopsis lyrata
  source 1..51: mol_type = unassigned DNA
atgacgtgtcctcttctctctctctctttccttctctctaagtatatttag

SEQ ID NO: 113 - DNA, 51 bp, Arabidopsis thaliana
  source 1..51: mol_type = unassigned DNA
atgacgtggcctcttctctctctctctttccttctctctaagtatgtttag

SEQ ID NO: 114 - DNA, 53 bp, Capsella rubella
  source 1..53: mol_type = unassigned DNA
atgacgtgtactctctctgctctatctctctctctaaatatgtttagggttaa

SEQ ID NO: 115 - DNA, 18 bp, Arabidopsis thaliana
  source 1..18: mol_type = unassigned DNA
atgttttatctttcataa

SEQ ID NO: 116 - DNA, 114 bp, Arabidopsis thaliana
  source 1..114: mol_type = unassigned DNA
atggttaatactagtagctttttcatttcaagttttatccttccattggttctttctgagtcaaattgtctcctgtttcgaaccatatataagttttcaatggttttgtattaa

SEQ ID NO: 117 - DNA, 75 bp, Arabidopsis thaliana
  source 1..75: mol_type = unassigned DNA
atgttttgtttgttgattcccatcttctcttttgtcttttcaccaaatcgtcatttaaggcttcaagaacagtaa

SEQ ID NO: 118 - DNA, 18 bp, Arabidopsis thaliana
  source 1..18: mol_type = unassigned DNA
atgttttcccctcaatga

SEQ ID NO: 119 - DNA, 72 bp, Arabidopsis thaliana
  source 1..72: mol_type = unassigned DNA
atgaaatacatacacattttgattttatttaaatcaagatcgacgtataagctatccaccaatcatatttaa

SEQ ID NO: 120 - DNA, 33 bp, Arabidopsis thaliana
  source 1..33: mol_type = unassigned DNA
atgaaaattccattgtttctgccgaagctttga

SEQ ID NO: 121 - DNA, 84 bp, Arabidopsis thaliana
  source 1..84: mol_type = unassigned DNA
atggtatctggtcaagaagattcctggttaaaactttcatctctctgtttcctttttctttctttgttggattcattaatttga

SEQ ID NO: 122 - DNA, 105 bp, Arabidopsis thaliana
  source 1..105: mol_type = unassigned DNA
atgtttcttttaatctttttgagattaataatgatttgtgtttgttcatcaaccgattttctcagatctgtcaattatttttgtttatttatttatgatttatga

SEQ ID NO: 123 - DNA, 30 bp, Arabidopsis thaliana
  source 1..30: mol_type = unassigned DNA
atgtccactactcaagagcataggtcttga

SEQ ID NO: 124 - DNA, 78 bp, Arabidopsis thaliana
  source 1..78: mol_type = unassigned DNA
atgatactaaagtgctggagttcccggttcctgagagtgagtccatatcaaaatgcgcattcgttatcacttggttga

SEQ ID NO: 125 - DNA, 42 bp, Arabidopsis lyrata
  source 1..42: mol_type = unassigned DNA
atgcccttagcagttattagacaagggattgtttggccctag

SEQ ID NO: 126 - DNA, 75 bp, Arabidopsis lyrata
  source 1..75: mol_type = unassigned DNA
atgccatcatggcatgacatggttcttttgccttacgtaaaacacactcacgccaacacacgccacataacataa

SEQ ID NO: 127 - DNA, 27 bp, Arabidopsis lyrata
  source 1..27: mol_type = unassigned DNA
atgacatggttcttttgccttacgtaa

SEQ ID NO: 128 - DNA, 114 bp, Arabidopsis thaliana
  source 1..114: mol_type = unassigned DNA
atgccatcatggcatggtatggttcttttgccttacgtaaaacacactcacgccagcacacacacacacacacataacatatacggatgtgcgtgtgagctagtcttccattaa

SEQ ID NO: 129 - DNA, 69 bp, Arabidopsis thaliana
  source 1..69: mol_type = unassigned DNA
atggcatggtatggttcttttgccttacgtaaaacacactcacgccagcacacacacacacacacataa

SEQ ID NO: 130 - DNA, 27 bp, Arabidopsis thaliana
  source 1..27: mol_type = unassigned DNA
atggtatggttcttttgccttacgtaa

SEQ ID NO: 131 - DNA, 24 bp, Brassica rapa
  source 1..24: mol_type = unassigned DNA
atgatgataattttgtggaaataa

SEQ ID NO: 132 - DNA, 75 bp, Brassica rapa
  source 1..75: mol_type = unassigned DNA
atgctttgggccaagctagtttcttttagcactcttcactcactagtttttcttctcagcccttcttttgcgtga

SEQ ID NO: 133 - DNA, 120 bp, Brassica rapa
  source 1..120: mol_type = unassigned DNA
atgccatcatggcatggcattgtcattttgcctttcgtaaaacacactcacgccaacatacattattcatattcatgtgtatgtatatgaatgccatcatggcatatgccatcatggcat

SEQ ID NO: 134 - DNA, 105 bp, Carica papaya
  source 1..105: mol_type = unassigned DNA
atgattgcatgccatccctacttgccttttccccttttcctttctctaacattttactcaatcttcttctccccctcccccccttccccctctctgccattataa

SEQ ID NO: 135 - DNA, 162 bp, Carica papaya
  source 1..162: mol_type = unassigned DNA
atgccatccctacttgccttttccccttttcctttctctaacattttactcaatcttcttctccccctcccccccttccccctctctgccattataaccataattaaacctctctccctctctctccctctctctctctctctctctgggttctcagtataa

SEQ ID NO: 136 - DNA, 48 bp, Capsella rubella
  source 1..48: mol_type = unassigned DNA
atggaattaaaaggtttgagaacttggcagttattagacaaggtatag

SEQ ID NO: 137 - DNA, 45 bp, Capsella rubella
  source 1..45: mol_type = unassigned DNA
atgccatcatggcatggcatggcatgtttctattgccttacgtaa

SEQ ID NO: 138 - DNA, 90 bp, Capsella rubella
  source 1..90: mol_type = unassigned DNA
atggcatggcatgtttctattgccttacgtaaaacacactcacgccaacacatactcactatacatgtaaataagtatgtgcgcgtgtga

SEQ ID NO: 139 - DNA, 108 bp, Gossypium raimondii
  source 1..108: mol_type = unassigned DNA
atgatgagatcaagaattttaaagtttcaatatagatttggcatgggtattggcggcagaaagcaattaaaaaaccagttatgtcaaattcaaggtcgtatcagttaa

SEQ ID NO: 140 - DNA, 33 bp, Gossypium raimondii
  source 1..33: mol_type = unassigned DNA
atgtcaaattcaaggtcgtatcagttaaaatga

SEQ ID NO: 141 - DNA, 54 bp, Gossypium raimondii
  source 1..54: mol_type = unassigned DNA
atgaatgaagatttagaaatttcaacaaggaagaggaccccacagctttgttaa

SEQ ID NO: 142 - DNA, 39 bp, Medicago truncatula
  source 1..39: mol_type = unassigned DNA
atgcccaaatttgatatttttttttatatatttgtatag

SEQ ID NO: 143 - DNA, 231 bp, Medicago truncatula
  source 1..231: mol_type = unassigned DNA
atgtcatatatctctctctctcctaagttgctacctataaatactaagcctttcccttggttggttcaattcaacttctacttctcatcaaacacaaagtgcaataagcttcatttcctgggtgagaagctccttgttggagaagcagggcacgtgcaaatcctctttctgattcattctctcataatgcatatcaatatcttttgcacgtgctccccttctccaactagg

SEQ ID NO: 144 - DNA, 63 bp, Oryza sativa
  source 1..63: mol_type = unassigned DNA
atgcaaacccactccaacactccacaatccacatactctctctctctctctctctctgagtag

SEQ ID NO: 145 - DNA, 48 bp, Oryza sativa
  source 1..48: mol_type = unassigned DNA
atgtgtgtgtgtgatatcaatatgcattcgatgttgatgctactgtag

SEQ ID NO: 146 - DNA, 78 bp, Arabidopsis lyrata
  source 1..78: mol_type = unassigned DNA
atgagaattaagctatttcagttgaggggaatgttgtctggatcgaggatattatacatatatacatgtgtatgttga

SEQ ID NO: 147 - DNA, 57 bp, Arabidopsis thaliana
  source 1..57: mol_type = unassigned DNA
atgagggttaagctatttcagttgaggggaatgttgtctggatcgaggatattatag

SEQ ID NO: 148 - DNA, 111 bp, Brassica carinata
  source 1..111: mol_type = unassigned DNA
atgagaatgaagctatttcagttgaggggaatgttgtctggatcgaggatattatatatacacaaatacgtatatatgttaatacaagtgtttgatcatatatgtatatag

SEQ ID NO: 149 - DNA, 84 bp, Brassica juncea
  source 1..84: mol_type = unassigned DNA
atgagaatgaagctatttcagttgaggggaatgttgtctggatcgaggatattatatatacacaaatatgtatatatatgttaa

SEQ ID NO: 150 - DNA, 111 bp, Brassica napus
  source 1..111: mol_type = unassigned DNA
atgagaatgaagctatttcagttgaggggaatgttgtctggatcgaggatattatatatacacaaatacgtatatatgataatacaagtgtttgatcatatatgtatatag

SEQ ID NO: 151 - DNA, 111 bp, Brassica oleracea
  source 1..111: mol_type = unassigned DNA
atgagaatgaagctatttcagttgaggggaatgttgtctggatcgaggatattatatatacacaagtacgtatatatgttaatacaagtgtttgatcatatatgtatatag

SEQ ID NO: 152 - DNA, 84 bp, Brassica rapa; identical to SEQ ID NO: 149
  source 1..84: mol_type = unassigned DNA

SEQ ID NO: 153 - DNA, 60 bp, Arabidopsis thaliana
  source 1..60: mol_type = unassigned DNA
atgttggatctctttcgatctaacaatcgaattgaaccttcagatttcagatttgattag

SEQ ID NO: 154 - DNA, 15 bp, Arabidopsis thaliana
  source 1..15: mol_type = unassigned DNA
atgagagatagataa

SEQ ID NO: 155 - DNA, 33 bp, Arabidopsis thaliana
  source 1..33: mol_type = unassigned DNA
atgaacagaaaaatctctctttctctttcttga

SEQ ID NO: 156 - DNA, 27 bp, Arabidopsis thaliana
  source 1..27: mol_type = unassigned DNA
atgatgggttgttttgtgggattttaa

SEQ ID NO: 157 - DNA, 27 bp, Arabidopsis thaliana
  source 1..27: mol_type = unassigned DNA
atgcaggaggaaacatatgaggggtga

SEQ ID NO: 158 - DNA, 180 bp, Arabidopsis thaliana
  source 1..180: mol_type = unassigned DNA
atgccacatacaaacttgaaagatctcttcatcttttctccaaatgtttttttttcgtttgctatttatctccacaattcttggaacaaaaactacattcacaaacgagagaattttcacaacacctcttttgctctcattttttttttttcgtccattatgagtattaattatggttag

SEQ ID NO: 159 - DNA, 120 bp, Arabidopsis thaliana
  source 1..120: mol_type = unassigned DNA
atgtttttttttcgtttgctatttatctccacaattcttggaacaaaaactacattcacaaacgagagaattttcacaacacctcttttgctctcattttttttttttcgtccattatga

SEQ ID NO: 160 - DNA, 21 bp, Arabidopsis thaliana
  source 1..21: mol_type = unassigned DNA
atgagacataaagagagttaa

SEQ ID NO: 161 - DNA, 105 bp, Arabidopsis thaliana
  source 1..105: mol_type = unassigned DNA
atgaacctcctcaagaaggaaagacagaggaggagacaaagaagtataggttcacattgcatagccagtttagttttgaaggatggatatatgaaaaaaatatga

SEQ ID NO: 162 - DNA, 30 bp, Arabidopsis thaliana
  source 1..30: mol_type = unassigned DNA
atggttctctccggtaaattaacattttag

SEQ ID NO: 163 - DNA, 63 bp, Medicago truncatula
  source 1..63: mol_type = unassigned DNA
atgcttcttcataggctctccaaattttgcaaaattgaaagagacatagtatatatatcttag

SEQ ID NO: 164 - DNA, 18 bp, Medicago truncatula
  source 1..18: mol_type = unassigned DNA
atgaagattgaagagtaa

SEQ ID NO: 165 - DNA, 105 bp, Zea mays
  source 1..105: mol_type = unassigned DNA
atgcatctgccttcaactccctctcgccccccaccccaacacacatctctctcttttctagggaaggaaatgacgaaggggacgacgacggcatgcttcggctag

SEQ ID NO: 166 - DNA, 33 bp, Arabidopsis thaliana
  source 1..33: mol_type = unassigned DNA
atgttgtctctttctcattttcatatctgctaa

SEQ ID NO: 167 - DNA, 72 bp, Medicago truncatula
  source 1..72: mol_type = unassigned DNA
atgatggtgtttgggaagccgaaaaaagcgatgttggtgaggttcaatccgaagacggatttacatgtatag

SEQ ID NO: 168 - DNA, 33 bp, Medicago truncatula
  source 1..33: mol_type = unassigned DNA
atggcttcagctgcaaaagtatacatggcgtga

SEQ ID NO: 169 - DNA, 21 bp, Arabidopsis thaliana
  source 1..21: mol_type = unassigned DNA
atggcttccaagatctggtaa

SEQ ID NO: 170 - DNA, 33 bp, Arabidopsis thaliana
  source 1..33: mol_type = unassigned DNA
atggttaggttccaactaagtatacgagattaa

SEQ ID NO: 171 - DNA, 39 bp, Arabidopsis thaliana
  source 1..39: mol_type = unassigned DNA
atgtgtacgtactattatctcataaataaatatttttaa

SEQ ID NO: 172 - DNA, 36 bp, Arabidopsis thaliana
  source 1..36: mol_type = unassigned DNA
atgtttccagcaaaatggtgccgtcttgagtcttga

SEQ ID NO: 173 - DNA, 69 bp, Arabidopsis thaliana
  source 1..69: mol_type = unassigned DNA
atgggatctctctctttatttaaaagtcaattagagatcttgatgctacttctgtccctttccaagtga

SEQ ID NO: 174 - DNA, 63 bp, Arabidopsis thaliana
  source 1..63: mol_type = unassigned DNA
atgagtgtatatattcatgtacctatctctctcaattgcttctcaccaaaatcatcttgctga

SEQ ID NO: 175 - DNA, 36 bp, Arabidopsis thaliana
  source 1..36: mol_type = unassigned DNA
atgggagttcccaactttagacctcgaaaccgataa

SEQ ID NO: 176 - DNA, 72 bp, Arabidopsis cebennensis
  source 1..72: mol_type = unassigned DNA
atgagatctagggtttctttctttttcttcaaaatcatgctttttcgcttgctaggttatagatccatgtaa

SEQ ID NO: 177 - DNA, 134 bp, Arabidopsis cebennensis
  source 1..134: mol_type = unassigned DNA
atgcatacatacatacataccatctctaatatttcatcaatcttcttttgttccaaacgcctttctctccatttacatacatacgaatcattgttgtcatagatccgtttagaattgctttaacttttagatga

SEQ ID NO: 178 - DNA, 69 bp, Arabidopsis halleri
  source 1..69: mol_type = unassigned DNA
atgagatctagggtttctttgtttctttcgttttcttcaaattttgctgcatattctccaagatcatga

SEQ ID NO: 179 - DNA, 147 bp, Arabidospis lyrata
  source 1..147: mol_type = unassigned DNA
atgcatacatacataccatcatcatcttttcccatctctaatatttcatcagtcttcttttgttacaaacgctctttctcgccatatacatacataagaatcattgttgtcatagatccgtttagaattgctttaacttttagatga

SEQ ID NO: 180 - DNA, 153 bp, Arabidopsis thaliana
  source 1..153: mol_type = unassigned DNA
atgaatatacatacataccatcatcttcttttcccatctctagtttttcatcaatcttctgatgttccaaacgctctatctcttcatatacatacatacgaatatattattgttgtcatagatccatttagaatcactttagcttttagatga

SEQ ID NO: 181 - DNA, 60 bp, Arabidopsis thaliana
  source 1..60: mol_type = unassigned DNA
atgttccaaacgctctatctcttcatatacatacatacgaatatattattgttgtcatag

SEQ ID NO: 182 - DNA, 138 bp, Brassica rapa
  source 1..138: mol_type = unassigned DNA
atgtttaagctctacttctcagcaattctctccacccaatacatgcatacataccatcatcgtatcgctctaatttttctatcaatcttgtatccttccacaaattatcttatgtctcccattttaaatcctacatag

SEQ ID NO: 183 - DNA, 63 bp, Carica papaya
  source 1..63: mol_type = unassigned DNA
atgaagattaaattaggttttagtcttattaagattattatattactagacaaaaacagttaa

SEQ ID NO: 184 - DNA, 66 bp, Capsella rubella
  source 1..66: mol_type = unassigned DNA
atgcatccacatacatacatacatataccatcatcttcttttctcatctctagtttttgtttataa

SEQ ID NO: 185 - DNA, 138 bp, Eucalyptus grandis
  source 1..138: mol_type = unassigned DNA
atgaagcatattcaaaggtggagatatggggagacttccggaaggcaaggggattggaaaaggctcgagatcaaagtgcatagcaacccttcgctaaaggtgaaaaagaatacgaataacttcagtagctcactttaa

SEQ ID NO: 186 - DNA, 87 bp, Gossypium raimondii
  source 1..87: mol_type = unassigned DNA
atgatccatttcaacctgtcacagtggagagcaatttgtatggctaatttccatctcacctattcttttctgtttggggttctctag

SEQ ID NO: 187 - DNA, 66 bp, Medicago truncatula
  source 1..66: mol_type = unassigned DNA
atgcatgtatatcttgaattgtttatggtaataaaggggttaggatttctccttttggtgaagtga

SEQ ID NO: 188 - DNA, 72 bp, Oryza sativa
  source 1..72: mol_type = unassigned DNA
atggaaatgatacaaaggccgtgtttaattttaaaattttttttcaaactttcaacactttacatcccataa

SEQ ID NO: 189 - DNA, 117 bp, Physcomitrella patens
  source 1..117: mol_type = unassigned DNA
atgttccaccgtcggagatcctcggtgctgctacccccgttcggccaaacccaacccaaccctaggtgtctgccggacctccgcttcccctcctgcttcaccccctgcaccgcttaa

SEQ ID NO: 190 - DNA, 129 bp, Thellungiella halophila
  source 1..129: mol_type = unassigned DNA
atgacgatatgtaaagtaagcaaggcatgtttttatgcagggaagattgaaaattcaagattaatcaagaaaattggaataccaaaaagagagggagctcccttcagtccaatcagagagaatcaatga

SEQ ID NO: 191 - DNA, 114 bp, Thellungiella halophila
  source 1..114: mol_type = unassigned DNA
atggagattcaaattaaaaagaaaaacttatatataatgaatacacaaaagctacctaatctgtatatatatatatataaatatgtcttcattaaattaatggtcgtggaatag

SEQ ID NO: 192 - DNA, 132 bp, Arabidopsis thaliana
  source 1..132: mol_type = unassigned DNA
atggtacctcaaattaatctatggtcatctagggttatcttgaagattagaattgattctagcacgcacagagaggaagatcattgcatccagaatcacaaacatggcctatcttttatcttttctttttga

SEQ ID NO: 193 - DNA, 51 bp, Arabidopsis thaliana
  source 1..51: mol_type = unassigned DNA
atgtctctccaattttatgagagggtttccttcaagaacacagtaaaatag

SEQ ID NO: 194 - DNA, 39 bp, Arabidopsis thaliana
  source 1..39: mol_type = unassigned DNA
atgacagagcaagaagaagaaagtcaaatgtccacatga

SEQ ID NO: 195 - DNA, 120 bp, Arabidopsis thaliana
  source 1..120: mol_type = unassigned DNA
atgtatctacaatatattgataatgtaatatctatatattcaaacaatcgtcgtgttggtcggatgttttctagagttcctctgagcacttcattggagatacaattttttataaaatag

SEQ ID NO: 196 - DNA, 39 bp, Arabidopsis thaliana
  source 1..39: mol_type = unassigned DNA
atgagcaaggagatatttttttcccctgggtttgaatga

SEQ ID NO: 197 - DNA, 133 bp, Arabidopsis thaliana
  source 1..133: mol_type = unassigned DNA
atgagaacacacgagcaatcaacggctataacgacgctacgtcattgttacagctctcgtttcatgtgttctcaggtcacccctgctgagctctttctctaccgtccatgttttatcaacgccgtggcccgtg

SEQ ID NO: 198 - DNA, 18 bp, Arabidopsis thaliana
  source 1..18: mol_type = unassigned DNA
atgaagagaaacatgtaa

SEQ ID NO: 199 - DNA, 18 bp, Arabidopsis thaliana
  source 1..18: mol_type = unassigned DNA
atgcaatgtgaaatatga

SEQ ID NO: 200 - DNA, 15 bp, Arabidopsis thaliana
  source 1..15: mol_type = unassigned DNA
atgttttgtgcttga

SEQ ID NO: 201 - DNA, 21 bp, Arabidopsis thaliana
  source 1..21: mol_type = unassigned DNA
atggtcatggctcatcattag

SEQ ID NO: 202 - DNA, 165 bp, Arabidopsis thaliana
  source 1..165: mol_type = unassigned DNA
atgatgaaacctcgatggaactgctctctttatggaatcacggaatggacaaataatcaaaatcagaaatcgaagcgaaaagggaggagaaaaacgcagatttggaggattggggacagattagatactgttgaatgcatcactctaatgctatcagcctattaa

SEQ ID NO: 203 - DNA, 33 bp, Arabidopsis thaliana
  source 1..33: mol_type = unassigned DNA
atgctgcttatcatcgtggagttggttctgtaa

SEQ ID NO: 204 - DNA, 45 bp, Arabidopsis thaliana
  source 1..45: mol_type = unassigned DNA
atgctttgtttcaatttcaggtgcgttagaaggtttgcagagtag

SEQ ID NO: 205 - DNA, 108 bp, Arabidopsis thaliana
  source 1..108: mol_type = unassigned DNA
atgtacacctaccagcttgataactctttttcgtggtttctgtgtactcgtttctgtttgtacagatacttcttgttcaatttcagatgctttagaaggttttcggag

SEQ ID NO: 206 - DNA, 267 bp, Drosophila melanogaster
  source 1..267: mol_type = unassigned DNA
atgtggcgcgaagtatgcgcacaaaaaagtcaaacaaaaaggcgcaattttattacgggcaaccaacgacgaaacaaaacaaaagccaaccgaaaagcagaaacaaagcagcaaaaagtttatgaattttttgtgcaggcgcgtgaaagatgcaaaacgagaaaaaaacatgaaaaaaaaacattaaaaaaaacaaaaaaaatccaaaacagataccgagctgtatccgaaaacgagtggggaaaggggtttcccagtcacatataa

SEQ ID NO: 207 - DNA, 111 bp, Drosophila melanogaster
  source 1..111: mol_type = unassigned DNA
atgcgcacaaaaaagtcaaacaaaaaggcgcaattttattacgggcaaccaacgacgaaacaaaacaaaagccaaccgaaaagcagaaacaaagcagcaaaaagtttatga

SEQ ID NO: 208 - DNA, 216 bp, Drosophila melanogaster
  source 1..216: mol_type = unassigned DNA
atggagcctggctttgtttttgttttatttccaacccacttgagcacacagcacacacagagagaaaaatcaatactcgttatgggattaaatttacaaagcgcaaagcaaagcgacaaacaaaattcaaaagaaagaaaaaaaaacactcaaataaactcacaaagaattccttatcgccaagggggccaatgttctaaggttctttcgccttga

SEQ ID NO: 209 - DNA, 614 bp, Arabidopsis thaliana
  source 1..614: mol_type = unassigned DNA
attcattgttcactctcaaatctcaagttcattgccatttttaggtctctctataaattcaaatgttctgttcaattcaatgcgtcgccagacatctgttccctttgcatgtaagagagataaagaaagcgacaagagccataaagaaaggtaagactctttgaaatagagagagataaggttttctcttatcttcttctcatcagatctttgtttctttaccctctttctttcttttttttgctttttatggttatgttttttctcgatttagacaaaaaccctagatttgatcttctaaagggtctcaaatggaatctcttctcttctcatatctctccctctctccctccctctctttgattctttgtcttctccagttaaaactcagatctaacacaaagcttaaaagattctcatcgtttcttgttttctttgtttcatcttgtagatctctgaagttggactaattgtgaatgaaagagttgggacaagagaaacgcaaagaaactgacagaagagagtgagcacacaaaggcaatttgcatatcattgcacttgcttctcttgcgtgctcactgctctttctgtcagattccggtgctgatctcttt

SEQ ID NO: 210 - DNA, 422 bp, Arabidopsis thaliana
  source 1..422: mol_type = unassigned DNA
ctctgcctttagttctttcttttttggtaatatatttatttttcgttacgatttggtcaaaaccctagatttgttttccaaaagcatatctgaaaatgaaggacaactttcctcttctccttcggttataaatattctctccggttttgcttgtttaacctaaaagcctcagatctaactccaacaccttcaaagtctgcctcctttccaatcttctttcttctgttcgatctctaatctcagaatttgtgtcggtaaggtaaaggtgataatgagtgatgactgatgagggagttttgggacaaattttaagagaaacgcatagaaactgacagaagagagtgagcacacaaaggcactttgcatgttcgatgcatttgcttctcttgcgtgctcactgctctatctgtcagattccggct

SEQ ID NO: 211 - DNA, 268 bp, Arabidopsis thaliana
  source 1..268: mol_type = unassigned DNA
tcccacatccaaagatagaaagatgtaaggtctagagtcttgttcttaatcccctaacagaacaatgatatatataaataaatatgggtcgatatcggctgtggaggacgactagctacggtttcgagcctggtcacatgcgtagagtgtgaaaggtaattaggaggtgacagaagagagtgagcacacatggtggtttcttgcatgcttttttgattagggtttcatgcttgaagctatgtgtgcttactctctctctgtcacccct

SEQ ID NO: 212 - DNA, 328 bp, Arabidopsis thaliana
  source 1..328: mol_type = unassigned DNA
tcccacagccaatgagccaaagataaagaaacacctatcctataataatttagagcaatatacctccataatggaacatctatatatataaaggtatccgtatatctctatatattatattcattgagtttaaagtggctagggtttatagatgtatgtgatattaagagatatgaaacatatttgtcgacggtttgagtggtgaggaattgatggtgacagaagagagtgagcacacatggtggctttcttgcatatttgaaggttccatgcttgaagctatgtgtgctcactctctatccgtcacccccttctctccctctccctc

SEQ ID NO: 213 - DNA, 592 bp, Arabidopsis lyrata
  source 1..592: mol_type = unassigned DNA
aaaaaatgacgtgtcctcttctctctctctctttccttctctctaagtatatttagggttaattattagggttctttatctctttcttcagtctttgaagtttcttcaatagctttaattgaagtgatttacctctctgggtgtttttagtatatatatcatgtacatgatcgaatttctttctatccaagttctcatcaaaccttctcatgttttgaagagttaaaggctttatagtttgcttaggtcagatccataacatactgtatttgacaagtttctttgtctcacgatagatcttggtctgaccaaaatgattttctcgagaaaaaaaaagatggaagtagagctccttgaagttcaaacgagagttgagcagggtaaagaaaagctgctaagctatggatcccataagccctaatccttataaagaaaaaaaaggatttggttatatggcttgcatatctcaggagctttaacttgccctttaatggcttttactcttctttggattgaagggagctctacatcttctttcaccttctctatttttctttctttattttctcctctacagtaatttatttggatt

SEQ ID NO: 214 - DNA, 599 bp, Arabidopsis thaliana
  source 1..599: mol_type = unassigned DNA
ttccaaaacatgacgtggcctcttctctctctctctttccttctctctaagtatgtttagggttaataattagggttcctcctctcttttgttctgtctttatatctccttcatagctctaatgtaagagatttacctcttttggtgtttttgttaatccacgttctcatcaaaactttctcattgttttatgaagagttaaaggtctttacagtttgcttatgtcagatccataatatatttgacaagatactttgtttttcgatagatcttgatctgacgatggaagtagagctccttaaagttcaaacatgagttgagcagggtaaagaaaagctgctaagctatggatcccataagccctaatccttgtaaagtaaaaaaggatttggttatatggattgcatatctcaggagctttaacttgccctttaatggcttttactcttctttggattgaagggagctctacatcttctttcaccttctctattttttatttttctttatttctactcaacaattatttattcggattcatctttaattttccgttataatttctttttggtaaggattattcgctataatttgagaat

SEQ ID NO: 215 - DNA, 623 bp, Capsella rubella
  source 1..623: mol_type = unassigned DNA
ttccaacgaatgacgtgtactctctctgctctatctctctctctaaatatgtttagggttaattagggttcttcatctgtctctctctctctctctcttcagagtctttatagcttcttccaagatttttaattgaaagtaatttacctcttttggagttctgtacatatagaatatcaggagtcgtgtttcttttttatcaaggttctcatctaacctttatagtattttcattagttgataaaggtcttcatagtttgcttagatcagatcttgtcttcgtcttttcgatagatcttgttctgtccaatatacagtgattttatttcgagagcaaaaaagatgagaggtagagctccttgaagttcaaacgagagtttagcagggtagagaaaagctgctaagctatggatcccataagccctaatccttgttaatgataaaggatttggttatatggcttgcatatctcaggagctttaacttgccctttaattgcttttactcttctttggattgaagggagctctacatcttctttgacttctctctctattaagtctttctttattttcttctctacaatagttgttttggatcggaagatctttaagtttccctta

SEQ ID NO: 216 - DNA, 612 bp, Arabidopsis thaliana
  source 1..612: mol_type = unassigned DNA
tttcacttttgttctcctcctccctttttttcttttcaggattcttcttttctatgttttatctttcataatagatctgataattttgatttttcactatatatattatggttaatactagtagctttttcatttcaagttttatccttccattggttctttctgagtcaaattgtctcctgtttcgaaccatatataagttttcaatggttttgtattaactcaagtattcaacattatgtctctctttttcttgcttggatctctaatgctgttcatattttaaagcataggtttaggttagatgcatgtaactgccaattaaaagaaggtcaagagttttttgattgtatgaatatatgagttagtcaaagcagatccacacgattatatagaaaaacaaaggaagaagaagaggaagagctccttgaagttcaatggagggtttagcagggtgaagtaaagctgctaagctatggatcccataagccttatcaaattcaatataattgatgataaggttttttttatggatgccatatctcaggagctttcacttacccctttaatggcttcactcttctttggattgaagggagctcttcatctctc

SEQ ID NO: 217 - DNA, 515 bp, Arabidopsis thaliana
  source 1..515: mol_type = unassigned DNA
catcccacccttaattgttttatataaaccatttctcctcctctctccatcaccttcaatctctctcgatctctctctggatccccaatctcacctccatgttttgtttgttgattcccatcttctcttttgtcttttcaccaaatcgtcatttaaggcttcaagaacagtaaccccaattcctccacaagagggagagaaaacaaaagatcttccaattccattctcgtacatgcaaatcacaatccatgccatagattgtttctattcctccttatttattgcttgtatctgttcatgcatggaccaggtggagagagcattacttaaaaatagaattagctatctgttttaggcgaattagtttccttacataaccatgtatatgtcatgacgcatatacatatgtagatgtatatgtattatatatgtatgcctggctccctgtatgccatatgctgagcccatcgagtatcgatgacctccgtggatggcgtatgaggagccatgcatat

SEQ ID NO: 218 - DNA, 276 bp, Arabidopsis thaliana
  source 1..276: mol_type = unassigned DNA
actcataactctccccaaattcttgaccaaaaatatccgccactttctctctggttcatgttttcccctcaatgaaatacatacacattttgattttatttaaatcaagatcgacgtataagctatccaccaatcatatttaagggttcccgtatacatatatactatatatatatatggaataatagtcgtgcctggctccctgtatgccacaagaaaacatcgatttagtttcaaaatcgatcactagtggcgtacagagtagtcaagcatgac

SEQ ID NO: 219 - DNA, 243 bp, Arabidopsis thaliana
  source 1..243: mol_type = unassigned DNA
ctctaactcatccttctcttctatgaaaattccattgtttctgccgaagctttgatcagtacttctcttttgcttgatctcggtttttgaccagtttattgcgtcgatcaatgcattgaaagtgactacatcggggttccgattttttttgttcttcatatgatgaagcggaaacagtaatcaaccctggtttagtcactttcactgcattaatcaatgcatttgtaaaaagagggaaaagca

SEQ ID NO: 220 - DNA, 537 bp, Arabidopsis thaliana
  source 1..537: mol_type = unassigned DNA
ctagaagaaaaaaccagatctataaagtttgttattaaaagatagagagagaggagggatgtagtaggccaataggcaaatcagagaatcacaaatggtatctggtcaagaagattcctggttaaaactttcatctctctgtttcctttttctttctttgttggattcattaatttgacatatctctatcatcacactgattctctttctcccagtttgtctgcagatgcatgtgtgtaatctagggtatatgtttttgtccatttggtttcataaggcaataaagatccagctatttactacttgtggtatagattttgactgttgaattttcagatctgatgtgtttcgtttgatccgattcggaaaatttatgtttcgttgacattttggagtttagttggaagaagagtgagagtcgctggaggcagcggttcatcgatctcttcctgtgaacacattaaaaatgtaaaagcatgaatagatcgataaacctctgcatccagcgtttgcctcttgtatctttcttattgactt

SEQ ID NO: 221 - DNA, 261 bp, Arabidopsis thaliana
  source 1..261: mol_type = unassigned DNA
ctgcatctatccacctctctctgtaaatttatctaaatgtttcttttaatctttttgagattaataatgatttgtgtttgttcatcaaccgattttctcagatctgtcaattatttttgtttatttatttatgatttatgaatgaggaaagagtgaagtcgctggaggcagcggttcatcgatcaattcctgtgaatatttatttttgtttacaaaagcaagaatcgatcgataaacctctgcatccagcgctgcttgctc

SEQ ID NO: 222 - DNA, 481 bp, Arabidopsis thaliana
  source 1..481: mol_type = unassigned DNA
tatcacagttctcatcaaatatttgaaagtatcaaacaaaaaaaggagagtgagaaaaataaagagagagatagagagagatcatgtccactactcaagagcataggtcttgattggtggaagacaagtaccttagataaaccgaccaaaacccggtggataaaatcgagttccaacctcttcaacgacaacgatttcaacactctcttccaggaacaacttcctccaggcagatgatactaaagtgctggagttcccggttcctgagagtgagtccatatcaaaatgcgcattcgttatcacttggttgaacccatttggggatttaaatttggaggtgaaatggaacgcgtaattgatgactcctacgtggaacctcttcttaggaagagcacggtcgaagaagtaactgcgcagtgcttaaatcgtagatgctaaagtcgttgaagaggacttggaacttcgatattatcccccgtgt

SEQ ID NO: 223 - DNA, 471 bp, Arabidopsis lyrata
  source 1..471: mol_type = unassigned DNA
agtagggttggaaaatttttttacatttttactctaaaatagaatagagttggagatgcccttagcagttattagacaagggattgtttggccctagcgatcctctcttcactctctcacttttgtagttcaacccttcttttgcgtgagatgccatcatggcatgacatggttcttttgccttacgtaaaacacactcacgccaacacacgccacataacataaataaattatatatacatatacgtatgtgcgtgtgagtcttccattaatgcaatctttgggcctatatatatatacaaaccttccataaccaaagttatcatactacaaaagctctctcgtacttggaaatgcgggtgagaatctccatgttggagaagcagggcacgtgcaaaccaacaaacacgaaatccgtctcatgtgttttgcacgtactccccttctccaacatgagctcctgacccattg

SEQ ID NO: 224 - DNA, 466 bp, Arabidopsis thaliana
  source 1..466: mol_type = unassigned DNA
agacaagcccccacactaaaaaaacagtaatatggaataaaaaaaagctttcaaaacttagcagttattagacaaggtattgtttggccctagctagcgatcgtttagctctcttcactctctcacttttttagttcaacccttcttttgcgtgagatgccatcatggcatggtatggttcttttgccttacgtaaaacacactcacgccagcacacacacacacacacataacatatacggatgtgcgtgtgagctagtcttccattaatgcaatctttgggcctatatatacaaacctttccataaccaaagttctcatactacaaacgcccctcatgtgcttggaaatgcgggtgagaatctccatgttggagaagcagggcacgtgcaaaccaacaaacacgaaatccgtctcatttgcttatttgcacgtacttaacttctccaacatgagctcttcaccc

SEQ ID NO: 225 - DNA, 450 bp, Brassica rapa
  source 1..450: mol_type = unassigned DNA
agacaaccccacgttttaaaataagaaatgatgataattttgtggaaataaaagctagtatacttttgcaataattagacaaggtattgatgctttgggccaagctagtttcttttagcactcttcactcactagtttttcttctcagcccttcttttgcgtgaaatgccatcatggcatggcattgtcattttgcctttcgtaaaacacactcacgccaacatacattattcatattcatgtgtatgtatatgaatgttccattaatgcaatctttggggcctatatatacgaagcttacatcaccaaagctctcatattacaaaagctcacatatatacttggaaatgtaggtgagaacctccatgttggagaagcagggcacgtgcaaaccaaaaaacatgaaatctgtttcatatgctttgcacgtgctcccctcctccaacatga

SEQ ID NO: 226 - DNA, 513 bp, Carica papaya
  source 1..513: mol_type = unassigned DNA
agacaacactcctctttgttcccttcctcacgtatccacttttgaaatttgtaatttgtgtgcaccaccatgattgcatgccatccctacttgccttttccccttttcctttctctaacattttactcaatcttcttctccccctcccccccttccccctctctgccattataaccataattaaacctctctccctctctctccctctctctctctctctctctgggttctcagtataaatgcagctctgcttatacttccacacctatatatatatacctgacccttcttcacctccttcatccacctcctccttcttccccaaaactttcttaactgttctctgcatacatatatatccacatacatatatatatatatagagagagagtgagacagagaggttaccgaggcaattgggtgagtagctccctgttggagaagcagggcacgtgcaaattctccatggctttcccctctttgcacgtgctccccttctccaacatgggttcc

SEQ ID NO: 227 - DNA, 464 bp, Capsella rubella
  source 1..464: mol_type = unassigned DNA
cggccacccccacatttaacaagaaaaaaactgatggaattaaaaggtttgagaacttggcagttattagacaaggtatagtttggccctagcttcttttaatttagctctctccactctcacacttttcaactttcacccttctcttgcgtgagtcgcgagatgccatcatggcatggcatggcatgtttctattgccttacgtaaaacacactcacgccaacacatactcactatacatgtaaataagtatgtgcgcgtgtgagtcttccatccatcaatgcaatctttggggctatatatatacaaaccttttccataaccaaagctctcatataaactacaaaaggctcacttgggaaatgcgggtgagaatctccacgttggagaagcagggcacgtgcaaaccaacaaacacgaaaccctcctcatgtgctttgcacgtactccccttctccaacatg

SEQ ID NO: 228 - DNA, 571 bp, Gossypium raimondii
  source 1..571: mol_type = unassigned DNA
gaaaacccaagttcaggctaacaagttatctgatgatgagatcaagaattttaaagtttcaatatagatttggcatgggtattggcggcagaaagcaattaaaaaaccagttatgtcaaattcaaggtcgtatcagttaaaatgaatgaagatttagaaatttcaacaaggaagaggaccccacagctttgttaaattaagtgtgtggtttttataattatcatctcgaaagtttcataatatcaattagattaaaacatctctgaatttcataattacaaaccagatagatagatacatgaaaacttagaccccagagatctgtctttaaagaatgcccacttctagactcaatctctattactctctttttttctctctctctctcttcggaaaaacttgtatataaataaatgacactttctttgctttctgcactcaactcatgaacttgaaaagctttacttggatgggttggttgggggtgagtatctcttgttggagaagcagggcacgtgcaagttcctatgtttaagtgaactttgcacgtgctccccttctccaccgtgag

SEQ ID NO: 229 - DNA, 592 bp, Medicago truncatula
  source 1..592: mol_type = unassigned DNA
gaagagaaaaaacctagtgtaaaatttgatatactctttatgtatagtacgaatgtttttttaaaaattatgtaaaaaatgataaaataataactaactaaattaacagtaaaattagaaaagtaaaatactatgcccaaatttgatatttttttttatatatttgtatagattattattatttgatatgtaaagtccaattaaaaatttgttttaactaagatttgaactaggttttcttaaaagactcatcttttacttcaaatttatttatcatttgaattcaatcactttctaatattattattattatttccaccatactcattgcttctgccacgttactttagttagatctcttatgtcatatatctctctctctcctaagttgctacctataaatactaagcctttcccttggttggttcaattcaacttctacttctcatcaaacacaaagtgcaataagcttcatttcctgggtgagaagctccttgttggagaagcagggcacgtgcaaatcctctttctgattcattctctcataatgcatatcaatatcttttgcacgtgctccccttctccaactagg

SEQ ID NO: 230 - DNA, 520 bp, Oryza sativa
  source 1..520: mol_type = unassigned DNA
atgcaaacccactccaacactccacaatccacatactctctctctctctctctctctgagtaggagtacatgtgtgtgtgtgatatcaatatgcattcgatgttgatgctactgtagccatcttgtggctatataaacccagcaggcagcagcacagcttagctagagagccatattgcatgcacactcgctaatctcttttctctactctacttgcattacaccacctctgcattgcacttcagttcattcattccactgatgcatggatcgatgttgctaccttcttctcttctcctcatgcatccatgcatcgatctcacctagcttcttcctcatcctctctcgatcgattacaagagaaaagtgtttgctgttcttgctatcgatctacaggtgagtaggttcttgttggagaagcagggtacgtgcaaaatgcacaccggttggtcgagctaattaacaagctctgacgaccatggtgatcgaatgcacgtgctccccttctccaccatggcct

SEQ ID NO: 231 - DNA, 501 bp, Arabidopsis lyrata
  source 1..501: mol_type = unassigned DNA
ctagggtttaggaatgacgacttgtttctgttgtgtcttattaaaagcccatcttcgtctccgccactcatcattccctcatcataacaccatcatcaccattcaccaacctctctctctttctctctctcctctcgatctacaacaaaatgtgaatctgctaagatcgattatcatgagaattaagctatttcagttgaggggaatgttgtctggatcgaggatattatacatatatacatgtgtatgttgatacatgtgatcatagagagtatcctcggaccaggcttcatcccccccaacatgttattgcctctgatcaccatatatatgtcgttacatttcatggttaattacttgcacaaatcacaaaagcttggtttgtaactttctatgaccttttttaatgactttgaatctttcatgcatgacttcttaagagtagatttacacatttgcggatccgtttatgctttttgcttttgtttcgtttatatatat

SEQ ID NO: 232 - DNA, 484 bp, Arabidopsis thaliana
  source 1..484: mol_type = unassigned DNA
ctagggtttaggaatgacgacctgtttctgttgtgtcttattaaaagcccatcttcgtctccgccactcatcattccctcatcataacaccatcatcaccattcaccaacctctctctctctctcctctatcactctctacaacaaaaatttgtgaatctgctaagatcgattatcatgagggttaagctatttcagttgaggggaatgttgtctggatcgaggatattatagatatatacatgtgtatgttaatgattcaagtgatcatagagagtatcctcggaccaggcttcatcccccccaacatgttattgcctctgatcaccatttattgttacatttttttttgttaattacttgcgcaaattacaaaagcttggtttttgtgatgactttgaatctttcttgcatggcttcttaagagtagatttacggatccgtctatgctttttgctttttgtttcgtttatttgtatttaaac

SEQ ID NO: 233 - DNA, 552 bp, Brassica carinata
  source 1..552: mol_type = unassigned DNA
gagatcaatgaaattatcctgccaaataaaacgtgtgacgtttattcaaaaatatatgcattagatgctttgatattaaaatatttccttttaaaagctagctagggtttaggaatgacgagttgtgtcttattaaaagcccttcttctcctccgccactcatcattccctcatcataacaccatcatcaccattcacccacctctcctctttctctctctctctctctctctctctctctagaacaacaagtgagaatctgctaaaatattgtgactattatcatgagaatgaagctatttcagttgaggggaatgttgtctggatcgaggatattatatatacacaaatacgtatatatgttaatacaagtgtttgatcatatatgtatatagattattctcggaccaggcttcatcccccctaacatgttattgcctctgatcaccagattctatcaactcttcgcttattattttgtcacaaacaagtaataagctcataattttctttgagtctttcagcatcgtttcattatgtttttcgaatccg

SEQ ID NO: 234 - DNA, 566 bp, Brassica juncea
  source 1..566: mol_type = unassigned DNA
gagatcaatgaaattatcctgccaaataaaacgtgtgacgtttattcaaaaatatatgcattaaatgctttgatattaaaatatttccttttaaaagctagctagggtttaggaatgacgagttgtgtcttattaaaagcccttcttctcctccgccactcatcattccctcatcataacaccatcatcaccattcatccacctcttctctcctctctctctctctctctctctctctctctctctctctctctagaacaacaagtgagaatctgctaaaatattgtgattattatcatgagaatgaagctatttcagttgaggggaatgttgtctggatcgaggatattatatatacacaaatatgtatatatatgttaatatcagtgtttgatcatatatatgtatatagattattctcggaccaggcttcatcccccctaacatgttattgcctctgatcaccagattctatcaactcttagcttattatttgtcacaaacaagtaataagctcaataatgtctttgagtctttcagcatcgtttcatatgttttcgaatccg

SEQ ID NO: 235 - DNA, 549 bp, Brassica napus
  source 1..549: mol_type = unassigned DNA
gatatcaatgaaattatcctgccaaataaaacgtgtgacgtttattcaaaaatatatgctttaaatgctttcatattaaaatatttccttttaaaagctagctagggtttaggaatgacgagttgtgtcttattaaaagcccttcttctcctccgccactcatcattccctcatcataacaccatcatcaccattcacccacctctcctctttctctctctctctctctctctctctctctagaacaacaagtgagaatctgctaaaatattgtgactattatcatgagaatgaagctatttcagttgaggggaatgttgtctggatcgaggatattatatatacacaaatacgtatatatgataatacaagtgtttgatcatatatgtatatagattattctcggaccaggcttcatcccccctaacatgttattgcctctgatcaccagattctatcaactcttcgcttattatttgtcacaaacaagtaataagctcaataatgtctttgagtctttcagcatcgtttcatatgttttcgaatccg

SEQ ID NO: 236 - DNA, 543 bp, Brassica oleracea
  source 1..543: mol_type = unassigned DNA
gggatcaatgaaaattatcctgccaaataaaaacgtgtgacgtttatccaaaaatatatgcattaaatgctgtgatatgaagtatttcctttaaaagctagctagggtttaggaattacgagttgtgttttattaaaagcccttcttctcctccgccactcatcattccctcatcataacaccatcatcaccattcacccacctctcctctttctctctctctctctctctctctagaacaacaagtgagaatctgctaaaatattgtgactattatcatgagaatgaagctatttcagttgaggggaatgttgtctggatcgaggatattatatatacacaagtacgtatatatgttaatacaagtgtttgatcatatatgtatatagattattctcggaccaggcttcatcccccctaacatgttattgcctctgatcaccagattctatcaactcttcgcttattatttgtcacaaacaagtagtaagctcaataatgtctttgagcctttcagcatcgtttcatatgttttcgaatccg

SEQ ID NO: 237 - DNA, 566 bp, Brassica rapa; identical to SEQ ID NO: 234
  source 1..566: mol_type = unassigned DNA

SEQ ID NO: 238 - DNA, 414 bp, Arabidopsis thaliana
  source 1..414: mol_type = unassigned DNA
catcatcaccactcacttatcttcttctccatctctctctctgcttctcccttaatcttagccgggtctcgtgggggacgaacatagaaagagagagatataaagatatatattcagaaaccctagattctataatttcgactgaaaagaaaaaggggctttctcttttgaggggactgttgtctggctcgaggactctggctcgctctattcatgttggatctctttcgatctaacaatcgaattgaaccttcagatttcagatttgattagggttttagcgtcttcggaccaggcttcattccccccaattgttgctccctgtttactccatatttcttccttcttttcaaattagggtttcagatccagtgaatgaacccttgttaaaggtttgatctcttaccttacttt

SEQ ID NO: 239 - DNA, 328 bp, Arabidopsis thaliana
  source 1..328: mol_type = unassigned DNA
tctcatcattctcttcatcatcaccacattcatctctctctctctctctctctctctctctttctcttccttgatcttagccggatctgttgggggacgaacacatgagagatagataaaatataagaaatttctcgaaaaaacctaatagaaaaaggtctgtttcttaaagaagaagaagaagaggatttaaagagggatttttcttttgaggggactgttgtctggctcgaggactcttattctaatacaatctcatttgaatacattcagatctgatgattgattagggttttagtgtcgtcggaccaggcttcattccccccaa

SEQ ID NO: 240 - DNA, 192 bp, Arabidopsis thaliana
  source 1..192: mol_type = unassigned DNA
tgatgaacagaaaaatctctctttctctttcttgatctgctacggtgaagtctatggtgcaccggcatctgatgaagctgccagcatgatctaattagctttctttatcctttgttgtgtttcatgacgatggttaagagatcagtctcgattagatcatgttcgcagtttcacccgttgactgtcgcaccc

SEQ ID NO: 241 - DNA, 846 bp, Arabidopsis thaliana
  source 1..846: mol_type = unassigned DNA
aaccacaaagtaccgctgctattttctttttacgtctttgtatttgcatcgtctaagagaatgatgggttgttttgtgggattttaatgcaggaggaaacatatgaggggtgattaaggcaaaaaccttaagatgtggtcatttagatacatggagtcaaactaagaatggaccttggcgaaagcttcttcacggtcaagatttaaaatcaggtacgacactgtgtacgtgagagagagagagagagagagaaagagattatagaaagaaagagatgtatcacaataaaggagtatatttagggtcacaggtggtggagatataggtatgcagggccaaggctctaatctcttcatagccctattgattttgtccctctctctctctctttcttcctctcttagctgtatgcattatgatgcgtcttttaattcactgtttcaggcttctttaattcgtggtgtctctctcctttttacccaaccatctcttaaaatttttaacatctgttcctcaaatcctctctcatctctttctataagtatctatagcgcctcttaaaccacaaagcatcacctctgtcttctctcatctcctttctgtattctctttcattgccttcacgtctgttgcaatttctccacttcttgagcttccgttttttacaattattgatccgtcaaatatgtgagatttgcacaacttgttgctcaggtattttgaagacaagtccacaagggaacaagtgaagctgccagcatgatctatctttggttaagagatgaatgtggaaacatattgcttaaacccaagctaggtcatgctctgacagcctcactccttcct

SEQ ID NO: 242 - DNA, 571 bp, Arabidopsis thaliana
  source 1..571: mol_type = unassigned DNA
gagcaagacaatgccacatacaaacttgaaagatctcttcatcttttctccaaatgtttttttttcgtttgctatttatctccacaattcttggaacaaaaactacattcacaaacgagagaattttcacaacacctcttttgctctcattttttttttttcgtccattatgagtattaattatggttagggaatcttacagaatgaaaatgaaggtgtgaatggattgtctcatctaaagccttgaatgtgggaaaaaggccattgttgttcagccaaggatgacttgccggtagcttgtattatgattactctatattcgatttatattatggagatgatggtttatatatatttacttatctacatagttttagttgattttttttcgtacgtaatataatacgaaaaagtatttacttatttatatatgtgtgttggggcaagaagtgtaaccaagctagcccggcaagtcatctatggctatgcaactgtctcttcctctcattctaggcttacgatgacacgtaaaaaatcccaaatatcactaatatgatatgaatatggatga

SEQ ID NO: 243 - DNA, 241 bp, Arabidopsis thaliana
  source 1..241: mol_type = unassigned DNA
aggcatgagacataaagagagttaaatataatgaagaagagaggtctaatatggcgaaaagagtcatgtttaatagccaaggatgacttgcctgatctttttcacctccatgattcaattttaagttcgtggattttggattattatgcgtttaaaaggtataataatttgagatcatgttgaatcttgcgggttaggtttcaggcagtctctttggctatcttgacatgctttcttcatc

SEQ ID NO: 244 - DNA, 689 bp, Arabidopsis thaliana
  source 1..689: mol_type = unassigned DNA
gaattttgatttatgaacctcctcaagaaggaaagacagaggaggagacaaagaagtataggttcacattgcatagccagtttagttttgaaggatggatatatgaaaaaaatatgaagagagagaagagagaagaagaggaggattaaagagggtgaggccagcttttgtgctttggtagtagatgaggtttaaatgctccataccttccatttccttctctcttaccctaatttaattcttcctctcctttataactccccacagacattctcacttctcctcctcacacttcacatcaacacttctttcttgttttttcattttacaatgtttcctttgatatccgcactttaagcatgagagagtccctttgatattggcctggttcactcagatcttacctgaccacacacgtagatatacattattctctctagattatctgattgagccgcgccaatatctcagtactctctcgtctctattttggactttgtggtcttgtagatcgatttgtatgtgtgtgttgaaatggagacaagtacttgtaacttctttgttgttatattgtttacctataggctgatgtcataaactcttttgatcttgtttctaacttccagattcttgaaaaatcaagtcgtgtgtgtgtctccatggaagccttttccatttcttcctttcca

SEQ ID NO: 245 - DNA, 452 bp, Arabidopsis thaliana
  source 1..452: mol_type = unassigned DNA
actcataaactttgctactggccgcatttctattttctccttcgattcttctaattcgtactttggtttctgacgtccctaaaatttttagacagtaagagtttctccaggatccgatggttctctccggtaaattaacattttagtgtcaatagtcatttatacatatttttatttcactttttgttttgtttattggttttctggagctaagtggagattatagtcgaacaagagtggttttatgcaaggtaacgcgagatattagtgcggttcaatcaaatagtcgtcctcttaactcatggagaacggtgttgttcgattgagccgtgccaatatcacgcggtaaaccaaaaatggcaaagatagttattataaccttaaaggtatgtatcattatcgttttattgtttcaattttgattaatggctttgatatttcatttttttttt

SEQ ID NO: 246 - DNA, 767 bp, Medicago truncatula
  source 1..767: mol_type = unassigned DNA
attggtcaaacatacatacagtagcactagctggtttcattattccactatgcttcttcataggctctccaaattttgcaaaattgaaagagacatagtatatatatcttagcaaggagaaattcaggatattgaggatgaagattgaagagtaatcagtgatgaagaaagcaagcaaggtattggcgcgcctcaatttgaatacatggctataaaaatgcatcatatcagccatgtagtttgattgagccgcgtcaatatcttgtttccatctccaaatttaccaatctcatcaaatcaaattaacaccacaatcaaggctttcatttaatgcagtcaaaataggttgaccttatcatcgaagaaattgttttctcattcctatcgaagttggacttgccgaaaatgctcgaaagcatgtgttttagttcgacaggcgaaaaagttaccgaaggacaatttggttgtggttcggataagatcaagcaacggatattttcaagacacgttcgaaattcaaatcaaatggataagtatcgttagtttactgcagttatagttttaaattcaaatctaggcagttgtttctatttgtataaatagtagtttttccctagggaaaaggggtcgcaattcaatcatacaaaaaacttacaatcaaattatccgcatggaagagagaaacgagtcacaagttgcaatgtatgaacatgtgtaccaatttacattcaatcagtacaatttaagttcatttccataaaaaaaaa

SEQ ID NO: 247 - DNA, 854 bp, Zea mays
  source 1..854: mol_type = unassigned DNA
accagggttaggtatccatccacacagcaatgcatctgccttcaactccctctcgccccccaccccaacacacatctctctcttttctagggaaggaaatgacgaaggggacgacgacggcatgcttcggctagctcgttggtgctaggacaagggcggaggtattggcgcgcctcaatccgaaggcgtggctgatagattggcgcggcagccatgttcttggattgagccgcgtcaatatctccccttgcctgtcccgtacctagctagcttgcttgcctcactgatcgatgtcgtccctatttcatggagaagctgatgattgattattctcacaagcaagaactgtctgatctgttgcctgcatcgatcaggatctatatgctggagagttcacaagaacatggacagaactcgcttcaacaaccgatcaatcgattgattaggtatgtacctacctcatatgcctcagctcttcgttatggatttcttcaaccgaagggtcagtaagctcttggttccatgccactgcgtgaactaagcgttcacaaaatccgttccmcggcatgaaccaagcactcaaaatcgcatgcagcatctttcgtttcaaaaaaaaattgacttytgaaaacaatagatgaatcagtttcaaacatatatgattatccattttctcaaccgggaatttatatctcgttgggatgcaaaaccgttcagtagtaaaactactccacgagtataaactgtttcagttattttactattaattagttacccgtatgctgttatggtttctatatatctataagtaaaccttacttaaataagatagttatacaaaaaaaaaaaaaaaa

SEQ ID NO: 248 - DNA, 660 bp, Arabidopsis thaliana
  source 1..660: mol_type = unassigned DNA
caagaaaaaacattgaaatagctcatgttgtctctttctcattttcatatctgctaaaaaaagaaccgtgttttctaaactggtttaacggtaagtacctgtctctagtaacttacctatcaatttgttccaatcatttacttgctttgacttatttggtttccttttgttttgtttttctttaatatgtggatggagtttggtgtaataagcaactgaagagtcgatgagcgcactatcggacatcaaatacgagatattggtgcggttcaatcagaaaaccgtactcttttgttttaaagatcggtttatttgattgagccgtgccaatatcacgcgtttaaatagtttaaagattctatgttagttgatgtgatcaatcaaggtatgaatctatatcaattctcttatgcatagttttatatttacagagatgaggtattatcaatgtctatcgtcgaggatcacgctcttacttatgttatatttctatataattttattaattagttttctaaaagagaaggacaatttaaaattattttaaagagttttttttaagtagttttgttttcatgtttatcttctgcaggctctgaagttaggatagtaacaagaaaaaagacagaaaaaaagaagaaaattcatatacattcgtga

SEQ ID NO: 249 - DNA, 225 bp, Medicago truncatula
  source 1..225: mol_type = unassigned DNA
gaataagtgaatattatcgatatttatatcatatatcaacttttcttctgtgcttgcttgcaaatttgcaattaagcttttttgatcttatgtaagagaatattattgatgatggtgtttgggaagccgaaaaaagcgatgttggtgaggttcaatccgaagacggatttacatgtatagagttgtaaaatacgatctcagattgagccgcgccaatatcacttt

SEQ ID NO: 250 - DNA, 367 bp, Medicago truncatula
  source 1..367: mol_type = unassigned DNA
ccacaaaactataactagctagaagctttaatcgccttatttattataataataataataaatatggcttcagctgcaaaagtatacatggcgtgatattgatccggctcatctatatcttcaagttcaatcatccatattcatatcaatttcagacgagccgaatcaatatcactcttgtttgcttcattgcatattaattatatacttcatttataagttatagtttgccatatatatattagattgattctgcagaagtagacaggagtggtgttgtttctgctcatcttattaaataatgaatgaatgaatgacatttgcttacttataagacgagccgaatcaatatcactccagtacacct

SEQ ID NO: 251 - DNA, 712 bp, Arabidopsis thaliana
  source 1..712: mol_type = unassigned DNA
ctctctctctctctctctctcatctgtgttctagatctcaccaggtctttctctggttaatatatggcttccaagatctggtaatatgttataaatacgtcatacttaagcttttttcaaatcaaaaatagaaatttgtgggtttgtctcgttttactattttagcagtatatattaagaagttcagatgttattcgatcatctgtttttgcttcccctctgccatctttatcttttagggtttcaattctttttcacttttttcttctggtttggagatggttaggttccaactaagtatacgagattaaatttgacatcttagttacttcaaaattccttcaatcaaaacaagtcatctcgactattccgccatgtttgtatatacatatttatatattatatatatgaaggtacgagtttctagtgtctataaattaagaaggttaagtaccatatagatgatatttgttaagtagtaagtcactcaaagtttgagtttgggtttgagtttgagtttgagtttgagtttgagagacaaaagattactacaagaagattgttaaacaaaaatggaagactaatttccggagccacggtcgttgttggctgctgtggcatcatcaagattcacatctgttgatggacggtggtgattcactctccacaaagttctctatgaaaatgagaatcttgatgatgctgcatcggc

SEQ ID NO: 252 - DNA, 714 bp, Arabidopsis thaliana
  source 1..714: mol_type = unassigned DNA
acttgcacctctcactccctttctctaactagtcttgtgtgcacccatttatgtgtacgtactattatctcataaataaatatttttaaaattagatgcatttattgatatgaaaaagttacaagattagtttgttgtgtgtgagactttggatcgacagatcgaaaaattaactaaccggtcagtattgaatatcaactattatatgctccatgcattcgcttatagtttcacacaatttgttttcttcacggtctaaaatcagaagattccatatattttcttatgacgtaaaaggaccacttataagttgacacgtcagcccttggattcgtgaggtttttctctctacttcacctatctacttttcctcatatcccactgcttttctccttcttgttcttgtttttctcgtttttttcttcttcttctccaagaaaatagagatcgaaaagattagatctattttgtgtagcaagaaattatcattttcgtttcttcattcatatattgttctattatgttgtacaataatagatactcgatctcttgtgcgtgcgtaaattttatacaagttgtcggcggatccatggaagaaagctcatctgtcgttgtttgtaggcgcagcaccattaagattcacatggaaattgataaataccctaaattagggttttgatatgtatatgagaatcttgatgatgctgcatcaac

SEQ ID NO: 253 - DNA, 324 bp, Arabidopsis thaliana
  source 1..324: mol_type = unassigned DNA
tcaccaaataggctcttctttatcgcttcatatatataaaagtctacatctatctctttctaggtcactagctagactctagattaaggattgaaattagggtttcatgtttccagcaaaatggtgccgtcttgagtcttgaaaagatccaagacaaaaccaaatcactacatacatccctatcatcaaccagctactgttcgctgttggagcatcatcaagattcacaaatcatcaagtattcgtgtaaataaacccatttatgattagatttttgatgtatgtatgagaatcttgatgatgctgcagctgcaatcagtggct

SEQ ID NO: 254 - DNA, 531 bp, Arabidopsis thaliana
  source 1..531: mol_type = unassigned DNA
tgtcatattgagaactctttagcctttggcttctgttcctgacacttgtatagtgaagtgggcttgtgttatatagatgggatctctctctttatttaaaagtcaattagagatcttgatgctacttctgtccctttccaagtgattttacgtcgaccaactagcttttttcatatgagtgtatatattcatgtacctatctctctcaattgcttctcaccaaaatcatcttgctgattcatttgctgtctgaatcctcttgctttcctctttgctttttcatttgttgatttaaaccatgggagttcccaactttagacctcgaaaccgataaggatctttctctgcggttgaaatagctaggttctcgatgaataggctagcctttggtggatgttatcagccagtagtcgcagatgcagcaccattaagattcacaagagatgtggttccctttgctttcgcctctcgatccgcagaaaagggttccttatcgagtgggaatcttgatgatgctgcatcagcaaatac

SEQ ID NO: 255 - DNA, 618 bp, Arabidopsis cebennensis
  source 1..618: mol_type = unassigned DNA
ttgtatccatagtgtatttcctcgcatctaccatccattttctacgcctctctctctctctttctccatcaaatcttgttttgttcaaactctctctctcatcaattctctccatacaatacatgcatacatacatacataccatctctaatatttcatcaatcttcttttgttccaaacgctctttctctccatttacatacatacgaatcattgttgtcatagatccgtttagaattgctttaacttttagatgagatctagggtttctttctttttcttcaaaatcatgctttttcgcttgctaggttatagatccatgtaagtttagagtagatgtacacacacacgctcggacacttattaaatacatgttgatacacttaatactcgctgttttgaattgatgttgtaggaatatataaatgtagagagagcttccttgagtccattcacaggtcggatatgatccaattagcttccgactcattcatccaaataccgagtcgccaaaattcaaattagactcgttaaatgaatgaatgatgcggtagacaaattggatcattgattctctttgattggactgaagggagctccctctctcttctgtattcc

SEQ ID NO: 256 - DNA, 660 bp, Arabidopsis halleri
  source 1..660: mol_type = unassigned DNA
ttgtatccatagtgtatttcctcgcatctaccatccattttctacgcctcactctctctttctccatcaaatcttgttttgatcaaactctctctctctctctctcatcaattgtctccatacaatacatacataccatcatctttcccatctctaatatttcatcaatcttcttttgttcaaacgctcttcctctccatatacatatacatacatacgaatcacattggtgtcatagatccgtttagaattgctttaacttttagatgagatctagggtttctttgtttctttcgttttcttcaaattttgctgcatattctccaagatcatgatttttcgcttgctaggttatagatccatgcaaatatagagtagatttacacacacacacgctcggacacttattacatacatgttgatacacttaatactcgctgtttttaattgatgttgtaggaatatatatatgtagagagagcttccttgagtccattcacaggtcgtgatatgatccaattagcttccgactcattcatccaaataccgagtcgccaaaattcgaactagactcgttaaatgaatgaatgatgcggtagacaaattggatcattgattctctttgattggactgaagggagctccctctctcttctgtat

SEQ ID NO: 257 - DNA, 632 bp, Arabidospis lyrata
  source 1..632: mol_type = unassigned DNA
ttgtatccatagtgtatttcctcgcatctaccatccattttctacgcctctctctttctccatcaaatcttgttttgttccaactctctctctcatcaattcattccatacaatacatgcatacatacataccatcatcatcttttcccatctctaatatttcatcagtcttcttttgttacaaacgctctttctcgccatatacatacataagaatcattgttgtcatagatccgtttagaattgctttaacttttagatgagatctagggtttctttctttttcttcaatttttgctgcatattcttcaaaatcatgatttttcgcttgctaggttatagatccatgcaaatatagagtagatgtacacacattcacgctcggacacttattacatacatgttgatacacttaatactcgctgttttgaatggatgttgtaggaatatatatgtagagagagcttccttgagtccattcacaggtcgtgatatgatccaattagcttccgactcattcatccaaataccgagtcgccaaaattcgaactagactcgttaaatgaatgaatgatgcggtagacaaattggatcattgattctctttgattggactgaagggagctccctct

SEQ ID NO: 258 - DNA, 646 bp, Arabidopsis thaliana
  source 1..646: mol_type = unassigned DNA
ttgtatccgcagtgtatttcctcgcatctaccatcccttttctacgcctctctccctctctctctttctccatcaaatcttgttttgttcaaactctctctctctcatctattctctccatacaatacatgaatatacatacataccatcatcttcttttcccatctctagtttttcatcaatcttctgatgttccaaacgctctatctcttcatatacatacatacgaatatattattgttgtcatagatccatttagaatcactttagcttttagatgagatctagggtttctttgttttctttcaaattttgttgcatattcttctaaatcatggtttttcgcttgctaggttatagatccatgcaaatatggagtagatgtacaaacacacgctcggacgcatattacacatgttcatacacttaatactcgctgttttgaattgatgttttaggaatatatatgtagagagagcttccttgagtccattcacaggtcgtgatatgattcaattagcttccgactcattcatccaaataccgagtcgccaaaattcaaactagactcgttaaatgaatgaatgatgcggtagacaaattggatcattgattctctttgattggactgaagggagctccctct

SEQ ID NO: 259 - DNA, 660 bp, Brassica rapa
  source 1..660: mol_type = unassigned DNA
ttgtatccattgtgtatttccttgcatccatcaataaattttatgttacgcctctctattatttctctctacatcacactgtcttatgtttaagctctacttctcagcaattctctccacccaatacatgcatacataccatcatcgtatcgctctaatttttctatcaatcttgtatccttccacaaattatcttatgtctcccattttaaatcctacatagatccacacatacgaattattcttgtctgaagatccatccatttacgattgctttaacttttacatgagatctagggcttctttatttttcttcaaatcttgctgcatatatttcaagatcatgcttttcggcttgctagggttctagatccatggatgtatagcgtacatacatacacgcactaattcatacctgtagtttgtacggagaacatcataaaatatcactgtttggaattaatcgtgtaggaaatatagataggtagggagcttcctttagtccattcacagatcatgatatgatccaattagcttccgactcattcatccaaataccgagtcacgaaaatttcaacttagactcgttaaatgaatgaatgatgcggtaaacaaattggatcattgattctctctgattggactgaagggagctccctc

SEQ ID NO: 260 - DNA, 932 bp, Carica papaya
  source 1..932: mol_type = unassigned DNA
ctatatccctgtcaaatagtacttggttttgttttagccacaaatcttctggtcttgcaaagttccttctgatctctccccctttctcattttttcctcctcttatatatggatcatcaatttgctgtacacacacacacatttactgtagtgataattagctagctaatttgttagtatgtaaattagatcccaagtaccctgttatatttttttaggcttatcctatgcatacctgatagtacaagaacttagtttgtaattaggtacttggtagtagggttagattaattactgtcttgaaagagaacttatccaacaaatagagctatgaagattaaattaggttttagtcttattaagattattatattactagacaaaaacagttaaatttttttaattgggtaattaggtacttagcaatagggttagattaattactgttttgaaagagaacctacctacaaatagagttgaaatgattatgttttagtcttactaagattgtcatatttctggaaaaaaacaaatcttgaaacagataattcagatagtcatgatcaatggaaaaaacatcatgggtgtgtgcttaattaagctaatatatatatatatgaagatataatgttatgcacactagctatgaatttgtaagaataatgaaggataaagatgatatatttagatgttataagtgtaagtaaggtggaatgggttgatgggtagtagtagtagtagtagagatgattggtggagagagcttccttcagcccactcatggatgggtatgaaggggtagaagtagctgccgactcattcattcagccactcagtatgtaaactcgtcccactgttgactgtatgaatgatgcgggagatatttttacatccatctttctctgtgcttggactgaagggagctccttctt

SEQ ID NO: 261 - DNA, 702 bp, Capsella rubella
  source 1..702: mol_type = unassigned DNA
ttgtatccatagtgtatttcctcgcatctaccatctactattttctacgcctctctctctttatccctctatctctttcttcatcaaatcttcttttgttcaaagtctctctcatcatttttctctatacacatacatgcatccacatacatacatacatataccatcatcttcttttctcatctctagtttttgtttataaattttgttccaaggatctgtatctctccaataaagatacatacaaattattgttgtcatagatctattagaattgctttaacttttatatgagatctaggatttcttccttttctttcaaaatttgctgcatatttttcaaaatcatgatttatcgcttgctaggttctagatccatgcaaatttagagtatttttacacacacacacgcttggacacaagtacatacatgtagttttcttttatgtggtgaaaagtacataacatgtagtttatagttactagtcgctatataatttaaaattgatgttataggaatatatgtacggagagagcttccttgagtccattcacaggtcgtgatatgatccaattagcttccgactcattcatccaaataccgagtctcaccaaaattggaacaagacttgttaaatgaatgaatgatgcggtagacaaattggatcattgattctctttgattggactgaagggagctcc

SEQ ID NO: 262 - DNA, 767 bp, Eucalyptus grandis
  source 1..767: mol_type = unassigned DNA
tcgtttcccatccccatttcatagaataatgccaccaaacaaagaagcattagctcaaagactaattaccatctgttttattgatagatacgtgcgaaacggtgattgttttttcccaaataagaaaccaaaatgaagcatattcaaaggtggagatatggggagacttccggaaggcaaggggattggaaaaggctcgagatcaaagtgcatagcaacccttcgctaaaggtgaaaaagaatacgaataacttcagtagctcactttaaattccgaaacattaaacaaatcaaatctccctcgccctccttgcctcctctctttacctatataaagccaccgccccttcaatgaaatccacgagtggaaggtcacagtatagtagggtcctgcaaagggagagcgagagcggctccactgtctacctataagcagttcctttcttttgtttacatgtctgttgcacctcaccgagtttttctattctctttcctctggttggttagcagatttctcaggggacttccctcctcccttgaggatccttctcttgaagcgatatgtctcgaatgggtaagagagagagaggaagggagctttcttcagtccacccatgggacgtgttgggttttaattagctgccgactcattcatccaaataccgagcgagagcaagtaacagagctccgtaaatgaatggatgatgcgggagtcttgttgattcccaagctttccgtgattggactgaagggagctccctctatct

SEQ ID NO: 263 - DNA, 612 bp, Gossypium raimondii
  source 1..612: mol_type = unassigned DNA
gcgtatccttccacttacggattcaccatagctgctatagcccgagtttgcttgtcataatagaaataaactaagggagaaaaaagctctccactctcgcctttttcttcttcgagtctctctctttgactggcctttgtgcaaagatcttccttttttaaacagtcgctttctttactctccctttcccttctttctcttaattgctaaagcagcttccacttccactcactttaacccatgatccatttcaacctgtcacagtggagagcaatttgtatggctaatttccatctcacctattcttttctgtttggggttctctagtctagggttgcatgacatgagagacatggctcttttttttttttttcagttcacaggtgtttatatatgttgttgtgttattttgtcttaaagctttgtgattgatgatctgataggtaagagagagctttcttcagtccactcatgggatggggatggggtttaattagctgccgactcattcatccaaatactgtgttacaaaacccagtaaatgagtgaatgatgcgggagacaaattgaatcctaatcttcctgtgcttggactgaagggagctccctccc

SEQ ID NO: 264 - DNA, 675 bp, Medicago truncatula
  source 1..675: mol_type = unassigned DNA
atttatccaatcatgttgctctcacttcttttcagcagttctgttgatatcacccttttagttgaagctttggattcatgactttatgagatggtaaatctttaaattaagtttaagtatgcacttgatttgtttatataatttgtttatttagattttaaaccctaagttgacttttttaatttgatttaaattatgatatgattgttatttggcttaccatggtcataatttagggtttagaagatgcatgtatatcttgaattgtttatggtaataaaggggttaggatttctccttttggtgaagtgagaaaatctcataattttgttctgaaggtagtttttaagatttagggttatgggttctttgtttgaatgctttttcaagtctttttcaatgatatttgcctagatctgttttaattttgaattaaaattctgggtttggattaatattattgaagattattattaattaatttattagaaatagatgaagagagcttccttcagtccactcatggaagggtaaggggtttgaatttacctgctgactcattgattcaaacacaatagacaattatggggttatgctattgtgaattgtgtgaatgatgcaggaggtgaatttcttccttttcttctttgcttggactgaagggagtctcccttt

SEQ ID NO: 265 - DNA, 866 bp, Oryza sativa
  source 1..866: mol_type = unassigned DNA
ttatatctgacgcgttgtaatcctgtttaattagggctttgcccatttcttttgaccccttccggacattcgctagttggaaccttgttttactcctagcagtgtactgtgtagtacttattacgagcaaacgtaaaaataaataaatggaaatgatacaaaggccgtgtttaattttaaaattttttttcaaactttcaacactttacatcccataaaaagcttactatacatacaaacttccaaccttttcgtcacatcgtatctaatttcaactaaacttttaattttagcgtgaactaaacacagccgaagcccggccacattctcactatttttattcattttatcatgcctgtgatgtcacgccttggccctatttaataggccttctccatttctctccatatgatgtcttctcttctctatccctcttgccatcttctatcttccctcttgcacccatctttgtgataacttctactagctcctctcctactaccagtcataccactctcacaaatcctccaagatccgcatggggagaagctccaaaagtttcgtggttagtttaatttcatgcttgtttgctgccgtttttcatgttgatctgatcttaatatatgtagactgctgttaacatattcttttaatttgatggaagaagcgatcgatggatggaagagagcgatccttcagtccactcatgggcggtgctagggtcgaattagctgccgactcattcacccacaatgccaagcaagaaacgcttgagatagcgaagcttagcagatgagtgaatgaagcgggagagtaacgttccgatctcgcgccgtctttgcttggactgaagggtgctccctcct

SEQ ID NO: 266 - DNA, 801 bp, Physcomitrella patens
  source 1..801: mol_type = unassigned DNA
tgggatccacacgagtaattattccctctctcatgcatcacaacttggacttgcccagcttttacctctctccatgttccaccgtcggagatcctcggtgctgctacccccgttcggccaaacccaacccaaccctaggtgtctgccggacctccgcttcccctcctgcttcaccccctgcaccgcttaaatttgcacctcgtagtatttatctatgccccatttcagatgacttgcatgacacccatctggattcgtctccaggcgccttgtcgtatctattcgctatcagtctttctttcagtctgtctttctgccaagcacaccgtgctggtggtggtgctatcagtcgaagcagtcgccgaagggtccttgtatccagccctcaccggagacagttcgcttcggggtggggagaactgttgagaccgtcgatgttgcatgcagcagcactggcgaaggtgcttctgatactgggtattccagcctcgccgctcggtgcactgcaggtacgtggttacatgacactactgtgtgggtagtctgggcacaagtagatcgacatgccagatttggcccgtatgcgtgtatgtgcggttatgttcccgtttcgattgcggataccttgattgtggagctccgtttcggtccaatagtggctgcgacggaaggtggtcccgctgccgaatcacacgtccgggttgcttatcggggcagggccccgatacggtatccgaacgtttgtcccgggaactggtcgaccttccgcccggcgtctcttggactgaagggagctccact

SEQ ID NO: 267 - DNA, 819 bp, Thellungiella halophila
  source 1..819: mol_type = unassigned DNA
ttttatacaaataatgttcgataacactaaaccctagccatccaactaatagacaaaaccctacttgtaatttacaaccgcaaattcccagagaacagagtaactacgagagagagatggagattcaaattaaaaagaaaaacttatatataatgaatacacaaaagctacctaatctgtatatatatatatataaatatgtcttcattaaattaatggtcgtggaatagaaaaaggaaaacctaatttgatcgctagggcttatcagagtaaagatggttaaccttcaaaagatgactaattaaccggggagataattaaaagattaaatacgccaacagagagttaagagataccagatttaaattccacaatttggtcatgttcttcttcacgtattcatgacgatgtctgaattatagagaaacccaaaatataaaatgttaattttaccagacatttacataccaataactctatgacgatatgtaaagtaagcaaggcatgtttttatgcagggaagattgaaaattcaagattaatcaagaaaattggaataccaaaaagagagggagctcccttcagtccaatcagagagaatcaatgacccaatttgtctaccgcatcattcattcatttaacaagtctagctcgaattcttggtgactcggtatttggatgaatgagtcggaagctaattggatcatatcacgacctatgaatggactcaaggaagctctctacaaatgtattcctaccacatcaacccaaatatagtgattacagatgctgttctcactgtagactacatttacgttt

SEQ ID NO: 268 - DNA, 503 bp, Arabidopsis thaliana
  source 1..503: mol_type = unassigned DNA
agacatctcttcttctctcatctctcttttcttctctcttttcctcacataaactctctttttttactattaaatccatatggtacctcaaattaatctatggtcatctagggttatcttgaagattagaattgattctagcacgcacagagaggaagatcattgcatccagaatcacaaacatggcctatcttttatcttttctttttgatctaagtcactgttttatgctatatatagtataatcaaattctttacatgtgcttgtatgtatgcgtatatatagtaacggaattgttaatatgcttatagatgttgagttggtggaggaagagagctttcttcggtccactcatggagtaatatgtgagatttaattgactctcgactcattcatccaaataccaaatgaaagaatttgttctcatatggtaaatgaatgaatgatgcgagagacaaattgagtcttcacttctctatgcttggactgaagggagctccct

SEQ ID NO: 269 - DNA, 365 bp, Arabidopsis thaliana
  source 1..365: mol_type = unassigned DNA
tcttattccatcacaatcatctagggttttaagccaagcttatatagcccgtcataaagagaactcatctgcctctctctcaataccaataaatatcaccaccgtccttctctcctatcactattcaatctatcgcaaactcctttatgtctctccaattttatgagagggtttccttcaagaacacagtaaaatagattggatctttaaacttttgttccttttcatgagggtttgacaaagattttcttacagtcatctttggcattctgtccacctccttctatacatatatgcatgtgtatatatatatgcgtttcgtgtgaaagaaggaggtgggtatactgccaatagagatctgttag

SEQ ID NO: 270 - DNA, 134 bp, Arabidopsis thaliana
  source 1..134: mol_type = unassigned DNA
ttgtatcatgacagagcaagaagaagaaagtcaaatgtccacatgagttccctttaacgcttcattgttgaatactcaaagccacattggtttgtatataacactgaagtgtttggggggactcttggtgtcat

SEQ ID NO: 271 - DNA, 229 bp, Arabidopsis thaliana
  source 1..229: mol_type = unassigned DNA
tttcaaaccctaacactcttataaaccgattcgccaaaatgtatctacaatatattgataatgtaatatctatatattcaaacaatcgtcgtgttggtcggatgttttctagagttcctctgagcacttcattggagatacaattttttataaaatagttttctactgaagtgtttgggggaactcccgggctgattcggtattttaaattcagtagactagctagctg

SEQ ID NO: 272 - DNA, 220 bp, Arabidopsis thaliana
  source 1..220: mol_type = unassigned DNA
tggtaatagaaatgagcaaggagatatttttttcccctgggtttgaatgaacatcattgagtgcatcgttgatgtaattttacttattttattccattgttgaattaattaaagaagtatatatcagcgttgcattcaattatgtttttctaattttcaggaaatacaaaaaaaatgaaaaaaaaaaatcacttaaaagaccttgagagttcttttgact

SEQ ID NO: 273 - DNA, 225 bp, Arabidopsis thaliana
  source 1..225: mol_type = unassigned DNA
ggatatcgaaactcaaactgtaacagtccttttattactggtttagaagatagataaatattgttaaggtagtggatctcgacagggttgatatgagaacacacgagcaatcaacggctataacgacgctacgtcattgttacagctctcgtttcatgtgttctcaggtcacccctgctgagctctttctctaccgtccatgttttatcaacgccgtggcccgtg

SEQ ID NO: 274 - DNA, 165 bp, Arabidopsis thaliana
  source 1..165: mol_type = unassigned DNA
tcttatagagatgaagagaaacatgtaaactcactagttttagggcgcctctccattggcaggtcctttacttccaaatatacacatacatatatgaatatcgaaaatttccgatgatcgatttataaatgacctgccaaaggagagttgccctgaaactggttc

SEQ ID NO: 275 - DNA, 187 bp, Arabidopsis thaliana
  source 1..187: mol_type = unassigned DNA
caataactcaaaatgcaatgtgaaatatgaagaatatattaaatagtagtgaagatgcatgtttatgaagacagagagataatgtatggttggattactgggcgaatactcctatggcagatcgcattggctagatatgcaagtaaaatgcttctctgccaaaggagatttgccccgcaattcatcc

SEQ ID NO: 276 - DNA, 235 bp, Arabidopsis thaliana
  source 1..235: mol_type = unassigned DNA
atttaggtctctcttcttcttcttctttttcttcttgagcgccggcgaaaaaagtctctgtgagaaaaagatacgacgattgtcattagaagagtcgtattacatgttttgtgcttgaatctaattcaacaggctttatgtaagagattctttaacaattcctataatctttgttgttggattagattcacgcacaaactcgtaatctgtcttttcgatttttaccagatctgtc

SEQ ID NO: 277 - DNA, 944 bp, Arabidopsis thaliana
  source 1..944: mol_type = unassigned DNA
aattatatccatggtcatggctcatcattagtcgcactgctctccttttctcaaagtttaaattcgacatttggtaaaatgatgaaacctcgatggaactgctctctttatggaatcacggaatggacaaataatcaaaatcagaaatcgaagcgaaaagggaggagaaaaacgcagatttggaggattggggacagattagatactgttgaatgcatcactctaatgctatcagcctattaatagcgtcctatattttcgaagacttttaatgtttagggttatggatttttcgagcgaagcatggagagatgttgaattggatactataggatttggtacaacacatacatatgttctgcttctgcaaaactaacatatcaagttcagagaaaccagtaagtcgttgaatattttattatccattcaacgctttcttcttttggatcatgtcttgtttgcttgaccacttcttcttgcttaagaggatggacaatatataaaaactggagccttctttttctatgaatgcttatcatcgcggagttgatctgttcaattcacctgccattggatgctttttttatatatacttcactgttcaatttcagatgctttagaaggtttgcggagtagctagagaatctggtatcttcagttcttcaatttcagctacttggtatcagcttcgtcattgtatatcaacacattcttaatatataatactactttttcatccattaaaccccttacaatgtcgagtaaacgaagcatctgtcccctggtattgtcttcgagcttggtgtttttttctagccaactccaagttctcgagttgatcattgtttgtattcttgagacattatttggggacgagatgttttgttgactcgatataagaaggggctttatggaagaaattgtagtattatatatcgagagtg

SEQ ID NO: 278 - DNA, 420 bp, Arabidopsis thaliana
  source 1..420: mol_type = unassigned DNA
ctataaatgctgcttatcatcgtggagttggttctgtaaacatttgaaaattctgaacagtttcacctgccattggatgctttgtttcaatttcaggtgcgttagaaggtttgcagagtagctagagaatctcgtatcttcactttctgctacttggtatcagcttcgtcactttatatcaacacattcttaatatacaatactactttttcatccattaatccccttacaatgtcgagtaaacgaagcatctgtcccctggtattgtcttcgagcttggtgtgtttttctagccagccccaagttctcgagttgatcattgtttgtattctgacacattatttggggacgagatgttttgttgactcgatataagaaggggctttatggaagaaattgtagtattatatattgagaatg

SEQ ID NO: 279 - DNA, 383 bp, Arabidopsis thaliana
  source 1..383: mol_type = unassigned DNA
tagtataaccgctgatgtacacctaccagcttgataactctttttcgtggtttctgtgtactcgtttctgtttgtacagatacttcttgttcaatttcagatgctttagaaggttttcggagtggctagagatctgttatctgtatgaacagctacttggtatcagcttcgtcattttatcaacacattcttaatatacaatacttctttttcatgcattaagccccttacaatgtcgagtaaacaaagcatgtgtccgctaatattgtcttcgagcttggtatttttgtattctgatacggtatttggggacgacatcttttgttgactcgatataagaagggggtttgtggaagaaattgtagtattatatatcaagaatg

SEQ ID NO: 280 - DNA, 2164 bp, Drosophila melanogaster
  source 1..2164: mol_type = unassigned DNA
tttgtggaacacattcgacccactgaaaaattgatataatttaatgaaagtgcataaaaatggtggacagtgcattaaactgagcattgaacacaaaggccgctcagcaaattgctaattaaaattcacgattgccatttcacctgacacgttgacgattttcattacaattcgattatgtttcgttgcagggaattttaaatgttaattgccaagaatgtttcaacaaattcatttctcattaatgtgtcttttcatttaattttatgttgtatgagctgcacgagaaatgagttgtacttttagttcgacggcagagtcatgaatgttcggcaaagaatgtaataataactatcctctttagacaaatatagatacaaatctatcagattctaaaagtagaataatcaattaatcagaaagctaaaaataaataggcatatttatattttaatgcggatttttgaagttcaacgggagaaatgaatcctttttaccagccacaggcgcaatttgcaacagaaagtgtagcagaagtactcctcgaatatttccctgctccaggagtcatccatgtggtttcgaggcacacatttgacaaactcatgccccgctatttgttgtaaaaacacaatcgcacacatggccgcatttcggcgacttccagagagcggtacacttaaggcggcctgggaaacgcctgcaatctgctggtcgcgaactgcagattgcatccatgtgccaggcgaccatgcgaccatgtgaccatgtgcccgcccgacgcctcgcagcccacatcctgcccatcgagggcacaactcagcgtgggtattgccgctccggctgcttcaagtaggtaaaaaccgagaagattgaggatgaatgtatgagtatgagaaaatactcggcggaacatatgctgccgggcttgacctgaccctgcctcatgtgtgggtctccgatttaattttaggcacctatataaacgcgtgtttacactgcagccagaacacagtcgccgtcttcagttcgcgccgtcaactcctcgatcgatcgatcgcatcgtctcggatcgaatagagctgggcttctgctccggagctacatcgccgtacttgtcggacgagtgtggtgatgaaaagtcgcttagtccgggattcctgccagatctctaagggatgagctggcatcccaggctggccatgtggcgcgaagtatgcgcacaaaaaagtcaaacaaaaaggcgcaattttattacgggcaaccaacgacgaaacaaaacaaaagccaaccgaaaagcagaaacaaagcagcaaaaagtttatgaattttttgtgcaggcgcgtgaaagatgcaaaacgagaaaaaaacatgaaaaaaaaacattaaaaaaaacaaaaaaaatccaaaacagataccgagctgtatccgaaaacgagtggggaaaggggtttcccagtcacatataaacacacttcagtgcgcttaaaaattgctttattgcagttggactataaaaacgcacggcagcgaacaccgcacaacaaaaaggacgagcagaagtgggcaaataaaacgaaagctcttaaacgaaaaacaggaaaatttgcatgccacaaaaataagcataaggatttgccgcgcacaaagtagaagcaaaaaggaattgcccaaatgcagccacaaaagactgtggcaaatgttttgcagcttgcccctttttccctgcaattaccgtcagtcgttgtcattattcagcagattatatggttttgcttattccggaccaccatcatcatcatcattatcatcatcttcggtaagttagacaatcccataaaaaactgtccaagtgagtagtgccaccaaaagttagccgcgttgtggaaaatccaaaacaaagaccatcccatattcagcctttgagagttccatgcttccttgcattcaatagttatattcaagcatatggaatgtaaagaagtatggagcgaaatctggcgagacatcggagttgaaactaaaactgaaatttgattgaaacagaagtagaaccgtaatgaaatgaatgaaatattaacccgtttctacaatccctgaataaaattattaattaattatagagcgggctaattttacaatatatattgatttttttttgaag

SEQ ID NO: 281 - DNA, 919 bp, Drosophila melanogaster
  source 1..919: mol_type = unassigned DNA
attcttttttggtgctcgatcgtgacggtttgctcgcgctctccgctgcgccgctctttccgttgcatatgtgtgcgggcgttattgtgcatgtttccggtggccgaaaaaaaatagtaaaataaaatatagaaaacagaaaccaagaataataacagccatacgataaacagtgtgccaatgtgtgtgtctgtgtgtgtgtgcatctcgcgtaacaacaataattgcatttatcggatggcgccagcttcaatttaattataaataacatgttcaactttttatactattttccctgcgtcaaagtgggcgttgcaactgcccccggaaaatcacgcgccccggttcaaagttaaagtttgctgggtaacgcacacacacacacacacaatcactcacacgcggtcacacgcacatttcaataaactaatggagcctggctttgtttttgttttatttccaacccacttgagcacacagcacacacagagagaaaaatcaatactcgttatgggattaaatttacaaagcgcaaagcaaagcgacaaacaaaattcaaaagaaagaaaaaaaaacactcaaataaactcacaaagaattccttatcgccaagggggccaatgttctaaggttctttcgccttgagaactttgagcttcctctggcaaaggagattataatgtacaaataatgttgcaataaccagttgaaaccaatggaataccgaatcttgctaattagcaaggacatctgttcacatcttaccgggcagcattagatcctttttataactctaatactgtcaggtaaagatgtcgtccgtgtccttaaccttcagtaccaccaacagcagcagcagcaccaaaaaaaaaaaaaaaaaaatgcgtaaaaatccaaacaaatcataaaagtcgaagga

SEQ ID NO: 282 - RNA, 20 nt, Arabidopsis thaliana
  source 1..20: mol_type = unassigned RNA
tgacagaagagagtgagcac

SEQ ID NO: 283 - RNA, 20 nt, Arabidopsis thaliana
  source 1..20: mol_type = unassigned RNA
tgacagaagagagtgagcac

SEQ ID NO: 284 - RNA, 20 nt, Arabidopsis thaliana
  source 1..20: mol_type = unassigned RNA
tgacagaagagagtgagcac

SEQ ID NO: 285 - RNA, 20 nt, Arabidopsis thaliana
  source 1..20: mol_type = unassigned RNA
tgacagaagagagtgagcac

SEQ ID NO: 286 - RNA, 21 nt, Arabidopsis lyrata
  source 1..21: mol_type = unassigned RNA
tttggattgaagggagctcta

SEQ ID NO: 287 - RNA, 21 nt, Arabidopsis thaliana
  source 1..21: mol_type = unassigned RNA
tttggattgaagggagctcta

SEQ ID NO: 288 - RNA, 21 nt, Capsella rubella
  source 1..21: mol_type = unassigned RNA
tttggattgaagggagctcta

SEQ ID NO: 289 - RNA, 21 nt, Arabidopsis thaliana
  source 1..21: mol_type = unassigned RNA
tttggattgaagggagctctt

SEQ ID NO: 290 - RNA, 21 nt, Arabidopsis thaliana
  source 1..21: mol_type = unassigned RNA
tgcctggctccctgtatgcca

SEQ ID NO: 291 - RNA, 21 nt, Arabidopsis thaliana
  source 1..21: mol_type = unassigned RNA
tgcctggctccctgtatgcca

SEQ ID NO: 292 - RNA, 21 nt, Arabidopsis thaliana
  source 1..21: mol_type = unassigned RNA
tcaatgcattgaaagtgacta

SEQ ID NO: 293 - RNA, 21 nt, Arabidopsis thaliana
  source 1..21: mol_type = unassigned RNA
tcgataaacctctgcatccag

SEQ ID NO: 294 - RNA, 21 nt, Arabidopsis thaliana
  source 1..21: mol_type = unassigned RNA
tcgataaacctctgcatccag

SEQ ID NO: 295 - RNA, 24 nt, Arabidopsis thaliana
  source 1..24: mol_type = unassigned RNA
ttgaagaggacttggaacttcgat

SEQ ID NO: 296 - RNA, 21 nt, Arabidopsis lyrata
  source 1..21: mol_type = unassigned RNA
tggagaagcagggcacgtgca

SEQ ID NO: 297 - RNA, 21 nt, Arabidopsis thaliana
  source 1..21: mol_type = unassigned RNA
tggagaagcagggcacgtgca

SEQ ID NO: 298 - RNA, 21 nt, Brassica rapa
  source 1..21: mol_type = unassigned RNA
tggagaagcagggcacgtgca

SEQ ID NO: 299 - RNA, 21 nt, Carica papaya
  source 1..21: mol_type = unassigned RNA
tggagaagcagggcacgtgca

SEQ ID NO: 300 - RNA, 21 nt, Capsella rubella
  source 1..21: mol_type = unassigned RNA
tggagaagcagggcacgtgca

SEQ ID NO: 301 - RNA, 21 nt, Gossypium raimondii
  source 1..21: mol_type = unassigned RNA
tggagaagcagggcacgtgca

SEQ ID NO: 302 - RNA, 21 nt, Medicago truncatula
  source 1..21: mol_type = unassigned RNA
tggagaagcagggcacgtgca

SEQ ID NO: 303 - RNA, 21 nt, Oryza sativa
  source 1..21: mol_type = unassigned RNA
tggagaagcagggtacgtgca

SEQ ID NO: 304 - RNA, 21 nt, Arabidopsis lyrata
  source 1..21: mol_type = unassigned RNA
tcggaccaggcttcatccccc

SEQ ID NO: 305 - RNA, 21 nt, Arabidopsis thaliana
  source 1..21: mol_type = unassigned RNA
tcggaccaggcttcatccccc

SEQ ID NO: 306 - RNA, 21 nt, Brassica carinata
  source 1..21: mol_type = unassigned RNA
tcggaccaggcttcatccccc

SEQ ID NO: 307 - RNA, 21 nt, Brassica juncea
  source 1..21: mol_type = unassigned RNA
tcggaccaggcttcatccccc

SEQ ID NO: 308 - RNA, 21 nt, Brassica napus
  source 1..21: mol_type = unassigned RNA
tcggaccaggcttcatccccc

SEQ ID NO: 309 - RNA, 21 nt, Brassica oleracea
  source 1..21: mol_type = unassigned RNA
tcggaccaggcttcatccccc

SEQ ID NO: 310 - RNA, 21 nt, Brassica rapa
  source 1..21: mol_type = unassigned RNA
tcggaccaggcttcatccccc

SEQ ID NO: 311 - RNA, 21 nt, Arabidopsis thaliana
  source 1..21: mol_type = unassigned RNA
tcggaccaggcttcattcccc

SEQ ID NO: 312 - RNA, 21 nt, Arabidopsis thaliana
  source 1..21: mol_type = unassigned RNA
tcggaccaggcttcattcccc

SEQ ID NO: 313 - RNA, 21 nt, Arabidopsis thaliana
  source 1..21: mol_type = unassigned RNA
tgaagctgccagcatgatcta

SEQ ID NO: 314 - RNA, 21 nt, Arabidopsis thaliana
  source 1..21: mol_type = unassigned RNA
tgaagctgccagcatgatcta

SEQ ID NO: 315 - RNA, 21 nt, Arabidopsis thaliana
  source 1..21: mol_type = unassigned RNA
cagccaaggatgacttgccgg

SEQ ID NO: 316 - RNA, 21 nt, Arabidopsis thaliana
  source 1..21: mol_type = unassigned RNA
tagccaaggatgacttgcctg

SEQ ID NO: 317 - RNA, 21 nt, Arabidopsis thaliana
  source 1..21: mol_type = unassigned RNA
tgattgagccgcgccaatatc

SEQ ID NO: 318 - RNA, 21 nt, Arabidopsis thaliana
  source 1..21: mol_type = unassigned RNA
ttgagccgtgccaatatcacg

SEQ ID NO: 319 - RNA, 21 nt, Medicago truncatula
  source 1..21: mol_type = unassigned RNA
tgattgagccgcgtcaatatc

SEQ ID NO: 320 - RNA, 21 nt, Zea mays
  source 1..21: mol_type = unassigned RNA
ggattgagccgcgtcaatatc

SEQ ID NO: 321 - RNA, 21 nt, Arabidopsis thaliana
  source 1..21: mol_type = unassigned RNA
ttgagccgtgccaatatcacg

SEQ ID NO: 322 - RNA, 21 nt, Medicago truncatula
  source 1..21: mol_type = unassigned RNA
agattgagccgcgccaatatc

SEQ ID NO: 323 - RNA, 21 nt, Medicago truncatula
  source 1..21: mol_type = unassigned RNA
cgagccgaatcaatatcactc

SEQ ID NO: 324 - RNA, 21 nt, Arabidopsis thaliana
  source 1..21: mol_type = unassigned RNA
agaatcttgatgatgctgcat

SEQ ID NO: 325 - RNA, 20 nt, Arabidopsis thaliana
  source 1..20: mol_type = unassigned RNA
gcagcaccattaagattcac

SEQ ID NO: 326 - RNA, 21 nt, Arabidopsis thaliana
  source 1..21: mol_type = unassigned RNA
agaatcttgatgatgctgcag

SEQ ID NO: 327 - RNA, 21 nt, Arabidopsis thaliana
  source 1..21: mol_type = unassigned RNA
ggaatcttgatgatgctgcat

SEQ ID NO: 328 - RNA, 21 nt, Arabidopsis cebennensis
  source 1..21: mol_type = unassigned RNA
ttggactgaagggagctccct

SEQ ID NO: 329 - RNA, 21 nt, Arabidopsis halleri
  source 1..21: mol_type = unassigned RNA
ttggactgaagggagctccct

SEQ ID NO: 330 - RNA, 21 nt, Arabidospis lyrata
  source 1..21: mol_type = unassigned RNA
ttggactgaagggagctccct

SEQ ID NO: 331 - RNA, 21 nt, Arabidopsis thaliana
  source 1..21: mol_type = unassigned RNA
ttggactgaagggagctccct

SEQ ID NO: 332 - RNA, 21 nt, Brassica rapa
  source 1..21: mol_type = unassigned RNA
ttggactgaagggagctccct

SEQ ID NO: 333 - RNA, 21 nt, Carica papaya
  source 1..21: mol_type = unassigned RNA
ttggactgaagggagctcctt

SEQ ID NO: 334 - RNA, 19 nt, Capsella rubella
  source 1..19: mol_type = unassigned RNA
ttggactgaagggagctcc

SEQ ID NO: 335 - RNA, 21 nt, Eucalyptus grandis
  source 1..21: mol_type = unassigned RNA
ttggactgaagggagctccct

SEQ ID NO: 336 - RNA, 21 nt, Gossypium raimondii
  source 1..21: mol_type = unassigned RNA
ttggactgaagggagctccct

SEQ ID NO: 337 - RNA, 22 nt, Medicago truncatula
  source 1..22: mol_type = unassigned RNA
ttggactgaagggagtctccct

SEQ ID NO: 338 - RNA, 21 nt, Oryza sativa
  source 1..21: mol_type = unassigned RNA
ttggactgaagggtgctccct

SEQ ID NO: 339 - RNA, 20 nt, Physcomitrella patens
  source 1..20: mol_type = unassigned RNA
cttggactgaagggagctcc

SEQ ID NO: 340 - RNA, 20 nt, Thellungiella halophila
  source 1..20: mol_type = unassigned RNA
tggactcaaggaagctctct

SEQ ID NO: 341 - RNA, 21 nt, Arabidopsis thaliana
  source 1..21: mol_type = unassigned RNA
ttggactgaagggagctccct

SEQ ID NO: 342 - RNA, 20 nt, Arabidopsis thaliana
  source 1..20: mol_type = unassigned RNA
ttggcattctgtccacctcc

SEQ ID NO: 343 - RNA, 21 nt, Arabidopsis thaliana
  source 1..21: mol_type = unassigned RNA
ctgaagtgtttggggggactc

SEQ ID NO: 344 - RNA, 21 nt, Arabidopsis thaliana
  source 1..21: mol_type = unassigned RNA
ctgaagtgtttgggggaactc

SEQ ID NO: 345 - RNA, 21 nt, Arabidopsis thaliana
  source 1..21: mol_type = unassigned RNA
tcattgagtgcatcgttgatg

SEQ ID NO: 346 - RNA, 21 nt, Arabidopsis thaliana
  source 1..21: mol_type = unassigned RNA
tgtgttctcaggtcacccctg

SEQ ID NO: 347 - RNA, 21 nt, Arabidopsis thaliana
  source 1..21: mol_type = unassigned RNA
tgccaaaggagagttgccctg

SEQ ID NO: 348 - RNA, 21 nt, Arabidopsis thaliana
  source 1..21: mol_type = unassigned RNA
tgccaaaggagatttgccccg

SEQ ID NO: 349 - RNA, 21 nt, Arabidopsis thaliana
  source 1..21: mol_type = unassigned RNA
ttagattcacgcacaaactcg

SEQ ID NO: 350 - RNA, 22 nt, Arabidopsis thaliana
  source 1..22: mol_type = unassigned RNA
ttggggacgagatgttttgttg

SEQ ID NO: 351 - RNA, 22 nt, Arabidopsis thaliana
  source 1..22: mol_type = unassigned RNA
ttggggacgagatgttttgttg

SEQ ID NO: 352 - RNA, 21 nt, Arabidopsis thaliana
  source 1..21: mol_type = unassigned RNA
ccccttacaatgtcgagtaaa

SEQ ID NO: 353 - RNA, 22 nt, Drosophila melanogaster
  source 1..22: mol_type = unassigned RNA
tggaatgtaaagaagtatggag

SEQ ID NO: 354 - RNA, 23 nt, Drosophila melanogaster
  source 1..23: mol_type = unassigned RNA
taatactgtcaggtaaagatgtc

SEQ ID NO: 355 - protein, 17 aa, Homo sapiens
MEMALMVAQTRKGKSVV

SEQ ID NO: 356 - DNA, 53 bp, Homo sapiens
  source 1..53: mol_type = unassigned DNA
tggagatggctctaatggtggcacaaaccaggaaggggaaatctgtggtttaa

SEQ ID NO: 357 - DNA, 1500 bp, Homo sapiens
  source 1..1500: mol_type = unassigned DNA
gccgagcccgggcccagcgccgcctgcagcctcgggaagggagcggatagcggagccccgagccgcccgcagagcaagcgcggggaaccaaggagacgctcctggcactgcagataacttgtctgcatttcaagaacaacctaccagagaccttacctgtcaccttggctctcccacccaatggagatggctctaatggtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaagataaaaagatttaatcaaaaaatttcatatttgaaatgaagtcccaaatctaggttcaagttcaatagcttagccacataatacggttgtgcgagcagagaatctacctttccacttctaagcctgtttcttcctccatatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgatttaaaataaaaaaaaaaaaaaaaaaaaaaaaaaaa

SEQ ID NO: 358 - RNA, 23 nt, Homo sapiens
  source 1..23: mol_type = unassigned RNA
taatactgtcaggtaaagatgtc

SEQ ID NO: 359 - DNA, 337 bp, Medicago truncatula
  source 1..337: mol_type = unassigned DNA
ttagggttttcagctcatggtaataaaaatgtcatctaatgtcttgcatgtgggaatgaggtcatatatgcagccaaggatgacttgccggcgagcctctttcgatacttttatgacataattaatcatgtggatagccaaggtactaaactcactttgcactaaaacaaatatttttgctttagtgcaaacttagtttaggcgcttcgcaacggctagtcaaatgtcctagttccaatgtgattggttgtccggcaagtcgtctctggctacgtaaaggcctccttttttcatgctagatttttgatgatttgatatagccacacatattttggaa

SEQ ID NO: 360 - DNA, 121 bp, Medicago truncatula
  source 1..121: mol_type = unassigned DNA
aagaggcagagagagtaatgcagccaaggatgacttgccgacaacattggcgaatgttcatgtgatttctgcctcattgtgccggcaagttgtccttggctatgttagtctctcatcttct

SEQ ID NO: 361 - DNA, 114 bp, Medicago truncatula
  source 1..114: mol_type = unassigned DNA
tgaattcccctccgctttttgatgttggcttgtctcaatcaaatcaaagttcttgaaatttgagttctttagtctgattgagtcgtgccaatatcatattaagcgataaaagtc

SEQ ID NO: 362 - DNA, 367 bp, Medicago truncatula; identical to SEQ ID NO: 250
  source 1..367: mol_type = unassigned DNA

SEQ ID NO: 363 - DNA, 135 bp, Medicago truncatula
  source 1..135: mol_type = unassigned DNA
aactgcaacttgaggaggcatccaaagggatcgcattgatcctataatatttcaactttagtcactttaattttctctcatataatacttaattgggatcatgccatccctttggatttctcctttagtagctac

SEQ ID NO: 364 - DNA, 119 bp, Medicago truncatula
  source 1..119: mol_type = unassigned DNA
aggcatccaaagggatcgcattgatcccaaatctaattaagtccctagctacttaattaacaacttaatttccttaatatctcataatatttgggatcatgctatccctttggattcat

SEQ ID NO: 365 - DNA, 93 bp, Medicago truncatula
  source 1..93: mol_type = unassigned DNA
tgcttttccacagctttcttgaacttctttcgtatcttaaatctgttttcaagattaaaagtcctagaagctcaagaaagctgtgggagaata

SEQ ID NO: 366 - DNA, 95 bp, Medicago truncatula
  source 1..95: mol_type = unassigned DNA
tattcttccacagctttcttgaactgcatccaaattgagttcctttgcattgccatggccattgttttgcggttcaataaagctgtgggaagata

SEQ ID NO: 367 - RNA, 21 nt, Medicago truncatula
  source 1..21: mol_type = unassigned RNA
cagccaaggatgacttgccgg

SEQ ID NO: 368 - RNA, 21 nt, Medicago truncatula
  source 1..21: mol_type = unassigned RNA
cagccaaggatgacttgccga

SEQ ID NO: 369 - RNA, 21 nt, Medicago truncatula
  source 1..21: mol_type = unassigned RNA
tgattgagtcgtgccaatatc

SEQ ID NO: 370 - RNA, 20 nt, Medicago truncatula
  source 1..20: mol_type = unassigned RNA
gagccgaatcaatatcactc

SEQ ID NO: 371 - RNA, 21 nt, Medicago truncatula
  source 1..21: mol_type = unassigned RNA
tccaaagggatcgcattgatc

SEQ ID NO: 372 - RNA, 21 nt, Medicago truncatula
  source 1..21: mol_type = unassigned RNA
tccaaagggatcgcattgatc

SEQ ID NO: 373 - RNA, 21 nt, Medicago truncatula
  source 1..21: mol_type = unassigned RNA
ttccacagctttcttgaactt

SEQ ID NO: 374 - RNA, 21 nt, Medicago truncatula
  source 1..21: mol_type = unassigned RNA
ttccacagctttcttgaactg

SEQ ID NO: 375 - protein, 15 aa, Arabidopsis thaliana
MMLHITHRFESDVGC

SEQ ID NO: 376 - protein, 4 aa, Arabidopsis thaliana
MLYV

SEQ ID NO: 377 - protein, 14 aa, Arabidopsis thaliana
MFMRRGLVYNNIYI

SEQ ID NO: 378 - protein, 17 aa, Arabidopsis thaliana
MMKVCDEQDGEAGHVHY

SEQ ID NO: 379 - protein, 28 aa, Arabidopsis thaliana
MKKRITRINLEEQIKKTLDDSRTRLHSP

SEQ ID NO: 380 - protein, 10 aa, Arabidopsis thaliana
MKKIGSIDSF

SEQ ID NO: 381 - protein, 6 aa, Arabidopsis thaliana
MTCRFK

SEQ ID NO: 382 - protein, 11 aa, Arabidopsis thaliana
MKNENLCGSQG

SEQ ID NO: 383 - protein, 45 aa, Arabidopsis thaliana
MKCMMKKRGLTWRKASCLVAKDDLPDLFRLHDSISNSCILDYYTF

SEQ ID NO: 384 - protein, 7 aa, Arabidopsis thaliana
MFPRESL

SEQ ID NO: 385 - protein, 29 aa, Arabidopsis thaliana
MTLSVFFHSFLELQNFFRFFFFSFDISYA

SEQ ID NO: 386 - protein, 23 aa, Arabidopsis thaliana
MSLAKGELPCHCFRLNTVYNRFC

SEQ ID NO: 387 - DNA, 48 bp, Arabidopsis thaliana
  source 1..48: mol_type = unassigned DNA
atgatgttgcatatcacacataggtttgagagtgatgttggttgttga

SEQ ID NO: 388 - DNA, 15 bp, Arabidopsis thaliana
  source 1..15: mol_type = unassigned DNA
atgctgtatgtatag

SEQ ID NO: 389 - DNA, 45 bp, Arabidopsis thaliana
  source 1..45: mol_type = unassigned DNA
atgttcatgcgtagaggtttggtatacaacaatatatacatataa

SEQ ID NO: 390 - DNA, 54 bp, Arabidopsis thaliana
  source 1..54: mol_type = unassigned DNA
atgatgaaggtgtgtgatgagcaagatggagaagcagggcacgtgcattactag

SEQ ID NO: 391 - DNA, 87 bp, Arabidopsis thaliana
  source 1..87: mol_type = unassigned DNA
atgaagaagagaatcactcgaattaatttggaagaacaaattaagaaaaccctagatgattctcggaccaggcttcattccccctaa

SEQ ID NO: 392 - DNA, 33 bp, Arabidopsis thaliana
  source 1..33: mol_type = unassigned DNA
atgaagaagatcggtagtattgattcattttaa

SEQ ID NO: 393 - DNA, 21 bp, Arabidopsis thaliana
  source 1..21: mol_type = unassigned DNA
atgacttgccgatttaaatga

SEQ ID NO: 394 - DNA, 12 bp, Arabidopsis thaliana
  source 1..12: mol_type = unassigned DNA
atggtgacatga

SEQ ID NO: 395 - DNA, 36 bp, Arabidopsis thaliana
  source 1..36: mol_type = unassigned DNA
atgaagaatgagaacttgtgtggtagccaaggatga

SEQ ID NO: 396 - DNA, 138 bp, Arabidopsis thaliana
  source 1..138: mol_type = unassigned DNA
atgaagtgtatgatgaagaagagaggtctaacatggcggaaagcgtcatgtttagtagccaaggatgacttgcctgatctttttcgcctccacgattcaatttcaaattcatgcattttggattattataccttttaa

SEQ ID NO: 397 - DNA, 24 bp, Arabidopsis thaliana
  source 1..24: mol_type = unassigned DNA
atgtttccgagagagtccctctga

SEQ ID NO: 398 - DNA, 90 bp, Arabidopsis thaliana
  source 1..90: mol_type = unassigned DNA
atgaccctctctgtattcttccacagctttcttgaactgcaaaacttcttcagatttttttttttttcttttgatatctcttacgcataa

SEQ ID NO: 399 - DNA, 72 bp, Arabidopsis thaliana
  source 1..72: mol_type = unassigned DNA
atgtcacttgccaaaggagagttgccctgtcactgcttccgcttaaacacagtctataaccggttctgctaa

SEQ ID NO: 400 - DNA, 331 bp, Arabidopsis thaliana
  source 1..331: mol_type = unassigned DNA
ctttgtcacttcatacactccctattgtctatatatatatatatacttacacatattcaaacattataatacttaattacacatacatactttatgatgttgcatatcacacataggtttgagagtgatgttggttgttgacagaagatagagagcactaaggatgacatgcaagtacatacatatatatcatcacaccgcatgtggatgataaaatatgtataacaaattcaaagaaagagagggagagaaagagagagaacctgcatctctactcttttgtgctctctatacttctgtcaccacctttatctcttcttctctctaacct

SEQ ID NO: 401 - DNA, 304 bp, Arabidopsis thaliana
  source 1..304: mol_type = unassigned DNA
atttactcttcaccgccctctctctatatatagtctctatcctcacatattatatatcaaaccgcaagaatgctgtatgtatagtggagggtgatagtgtggttgctgacagaagatagagagcactaaggatgctatgcaaaacagacacagatatgtgtttctaattgtatttcatactttaacctcaaagttgatataaaaaaagaaagaaagatagaagagctagaagactatctgcatctctattcctatgtgctctctatgcttctgtcatcacctttctttctctatttctctctac

SEQ ID NO: 402 - DNA, 245 bp, Arabidopsis thaliana
  source 1..245: mol_type = unassigned DNA
aaccaaaactcttcaacatttctctctgactacttcatttcctcttcccaacagttaaaaaaagttctgattcgattcaagccaagatccacgtataaagatatgttcatgcgtagaggtttggtatacaacaatatatacatataatagtttgtcgttatgcctggctccctgtatgccacgagtggataccgattttggttttaaaatcggctgccggtggcgtacaaggagtcaagcatgac

SEQ ID NO: 403 - DNA, 240 bp, Arabidopsis thaliana
  source 1..240: mol_type = unassigned DNA
atacattctctctttctctctctctctctctctcatcccggcccagttatgtggtcggagagaatgatgaaggtgtgtgatgagcaagatggagaagcagggcacgtgcattactagctcatatatacactctcaccacaaatgcgtgtatatatgcggaattttgtgatatagatgtgtgtgtgtgttgagtgtgatgatatggatgagttagttcttcatgtgcccatcttcaccatc

SEQ ID NO: 404 - DNA, 280 bp, Arabidopsis thaliana
  source 1..280: mol_type = unassigned DNA
tcacacatacctttctttctcttcttcttcttacgaaaagtttcatcacattcacattatctttaactttggtctcttttcttttttgtctcttttctcttcttgataacgtggttctagtcttgattaattcattgttgtgcgatttagtgttgagaggattgttgtctggctcgaggtcatgaagaagagaatcactcgaattaatttggaagaacaaattaagaaaaccctagatgattctcggaccaggcttcattccccctaacctacttatcgc

SEQ ID NO: 405 - DNA, 179 bp, Arabidopsis thaliana
  source 1..179: mol_type = unassigned DNA
atttagcttcttcttcttcttcttcttctgtctacttacataaagttatccttgctttggtttagggttgagaggaatattgtctggctcgaggtcatgaagaagatcggtagtattgattcattttaaagagtgaaatccctaaatgattctcggaccaggcttcattccccccaacc

SEQ ID NO: 406 - DNA, 376 bp, Arabidopsis thaliana
  source 1..376: mol_type = unassigned DNA
tagtattcataagcaccaaaacaaatatgtagagatctcctcttccattctctattgttactttcgagaagaaacatacaaaacacatacatttttcttttgtttgtggttttcatatatacaagtgggtatagctagtgaaacgcgaatgtgacgaaagtagtgtgcagccaaggatgacttgccgatttaaatgatctttctttatactctattaagacaatttagtttcaaacttttttttttttttttttttgaaggattcaggaagaaattaggatatattattccgtataaaatacaagatatataaaaccaaaaagaaaaagtaacatgatcggcaagttgtccttggctacacgttactttgtgtcgc

SEQ ID NO: 407 - DNA, 77 bp, Arabidopsis thaliana
  source 1..77: mol_type = unassigned DNA
actcatcaacaacctcttcataaatacataaatcatataagagaaaatggtgacatgaagaatgagaacttgtgtgg

SEQ ID NO: 408 - DNA, 248 bp, Arabidopsis thaliana
  source 1..248: mol_type = unassigned DNA
aggcaaaaacatatagagagtaatgaagtgtatgatgaagaagagaggtctaacatggcggaaagcgtcatgtttagtagccaaggatgacttgcctgatctttttcgcctccacgattcaatttcaaattcatgcattttggattattataccttttaaagtataataggtcaaatatcatgttgaatcttgcgggttaggtttcaggcagtctctttggctatcttgacatgctttttccatccat

SEQ ID NO: 409 - DNA, 187 bp, Arabidopsis thaliana
  source 1..187: mol_type = unassigned DNA
attcactcccttcttcttcttaatctccttacagttacagacattctctcacttgcgttcttgtttcttttacaaaacagatacactatgtttccgagagagtccctctgatattggcctggttcactcagattctcttttactaactcatctgattgagccgtgtcaatatctcagtcctctctcg

SEQ ID NO: 410 - DNA, 246 bp, Arabidopsis thaliana
  source 1..246: mol_type = unassigned DNA
tctcacaacttcaacttccctctttctctatattacgcttttgcccctcactccctctttccacaattagggtttcgtctgctctacatgaccctctctgtattcttccacagctttcttgaactgcaaaacttcttcagatttttttttttttcttttgatatctcttacgcataaaatagtgattttcttcatatctctgctcgattgatttgcggttcaataaagctgtgggaagatacagac

SEQ ID NO: 411 - DNA, 631 bp, Arabidopsis thaliana
  source 1..631: mol_type = unassigned DNA
gaataaccaaccagccttctctcaaagcaaaccaaaaagaaaaaccaacattgaaagaggaagttacgataagcggagcagtaatagggcatctttctattggcaggcgacttggctatttgtatcttttgtgttcttgactattggctatgtcacttgccaaaggagagttgccctgtcactgcttccgcttaaacacagtctataaccggttctgctaatatcaatccttcttttggacatgtccaaagccgagattgattgatagagaattggtctctctggctacaaaactagtgcggttctctcgatttaagttttaatagcattcactttgcacattgcatctttcacatcaaatttccatttcatcaaccatctaaacctctttgttagctttgatataagcaacgatctaaagtctaaaaaccattaatcctctgaaaaaaaagacaatttcgatggttctattatgtttctccaatgcagaaattgtatcgtctgaattatagtagattttttctagactaaagtgtaaaccaagacgaatctgcactaacaagacacaccaatagactttacagagaaaggttacgagttttgaaaatattaacggaccatagtcatcgcg

SEQ ID NO: 412 - RNA, 21 nt, Arabidopsis thaliana
  source 1..21: mol_type = unassigned RNA
ttgacagaagatagagagcac

SEQ ID NO: 413 - RNA, 20 nt, Arabidopsis thaliana
  source 1..20: mol_type = unassigned RNA
tgacagaagatagagagcac

SEQ ID NO: 414 - RNA, 21 nt, Arabidopsis thaliana
  source 1..21: mol_type = unassigned RNA
tgcctggctccctgtatgcca

SEQ ID NO: 415 - RNA, 21 nt, Arabidopsis thaliana
  source 1..21: mol_type = unassigned RNA
tggagaagcagggcacgtgca

SEQ ID NO: 416 - RNA, 21 nt, Arabidopsis thaliana
  source 1..21: mol_type = unassigned RNA
tcggaccaggcttcattcccc

SEQ ID NO: 417 - RNA, 21 nt, Arabidopsis thaliana
  source 1..21: mol_type = unassigned RNA
tcggaccaggcttcattcccc

SEQ ID NO: 418 - RNA, 21 nt, Arabidopsis thaliana
  source 1..21: mol_type = unassigned RNA
cagccaaggatgacttgccga

SEQ ID NO: 419 - RNA, 21 nt, Arabidopsis thaliana
  source 1..21: mol_type = unassigned RNA
tagccaaggatgacttgcctg

SEQ ID NO: 420 - RNA, 21 nt, Arabidopsis thaliana
  source 1..21: mol_type = unassigned RNA
tagccaaggatgacttgcctg

SEQ ID NO: 421 - RNA, 21 nt, Arabidopsis thaliana
  source 1..21: mol_type = unassigned RNA
tgattgagccgtgtcaatatc

SEQ ID NO: 422 - RNA, 21 nt, Arabidopsis thaliana
  source 1..21: mol_type = unassigned RNA
ttccacagctttcttgaactg

SEQ ID NO: 423 - RNA, 21 nt, Arabidopsis thaliana
  source 1..21: mol_type = unassigned RNA
tgccaaaggagagttgccctg

(19) United States
(12) Patent Application Publication
(10) Pub. No.: US 2024/0301014 A1
(43) Pub. Date: Sep. 12, 2024

(54) MICROPEPTIDES AND USE OF SAME FOR MODULATING GENE EXPRESSION

(71) Applicants: UNIVERSITE PAUL SABATIER TOULOUSE III, Toulouse Cedex 9 (FR); CENTRE NATIONAL DE LA RECHERCHE SCIENTIFIQUE, Paris Cedex 16 (FR)

(72) Inventors: Jean-Philippe COMBIER, Castanet Tolosan (FR); Dominique LAURESSERGUES, Toulouse (FR); Guillaume BECARD, Odars (FR); François PAYRE, Pompertuzat (FR); Serge PLAZA, Ramonville Saint Agne (FR); Jérôme CAVAILLE, Ramonville Saint Agne (FR)

(73) Assignees: UNIVERSITE PAUL SABATIER TOULOUSE III, Toulouse Cedex 9 (FR); CENTRE NATIONAL DE LA RECHERCHE SCIENTIFIQUE, Paris Cedex 16 (FR)

(21) Appl. No.: 18/668,366

(22) Filed: May 20, 2024

Related U.S. Application Data
(63) Continuation of application No. 16/595,926, filed on Oct. 8, 2019, now Pat. No. 12,024,543.
Application No. 16/595,926 is a continuation of application No. 14/320,703, filed on Jul. 1, 2014, now abandoned.

(30) Foreign Application Priority Data
Oct. 31, 2013 (FR) 1360727
Jun. 3, 2014 (FR) 1455044

(51) Int. Cl. C07K 14/415; A01N 37/46; A01N 57/16; A01N 65/00; A01N 65/08; A01N 65/20; A01N 65/28; A01N 65/44; A61K 38/00; A61K 48/00; C07K 5/10; C07K 5/103; C07K 7/06; C07K 7/08; C12N 15/113; C12N 15/63; C12N 15/82; C12Q 1/68; C12Q 1/6895

(52) U.S. Cl. CPC C07K 14/415; A01N 37/46; A01N 57/16; A01N 65/00; A01N 65/08; A01N 65/20; A01N 65/28; A01N 65/44; C07K 5/10; C07K 5/1013; C07K 7/06; C07K 7/08; C12N 15/113; C12N 15/63; C12N 15/8218; C12N 15/8261; C12Q 1/68; C12Q 1/6895; A61K 38/00; A61K 48/00; C12Q 2600/13; C12Q 2600/158; C12Q 2600/178; Y02A 40/146

(57) ABSTRACT

Process for detecting and identifying micropeptides (miPEPs) encoded by a nucleotide sequence contained in the sequence of the primary transcript of a microRNA and use thereof for modulating gene expression.

### RELATED APPLICATIONS

This application is a continuation of U.S. patent application Ser. No. 16/595,926, filed on Oct. 8, 2019, which is a continuation of U.S. patent application Ser. No. 14/320,703, filed on Jul. 1, 2014, which claims priority under 35 U.S.C. § 119, to French Patent Application Nos. 1360727, filed Oct. 31, 2013, and U.S. Pat. No. 1,455,044, filed Jun. 3, 2014, the disclosures of which are incorporated by reference herein in their entireties.

### REFERENCE TO A SEQUENCE LISTING

In accordance with 37 CFR § 1.831, the present specification makes reference to a Sequence Listing submitted electronically as an .xml file named “USCC IPEP2-Sequence Listing.xml”, created on May 17, 2024, which is 425,444 bytes in size. The entire contents of the Sequence Listing are hereby incorporated by reference.

### FIELD OF THE INVENTION

The present invention relates to micropeptides (peptides encoded by microRNAs or “miPEPs”) and use thereof for modulating gene expression.

### BACKGROUND OF THE INVENTION

The microRNAs (miRNAs) are small non-coding RNAs, about 21 nucleotides in length after maturation, which control expression of target genes at the post-transcriptional level, by degrading the target mRNA or by inhibiting its translation. The miRNAs occur in plants and animals.

The target genes are often key genes in developmental processes. For example they encode transcription factors or proteins of the proteasome.

The regulation of expression of the miRNAs is very poorly understood, but it is known in particular that the latter involves, like most coding genes, an RNA polymerase II: this enzyme produces a primary transcript, called “pri-miRNA”, which is then matured by a protein complex in particular containing the Dicer type enzymes. This maturation leads firstly to the formation of a precursor of miRNA called “pre-miRNA”, having a stem-loop secondary structure containing the miRNA and its complementary sequence miRNA*. Then the precursor is matured, which leads to formation of a shorter double-stranded RNA containing the miRNA and the miRNA*. The miRNA is then manipulated by the RISC complex, which cleaves the mRNA of the target gene or inhibits its translation.

Moreover, it has been shown that the presence of introns in the primary transcript of the microRNA increases the expression of the mature microRNA (Schwab et al, EMBO Rep., 14(7): 615-21, 2013). However, owing to experimental difficulties, the primary transcripts of microRNAs, or pri-miRNAs, have received very little study.

About 50% of eukaryotic genes have small open reading frames within their 5′UTR region (5′ UnTranslated Region) upstream of the coding sequence. These small open reading frames (or “uORFs” for upstream ORFs) may perform a role of translation regulator, mainly in cis, by modulating the fixation and the rate of the ribosomes on the mRNA, but also in trans by an as yet unknown mechanism, by means of peptides encoded by said uORFs (Combier et al., Gene Dev, 22: 1549-1559, 2008). By definition, the uORFS are present upstream of coding genes.

Recently, small ORFs have also been discovered in long intergenic non-coding RNAs (lincRNAs), the putative function of which, if it exists, is not known (Ingolia et al., Cell, 147(4): 789-802, 2011; Guttman & Rinn, Nature, 482(7385): 339-46, 2012).

However, no example has yet been reported concerning the existence of ORFs encoding peptides within non-coding microRNAs. Until now, the microRNAs, and by extension their primary transcript, have always been regarded, based on their particular mode of action, as non-coding regulatory RNAs that do not produce any peptide.

### SUMMARY OF THE INVENTION

One of the aspects of the invention is to propose peptides capable of modulating the expression of microRNAs.

Another aspect of the invention is to propose a means for modulating the expression of one or more target genes of a microRNA.

The present invention offers the advantage of allowing easier and more effective control of the expression of genes targeted by the microRNAs, through a means other than the microRNA.

The invention thus relates to a process for detecting and identifying a micropeptide (miPEP) encoded by a nucleotide sequence contained in the sequence of the primary transcript of a microRNA, comprising:
a) a step of detecting an open reading frame from 12 to 303 nucleotides in length contained in the sequence of the primary transcript of said microRNA, then
b) a step of comparison between:
   the accumulation of said microRNA in a specified eukaryotic cell expressing said microRNA,
   in the presence of a peptide encoded by a nucleotide sequence that is identical or degenerate relative to that of said open reading frame, said peptide being present in the cell independently of transcription of the primary transcript of said microRNA, and
   the accumulation of said microRNA in a eukaryotic cell of the same type as the aforesaid specified eukaryotic cell expressing said microRNA,
   in the absence of said peptide,
      in which a modulation of the accumulation of said microRNA in the presence of said peptide relative to the accumulation of said microRNA in the absence of said peptide indicates the existence of a micropeptide encoded by said open reading frame.

The invention relates in particular to a process for detecting and identifying a micropeptide (miPEP) encoded by a nucleotide sequence contained in the sequence of the primary transcript of a microRNA,

comprising:
a) a step of detecting an open reading frame from 15 to 303 nucleotides in length contained in the sequence of the primary transcript of said microRNA, then
b) a step of comparison between:
   the accumulation of said microRNA in a specified eukaryotic cell expressing said microRNA,
   in the presence of a peptide encoded by a nucleotide sequence that is identical or degenerate relative to that of said open reading frame, said peptide being present in the cell independently of transcription of the primary transcript of said microRNA, and
   the accumulation of said microRNA in a eukaryotic cell of the same type as the aforesaid specified eukaryotic cell expressing said microRNA,
   in the absence of said peptide,
      in which a modulation of the accumulation of said microRNA in the presence of said peptide relative to the accumulation of said microRNA in the absence of said peptide indicates the existence of a micropeptide encoded by said open reading frame.

### BRIEF DESCRIPTION OF THE DRAWINGS

The following figures and examples will illustrate the invention better, but without limiting its scope.

FIG. 1. Effects of overexpression of MtmiR171b (miR171b identified in Medicago truncatula) on the expression of the HAM1 and HAM2 genes (A) or on the number of lateral roots (B) in M. truncatula.

(A) The y-axis indicates the relative expression of MtmiR171b (left-hand columns), of HAM1 (middle columns) or of HAM2 (right-hand columns) in a control plant (white columns) or in a plant in which MtmiR171b is overexpressed (black columns). The error bar corresponds to the standard error of the mean (number of individuals=10). The overexpression of MtmiR171b induces a decrease in the expression of the HAM1 and HAM2 genes.

(B) The y-axis indicates the mean number of lateral roots observed in a control plant (white column) or in a plant in which MtmiR171b is overexpressed (black column). The error bar corresponds to the standard error of the mean (number of individuals=100). The overexpression of MtmiR171b leads to a reduction in the number of lateral roots.

FIG. 2. Effects of overexpression of MtmiPEP171b1 on the expression of MtmiR171b and of the HAM1 and HAM2 genes (A) or on the number of lateral roots (B) in M. truncatula.

(A) The y-axis indicates the relative expression of MtmiPEP171b1 (graph on left), miR171b (graph on right, left-hand columns), of HAM1 (accession No. MtGI9-TC114268) (graph on right, middle columns) or of HAM2 (accession No. MtGI9-TC120850) (graph on right, right-hand columns) in a control plant (white columns) or in a plant in which MtmiPEP171b1 is overexpressed (black columns). The error bar corresponds to the standard error of the mean (number of individuals=10). The overexpression of MtmiPEP171b1 induces an increase in the accumulation of MtmiR171b, as well as a decrease in the expression of the HAM1 and HAM2 genes.

(B) The y-axis indicates the mean number of lateral roots observed in a control plant (white column) or in a plant in which MtmiPEP171b1 is overexpressed (black column). The error bar corresponds to the standard error of the mean (number of individuals=100). The overexpression of MtmiPEP171b1 leads to a reduction in the number of lateral roots.

FIG. 3. Effects of MtmiPEP171b1 on the expression of MtmiR171b and the HAM1 and HAM2 genes (A) and on the number of lateral roots (B) in M. truncatula.

(A) The y-axis indicates the relative expression of MtmiR171b (left-hand columns), of HAM1 (middle columns) or of HAM2 (right-hand columns) in a control plant (white columns) or in a plant treated by watering once daily for 5 days with MtmiPEP171b1 at 0.01 μM (light grey columns), 0.1 μM (dark grey columns) or 1 μM (black columns). The error bar corresponds to the standard error of the mean (number of individuals=10). Application of MtmiPEP171b1 at different concentrations induces an increase in the accumulation of MtmiR171b, as well as a decrease in the expression of the HAM1 and HAM2 genes.

(B) The y-axis indicates the mean number of lateral roots observed in a control plant (white column) or in a plant treated by watering with MtmiPEP171b1 at 0.1 μM once daily for 5 days (black column). The error bar corresponds to the standard error of the mean (number of individuals=100). Application of MtmiPEP171b1 at 0.1 μM leads to a reduction in the number of lateral roots.

(C) The y-axis indicates the relative expression of MtmiR171b (left-hand columns), of HAM1 (middle columns) or of HAM2 (right-hand columns) in a control plant (white columns) or in a plant treated by watering once daily for 5 days with MtmiPEP171b1 at 0.01 μM (grey columns), 0.1 μM (dark grey columns) or 1 μM (black columns) or with 0.01 μM of a mixed peptide (light grey columns) the amino acid composition of which is identical to miPEP171b but the sequence of which is different. The error bar corresponds to the standard error of the mean (number of individuals=10).

FIG. 4. Effects of MtmiPEP171b1 on the expression of pre-MtmiR171b (A) and of MtmiR171b (B) in M. truncatula.

The y-axis indicates the relative expression of the precursors of the different forms of the microRNA in control plants (left-hand column) or in plants treated by watering once daily for 5 days with MtmiPEP171b1 at 0.01 μM, 0.1 μM or 1 μM (right-hand columns). The error bar corresponds to the standard error of the mean (number of individuals=200). Application of MtmiPEP171b1 at different concentrations leads to an increase in the accumulation of pre-MtmiR171b (A) and of MtmiR171b (B).

FIG. 5. Effects of overexpression of MtmiPEP171b1 (A) and effects of MtmiPEP171b1 (B) on the expression of different precursors of microRNAs in M. truncatula.

The y-axis indicates the ratio of the expression of the precursors of microRNAs in plants overexpressing MtmiPEP171b1 to the expression of these same precursors in control roots (A), or the ratio of the expression of the precursors of microRNAs in plants treated with MtmiPEP171b1 (0.1 μM) to the expression of these same precursors in control roots (B). The different precursors of microRNAs tested are indicated from left to right on the x-axis, namely pre-MtmiR171b (SEQ ID NO: 246), pre-MtmiR169 (SEQ ID NO: 359), pre-MtmiR169a (SEQ ID NO: 360), pre-MtmiR171a (SEQ ID NO: 361), pre-MtmiR171h (SEQ ID NO: 362), pre-MtmiR393a (SEQ ID NO: 363), pre-MtmiR393b (SEQ ID NO: 364), pre-MtmiR396a (SEQ ID NO: 365) and pre-MtmiR396b (SEQ ID NO: 366). The error bar corresponds to the standard error of the mean (number of individuals=10). It is noted that MtmiPEP171b1 only leads to an effect on the accumulation of MtmiR171b and not on the other miRNAs.

FIG. 6. Effects of translation of MtmiPEP171b1 on the expression of MtmiR171b demonstrated in the model plant Nicotiana benthamiana. The y-axis indicates the relative expression of MtmiR171b in tobacco plants transformed in order to express pri-MtmiR171b (white column) or a mutated pri-MtmiR171b in which the codon ATG has been replaced with ATT (black column). The mutated pri-MtmiR171b is therefore incapable of producing MtmiPEP171b1. The error bar corresponds to the standard error of the mean (number of individuals=30). It is noted that the absence of translation of MtmiPEP171b1 leads to a marked decrease in the accumulation of miR171b.

FIG. 7. Effects of overexpression of MtmiPEP171b1 on the expression of pre-MtmiR171b demonstrated in the model plant Nicotiana benthamiana.

The y-axis indicates the relative expression of pre-MtmiR171b in tobacco plants that have been transformed in order to express MtmiR171b (left-hand column), MtmiR171b and MtmiPEP171b1 (middle column), or MtmiR171b and a mutated version of MtmiORF171b in which the start codon ATG has been replaced with ATT (right-hand column). The error bar corresponds to the standard error of the mean (number of individuals=30). It is noted that the expression of MtmiPEP171b1 increases the expression of MtmiR171b, and this effect is dependent on the translation of MtmiORF171b to MtmiPEP171b1.

FIG. 8. Effects of MtmiPEP171b1 on the expression of pre-MtmiR171b demonstrated in the model plant Nicotiana benthamiana.

The y-axis indicates the relative expression of MtmiR171b in tobacco plants transformed in order to express MtmiR171b onto which MtmiPEP171b1 has been sprayed (0.1 μM) twice, 12 h and then 30 min before sampling (right-hand column) or not (left-hand column). The error bar corresponds to the standard error of the mean (number of individuals=6). The peptide MtmiPEP171b1 applied by spraying induces an increase in the accumulation of MtmiR171b.

FIG. 9. Effects of MtmiPEP171b1 on the expression of pri-miR171b (A), pre-MtmiR171b (B) and MtmiR171b (C) demonstrated in the model plant Nicotiana benthamiana.

The y-axis indicates the relative expression of the precursors of the different forms of the microRNA in tobacco plants modified in order to express MtmiR171b (left-hand column) or modified in order to express MtmiR171b and overexpress MtmiPEP171b1 (right-hand columns, FIG. 9A) or treated with 0.1 μM of miPEP171b1 (FIGS. 9B and C). The error bar corresponds to the standard error of the mean (number of individuals=30). The overexpression of MtmiPEP171b1 or application of miPEP171b1 increases the accumulation of pri-MtmiR171b (A), pre-MtmiR171b (B) and MtmiR171b (C).

FIG. 10. Localization of MtmiPEP171b1 in tobacco leaf cells that have been modified in order to express MtmiPEP171b1.

The photographs show tobacco leaf cells modified in order to express the protein GFP alone (left panel) or the protein GFP fused to MtmiPEP171b1 (right panel). These observations indicate that MtmiPEP171b1 is localized in small nuclear bodies.

FIG. 11. Effects of the expression of AtmiPEP165a (identified in Arabidopsis thaliana) on the expression of AtmiR165a (A), and of the expression of AtmiPEP319a2 (identified in Arabidopsis thaliana) on AtmiR319a (B), demonstrated in the model plant of tobacco.

(A) The y-axis indicates the relative expression of AtmiR165a in tobacco plants modified in order to express AtmiR165a (left-hand column) or to express AtmiR165a and AtmiPEP165a (right-hand column).

(B) The y-axis indicates the relative expression of AtmiR319a in tobacco plants modified in order to express AtmiR319a (left-hand column) or in order to express AtmiR319a and AtmiPEP319a (right-hand column).

The error bar corresponds to the standard error of the mean (number of individuals=30).

In both cases, it is noted that the expression of miORF, and therefore the production of miPEP, leads to an increase in the accumulation of pre-miRNA.

FIG. 12. Effects of treatment with AtmiPEP165a on root growth in Arabidopsis thaliana.

The photograph shows two plants of the same age: a control plant (plant on the left) and a plant treated with AtmiPEP165a (plant on the right). The treatment with AtmiPEP165a leads to a phenotype with greatly accelerated root growth in Arabidopsis thaliana. The graph shows the expression of pre-miR165 in response to treatment with increasing doses of AtmiPEP165a.

FIG. 13. Conservation of the sequence of miPEP8 identified in Drosophila.

The sequences of miPEP8 (SEQ ID NO: 104) were deduced from the sequences of miORF8 (SEQ ID NO: 208) of 12 different Drosophila species and were aligned. A histogram shows the conservation of each amino acid between the sequences of miORF8 in the 12 species analysed.

FIG. 14. Evolution of the mass (kDa) and isoelectric point (pI) of miPEP8 in the Drosophila species.

The y-axis on the left indicates the size of the miPEP8 (in kD). The y-axis on the right indicates the isoelectric point of the miPEP. The x-axis indicates the origin of the miPEP, i.e. the Drosophila species. It is noted that despite a significant change in their size (by more than a factor of 3), the charge of the miPEPs is still very basic (>9.8) in the 12 species studied.

FIG. 15. Effect of the addition of sequences on the function of miPEP.

The tobacco leaves were transformed in order to overexpress miPEP171b. These graphs show that the addition of sequences (tag His, HA or GFP) does not alter the function of miPEP. The y-axis indicates the relative expression of pre-MtmiR171b in tobacco plants that have been transformed in order to express MtmiR171b (left-hand column), MtmiR171b and MtmiPEP171b1 with or without addition of protein tags (right-hand columns). The error bar corresponds to the standard error of the mean (number of individuals=6). It is noted that the expression of MtmiPEP171b1 increases the expression of MtmiR171b, and this effect is independent of the presence of tags.

FIG. 16. Expression of MtmiPEP171b1 in the root system of Medicago truncatula.

The roots of Medicago truncatula were transformed in order to express fusions between GUS protein (in blue) and the promoter of miR17b (A, E), the ATG of miPEP171b1 (B, F), whole miPEP171b1 (C, G) or ATG2 (second ATG located on the precursor, after miPEP) (D, H). It is clear that there is expression of miRNA in the root tips (A) as well as the lateral roots (E). The transcriptional (B, F) and translational (C, G) fusions show an expression of miPEP171b in the same tissues, whereas the next ATG is not active (D, H).

FIG. 17. Expression of DmmiPEP8 in cells of Drosophila melanogaster

The cells of Drosphila melanogaster were transfected in order to overexpress DmmiPEP8 (OE miPEP8) or miPEP8 of which the translation start codons have been mutated (OE miPEP8 mut). The y-axis indicates the relative expression of Pre-miR8. The error bar corresponds to the standard error of the mean (number of independent experiments=6). It is noted that the expression of DmmiPEP8 increases the expression of DmmiR8, and this effect is linked to the translation of the mRNA.

FIG. 18. Impact of DmmiPEP8 on accumulation of DmmiR8 in cells of D. melanogaster

The cells of Drosphila melanogaster were transfected in order to overexpress wild-type DmmiR8 (OE miR8) or DmmiR8 the translation start codons of which have been mutated (OE miR8 miPEP8 mut). The y-axis indicates the relative expression of Pre-miR8. The error bar corresponds to the standard error of the mean (number of independent experiments=2). It is noted that the presence of DmmiPEP8 increases the expression of DmmiR8.

FIG. 19. Impact of HsmiPEP155 on accumulation of HsmiR155 in cells of Homo sapiens

HeLa cells of Homo sapiens had been transfected in order to overexpress HsmiPEP155 (OE miPEP155). The y-axis indicates the relative expression of Pre-miR155. The error bar corresponds to the standard error of the mean (number of independent experiments=2). It is noted that the expression of HsmiPEP155 increases the expression of HsmiR155.

FIG. 20. Effects of translation of MtmiPEP171b1 on the expression of MtmiR171b demonstrated in the model plant Nicotiana benthamiana.

The y-axis indicates the relative expression of MtmiR171b in tobacco plants transformed in order to express pri-miR171b (left-hand column), a pri-miR171b in which the miORF171b has been deleted (middle column) or a mutated pri-miR171b in which the codon ATG has been replaced with ATT (right-hand column). The mutated pri-miR171b is therefore incapable of producing miPEP171b1. The error bar corresponds to the standard error of the mean (number of individuals=30). It is noted that the absence of translation of miPEP171b1 leads to a marked decrease in the accumulation of miR171b.

FIG. 21. Effects of overexpression of MtmiPEP171b1 on the expression of MtmiR171b demonstrated in the model plant Nicotiana benthamiana.

The y-axis indicates the relative expression of MtmiR171b in tobacco plants that had been transformed with a vector allowing the expression of miPEP171b and either a second empty vector (white column), or a vector allowing the expression of mtmiPEP171b (left black column), or a vector in which the codon ATG of the ORF encoding mtmiPEP171b has been replaced with ATT (middle black column), or a vector in which the nucleotide sequence of the ORF has been mutated without modifying the amino acid sequence of the translated peptide (miPEP encoded by a degenerate ORF) (right black column). The error bar corresponds to the standard error of the mean (number of individuals=30). It is noted that the expression of MtmiPEP171b1 increases the expression of MtmiR171b, and this effect is dependent on the translation of MtmiORF171b to MtmiPEP171b1.

FIG. 22. Effects of AtmiPEP165a on accumulation of AtmiR165a and of its target genes (PHAVOLUTA: PHV, PHABOLUSA: PHB and REVOLUTA: REV).

The y-axis indicates the relative expression of AtmiR165a, PHV, PHB and REV in roots of Arabidopsis thaliana treated with water (control) or different concentrations of AtmiPEP165a (0.01 μM, 0.1 μM, 1 μM or 10 μM). The error bar corresponds to the standard error of the mean (number of individuals=10).

The treatment of plants with higher and higher concentrations of AtmiPEP165a demonstrates a dose-dependent effect of the accumulation of AtmiR165a and the negative regulation of its target genes as a function of the quantity of AtmiPEP165a.

FIG. 23. Effects of treatment with AtmiPEP164a on the expression of AtmiR164a in A. thaliana.

The photographs show the results of a Northern blot analysis of the accumulation of AtmiR164a in roots treated with water (control, photograph on left) or with 0.1 μM of a synthetic peptide, having a sequence identical to that of AtmiPEP164a, dissolved in water (0.1 μM miPEP164a). The RNA U6 is used as loading control making it possible to quantify the quantity of AtmiR164a.

This experiment was repeated 4 times independently and led to similar results.

Treatment of shoots of A. thaliana with 0.1 μM of miPEP164a leads to an increase in the accumulation of miR164a.

FIG. 24. Effects of treatment with AtmiPEP164a on the growth of Arabidopsis thaliana.

The photographs show two plants (top views and side views) after 3 weeks of growth: a control plant watered with water (A), and a plant watered with a composition of 0.1 μM of synthetic peptide corresponding to AtmiPEP164a (B). Watering plants of Arabidopsis thaliana with AtmiPEP164a increases plant growth significantly.

FIG. 25. Effects of treatment with AtmiPEP165a on the expression of AtmiR165a in A. thaliana.

The photographs show the results of a Northern blot analysis of the accumulation of AtmiR165a in roots treated with water (control, photograph on left) or with 0.1 μM of a synthetic peptide, having a sequence identical to that of AtmiPEP165a, dissolved in water (0.1 μM miPEP165a). The RNA U6 is used as loading control making it possible to quantify the quantity of AtmiR165a.

This experiment was repeated 4 times independently and led to similar results.

Treatment of A. thaliana shoots with 0.1 μM of miPEP165a leads to an increase in the accumulation of miR165a.

FIG. 26. Effects of overexpression of AtmiPEP319a1 on the expression of AtmiR319a in A. thaliana.

The y-axis indicates the relative expression of AtmiR319a in a control plant (left-hand column) or in a plant in which AtmiPEP319a1 is overexpressed (right-hand column). The error bar corresponds to the standard error of the mean (number of individuals=10). The overexpression of AtmiPEP319a1 induces an increase in the accumulation of AtmiR319a.

FIG. 27. Effects of treatment with AtmiPEP319a on the growth of Arabidopsis thaliana.

The photographs show two plants (top views and side views) after 3 weeks of growth: a control plant watered with water (A), and a plant watered with a composition of 0.1 μM of synthetic peptide corresponding to AtmiPEP319a1 (B). Watering of the plants of Arabidopsis thaliana with AtmiPEP319a1 increases plant growth significantly.

FIG. 28. Immunolocalization.

The roots of Medicago truncatula were transformed in order to express fusions between the GUS protein (in blue) and the ATG of miPEP171b (PromiR171b-ATG1:GUS) or ATG2 (second ATG located on the precursor, after miPEP) (PromiR171b-ATG2:GUS). Labelling was also carried out with an anti-miPEP171b antibody (miPEP171b). Immunolocalization of miPEP171b in the roots of M. truncatula reveals the presence of miPEP171b in the lateral root initiation sites, showing a co-localization between the microRNA and the corresponding miPEP.

### DETAILED DESCRIPTION OF THE INVENTION

In a first step, the process for detecting and identifying a micropeptide therefore consists of detecting, on the primary transcript of a microRNA, the existence of an open reading frame potentially encoding a peptide.

For its part, the second step makes it possible to characterize said peptide, i.e. to determine whether said peptide corresponds to a peptide really produced in the cell, by searching for an effect of said peptide on the accumulation of said microRNA.

In order to demonstrate an effect of the peptide on the accumulation of the microRNA, a large quantity of peptide is introduced into a first cell expressing said microRNA. The accumulation of the microRNA in this first cell is then measured and compared with the accumulation of the microRNA in a second cell identical to the first, but not containing said peptide.

Observation of a variation of the quantities of microRNA between the cells in the presence and in the absence of the peptide thus indicates (i) that there is a peptide encoded on the primary transcript of said microRNA, (ii) that the sequence of this peptide is encoded by the open reading frame identified on the primary transcript of said microRNA, and (iii) that said peptide has an effect on the accumulation of said microRNA.

The invention is therefore based on the unexpected double observation made by the inventors that, on the one hand, there are open reading frames that are able to encode micropeptides present on the primary transcripts of microRNAs, and on the other hand that said micropeptides are capable of modulating the accumulation of said microRNAs.

In particular, the invention relates to a process for detecting and identifying a micropeptide (miPEP) encoded by a nucleotide sequence contained in the sequence of the primary transcript of a microRNA, comprising:
a) a step of detecting an open reading frame from 15 to 303 nucleotides in length contained in the sequence of the primary transcript of said microRNA, then
b) a step of comparison between:
   the accumulation of said microRNA in a specified eukaryotic cell expressing the primary transcript of said microRNA,
   in the presence of a peptide encoded by a nucleotide sequence that is identical or degenerate relative to that of said open reading frame, said peptide being present in the cell independently of transcription of the primary transcript of said microRNA, and
   the accumulation of said microRNA in a eukaryotic cell of the same type as the aforesaid specified eukaryotic cell expressing the primary transcript of said microRNA,
   in the absence of said peptide,
      in which a modulation of the accumulation of said microRNA in the presence of said peptide relative to the accumulation of said microRNA in the absence of said peptide indicates the existence of a micropeptide encoded by said open reading frame.

In the invention, the terms “microRNA”, “non-coding microRNA” and “miRNA” are equivalent and may be used interchangeably. They define small molecules of RNA of about 21 nucleotides, which are not translated and do not lead to a peptide or a protein.

However, in this mature form, the microRNAs perform a function of regulation of certain genes via post-transcriptional mechanisms, for example by means of the RISC complex.

The primary transcript of the microRNA or “pri-miRNA” corresponds to the RNA molecule obtained directly from transcription of the DNA molecule. Generally, this primary transcript undergoes one or more post-transcriptional modifications, involving for example a particular structure of the RNA or cleavage of certain portions of the RNA by splicing phenomena, and which lead to the precursor form of the microRNA or “pre-miRNA”, then to the mature form of the microRNA or “miRNA”.

The terms “micropeptides” and “miPEPs” (microRNA encoded PEPtides) are equivalent and may be used interchangeably. They define a peptide that is encoded by an open reading frame present on the primary transcript of a microRNA, and which is capable of modulating the accumulation of said microRNA.

In view of the above definitions, it is important to distinguish on one side, the miRNA which does not encode any peptide and on the other side, the primary transcript of such a miRNA which may encode a miPEP.

This distinction derives from the teaching of the invention and is original in view of the current knowledge about miRNAs.

The micropeptides within the meaning of the present invention are not to be understood as necessarily being small peptides, as “micro” does not correspond to the size of the peptide.

Taking into account the degeneracy of the genetic code, one and the same micropeptide is or may be encoded by several nucleotide sequences. Nucleotide sequences of this kind, differing from one another by at least one nucleotide but encoding one and the same peptide, are called “degenerate sequences”.

The terms “open reading frame” or “ORF” are equivalent and may be used interchangeably. They correspond to a nucleotide sequence in a DNA or RNA molecule that may potentially encode a peptide or a protein: said open reading frame begins with a start codon (the start codon generally encoding a methionine), followed by a series of codons (each codon encoding an amino acid), and ends with a stop codon (the stop codon not being translated).

In the invention, the ORFs may be called specifically “miORFs” when they are present on the primary transcripts of microRNA.

The miORFs as defined in the particular invention may have a size from 12 to 303 nucleotides and may encode peptides from 3 to 100 amino acids.

In particular, the miORFs as defined in the invention may have a size from 15 to 303 nucleotides. As an amino acid is encoded by a codon of 3 nucleotides, the miORFs from 15 to 303 nucleotides encode miPEPS from 4 to 100 amino acids.

In particular, the miORFs have a size of:
15, 18, 21, 24, 27, 30, 33, 36, 39, 42, 45, 47, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300 or 303 nucleotides, and encode respectively miPEPs having a size of:
4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 amino acids.

In the invention, “accumulation” means the production of a molecule, such as a microRNA or a micropeptide, in the cell.

Thus, “modulation” of the accumulation of a molecule in a cell corresponds to a modification of the quantity of this molecule present in the cell.

In an embodiment, the invention relates to a process for detecting and identifying a miPEP as defined above, in which the modulation of the accumulation of said microRNA is a decrease or an increase in the accumulation of said microRNA, in particular an increase.

A “decrease in the accumulation” corresponds to a decrease in the quantity of said molecule in the cell.

Conversely, an “increase in the accumulation” corresponds to an increase in the quantity of said molecule in the cell.

In an advantageous embodiment, the invention relates to a process for detecting and identifying a miPEP as defined above, in which the modulation of the accumulation of said microRNA is an increase in the accumulation of said microRNA.

In an embodiment, the invention relates to a process for detecting and identifying a miPEP as defined above, in which the presence of said peptide in the cell results from:
the introduction of a nucleic acid encoding said peptide into the cell, or
the introduction of said peptide into the cell.

In order to characterize a miPEP, it is necessary to have a cellular model expressing a microRNA in which said peptide to be tested is present. For this, it is possible to introduce a peptide into the cell, either by bringing the cell into contact with said peptide, or by introducing a nucleic acid encoding said peptide into the cell, and this nucleic acid will then be translated into peptide within the cell.

In an embodiment, the invention relates to a process for detecting and identifying a miPEP as defined above, in which said open reading frame in step a) is contained in the 5′ or 3′ portion of said primary transcript of the microRNA, preferably in the 5′ portion.

The 5′ or 3′ portions of the primary transcript of the microRNA correspond to the terminal portions of the RNA molecule that are cleaved during maturation of the microRNA.

In an embodiment, the invention relates to a process for detecting and identifying a miPEP as defined above, in which said microRNA is present in a wild-type plant cell.

In the invention, a wild-type plant cell corresponds to a plant cell that has not been genetically modified by humans.

In an embodiment, the invention relates to a process for detecting and identifying a miPEP as defined above, in which said microRNA is present in a wild-type animal cell, and in particular a wild-type human cell or a wild-type Drosophila cell.

In the invention, a wild-type animal cell corresponds to an animal cell, and in particular a human cell, that has not been modified genetically by humans.

In an embodiment, the invention relates to a process for detecting and identifying a miPEP as defined above, in which said specified eukaryotic cell and said eukaryotic cell of the same type as the aforesaid specified eukaryotic cell, used in step b, are plant cells of a cruciferous plant such as Arabidopsis thaliana, of a leguminous plant such as Glycine max (soya), Medicago truncatula and Medicago sativa (alfalfa) or of a plant of the Solanaceae family such as Nicotiana benthamiana (tobacco), Solanum tuberosum (potato), Solanum lycopersicum (tomato) or Solanum melongena (aubergine).

In an embodiment, the invention relates to a process for detecting and identifying a miPEP as defined above, in which said specified eukaryotic cell and said eukaryotic cell of the same type as the aforesaid specified eukaryotic cell, used in step b, are plant cells, preferably cells of Medicago truncatula, Nicotiana benthamiana or Arabidopsis thaliana.

In an embodiment, the invention relates to a process for detecting and identifying a miPEP as defined above, in which said specified eukaryotic cell and said eukaryotic cell of the same type as the aforesaid specified eukaryotic cell, used in step b, are animal cells, preferably human cells or Drosophila cells.

In the process for detecting and identifying a micropeptide as defined above, after identifying an ORF that is able to encode a peptide on the primary transcript of a microRNA, it is necessary to have a cellular model having said microRNA and said peptide, so as to be able to demonstrate a possible effect of the peptide on said microRNA.

Two options are therefore conceivable:
the cellular model in which the miORF has been identified and that in which the effect of the peptide on the miRNA has been demonstrated are identical, or
the cellular model in which the miORF has been identified and that in which the effect of the peptide on the miRNA has been demonstrated are different.

In the first option, the cellular model used for observing an effect of the peptide is the same as that in which the primary transcript of said microRNA was isolated. In this cellular model, the specified eukaryotic cells contain said microRNA naturally and only the peptide to be tested has to be introduced into these cells. In this context, said microRNA is qualified as “of endogenous origin” as it exists naturally in the cells. Nevertheless, other copies of a microRNA of endogenous origin may be added to a cell, for example by introducing a vector encoding said microRNA of endogenous origin into the cell.

In the second option, the cellular model used for observing an effect of the peptide is different from that in which the primary transcript of said microRNA was isolated. In this cellular model, the specified eukaryotic cells contain neither the microRNA, nor the peptide to be tested. These two elements must therefore be introduced into these cells. In this context, said microRNA is qualified as “of exogenous origin” as it does not exist naturally in the cells.

In an embodiment, the invention relates to a process for detecting and identifying a miPEP as defined above, in which said microRNA is of endogenous origin in said eukaryotic cell and in said eukaryotic cell of the same type as the aforesaid specified eukaryotic cell, used in step b).

In an embodiment, the invention relates to a process for detecting and identifying a miPEP as defined above in which said microRNA is of exogenous origin in said eukaryotic cell and in said eukaryotic cell of the same type as the aforesaid specified eukaryotic cell, used in step b), said eukaryotic cells containing a vector allowing the expression of said microRNA.

In an embodiment, the invention relates to a process for detecting and identifying a miPEP as defined above, in which the accumulation of said microRNA is determined using quantitative RT-PCR or Northern blot.

In an embodiment, the invention relates to a process for detecting and identifying a miPEP as defined above, in which the accumulation of said microRNA is determined using a DNA or RNA chip.

The accumulation of said microRNA may be determined using the techniques of molecular biology for assaying specific nucleic acid molecules.

In another aspect, the invention also relates to a process for detecting and identifying a microRNA in which the sequence of the primary transcript contains a nucleotide sequence encoding a miPEP, comprising:
a) a step of detecting an open reading frame from 15 to 303 nucleotides in length contained in the sequence of the primary transcript of said microRNA, then
b) a step of comparison between:
   the accumulation of said microRNA in a specified eukaryotic cell expressing said microRNA,
   in the presence of a peptide encoded by a nucleotide sequence that is identical or degenerate relative to that of said open reading frame, said peptide being present in the cell independently of transcription of the primary transcript of said microRNA, and
   the accumulation of said microRNA in a eukaryotic cell, of the same type as the aforesaid specified eukaryotic cell expressing said microRNA,
   in the absence of said peptide,
      in which a modulation of the accumulation of said microRNA in the presence of said peptide relative to the accumulation of said microRNA in the absence of said peptide indicates the existence of a microRNA the primary transcript of which contains a nucleotide sequence encoding a micropeptide.

In particular, the invention relates to a process for detecting and identifying a microRNA in which the sequence of the primary transcript contains a nucleotide sequence encoding a miPEP, comprising:
a) a step of detecting an open reading frame from 15 to 303 nucleotides in length contained in the sequence of the primary transcript of said microRNA, then
b) a step of comparison between:
   the accumulation of said microRNA in a specified eukaryotic cell expressing the primary transcript of said microRNA,
   in the presence of a peptide encoded by a nucleotide sequence that is identical or degenerate relative to that of said open reading frame, said peptide being present in the cell independently of transcription of the primary transcript of said microRNA, and
   the accumulation of said microRNA in a eukaryotic cell, of the same type as the aforesaid specified eukaryotic cell expressing the primary transcript of said microRNA,
   in the absence of said peptide,
      in which a modulation of the accumulation of said microRNA in the presence of said peptide relative to the accumulation of said microRNA in the absence of said peptide indicates the existence of a microRNA the primary transcript of which contains a nucleotide sequence encoding a micropeptide.

In an embodiment, the invention relates to a process for detecting and identifying a microRNA as defined above, in which the modulation of the accumulation of said microRNA is a decrease or an increase in the accumulation of said microRNA, in particular an increase.

In an embodiment, the invention relates to a process for detecting and identifying a microRNA as defined above, in which the presence of said peptide in the cell results from:
the introduction of a nucleic acid encoding said peptide into the cell, or
the introduction of said peptide into the cell.

In an embodiment, the invention relates to a process for detecting and identifying a microRNA as defined above, in which said open reading frame in step a) is contained in the 5′ or 3′ portion of said primary transcript of the microRNA, preferably in the 5′ portion.

In an embodiment, the invention relates to a process for detecting and identifying a microRNA as defined above, in which said microRNA is present in a wild-type plant cell.

In an embodiment, the invention relates to a process for detecting and identifying a microRNA as defined above, in which said microRNA is present in a wild-type animal cell, and in particular a wild-type human cell.

In an embodiment, the invention relates to a process for detecting and identifying a microRNA as defined above, in which said eukaryotic cell, and said eukaryotic cell of the same type as the aforesaid specified eukaryotic cell, used in step b) are plant cells, preferably cells of Medicago truncatula.

In an embodiment, the invention relates to a process for detecting and identifying a microRNA as defined above, in which said eukaryotic cell, and said eukaryotic cell of the same type as the aforesaid specified eukaryotic cell, used in step b) are animal cells, preferably Drosophila cells.

In an embodiment, the invention relates to a process for detecting and identifying a microRNA as defined above, in which said microRNA is of endogenous origin in said eukaryotic cell and in said eukaryotic cell of the same type as the aforesaid specified eukaryotic cell, used in step b).

In an embodiment, the invention relates to a process for detecting and identifying a microRNA as defined above in which said microRNA is of exogenous origin in said eukaryotic cell and in said eukaryotic cell of the same type as the aforesaid specified eukaryotic cell, used in step b), said eukaryotic cells containing a vector allowing the expression of said microRNA.

In an embodiment, the invention relates to a process for detecting and identifying a microRNA as defined above, in which the accumulation of said microRNA is determined using quantitative RT-PCR or Northern blot.

In an embodiment, the invention relates to a process for detecting and identifying a microRNA as defined above, in which the accumulation of said microRNA is determined using a DNA or RNA chip.

In another aspect, the invention relates to a miPEP as obtained by implementing the process as defined above.

More particularly, the invention relates to a miPEP encoded by a nucleotide sequence as obtained by implementing the process as defined above. In other words, the invention relates to a miPEP encoded by a nucleotide sequence detected and identified by implementing the process as defined above.

Another aspect of the invention also relates to a miPEP of 3 to 100 amino acids, in particular of 4 to 100 amino acids, in particular of 4 to 60 amino acids, preferably of 4 to 40 amino acids, encoded by a nucleotide sequence contained in the primary transcript of a microRNA, said miPEP being capable of modulating the accumulation of said microRNA in a eukaryotic cell.

In particular, the miPEP as defined in the invention is encoded by a miORF of 15 to 303 nucleotides and has a size of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 amino acids, in particular 5, 8, 10, 18, 19, 23, 37, 50 or 59 amino acids.

In particular, the miPEP of the invention has a size in the range from 4 to 10 amino acids, 4 to 20 amino acids, 4 to 30 amino acids, 4 to 40 amino acids, 4 to 50 amino acids, 4 to 60 amino acids, 4 to 70 amino acids, 4 to 80 amino acids, 4 to 90 amino acids, or 4 to 100 amino acids.

Moreover, it should be noted that several miORFS may be identified on the primary transcript of a microRNA, indicating that a primary transcript of microRNA may potentially encode several miPEPs.

It should also be noted that the effect of a miPEP is generally specific to a single microRNA, namely that resulting from the primary transcript encoding said miPEP.

The modulation of the microRNA by said miPEP may be demonstrated after observing a variation in quantities of microRNA between the cells in the presence and in the absence of the miPEP.

In an embodiment, the invention relates to a miPEP as defined above, said nucleotide sequence being contained in the 5′ or 3′ portion of said primary transcript of a microRNA, preferably in the 5′ portion.

In an embodiment, the invention relates to a miPEP as defined above, said nucleotide sequence corresponding to the first open reading frame present on said primary transcript of a microRNA.

In an embodiment, the invention relates to a miPEP as defined above, said miPEP having a basic isoelectric point, preferably above 8.

In an embodiment, the invention relates to a miPEP as defined above, said miPEP having an acidic isoelectric point.

In an embodiment, the invention relates to a miPEP as defined above, said miPEP being selected from the group of peptides consisting of SEQ ID NO: 1 to SEQ ID NO: 104, SEQ ID NO: 375 to SEQ ID NO: 386, and SEQ ID NO: 355 (Table 1).

In an embodiment, the invention relates to a miPEP as defined above, consisting of the amino acid sequence MVT.

In another aspect, the invention relates to a nucleic acid molecule encoding a miPEP as defined above.

In an embodiment, the invention relates to a nucleic acid molecule as defined above, said molecule being selected from the group of nucleic acids consisting of SEQ ID NO: 105 to SEQ ID NO 208, SEQ ID NO: 387 to SEQ ID NO: 399 and SEQ ID NO: 356 (Table 2).

In a particular embodiment, the invention relates to MtmiPEP171b1 (SEQ ID NO: 59) encoded by the nucleotide sequence (SEQ ID NO: 163) contained in the primary transcript of miR171b (SEQ ID NO: 319), said MtmiPEP171b1 being capable of modulating the accumulation of said miR171b in a eukaryotic cell.

In a particular embodiment, the invention relates to AtmiPEP164a1 (SEQ ID NO: 24) encoded by the nucleotide sequence (SEQ ID NO: 128) contained in the primary transcript of miR164a (SEQ ID NO: 297), said AtmiPEP164a1 being capable of modulating the accumulation of said miR164a in a eukaryotic cell.

In a particular embodiment, the invention relates to AtmiPEP165a (SEQ ID NO: 43) encoded by the nucleotide sequence (SEQ ID NO: 147) contained in the primary transcript of miR165a (SEQ ID NO: 305), said miPEP165a being capable of modulating the accumulation of said miR165a in a eukaryotic cell.

In a particular embodiment, the invention relates to AtmiPEP319a1 (SEQ ID NO: 76) encoded by the nucleotide sequence (SEQ ID NO: 180) contained in the primary transcript of miR319a (SEQ ID NO: 331), said AtmiPEP319a1 being capable of modulating the accumulation of said miR319a in a eukaryotic cell.

In a particular embodiment, the invention relates to AtmiPEP319a2 (SEQ ID NO: 77) encoded by the nucleotide sequence (SEQ ID NO: 181) contained in the primary transcript of miR319a (SEQ ID NO: 331), said AtmiPEP319a2 being capable of modulating the accumulation of said miR319a in a eukaryotic cell.

In a particular embodiment, the invention relates to DmmiPEP1a (SEQ ID NO: 102) encoded by the nucleotide sequence (SEQ ID NO: 206) contained in the primary transcript of miR1 (SEQ ID NO: 353), said dmmiPEP1a being capable of modulating the accumulation of said miR1 in a eukaryotic cell.

In a particular embodiment, the invention relates to DmmiPEP1b (SEQ ID NO: 103) encoded by the nucleotide sequence (SEQ ID NO: 207) contained in the primary transcript of miR1 (SEQ ID NO: 353), said dmmiPEP1b being capable of modulating the accumulation of said miR1 in a eukaryotic cell.

In a particular embodiment, the invention relates to dmmiPEP8 (SEQ ID NO: 104) encoded by the nucleotide sequence (SEQ ID NO: 208) contained in the primary transcript of miR8 (SEQ ID NO: 354), said dmmiPEP8 being capable of modulating the accumulation of said miR8 in a eukaryotic cell.

In a particular embodiment, the invention relates to HsmiPEP155 (SEQ ID NO: 355) encoded by the nucleotide sequence (SEQ ID NO: 356) contained in the primary transcript of miR155 (SEQ ID NO: 358), said HsmiPEP155 being capable of modulating the accumulation of said miR155 in a eukaryotic cell.

In another aspect, the invention relates to an isolated peptide, or an isolated and purified peptide, or a synthetic peptide or a recombinant peptide, comprising or consisting of a sequence identical to that of a miPEP, said miPEP in particular being present naturally in a plant, or in an animal, such as humans.

In another aspect, the invention relates to a vector comprising at least one nucleic acid molecule as defined above.

In another aspect, the invention also relates to the use of at least one:
miPEP as defined above,
nucleic acid encoding said miPEP, or
vector containing said nucleic acid,
   for modulating the expression of at least one gene in a specified eukaryotic cell,
   said specified eukaryotic cell being capable of expressing a microRNA, the primary transcript of which contains at least one nucleotide sequence encoding said at least one miPEP and the accumulation of which is modulated by said at least one miPEP,
   the expression of said at least one gene being regulated by said microRNA.

In another aspect, the invention also relates to the use of at least one:
miPEP of 4 to 100 amino acids, preferably of 4 to 40 amino acids, encoded by a nucleotide sequence contained in the primary transcript of a microRNA, said miPEP being capable of modulating the accumulation of said microRNA in a eukaryotic cell,
nucleic acid encoding said miPEP, or
vector containing said nucleic acid,
   for modulating the expression of at least one gene in a specified eukaryotic cell,
   said specified eukaryotic cell being capable of expressing a microRNA, the primary transcript of which contains at least one nucleotide sequence encoding said at least one miPEP and the accumulation of which is modulated by said at least one miPEP,
   the expression of said at least one gene being regulated by said microRNA.

The invention is based on the surprising observation made by the inventors that it is possible to modulate the expression of one or more target genes of one and the same microRNA by modulating the accumulation of said microRNA using a miPEP.

In an embodiment, the invention relates to the use as defined above in which said specified eukaryotic cell is a plant cell.

In an embodiment, the invention relates to the use as defined above in which said specified eukaryotic cell is a plant cell of a crucifer such as Arabidopsis thaliana, of a leguminous plant such as Glycine max (soya), Medicago truncatula and Medicago sativa (alfalfa) or of a plant of the Solanaceae family such as Nicotiana benthamiana (tobacco), Solanum tuberosum (potato), Solanum lycopersicum (tomato) or Solanum melongena (aubergine).

In an embodiment, the invention relates to the use as defined above in which said specified eukaryotic cell is an animal cell, in particular human.

In an embodiment, the invention relates to the use as defined above in which said specified eukaryotic cell is an animal cell, in particular human, said miPEP not being used for surgical or therapeutic treatment of the human body or animal body, nor for modifying the genetic identity of a human being.

In an embodiment, the invention relates to the use as defined above in which said specified eukaryotic cell is an animal cell, said miPEP being used for surgical or therapeutic treatment of the human body or animal body.

In an embodiment, the invention relates to the use as defined above in which said microRNA and said gene are of endogenous origin in said specified eukaryotic cell.

In an embodiment, the invention relates to the use as defined above in which said microRNA and said gene are of exogenous origin in said specified eukaryotic cell, said specified eukaryotic cell containing at least one vector allowing the expression of said microRNA and of said gene.

In the invention, the expressions “of endogenous origin” and “of exogenous origin” are used for distinguishing said microRNAs and/or the genes of different species, in view of the conservation of the sequences between species.

Thus, the term “of endogenous origin” indicates that the microRNA and/or gene may be present naturally in the cell in question. Other copies of the microRNA and/or of the gene of endogenous origin may nevertheless be added artificially to the cell in question, for example by cloning.

Conversely, the term “of exogenous origin” indicates that the microRNA and/or the gene are never present naturally in the cell in question. It is a microRNA and/or a gene identified in another cellular type or in an organism of another species; this microRNA and/or this gene are therefore necessarily introduced artificially into the cell in question.

In the invention, a genetically transformed cell may therefore contain 2 groups of microRNAs and/or of genes potentially similar in terms of sequence, one of endogenous origin and the other of exogenous origin.

In an embodiment, the invention relates to the use as defined above in which the primary transcript of the miRNA and said gene are of exogenous origin in said specified eukaryotic cell, said specified eukaryotic cell containing at least one vector allowing the expression of the primary transcript of the microRNA.

In an embodiment, the invention relates to the use as defined above in which the primary transcript of the miRNA is encoded by a vector introduced into the cell artificially.

In an embodiment, the invention relates to the use as defined above in which said miPEP is selected from the group of peptides consisting of SEQ ID NO: 1 to SEQ ID NO: 104, SEQ ID NO: 375 to SEQ ID NO: 386 and SEQ ID NO: 355 (Table 1).

In an embodiment, the invention relates to the use as defined above in which said miPEP is selected from MtmiPEP171b1 (SEQ ID NO: 59), AtmiPEP164a1 (SEQ ID NO: 24), AtmiPEP165a (SEQ ID NO: 43), AtmiPEP319a1 (SEQ ID NO: 76) and AtmiPEP319a2 (SEQ ID NO: 77).

In an embodiment, the invention relates to the use as defined above in which said miPEP is selected from DmmiPEP1a (SEQ ID NO: 102), DmmiPEP1b (SEQ ID NO: 103) and DmmiPEP8 (SEQ ID NO: 104).

In an embodiment, the invention relates to the use as defined above in which said miPEP is HsmiPEP155a (SEQ ID NO: 355).

In an embodiment, the invention relates to the use as defined above in which said nucleic acid is selected from the group of nucleic acids consisting of SEQ ID NO: 105 to SEQ ID NO: 208 and SEQ ID NO: 356 (Table 2).

In an embodiment, the invention relates to the use as defined above in which said nucleic acid is selected from miORF171b (SEQ ID NO: 163), miORF164a1 (SEQ ID NO: 128), miORF165a (SEQ ID NO: 147), miORF319a1 (SEQ ID NO: 180) and miORF319a2 (SEQ ID NO: 181).

In an embodiment, the invention relates to the use as defined above in which said nucleic acid is selected from miORF1a (SEQ ID NO: 206), miORF1b (SEQ ID NO: 207) and miORF8 (SEQ ID NO: 208).

In an embodiment, the invention relates to the use as defined above in which said nucleic acid is selected from miORF155 (SEQ ID NO: 356).

In an embodiment, the invention relates to the use as defined above in which said microRNA is selected from the group of nucleic acids consisting of SEQ ID NO: 282 to SEQ ID NO: 354 and SEQ ID NO: 358.

In an embodiment, the invention relates to the use as defined above in which said microRNA is selected from miR171b (SEQ ID NO: 319), miR165a (SEQ ID NO: 305) and miR319a (SEQ ID NO: 331).

In an embodiment, the invention relates to the use as defined above in which said microRNA is selected from miR1a (SEQ ID NO: 353) and miR8 (SEQ ID NO: 354).

In an embodiment, the invention relates to the use as defined above in which said microRNA is selected from miR155 (SEQ ID NO: 358).

In another aspect, the invention relates in particular to a process for modulating the expression of a gene regulated by a microRNA in a eukaryotic cell,

comprising carrying out a step of accumulation of a miPEP in said eukaryotic cell,
said miPEP having:
   a size from 3 to 100 amino acids, preferably 4 to 20 amino acids, and
   a peptide sequence identical to that encoded by a nucleotide sequence contained in the primary transcript of a microRNA regulating the expression of said gene, and
   being capable of modulating the accumulation of said microRNA,
      in which the accumulation of said miPEP in said eukaryotic cell induces a modulation of the expression of said gene relative to the expression of said gene without accumulation of said miPEP.

In particular, the invention relates to a process for modulating the expression of a gene regulated by a microRNA in a eukaryotic cell,

comprising carrying out a step of accumulation of a miPEP in said eukaryotic cell,
said miPEP having:
   a size from 4 to 100 amino acids, preferably 4 to 20 amino acids, and
   a peptide sequence identical to that encoded by a nucleotide sequence contained in the primary transcript of a microRNA regulating the expression of said gene, and
   being capable of modulating the accumulation of said microRNA,
      in which the accumulation of said miPEP in said eukaryotic cell induces a modulation of the expression of said gene relative to the expression of said gene without accumulation of said miPEP.

In an embodiment, the invention relates to a process for modulating the expression of a gene as defined above, in which the accumulation of said miPEP in the cell results from:
introduction of a nucleic acid encoding said miPEP into the cell, or
introduction of said miPEP into the cell.

In an embodiment, the invention relates to a process for modulating the expression of a gene as defined above in which said eukaryotic cell is a plant cell.

In an embodiment, the invention relates to a process for modulating the expression of a gene as defined above in which said eukaryotic cell is an animal cell and in particular a human cell.

In an embodiment, the invention relates to a process for modulating the expression of a gene as defined above in which said eukaryotic cell is an animal cell and in particular a human cell, said process not being used for surgical or therapeutic treatment of the human body or animal body, nor for modifying the genetic identity of a human being.

In an embodiment, the invention relates to a process for modulating the expression of a gene as defined above in which said microRNA and said gene are of endogenous origin in said eukaryotic cell.

In an embodiment, the invention relates to a process for modulating the expression of a gene as defined above in which said microRNA and said gene are of exogenous origin in said eukaryotic cell, said eukaryotic cell containing at least one vector allowing the expression of said microRNA and of said gene.

In an embodiment, the invention relates to a process for modulating the expression of a gene as defined above in which said miPEP is selected from the group of peptides consisting of SEQ ID NO: 1 to SEQ ID NO: 104, SEQ ID NO: 375 to SEQ ID NO: 386 and SEQ ID NO: 355.

In a particular embodiment, the invention relates to a process for modulating the expression of a gene regulated by miR171b (SEQ ID NO: 319) in a eukaryotic cell,

comprising carrying out a step of accumulation of MtmiPEP171b1 (SEQ ID NO: 59) in said eukaryotic cell,
in which the accumulation of said MtmiPEP171b1 in said eukaryotic cell induces a modulation of the expression of said gene relative to the expression of said gene without accumulation of said MtmiPEP171b1.

In a particular embodiment, the invention relates to a process for modulating the expression of a gene regulated by miR171b (SEQ ID NO: 319) in a eukaryotic cell,

comprising carrying out a step of accumulation of MtmiPEP171b1 (SEQ ID NO: 59) in said eukaryotic cell,
in which the accumulation of said MtmiPEP171b1 in said eukaryotic cell induces a modulation of the expression of said gene relative to the expression of said gene without accumulation of said MtmiPEP171b1,
in which said gene is selected from the genes HAM1 (accession No. MtGI9-TC114268) and HAM2 (accession No. MtGI9-TC120850) (accession numbers according to the database Medicago truncatula Gene Expression Atlas “MtGEA”).

In a particular embodiment, the invention relates to a process for modulating the expression of a gene regulated by miR164a (SEQ ID NO: 297) in a eukaryotic cell, comprising carrying out a step of accumulation of AtmiPEP165a1 (SEQ ID NO: 24) in said eukaryotic cell,

in which the accumulation of said AtmiPEP164a1 in said eukaryotic cell induces a modulation of the expression of said gene relative to the expression of said gene without accumulation of said AtmiPEP164a1.

In a particular embodiment, the invention relates to a process for modulating the expression of a gene regulated by miR165a (SEQ ID NO: 305) in a eukaryotic cell,

comprising carrying out a step of accumulation of AtmiPEP165a (SEQ ID NO: 43) in said eukaryotic cell,
in which the accumulation of said AtmiPEP165a in said eukaryotic cell induces a modulation of the expression of said gene relative to the expression of said gene without accumulation of said AtmiPEP165a.

In a particular embodiment, the invention relates to a process for modulating the expression of a gene regulated by miR319a (SEQ ID NO: 331) in a eukaryotic cell,

comprising carrying out a step of accumulation of AtmiPEP319a1 (SEQ ID NO: 76) in said eukaryotic cell,
in which the accumulation of said AtmiPEP319a1 in said eukaryotic cell induces a modulation of the expression of said gene relative to the expression of said gene without accumulation of said AtmiPEP319a1.

In a particular embodiment, the invention relates to a process for modulating the expression of a gene regulated by miR319a (SEQ ID NO: 331) in a eukaryotic cell,

comprising carrying out a step of accumulation of AtmiPEP319a2 (SEQ ID NO: 77) in said eukaryotic cell,
in which the accumulation of said AtmiPEP319a2 in said eukaryotic cell induces a modulation of the expression of said gene relative to the expression of said gene without accumulation of said AtmiPEP319a2.

In a particular embodiment, the invention relates to a process for modulating the expression of a gene regulated by miR1 (SEQ ID NO: 353) in a eukaryotic cell,

comprising carrying out a step of accumulation of DmmiPEP1a (SEQ ID NO: 102) in said eukaryotic cell,
in which the accumulation of said DmmiPEP1a in said eukaryotic cell induces a modulation of the expression of said gene relative to the expression of said gene without accumulation of said DmmiPEP1a.

In a particular embodiment, the invention relates to a process for modulating the expression of a gene regulated by miR1 (SEQ ID NO: 353) in a eukaryotic cell,

comprising carrying out a step of accumulation of DmmiPEP1b (SEQ ID NO: 103) in said eukaryotic cell,
in which the accumulation of said DmmiPEP1b in said eukaryotic cell induces a modulation of the expression of said gene relative to the expression of said gene without accumulation of said DmmiPEP1b.

In a particular embodiment, the invention relates to a process for modulating the expression of a gene regulated by miR8 (SEQ ID NO: 354) in a eukaryotic cell,

comprising carrying out a step of accumulation of DmmiPEP8 (SEQ ID NO: 104) in said eukaryotic cell,
in which the accumulation of said DmmiPEP8 in said eukaryotic cell induces a modulation of the expression of said gene relative to the expression of said gene without accumulation of said DmmiPEP8.

In a particular embodiment, the invention relates to a process for modulating the expression of a gene regulated by miR155 (SEQ ID NO: 358) in a eukaryotic cell,

comprising carrying out a step of accumulation of hsmiPEP155 (SEQ ID NO: 355) in said eukaryotic cell,
in which the accumulation of said hsmiPEP155 in said eukaryotic cell induces a modulation of the expression of said gene relative to the expression of said gene without accumulation of said hsmiPEP155.

In another aspect, the invention relates to a modified eukaryotic cell containing a peptide identical to a miPEP as defined above, said peptide being present in said eukaryotic cell independently of transcription of the primary transcript of the microRNA bearing the nucleotide sequence encoding said miPEP.

In the invention, by the term “modified eukaryotic cell” is meant that said eukaryotic cell contains a miPEP introduced into the cell artificially, whether as a peptide, or via a vector encoding said miPEP.

In an embodiment, the invention relates to a modified eukaryotic cell as defined above, in which said microRNA is of endogenous origin.

In another embodiment, the invention relates to a modified eukaryotic cell as defined above in which said microRNA is of exogenous origin, said modified eukaryotic cell containing a vector allowing the expression of said microRNA.

In an embodiment, the invention relates to a modified eukaryotic cell as defined above, said cell being a plant cell.

In an embodiment, the invention relates to a modified eukaryotic cell as defined above, in which said plant cell is a cell of Medicago truncatula or of Arabidopsis thaliana, and said peptide is selected from the group of peptides consisting of SEQ ID NO: 43, SEQ ID NO: 59 and SEQ ID NO: 77.

In an embodiment, the invention relates to a modified eukaryotic cell as defined above, said cell being an animal cell.

In an embodiment, the invention relates to a modified eukaryotic cell as defined above, in which said animal cell is a Drosophila cell and said peptide is selected from the group of peptides consisting of SEQ ID NO: 102, SEQ ID NO: 103 and SEQ ID NO: 104.

In an embodiment, the invention relates to a modified eukaryotic cell as defined above, in which said animal cell is a human cell and said peptide consists of SEQ ID NO: 355.

In another aspect, the invention relates to a plant comprising at least one modified eukaryotic cell as defined above.

In another aspect, the invention also relates to a non-human animal organism comprising at least one modified eukaryotic cell as defined above.

In another aspect, the invention relates to a composition comprising at least one:
miPEP as defined above,
nucleic acid encoding said miPEP, or
vector containing said nucleic acid.

In another aspect, the invention relates to a pesticide composition comprising at least one:
miPEP as defined above,
nucleic acid encoding said miPEP, or
vector containing said nucleic acid.

In another aspect, the invention relates to a phytopharmaceutical composition comprising at least one:
miPEP as defined above,
nucleic acid encoding said miPEP, or
vector containing said nucleic acid.

In another aspect, the invention relates to an elicitor composition comprising at least one:
miPEP as defined above,
nucleic acid encoding said miPEP, or
vector containing said nucleic acid.

“Elicitor composition” denotes a composition capable of endowing the plant with better capacity for symbiosis or better resistance to different stresses whether they are of the nature of thermal stress, water stress or chemical stress.

For this purpose, the invention also relates to compositions acting on the growth (inhibition of growth or conversely growth promotion) and the physiology (better capacity for mycorrhization, nodule formation, better tolerance of different stresses) of the plant.

In particular, the invention relates to compositions for promoting plant growth.

In another aspect, the invention relates to a herbicide composition comprising at least one:
miPEP as defined above,
nucleic acid encoding said miPEP, or
vector containing said nucleic acid.

In another aspect, the invention relates to an insecticide composition comprising at least one:
miPEP as defined above,
nucleic acid encoding said miPEP, or
vector containing said nucleic acid.

In another aspect, the invention relates to a composition, in particular a phytosanitary composition, comprising miPEP164a as active ingredient, said miPEP164a preferably consisting of SEQ ID NO: 24.

In another aspect, the invention relates to a composition, in particular a phytosanitary composition, comprising miPEP319a as active ingredient, said miPEP319a preferably consisting of SEQ ID NO: 76.

In another aspect, the invention relates to a composition, in particular a phytosanitary composition, comprising miPEP171b as active ingredient, said miPEP171b preferably consisting of SEQ ID NO: 59.

The solubility properties of the miPEPs are in particular determined by their amino acid composition. The hydrophilic miPEPs can be dissolved and packaged in aqueous solutions, such as water. The hydrophobic miPEPs can be dissolved and packaged in solvents, such as organic solvents.

For treatment of plants with the miPEPS, the organic solvents are solvents that are non-toxic to the plants in small quantities, i.e. they do not have any harmful effect on the development of the plant. Non-limitatively, the organic solvents may be selected from acetonitrile and acetic acid.

The miPEPs can also be dissolved and packaged in mixtures of organic solvents, such as for example a mixture of acetonitrile and acetic acid. In particular, the miPEPs may be dissolved in a solution comprising 50% acetonitrile, 10% acetic acid and 40% water (volume/volume/volume).

Preferably, miPEPs 164a and 165a are dissolved in water, and miPEPs 171b and 319a are dissolved in a solution comprising 50% acetonitrile, 10% acetic acid and 40% water (volume/volume/volume).

Non-limitatively, the compositions, the pesticide compositions, the phytopharmaceutical compositions, the herbicide compositions and the insecticide compositions as defined above may comprise 10⁻⁹ M to 10⁻⁴ M of miPEP, in particular 10⁻⁹ M, 10⁻⁸ M, 10⁻⁷ M, 10⁻⁶ M, 10⁻⁵ M or 10⁻⁴ M of miPEP.

Compositions of higher or lower concentration may also be provided depending on the applications envisaged. For example, compositions comprising 10⁻¹ M to 10⁻³ M of miPEP, in particular 10⁻¹ M, 10⁻² M or 10⁻³ M of miPEP, may be envisaged in the case where the miPEP has to be administered to the plant by spreading.

In another aspect, the invention relates to the use of a composition as defined above, as a herbicide for eradicating plants or slowing their growth, preferably as a herbicide specific to a species or to a genus of plants.

In another aspect, the invention relates to the use of a composition as defined above, as a phytopharmaceutical agent,
for promoting the growth and/or development of plants,
   in particular for modulating the physiological parameters of a plant, in particular the biomass, foliar surface area, flowering, fruit size, production and/or selection of plant seeds, in particular for controlling the parthenocarpy or the monoecism of a plant, or for modifying the physiological parameters of plant seeds, in particular germination, establishment of the root system and resistance to water stress,
or for preventing or treating plant diseases,
   in particular for promoting resistance to infectious diseases.

In another aspect, the invention relates to the use of a composition as defined above, for modulating the physiological parameters of a plant, in particular biomass, foliar surface area, or fruit size.

In an embodiment, the invention relates to the use of a composition as defined above, for thinning of orchards in order to increase fruit size.

In an embodiment, the invention relates to the use of a composition as defined above, for production and/or selection of plant seeds, said composition being used for controlling the parthenocarpy or the monoecism of a plant.

In an embodiment, the invention relates to the use of a composition as defined above, said composition being administered to said plant via the leaves or via the roots.

In an embodiment, the invention relates to the use of a composition as defined above, for production and/or selection of plant seeds.

In an embodiment, the invention relates to the use of a composition as defined above, in which said composition is used for modifying the physiological parameters of said plant seeds, in particular establishment of the root system, germination and resistance to water stress.

In an embodiment, the invention relates to the use of a composition as defined above, in which said composition is applied by dressing or film-coating of said plant seeds.

In another aspect, the invention relates to the use of a composition as defined above, as a pesticide, for eradicating organisms that are harmful to plants or that might be classified as such,

in particular as insecticide, arachnicide, molluscicide or rodenticide.

In an embodiment, the invention relates to the use of a composition as defined above, as insecticide.

In an embodiment, the invention relates to the use of a composition as defined above, for eradicating insect pests.

In an embodiment, the invention relates to the use of a composition as defined above, for eradicating animal species classified as harmful or liable to be classified as such, in particular the Muridae, in particular the rat.

In an embodiment, the invention relates to the use of a composition as defined above, as pesticide for eradicating organisms harmful to plants or liable to be classified as such,

in particular as insecticide, arachnicide, molluscicide, or rodenticide,
in particular by application of said composition to a plant or to a support in contact with the plant.

In another aspect, the invention relates to the use of a composition as defined above, in which said composition is applied to a plant to protect it against insect pests.

In another aspect, the invention relates to the use of a peptide for promoting the growth of a plant, said peptide being introduced into the plant, said peptide having an amino acid sequence comprising or consisting of a sequence identical to that of a miPEP naturally present in said plant,

said miPEP naturally present in said plant being a peptide of 4 to 100 amino acids the sequence of which is encoded by an open reading frame located on the primary transcript of a miRNA, said miPEP being capable of modulating the accumulation of said miRNA in said plant, said miRNA regulating the expression of at least one gene involved in the development of the vegetative or reproductive parts of the plant, in particular the roots, stem, leaves or flowers.

The inventors have surprisingly found that the use of peptides the sequence of which comprises or consists of a sequence identical to that of miPEPs encoded on the primary transcripts of miRNAs makes it possible to promote the growth of the plants.

In the invention, the term “plant” refers generally to the whole or part of a plant irrespective of its stage of development (including the plant in the form of a seed or a young shoot), to one or more organs of the plant (for example the leaves, roots, stem, flowers), to one or more cells of the plant, or to a cluster of cells of the plant.

In the invention, the term “growth” refers to the development of the whole or part of a plant over time. The growth of the plant may thus be determined and quantified by monitoring developmental parameters observable for certain parts, cells or organs of the plant, such as the leaves, roots, stems or flowers.

Non-limitatively, the parameters for determining and quantifying the growth of a plant may in particular be:
the size, surface area, volume, mass and the number of leaves,
the size and number of flowers,
the size of the stem (or spike),
the length and number of roots,
the earliness of germination,
the earliness of budding,
the earliness of floral induction (or floral transition),
or also the number of cells.

In the case of leguminous plants, plant growth may also be linked to the rate of nodulation, or also to the size and number of nodules on the roots.

Moreover, in the invention, the expression “promote plant growth”, or “improve plant growth”, indicates:
either an acceleration of development (such as for example a larger leaf size for a plant at a given point in time relative to a reference plant),
or an increase in development (such as for example a larger leaf size for a plant that cannot be attained by a reference plant),
or an acceleration and an increase in the development of the plant.

It is important to note that the use according to the invention has the advantage of being ecological, in comparison with the chemical methods used conventionally in horticulture or in agriculture, as the miPEP is a peptide that is present naturally in the plant.

The invention also relates to the use of a miPEP introduced into a plant for promoting its growth,

said miPEP introduced being a peptide comprising, or consisting of, a sequence identical to that of a miPEP naturally present in said plant,
said miPEP naturally present is a peptide of 4 to 100 amino acids, the sequence of which is encoded by an open reading frame located at 5′ on the primary transcript of a miRNA, said miPEP being capable of modulating the accumulation of said miRNA in said plant, said miRNA regulating the expression of at least one gene involved in the development of the vegetative or reproductive parts of the plant, in particular the roots, stem, leaves or flowers,
the sum total of the quantity of said miPEP introduced and that of said miPEP naturally present being strictly greater than the quantity of said miPEP naturally present.

In the invention, the expression “miPEP introduced” refers to a miPEP introduced into the plant artificially as opposed to the “miPEP present naturally in the plant”. The introduction of a miPEP into the plant therefore involves a technological step, which is not a natural phenomenon and corresponds neither to crossing, nor to selection.

The miPEP introduced may be either a peptide produced outside of the plant (for example an isolated and/or purified peptide, a synthetic peptide or a recombinant peptide), or a peptide produced in the plant following the non-natural introduction of a nucleic acid encoding said miPEP into said plant.

The plant into which the miPEP has not been introduced has a basal quantity of said miPEP, which corresponds to the quantity of said miPEP naturally present. The use of a miPEP comprising, or consisting of, a sequence identical to that of said miPEP leads to an increase in the total quantity of miPEP, which modulates the accumulation of the miRNA the primary transcript of which contains the sequence encoding said miPEP.

Moreover, the miPEP introduced is present in the plant and its introduction has no impact on its stability.

In an embodiment, the invention relates to the use as defined above, in which said gene, involved in the development of the vegetative or reproductive parts of the plant, is selected from the group consisting of: NAC1 (Accession No. AT1G56010.1), NAC4 (Accession No. AT5G07680.1), NAC5 (Accession No. AT5G61430.1), CUC1 (Accession No. AT3G15170.1), CUC2 (Accession No. AT5G53950.1), TCP3 (Accession No. AT1G53230.1) and TCP4 (Accession No. AT3G15030.1) (accession numbers according to the database The Arabidopsis Information Resource “TAIR”).

In particular, the invention relates to the use as defined above, in which said gene involved in the development of the vegetative or reproductive parts of the plant is selected from the group consisting of: NAC1, NAC4, NAC5, CUC1 and CUC2.

In an embodiment, the invention relates to the use as defined above, in which said gene involved in the development of the vegetative or reproductive parts of the plant is selected from the group consisting of: TCP3 and TCP4,

In an embodiment, the invention relates to the use as defined above, in which said miRNA is selected from miR164a and mir319a.

In particular, the invention relates to the use as defined above, in which said miR164a has a nucleotide sequence consisting of SEQ ID NO: 297.

In particular, the invention relates to the use as defined above, in which said miR164a has a nucleotide sequence having at least 80% identity, preferably at least 90% identity, with the nucleotide sequence SEQ ID NO: 297.

In an embodiment, the invention relates to the use as defined above, in which said miPEP is AtmiPEP164a1, in particular in which said AtmiPEP164a1 has an amino acid sequence consisting of SEQ ID NO: 24.

In particular, the invention relates to the use as defined above, in which said miR319a has a nucleotide sequence consisting of SEQ ID NO: 331.

In particular, the invention relates to the use as defined above, in which said miR319a has a nucleotide sequence having at least 80% identity, preferably at least 90% identity, with the nucleotide sequence SEQ ID NO: 331.

In an embodiment, the invention relates to the use as defined above, in which said miPEP is AtmiPEP319a1, in particular in which said AtmiPEP319a1 has an amino acid sequence consisting of SEQ ID NO: 76.

In an embodiment, the invention relates to the use as defined above, in which said plant is a crucifer such as Arabidopsis thaliana, a leguminous plant such as Glycine max (soya), Medicago truncatula and Medicago sativa (alfalfa) or a plant of the Solanaceae family such as Nicotiana benthamiana (tobacco), Solanum tuberosum (potato), Solanum lycopersicum (tomato) or Solanum melongena (aubergine).

In an embodiment, the invention relates to the use as defined above, in which said plant is a crucifer.

In an embodiment, the invention relates to the use as defined above, in which said plant is Arabidopsis thaliana.

In an embodiment, the invention relates to the use as defined above, for promoting the growth of an Arabidopsis thaliana plant, in which AtmiPEP164a1 is introduced into said Arabidopsis thaliana plant, said AtmiPEP164a1 also being naturally present in said Arabidopsis thaliana plant,

said AtmiPEP164a1 introduced being a peptide the sequence of which comprises or consists of a sequence identical to that of said AtmiPEP164a1 naturally present, said sequence of AtmiPEP164a1 naturally present being encoded by an open reading frame located at 5′ on the primary transcript of the miR164a, which miR164a controls the expression of at least one gene involved in the development of the vegetative or reproductive parts of Arabidopsis thaliana,
the sum total of the quantity of said AtmiPEP164a1 introduced and that of said AtmiPEP164a1 naturally present being strictly greater than the quantity of said AtmiPEP164a1 naturally present in said Arabidopsis thaliana plant.

In an embodiment, the invention relates to the use as defined above, for promoting the growth of an Arabidopsis thaliana plant, in which the AtmiPEP319a1 is introduced into said Arabidopsis thaliana plant, said AtmiPEP319a1 also being naturally present in said Arabidopsis thaliana plant,

said AtmiPEP319a1 introduced being a peptide the sequence of which comprises or consists of a sequence identical to that of said AtmiPEP319a1 naturally present, said sequence of the AtmiPEP319a1 naturally present being encoded by an open reading frame located at 5′ on the primary transcript of the miR319a, which miR319a controls the expression of at least one gene involved in the development of the vegetative or reproductive parts of Arabidopsis thaliana,
the sum total of the quantity of said AtmiPEP319a1 introduced and that of said AtmiPEP319a1 naturally present being strictly greater than the quantity of said AtmiPEP319a1 naturally present in said Arabidopsis thaliana plant.

In an embodiment, the invention relates to the use as defined above, in which said miPEP is introduced into the plant externally, preferably by watering, by spraying or by adding a fertilizer, a compost, a culture substrate or an inert support.

In an embodiment, the invention relates to the use as defined above, in which said miPEP is introduced by watering and by spraying.

In an embodiment, the invention relates to the use as defined above, in which said miPEP is introduced by watering and by adding a fertilizer.

In an embodiment, the invention relates to the use as defined above, in which said miPEP is introduced by spraying and by adding a fertilizer.

In an embodiment, the invention relates to the use as defined above, in which said miPEP is introduced, by watering, by spraying and by adding a fertilizer.

The inventors have in fact unexpectedly found that it is possible to apply a composition comprising a miPEP directly to the plant in order to modulate the accumulation of the corresponding miRNA in the plant, which indicates that the miPEP is captured by the plant.

In an embodiment, the invention relates to the use as defined above, in which the plant is treated with a composition comprising 10⁻⁹ M to 10⁻⁴ M of said miPEP, in particular 10⁻⁹ M, 10⁻⁸ M, 10⁻⁷ M, 10⁻⁶ M, 10⁻⁵ M or 10⁻⁴ M of said miPEP.

Preferably, the compositions have a concentration from 10⁻⁸ M to 10⁻⁵ M for application by watering or by spraying on the plant.

In addition, compositions of higher or lower concentration may be envisaged for treating the plant with the miPEP. As a non-limitative example, compositions of higher concentration comprising 10⁻¹ M to 10⁻³ M, in particular 10⁻² M of miPEP, may be used in the case where the miPEP introduced exogenously is administered to the plant by spreading.

In an embodiment, the invention relates to the use as defined above, in which said miPEP is introduced into the plant by means of a nucleic acid encoding said miPEP, said nucleic acid being introduced into the plant.

In an embodiment, the invention relates to the use as defined above, in which the size of the stem is increased in the plant into which said miPEP has been introduced relative to the size of the stem of an identical plant of the same age into which no miPEP has been introduced, or relative to the size of the stem of an identical plant of the same age into which said miPEP has not been introduced.

In an embodiment, the invention relates to the use as defined above, in which the number of leaves is increased in the plant into which said miPEP has been introduced relative to the number of leaves of an identical plant of the same age into which no miPEP has been introduced, or relative to the number of leaves of an identical plant of the same age into which said miPEP has not been introduced.

In an embodiment, the invention relates to the use as defined above, in which the size of the leaves is increased in the plant into which said miPEP has been introduced relative to the size of the leaves of an identical plant of the same age into which no miPEP has been introduced, or relative to the size of the leaves of an identical plant of the same age into which said miPEP has not been introduced.

In an embodiment, the invention relates to the use as defined above, in which the number of roots is increased in the plant into which said miPEP has been introduced relative to the number of roots of an identical plant of the same age into which no miPEP has been introduced, or relative to the number of roots of an identical plant of the same age into which said miPEP has not been introduced.

In an embodiment, the invention relates to the use as defined above, in which the length of the roots is increased in the plant into which said miPEP has been introduced relative to the length of the roots of an identical plant of the same age into which no miPEP has been introduced, or relative to the length of the roots of an identical plant of the same age into which said miPEP has not been introduced.

In an embodiment, the invention relates to the use as defined above, in which the rate of nodulation is increased in the plant into which said miPEP has been introduced relative to the rate of nodulation of an identical plant of the same age into which no miPEP has been introduced, or relative to the rate of nodulation of an identical plant of the same age into which said miPEP has not been introduced.

In an embodiment, the invention relates to the use as defined above, in which the number of nodules is increased in the plant into which said miPEP has been introduced relative to the number of nodules of an identical plant of the same age into which no miPEP has been introduced, or relative to the number of nodules of an identical plant of the same age into which said miPEP has not been introduced.

The increase in the parameters for determining and quantifying growth in the plant into which the miPEP has been introduced (such as the size of the stem, the number and size of the leaves, the number and length of the roots, the rate of nodulation or also the number of nodules on the roots) is preferably demonstrated by comparison with an identical plant (i.e. a plant of the same species and/or variety), of the same age and grown under the same conditions but into which no miPEP has been introduced.

In another aspect, the invention relates to a process for promoting the growth of a plant, comprising a step of introducing a miPEP into a plant, said miPEP also being present naturally in said plant,

said miPEP introduced being a peptide of 4 to 100 amino acids the sequence of which comprises or consists of a sequence identical to that of said miPEP naturally present, said sequence of the miPEP naturally present being encoded by an open reading frame located at 5′ on the primary transcript of a miRNA, said miPEP being capable of modulating the accumulation of said miRNA, said miRNA regulating the expression of at least one gene involved in the development of the vegetative or reproductive parts of the plant, in particular the roots, stem, leaves or flowers,
the sum total of the quantity of said miPEP introduced and that of said miPEP naturally present being strictly greater than the quantity of said miPEP naturally present.

In an embodiment, the invention relates to a process as defined above, in which said gene involved in the development of the vegetative or reproductive parts of the plant is selected from the group consisting of: NAC1 (Accession No. AT1G56010.1), NAC4 (Accession No. AT5G07680.1), NAC5 (Accession No. AT5G61430.1), CUC1 (Accession No. AT3G15170.1), CUC2 (Accession No. AT5G53950.1), TCP3 (Accession No. AT1G53230.1) and TCP4 (Accession No. AT3G15030.1).

In particular, the invention relates to a process as defined above, in which said gene involved in the development of the vegetative or reproductive parts of the plant is selected from the group consisting of: NAC1, NAC4, NAC5, CUC1 and CUC2.

In an embodiment, the invention relates to a process as defined above, in which said gene involved in the development of the vegetative or reproductive parts of the plant is selected from the group consisting of: TCP3 and TCP4.

In an embodiment, the invention relates to a process as defined above, in which said miRNA is miR164a, in particular in which said miR164a has a nucleotide sequence consisting of SEQ ID NO: 297.

In an embodiment, the invention relates to a process as defined above, in which said miPEP is AtmiPEP164a1, in particular in which said AtmiPEP164a1 has an amino acid sequence consisting of SEQ ID NO: 24.

In an embodiment, the invention relates to a process as defined above, in which said miRNA is miR319a, in particular in which said miR319a has a nucleotide sequence consisting of SEQ ID NO: 331.

In an embodiment, the invention relates to a process as defined above, in which said miPEP is AtmiPEP319a1, in particular in which said AtmiPEP319a1 has an amino acid sequence consisting of SEQ ID NO: 76.

In an embodiment, the invention relates to a process as defined above, in which said plant is a crucifer such as Arabidopsis thaliana, a leguminous plant such as Glycine max (soya), Medicago truncatula and Medicago sativa (alfalfa) or a plant of the Solanaceae family such as Nicotiana benthamiana (tobacco), Solanum tuberosum (potato), Solanum lycopersicum (tomato) or Solanum melongena (aubergine).

In an embodiment, the invention relates to a process as defined above, in which said plant is a crucifer.

In an embodiment, the invention relates to a process as defined above, in which said plant is Arabidopsis thaliana.

In an embodiment, the invention relates to a process as defined above, for promoting the growth of an Arabidopsis thaliana plant, in which AtmiPEP164a1 is introduced into said Arabidopsis thaliana plant, said AtmiPEP164a1 also being naturally present in said Arabidopsis thaliana plant,

said AtmiPEP164a1 introduced being a peptide comprising or consisting of a sequence identical to that of said AtmiPEP164a1 naturally present, where the AtmiPEP164a1 naturally present is a peptide of 4 to 100 amino acids the sequence of which is encoded by an open reading frame located at 5′ on the primary transcript of the miR164a,
said AtmiPEP164a1 being capable of increasing the accumulation of said miR164a, where said miR164a regulates the expression of at least one gene involved in the development of the vegetative or reproductive parts of Arabidopsis thaliana,
the sum total of the quantity of said AtmiPEP164a1 introduced and that of said AtmiPEP164a1 naturally present being strictly greater than the quantity of said AtmiPEP164a1 naturally present.

In an embodiment, the invention relates to a process as defined above, for promoting the growth of an Arabidopsis thaliana plant, in which AtmiPEP319a1 is introduced into said Arabidopsis thaliana plant, said AtmiPEP319a1 also being naturally present in said Arabidopsis thaliana plant,

said AtmiPEP319a1 introduced being a peptide comprising or consisting of a sequence identical to that of said AtmiPEP319a1 naturally present, where the AtmiPEP319a1 naturally present is a peptide of 4 to 100 amino acids the sequence of which is encoded by an open reading frame located at 5′ on the primary transcript of the miR319a,
said AtmiPEP319a1 being capable of increasing the accumulation of said miR319a, where miR319a regulates the expression of at least one gene involved in the development of the vegetative or reproductive parts of Arabidopsis thaliana,
the sum total of the quantity of said AtmiPEP319a1 introduced and that of said AtmiPEP319a1 naturally present being strictly greater than the quantity of said AtmiPEP319a1 naturally present.

In an embodiment, the invention relates to a process as defined above, in which said miPEP is introduced into the plant externally, preferably by watering, by spraying or by adding a fertilizer, a compost, a culture substrate or an inert support.

In an embodiment, the invention relates to a process as defined above, in which said miPEP is administered to the plant in the form of a composition comprising 10⁻⁹ M to 10⁻⁴ M of said miPEP, in particular 10⁻⁹, 10⁻⁸, 10⁻⁷, 10⁻⁶, 10⁻⁵ or 10⁻⁴ M of said miPEP.

In an embodiment, the invention relates to a process as defined above, in which said miPEP is introduced into the plant by means of a nucleic acid encoding said miPEP, said nucleic acid being introduced into the plant.

In an embodiment, the invention relates to a process as defined above, in which the size of the stem is increased in the plant into which said miPEP has been introduced relative to the size of the stem of an identical plant of the same age into which no miPEP has been introduced, or relative to the size of the stem of an identical plant of the same age into which said miPEP has not been introduced.

In an embodiment, the invention relates to a process as defined above, in which the number of leaves is increased in the plant into which said miPEP has been introduced relative to the number of leaves of an identical plant of the same age into which no miPEP has been introduced, or relative to the number of leaves of an identical plant of the same age into which said miPEP has not been introduced.

In an embodiment, the invention relates to a process as defined above, in which the size of the leaves is increased in the plant into which said miPEP has been introduced relative to the size of the leaves of an identical plant of the same age into which no miPEP has been introduced, or relative to the size of the leaves of an identical plant of the same age into which said miPEP has not been introduced.

In an embodiment, the invention relates to a process as defined above, in which the number of roots is increased in the plant into which said miPEP has been introduced relative to the number of roots of an identical plant of the same age into which no miPEP has been introduced, or relative to the number of roots of an identical plant of the same age into which said miPEP has not been introduced.

In an embodiment, the invention relates to a process as defined above, in which the length of the roots is increased in the plant into which said miPEP has been introduced relative to the length of the roots of an identical plant of the same age into which no miPEP has been introduced, or relative to the length of the roots of an identical plant of the same age into which said miPEP has not been introduced.

In an embodiment, the invention relates to a process as defined above, in which the rate of nodulation is increased in the plant into which said miPEP has been introduced relative to the rate of nodulation of an identical plant of the same age into which no miPEP has been introduced, or relative to the rate of nodulation of an identical plant of the same age into which said miPEP has not been introduced.

In an embodiment, the invention relates to a process as defined above, in which the number of nodules is increased in the plant into which said miPEP has been introduced relative to the number of nodules of an identical plant of the same age into which no miPEP has been introduced, or relative to the number of nodules of an identical plant of the same age into which said miPEP has not been introduced.

In another aspect, the invention relates to a plant into which a miPEP has been introduced according to the use or the process for promoting the growth of a plant described above.

In another aspect, the invention relates to a process for producing a transgenic plant comprising:
a) a step of introducing a nucleic acid encoding a miPEP of 4 to 100 amino acids into a plant, or into at least one cell of said plant, under conditions allowing the expression of said miPEP,
   said miPEP also being naturally present in said plant, said miPEP naturally present being a peptide the sequence of which is encoded by an open reading frame located at 5′ on the primary transcript of a miRNA, said miPEP being capable of modulating the accumulation of said miRNA in the plant, said miRNA regulating the expression of at least one gene involved in the development of the vegetative or reproductive parts of the plant, in particular the roots, stem, leaves or flowers, and
b) a step of culturing the plant, or at least one cell of said plant, obtained in step a) under conditions allowing a transgenic plant to be obtained.

In an embodiment, the invention relates to a process for producing a transgenic plant as defined above, in which said transgenic plant obtained in step b) has improved growth relative to an identical plant in which said nucleic acid has not been introduced.

In an embodiment, the invention relates to a process for producing a transgenic plant as defined above, in which step a) is carried out using a vector containing said nucleic acid, preferably a plasmid.

In an embodiment, the invention relates to a process for producing a transgenic plant as defined above, in which the expression of said nucleic acid of step a) is placed under the control of a strong promoter, preferably a constitutive strong promoter such as the 35S promoter.

In an embodiment, the invention relates to a process for producing a transgenic plant as defined above, in which said gene involved in the development of the vegetative or reproductive parts of the plant is selected from the group consisting of: NAC1 (Accession No. AT1G56010.1), NAC4 (Accession No. AT5G07680.1), NAC5 (Accession No. AT5G61430.1), CUC1 (Accession No. AT3G15170.1), CUC2 (Accession No. AT5G53950.1), TCP3 (Accession No. AT1G53230.1) and TCP4 (Accession No. AT3G15030.1).

In an embodiment, the invention relates to a process for producing a transgenic plant as defined above, in which said miPEP has an amino acid sequence comprising or consisting of a sequence selected from the group consisting of SEQ ID NO: 1 to SEQ ID NO: 101 and SEQ ID NO: 375 to SEQ ID NO: 386.

In an embodiment, the invention relates to a process for producing a transgenic plant as defined above, in which said miRNA is miR164a, in particular in which said miR164a has a nucleotide sequence consisting of SEQ ID NO: 297.

In an embodiment, the invention relates to a process for producing a transgenic plant as defined above, in which said miPEP is AtmiPEP164a1, in particular in which said AtmiPEP164a1 has an amino acid sequence consisting of SEQ ID NO: 24.

In an embodiment, the invention relates to a process for producing a transgenic plant as defined above, in which said miRNA is miR319a, in particular in which said miR319a has a nucleotide sequence consisting of SEQ ID NO: 331.

In an embodiment, the invention relates to a process for producing a transgenic plant as defined above, in which said miPEP is AtmiPEP319a1, in particular in which said AtmiPEP319a1 has an amino acid sequence consisting of SEQ ID NO: 76.

In an embodiment, the invention relates to a process for producing a transgenic plant as defined above, in which said nucleic acid introduced in step a) comprises a nucleotide sequence selected from SEQ ID NO: 128 and SEQ ID NO: 180.

In an embodiment, the invention relates to a process for producing a transgenic plant as defined above, in which said plant is a crucifer such as Arabidopsis thaliana, a leguminous plant such as Glycine max (soya), Medicago truncatula and Medicago sativa (alfalfa) or a plant of the Solanaceae family such as Nicotiana benthamiana (tobacco), Solanum tuberosum (potato), Solanum lycopersicum (tomato) or Solanum melongena (aubergine).

In an embodiment, the invention relates to a process for producing a transgenic plant as defined above, in which said transgenic plant is a crucifer.

In an embodiment, the invention relates to a process for producing a transgenic plant as defined above, in which said transgenic plant is Arabidopsis thaliana.

In an embodiment, the invention relates to a process for producing a transgenic plant as defined above, comprising:
a) a step of introducing a nucleic acid containing the nucleotide sequence SEQ ID NO: 128, encoding AtmiPEP164a1 consisting of the amino acid sequence SEQ ID NO: 24, into an Arabidopsis thaliana plant, or into at least one cell of said Arabidopsis thaliana plant, under conditions allowing the expression of AtmiPEP164a1,
   said AtmiPEP164a1 also being naturally present in said Arabidopsis thaliana plant, said miPEP naturally present being a peptide the sequence of which is encoded by an open reading frame located at 5′ on the primary transcript of miR164a, said AtmiPEP164a1 being capable of modulating the accumulation of said miR164, where miR164a controls the expression of at least one gene involved in the development of the vegetative or reproductive parts of Arabidopsis thaliana, and
b) a step of culturing the plant, or at least one cell of said plant, obtained in step a) under conditions allowing a transgenic Arabidopsis thaliana plant to be obtained.

In an embodiment, the invention relates to a process for producing a transgenic plant as defined above, comprising:
a) a step of introducing a nucleic acid containing the nucleotide sequence SEQ ID NO: 180, encoding AtmiPEP319a1 consisting of the amino acid sequence SEQ ID NO: 76, into an Arabidopsis thaliana plant, or into at least one cell of said Arabidopsis thaliana plant, under conditions allowing the expression of AtmiPEP319a1,
   said AtmiPEP319a1 also being naturally present in said Arabidopsis thaliana plant, said miPEP naturally present being a peptide the sequence of which is encoded by an open reading frame located at 5′ on the primary transcript of the miR319a, said AtmiPEP319a1 being capable of modulating the accumulation of said miR319, which miR319a regulates the expression of at least one gene involved in the development of the vegetative or reproductive parts of Arabidopsis thaliana, and
b) a step of culturing the plant, or at least one cell of said plant, obtained in step a) under conditions allowing a transgenic Arabidopsis thaliana plant to be obtained.

In an embodiment, the invention relates to a process of production as defined above, in which said miPEP is introduced into the plant by means of a nucleic acid encoding said miPEP, said nucleic acid being introduced into the plant.

In an embodiment, the invention relates to a process for producing a transgenic plant as defined above, in which the size of the stem is increased in the plant into which said miPEP has been introduced relative to the size of the stem of an identical plant of the same age into which no miPEP has been introduced, or relative to the size of the stem of an identical plant of the same age into which said miPEP has not been introduced.

In an embodiment, the invention relates to a process for producing a transgenic plant as defined above, in which the number of leaves is increased in the plant into which said miPEP has been introduced relative to the number of leaves of an identical plant of the same age into which no miPEP has been introduced, or relative to the number of leaves of an identical plant of the same age into which said miPEP has not been introduced.

In an embodiment, the invention relates to a process for producing a transgenic plant as defined above, in which the size of the leaves is increased in the plant into which said miPEP has been introduced relative to the size of the leaves of an identical plant of the same age into which no miPEP has been introduced, or relative to the size of the leaves of an identical plant of the same age into which said miPEP has not been introduced.

In an embodiment, the invention relates to a process for producing a transgenic plant as defined above, in which the number of roots is increased in the plant into which said miPEP has been introduced relative to the number of roots of an identical plant of the same age into which no miPEP has been introduced, or relative to the number of roots of an identical plant of the same age into which said miPEP has not been introduced.

In an embodiment, the invention relates to a process for producing a transgenic plant as defined above, in which the length of the roots is increased in the plant into which said miPEP has been introduced relative to the length of the roots of an identical plant of the same age into which no miPEP has been introduced, or relative to the length of the roots of an identical plant of the same age into which said miPEP has not been introduced.

In an embodiment, the invention relates to a process for producing a transgenic plant as defined above, in which the rate of nodulation is increased in the plant into which said miPEP has been introduced relative to the rate of nodulation of an identical plant of the same age into which no miPEP has been introduced, or relative to the rate of nodulation of an identical plant of the same age into which said miPEP has not been introduced.

In an embodiment, the invention relates to a process for producing a transgenic plant as defined above, in which the number of nodules is increased in the plant into which said miPEP has been introduced relative to the number of nodules of an identical plant of the same age into which no miPEP has been introduced, or relative to the number of nodules of an identical plant of the same age into which said miPEP has not been introduced.

In an aspect, the invention also relates to a transgenic plant as obtained by the process of production as defined above.

In another aspect, the invention relates to a composition comprising, in combination, a quantity of seeds of a plant and a quantity of a peptide the sequence of which comprises or consists of a sequence identical to that of a miPEP naturally present in said plant.

In an embodiment, the invention relates to a composition comprising, in combination, a quantity of seeds of a plant, in particular A. thaliana, and a quantity of a peptide the sequence of which comprises or consists of a sequence identical to that of AtmiPEP164a1.

In an embodiment, the invention relates to a composition comprising, in combination, a quantity of seeds of a plant, in particular A. thaliana, and a quantity of a peptide the sequence of which comprises or consists of a sequence identical to that of AtmiPEP319a1.

In an embodiment, the invention relates to a composition comprising, in combination, a quantity of seeds of a plant, in particular M. truncatula, and a quantity of a peptide the sequence of which comprises or consists of a sequence identical to that of MtmiPEP171b.

In an embodiment, the invention relates to a composition as defined above, formulated so as to form a dressed seed.

Dressing may be carried out by the processes used conventionally in the agri-food industry and may be obtained using a material able to disaggregate in a solvent or in the ground, such as a binder or clay.

According to the invention, dressing may be used for example for conferring particular properties on a composition of miPEP, or on a composition of seeds in combination with a miPEP.

In an embodiment, the invention relates to a composition as defined above, formulated so as to form a dressed seed comprising MtmiPEP171b.

In an embodiment, the invention relates to a composition as defined above, formulated so as to form a dressed seed comprising AtmiPEP164a1.

In an embodiment, the invention relates to a composition as defined, above formulated so as to form a dressed seed comprising AtmiPEP319a1.

In another aspect, the invention relates to a composition comprising at least one:
miPEP as defined above
nucleic acid encoding said miPEP, or
vector containing said nucleic acid.
   for use as a medicament, in particular for humans or for animals.

The use of the compositions of the invention is applicable in human medicine and in veterinary medicine.

In another aspect, the invention relates to a composition comprising at least one:
miPEP as defined above
nucleic acid encoding said miPEP, or
vector containing said nucleic acid,
   for use in the prevention and/or treatment of a disease involving deregulation of the expression of a gene of the patient,
   the expression of said gene being regulated by a microRNA the accumulation of which is modulated by said miPEP.

In an embodiment, the invention relates to the composition as defined above in which said disease is selected from cancer, diabetes, obesity, infectious diseases and neurodegenerative diseases.

In another aspect, the invention relates to a composition comprising at least one:
miPEP as defined above,
nucleic acid encoding said miPEP, or
vector containing said nucleic acid,
   for use in the prevention and/or treatment of an infection of an animal or of a human with a parasitic organism,
   said parasitic organism having a gene the expression of which is regulated by a microRNA the accumulation of which is modulated by said miPEP.

In another aspect, the invention relates to an antibody specifically recognizing a miPEP.

In particular, the invention relates to an antibody specifically recognizing AtmiPEP165a.

In particular, the invention relates to an antibody specifically recognizing MtmiPEP171b.

In particular, the invention relates to an antibody specifically recognizing AtmiPEP164a1. In particular, the invention relates to an antibody specifically recognizing AtmiPEP319a1.

Such an antibody may be obtained by a process known to a person skilled in the art, such as for example by injecting said miPEP into a non-human animal in order to trigger an immunization reaction and the production of antibodies by said animal.

In another aspect, the invention relates to a process of immunolocalization of a miPEP comprising a step of labelling a biological sample from a plant with an antibody specifically recognizing a miPEP.

In particular, the invention relates to a process of immunolocalization of AtmiPEP165a using an antibody specifically recognizing AtmiPEP165a.

In particular, the invention relates to a process of immunolocalization of MtmiPEP171b using an antibody specifically recognizing MtmiPEP171b.

In particular, the invention relates to a process of immunolocalization of AtmiPEP164a1 using an antibody specifically recognizing AtmiPEP164a1.

In particular, the invention relates to a process of immunolocalization of MtmiPEP319a1 using an antibody specifically recognizing AtmiPEP319a1.

In another aspect, the invention relates to a protocol for producing a recombinant peptide, the sequence of which comprises or consists of a sequence identical to that of a miPEP as defined above, comprising a step of transforming an organism with an expression vector encoding said recombinant peptide.

In an embodiment, said organism is selected from the group comprising bacteria, yeasts, fungi (other than yeasts), animal cells, plants and animals.

In an embodiment, said organism is Escherichia coli.

In particular, the invention relates to a protocol for producing a recombinant peptide as defined above, comprising the following steps:
binding the nucleic acid encoding said recombinant peptide to a nucleic acid encoding a tag, such as GST,
introducing the expression vector containing said nucleic acid encoding said recombinant peptide into the bacterium E. coli,
culturing the bacterium E. coli containing the expression vector in LB medium preferably up to an OD between 0.2 and 0.4,
inducing production of the recombinant peptide with IPTG, preferably for 4 to 5 hours,
centrifuging and lysing the E. coli bacteria,
filtering the supernatant,
purifying said recombinant peptide on a glutathione sepharose affinity column,
if necessary, cleaving the GST with a protease.

All the sequences of the miPEPs, miORFs, miRNAs and primary transcripts of miRNAs are presented in Tables 1, 2, 3, 4, 5 and 6.

Table 7 presents an analysis of the polymorphism of the DNA sequence of the different regions of pri-miR171b (a haplotype is defined when it differs by at least one amino acid from the other haplotypes).

TABLE 1
List of potential miPEPs (miPEPs)

| miPEP | | | Target genes of the | | |
|---|---|---|---|---|---|
| (pI/size MW) | miRNA | Organism | miRNA | Sequence of the miPEP | SEQ ID |
| AtmiPEP156a1 | miR156a | Arabidopsis thaliana | SPL gene family, | MFCSIQCVARHLFPLHVREIKKATRAI | SEQ ID NO: 1 |
| (10.57 / 3824) | | | involved in | KKGKTL | |
| AtmiPEP156a2 | miR156a | Arabidopsis thaliana | development of the | MRRQTSVPFACKRDKESDKSHKER | SEQ ID NO: 2 |
| AtmiPEP156a3 | miR156a | Arabidopsis thaliana | stem and flowering | MVMFFLDLDKNPRFDLLKGLKWNLF | SEQ ID NO: 3 |
| | | | | SSHISPSLPPSL | |
| AtmiPEP156e1 | miR156c | Arabidopsis thaliana | | MKDNFPLLLRL | SEQ ID NO: 4 |
| (8.5 / 1359) | | | | | |
| AtmiPEP156e2 | miR156c | Arabidopsis thaliana | | MSDD | SEQ ID NO: 5 |
| AtmiPEP156e1 | miR156c | Arabidopsis thaliana | | MIYINKYGSISAVEDD | SEQ ID NO: 6 |
| (4.03 / 1818) | | | | | |
| AtmiPEP156f1 | miR156f | Arabidopsis thaliana | | MSQR | SEQ ID NO: 7 |
| (9.5 / 520) | | | | | |
| AlmiPEP159a | miR159a | Arabidopsis lyrata | MYB gene family, | MTCPLLSLSFLLSKYI | SEQ ID NO: 8 |
| AtmiPEP159a1 | miR159a | Arabidopsis thaliana | involved in | MTWPLLSLSFLLSKYV | SEQ ID NO: 9 |
| (8.34 / 1898) | | | germination and | | |
| CrmiPEP159a | miR159a | Capsella rubella | flowering | MTCTLSALSLSLNMFRVN | SEQ ID NO: 10 |
| AtmiPEP159b1 | miR159b | Arabidopsis thaliana | | MFYLS | SEQ ID NO: 11 |
| (5.27 / 659) | | | | | |
| AtmiPEP159b2 | miR159b | Arabidopsis thaliana | | MVNTSSFFISSFILPLVLSESNCLLFRTI | SEQ ID NO: 12 |
| | | | | YKFSMVLY | |
| AtmiPEP160a1 | miR160a | Arabidopsis thaliana | ARF gene family, | MFCLLIPIFSFVFSPNRHLRLQEQ | SEQ ID NO: 13 |
| (8.02 / 2936) | | | involved in | | |
| AtmiPEP160b1 | miR160b | Arabidopsis thaliana | germination, | MFSPQ | SEQ ID NO: 14 |
| (5.28 / 608) | | | development and | | |
| AtmiPEP160b2 | miR160b | Arabidopsis thaliana | flowering | MKYIHILILFKSRSTYKLSTNHI | SEQ ID NO: 15 |
| AtmiPEP161 | miR161 | Arabidopsis thaliana | PPR gene family | MKIPLFLPKL | SEQ ID NO: 16 |
| (10/ 1199) | | | | | |
| AtmiPEP162a1 | miR162a | Arabidopsis thaliana | DCL1 gene, involved | MVSGQEDSWLKLSSLCFLFLSLLDSLI | SEQ ID NO: 17 |
| (4.03 / 3045) | | | in development | | |
| AtmiPEP162b1 | miR162b | Arabidopsis thaliana | | MFLLIFLRLIMICVCSSTDFLRSVNYFC | SEQ ID NO: 18 |
| (5.71 /4114) | | | | LFIYDL | |
| AtmiPEP163-1 | miR163 | Arabidopsis thaliana | SAMT gene family, | MSTTQEHRS | SEQ ID NO: 19 |
| (6.5 / 1076) | | | involved in the | | |
| AtmiPEP163-2 | miR163 | Arabidopsis thaliana | production of | MILKCWSSRFLRVSPYQNAHSLSLG | SEQ ID NO: 20 |
| | | | secondary metabolites | | |
| AlmiPEP164a1 | miR164a | Arabidopsis lyrata | NAC gene family, | MPLAVIRQGIVWP | SEQ ID NO: 21 |
| AlmiPEP164a2 | miR164a | Arabidopsis lyrata | involved in root, foliar | MPSWHDMVLLPYVKHTHANTRHIT | SEQ ID NO: 22 |
| AlmiPEP164a3 | miR164a | Arabidopsis lyrata | and floral | MTWFFCLT | SEQ ID NO: 23 |
| AtmiPEP164a1 | miR164a | Arabidopsis thaliana | development | MPSWHGMVLLPYVKHTHASTHTHTH | SEQ ID NO: 24 |
| (7.05 / 4256) | | | | NIYGCACELVFH | |
| AtmiPEP164a2 | miR164a | Arabidopsis thaliana | | MAWYGSFALRKTHSRQHTHTHT | SEQ ID NO: 25 |
| AtmiPEP164a3 | miR164a | Arabidopsis thaliana | | MVWFFCLT | SEQ ID NO: 26 |
| BrmiPEP164a1 | miR164a | Brassica rapa | | MMIILWK | SEQ ID NO: 27 |
| BrmiPEP164a2 | miR164a | Brassica rapa | | MLWAKLVSFSTLHSLVFLLSPSFA | SEQ ID NO: 28 |
| BrmiPEP164a3 | miR164a | Brassica rapa | | MPSWHGIVILPFVKHTHANIHYSYSC | SEQ ID NO: 29 |
| | | | | VCI | |
| CpmiPEP164a1 | miR164a | Carica papaya | | MIACHPYLPFPLFLSLTFYSIFFSPSPPS | SEQ ID NO: 30 |
| | | | | PSLPL | |
| CpmiPEP164a2 | miR164a | Carica papaya | | MPSLLAFSPFPFSNILLNLLLPLPPFPLS | SEQ ID NO: 31 |
| | | | | AIITIIKPLSLSLPLSLSLSGFSV | |
| CrmiPEP164a1 | miR164a | Capsella rubella | | MELKGLRTWQLLDKV | SEQ ID NO: 32 |
| CrmiPEP164a2 | miR164a | Capsella rubella | | MPSWHGMACFYCLT | SEQ ID NO: 33 |
| CrmiPEP164a3 | miR164a | Capsella rubella | | MAWHGMFLLPYVKHTHANTYSLYM | SEQ ID NO: 34 |
| GrmiPEP164a1 | miR164a | Gossypium raimondii | | MMRSRILKFQYRFGMGIGGRKQLKN | SEQ ID NO: 35 |
| | | | | QLCQIQGRIS | |
| GrmiPEP164a2 | miR164a | Gossypium raimondii | | MSNSRSYQLK | SEQ ID NO: 36 |
| GrmiPEP164a3 | miR164a | Gossypium raimondii | | MNEDLEISTRKRTPQLC | SEQ ID NO: 37 |
| MtmiPEP164a1 | miR164a | Medicago truncatula | | MPKFDIFFYIFV | SEQ ID NO: 38 |
| MtmiPEP164a2 | miR164a | Medicago truncatula | | MSYISLSPKLLPINTKPFPWLVQFNFY | SEQ ID NO: 39 |
| | | | | FSSNTKCNKLHFLGEKLLVGEAGHVQ | |
| | | | | ILFLIHSLIMHINIFCTCSPSPTRLPHPSL | |
| OsmiPEP164a1 | miR164a | Oryza sativa | | MQTHSNTPQSTYSLSLSLSE | SEQ ID NO: 40 |
| OsmiPEP164a2 | miR164a | Oryza sativa | | MCVCDINMHSMLMLL | SEQ ID NO: 41 |
| AlmiPEP165a | miR165a | Arabidopsis lyrata | HD-ZIPIII gene | MRIKLFQLRGMLSGSRILYIYTCVC | SEQ ID NO: 42 |
| AtmiPEP165a | miR165a | Arabidopsis thaliana | family, involved in | MRVKLFQLRGMLSGSRIL | SEQ ID NO: 43 |
| (12.3 /2105) | | | vascular, root, foliar | | |
| BcmiPEP165a | miR165a | Brassica carinata | and floral | MRMKLFQLRGMLSGSRILYIHKYVY | SEQ ID NO: 44 |
| | | | development, and | MLIQVFDHICI | |
| BjmiPEP165a | miR165a | Brassica juncea | nodulation | MRMKLFQLRGMLSGSRILYIHKYVYI | SEQ ID NO: 45 |
| | | | | C | |
| BnmiPEP165a | miR165a | Brassica napus | | MRMKLFQLRGMLSGSRILYIHKYVY | SEQ ID NO: 46 |
| | | | | MIIQVFDHICI | |
| BomiPEP165a | miR165a | Brassica oleracea | | MRMKLFQLRGMLSGSRILYIHKYVY | SEQ ID NO: 47 |
| | | | | MLIQVFDHICI | |
| BrmiPEP165a | miR165a | Brassica rapa | | MRMKLFQLRGMLSGSRILYIHKYVYI | SEQ ID NO: 48 |
| | | | | C | |
| AtmiPEP166a | miR166a | Arabidopsis thaliana | | MLDLFRSNNRIEPSDFRFD | SEQ ID NO: 49 |
| (4.68 / 2372) | | | | | |
| AtmiPEP166b | miR166b | Arabidopsis thaliana | | MRDR | SEQ ID NO: 50 |
| (9.35 / 576) | | | | | |
| AtmiPEP167a | miR167a | Arabidopsis thaliana | ARF gene family, | MNRKISLSLS | SEQ ID NO: 51 |
| (11 / 1148) | | | involved in root and | | |
| AtmiPEP167b1 | miR167b | Arabidopsis thaliana | floral development | MMGCFVGF | SEQ ID NO: 52 |
| (5.27 / 891) | | | gene family of | | |
| AtmiPEP167b2 | miR167b | Arabidopsis thaliana | CCAAT-bing factor, | MQEETYEG | SEQ ID NO: 53 |
| AtmiPEP169c1 | miR169c | Arabidopsis thaliana | involved in | MPHTNLKDLFIFSPNVFFSFAIYLHNS | SEQ ID NO: 54 |
| (9.3 / 7110) | | | nodulation, drought | WNKNYIHKRENFHNTSFALIFFFSSIM | |
| | | | resistance, resistance | SINYG | |
| AtmiPEP169c2 | miR169c | Arabidopsis thaliana | to nitrogen deficiency | MFFFRLLFISTILGTKTTFTNERIFTTPL | SEQ ID NO: 55 |
| | | | | LLSFFFFRPL | |
| AtmiPEP1691 | miR1691 | Arabidopsis thaliana | | MRHKES | SEQ ID NO: 56 |
| (8.52 / 786) | | | | | |
| AtmiPEP171a1 | miR171a | Arabidopsis thaliana | GRAS gene family, | MNLLKKERQRRRQRSIGSHCIASLVL | SEQ ID NO: 57 |
| (11.05 / 4057) | | | involved in floral, | KDGYMKKI | |
| AtmiPEP17 lb | miR171b | Arabidopsis thaliana | foliar, and root | MVLSGKLTF | SEQ ID NO: 58 |
| (8.5 / 995) | | | development, | | |
| MtmiPEP171b1 | miR171b | Medicago truncatula | mycorrhization, | MLLHRLSKFCKIERDIVYIS | SEQ ID NO: 59 |
| | | | nodulation | | |
| MtmiPEP171b2 | miR171b | Medicago truncatula | | MKIEE | SEQ ID NO: 60 |
| ZmmiPEP171b | miR171b | Zea mays | | MHLPSTPSRPPPQHTSLSFLGKEMTKG | SEQ ID NO: 61 |
| | | | | TTTACFG | |
| AtmiPEP171c1 | miR171c | Arabidopsis thaliana | | MLSLSHFHIC | SEQ ID NO: 62 |
| (6.68 / 1187) | | | | | |
| MtmiPEP171e | miR171e | Medicago truncatula | | MMVFGKPKKAMLVRFNPKTDLHV | SEQ ID NO: 63 |
| MtmiPEP171h | miR171h | Medicago truncatula | | MASAAKVYMA | SEQ ID NO: 64 |
| AtmiPEP172a1 | miR172a | Arabidopsis thaliana | AP2 gene family, | MASKIW | SEQ ID NO: 65 |
| (8.5 / 734) | | | involved in floral | | |
| AtmiPEP172a3 | miR172a | Arabidopsis thaliana | development | MVRFQLSIRD | SEQ ID NO: 66 |
| AtmiPEP172b1 | miR172b | Arabidopsis thaliana | | MCTYYYLINKYF | SEQ ID NO: 67 |
| (7.9 / 1621) | | | | | |
| AtmiPEP172c1 | miR172c | Arabidopsis thaliana | | MFPAKWCRLES | SEQ ID NO: 68 |
| (7.98 / 1367) | | | | | |
| AtmiPEP172e1 | miR172e | Arabidopsis thaliana | | MGSLSLFKSQLEILMLLLSLSK | SEQ ID NO: 69 |
| (8.35 / 2452) | | | | | |
| AtmiPEP172e2 | miR172e | Arabidopsis thaliana | | MSVYIHVPISLNCFSPKSSC | SEQ ID NO: 70 |
| AtmiPEP172e3 | miR172e | Arabidopsis thaliana | | MGVPNFRPRNR | SEQ ID NO: 71 |
| AcmiPEP319a1 | miR319a | Arabidopsis cebennensis | TCP gene family, | MRSRVSFFFFKIMLFRLLGYRSM | SEQ ID NO: 72 |
| AcmiPEP319a2 | miR319a | Arabidopsis cebennensis | involved in floral and | MHTYIHTISNISSIFFCSKRSFSPFTYIRI | SEQ ID NO: 73 |
| | | | foliar development | IVVIDPFRIALTFR | |
| AhmiPEP319a | miR319a | Arabidopsis halleri | | MRSRVSLFLSFSSNFAAYSPRS | SEQ ID NO: 74 |
| AlmiPEP319a | miR319a | Arabidopsis lyrata | | MHTYIPSSSFPISNISSVFFCYKRSFSPY | SEQ ID NO: 75 |
| | | | | TYIRIIVVIDPFRIALTFR | |
| AtmiPEP319a1 | miR319a | Arabidopsis thaliana | | MNIHTYHHLLFPSLVFHQSSDVPNALS | SEQ ID NO: 76 |
| (6.56 / 5917) | | | | LHIHTYEYIIVVIDPFRITLAFR | |
| AtmiPEP319a2 | miR319a | Arabidopsis thaliana | | MFQTLYLFIYIHTNILLLS | SEQ ID NO: 77 |
| BrmiPEP319a | miR319a | Brassica rapa | | MFKLYFSAILSTQYMHTYHHRIALIFL | SEQ ID NO: 78 |
| | | | | SILYPSTNYLMSPILNPT | |
| CpmiPEP319a | miR319a | Carica papaya | | MKIKLGFSLIKIIILLDKNS | SEQ ID NO: 79 |
| CrmiPEP319a | miR319a | Capsella rubella | | MHPHTYIHIPSSSFLISSFCL | SEQ ID NO: 80 |
| EgmiPEP319a | miR319a | Eucalyptus grandis | | MKHIQRWRYGETSGRQGDWKRLEIK | SEQ ID NO: 81 |
| | | | | VHSNPSLKVKKNTNNFSSSL | |
| GrmiPEP319a | miR319a | Gossypium raimondii | | MIHFNLSQWRAICMANFHLTYSFLFG | SEQ ID NO: 82 |
| | | | | VL | |
| MtmiPEP319a | miR319a | Medicago truncatula | | MHVYLELFMVIKGLGFLLLVK | SEQ ID NO: 83 |
| OsmiPEP319a | miR319a | Oryza sativa | | MEMIQRPCLILKFFFKLSTLYIP | SEQ ID NO: 84 |
| PpmiPEP319a | miR319a | Physcomitrella patens | | MFHRRRSSVLLPPFGQTQPNPRCLPDL | SEQ ID NO: 85 |
| | | | | RFPSCFTPCTA | |
| ThmiPEP319a1 | miR319a | Thellungiella halophila | | MTICKVSKACFYAGKIENSRLIKKIGIP | SEQ ID NO: 86 |
| | | | | KREGAPFSPIRENQ | |
| ThmiPEP319a2 | miR319a | Thellungiella halophila | | MEIQIKKKNLYIMNTQKLPNLYIYIYK | SEQ ID NO: 87 |
| | | | | YVFIKLMVVE | |
| AtmiPEP319b1 | miR319b | Arabidopsis thaliana | | MVPQINLWSSRVILKIRIDSSTHREED | SEQ ID NO: 88 |
| (8.04 / 5120) | | | | HCIQNHKHGLSFIFSF | |
| AtmiPEP394 al | miR394a | Arabidopsis thaliana | F-box gene family, | MSLQFYERVSFKNTVK | SEQ ID NO: 89 |
| (9.7 / 1977) | | | involved in foliar | | |
| | | | development and | | |
| | | | drought resistance | | |
| AtmiPEP395e1 | miR395c | Arabidopsis thaliana | Family of the APS and | MTEQEEESQMST | SEQ ID NO: 90 |
| (3.58 / 1429) | | | AST genes, involved | | |
| AtmiPEP395e1 | miR395e | Arabidopsis thaliana | in germination and | MYLQYIDNVISIYSNNRRVGRMFSRV | SEQ ID NO: 91 |
| (9.98 / 4700) | | | sulphur metabolism | PLSTSLEIQFFIK | |
| AtmiPEP397b1 | miR397b | Arabidopsis thaliana | Family of the genes of | MSKEIFFSPGFE | SEQ ID NO: 92 |
| (4.53 / 1418) | | | laccases, involved in | | |
| | | | copper metabolism, | | |
| | | | their overexpression | | |
| | | | improves growth | | |
| AtmiPEP398c1 | miR398c | Arabidopsis thaliana | CSD gene family, | MIRTHEQSTAITTLRHCYSSRFMCSQV | SEQ ID NO: 93 |
| | | | involved in copper | TPAELFLYRPCFINAVAR | |
| | | | metabolism, its | | |
| | | | overexpression | | |
| | | | improves growth | | |
| AtmiPEP399b | miR399b | Arabidopsis thaliana | PHO2 gene family, | MKRNM | SEQ ID NO: 94 |
| (11 /678) | | | involved in | | |
| AtmiPEP399d1 | miR399d | Arabidopsis thaliana | phosphorus | MQCEI | SEQ ID NO: 95 |
| (4 / 622) | | | metabolism | | |
| AtmiPEP403 | miR403 | Arabidopsis thaliana | AGO gene family | MFCA | SEQ ID NO: 96 |
| (5.27470) | | | | | |
| AtmiPEP447a1 | miR447a | Arabidopsis thaliana | Family of the genes of | MVMAHH | SEQ ID NO: 97 |
| (6.69 / 724) | | | phosphoglycerate | MMKPRWNCSLYGITEWTNNQNQKSK | SEQ ID NO: 98 |
| AtmiPEP447a2 | miR447a | Arabidopsis thaliana | kinase | RKGRRKTQIWRIGDRLDTVECITLML | |
| | | | | SAY | |
| AtmiPEP447b1 | miR447b | Arabidopsis thaliana | | MLLIIVELVL | SEQ ID NO: 99 |
| (4 / 1155) | | | | | |
| AtmiPEP447b2 | miR447b | Arabidopsis thaliana | | MLCFNFRCVRRFAE | SEQ ID |
| | | | | | NO: 100 |
| AtmiPEP447c | miR447c | Arabidopsis thaliana | | MYTYQLDNSFSWFLCTRFCLYRYFLF | SEQ ID |
| | | | | NFRCFRRFSE | NO: 101 |
| DmmiPEP1a | miR1 | Drosophila melanogaster | Muscular | MWREVCAQKSQTKRRNFITGNQRRN | SEQ ID |
| | | | differentiation | KTKANRKAETKQQKVYEFFVQARER | NO: 102 |
| | | | | CKTRKKHEKKTLKKTKKIQNRYRAV | |
| | | | | SENEWGKGFPSHI | |
| DmmiPEP1b | miR1 | Drosophila melanogaster | Muscular | MRTKKSNKKAQFYYGQPTTKQNKSQ | SEQ ID |
| | | | differentiation | PKSRNKAAKSL | NO: 103 |
| DmmiPEP8 | miR8 | Drosophila melanogaster | Growth | MEPGFVFVLFPTHLSTQHTQREKSILV | SEQ ID |
| | | | | MGLNLQSAKQSDKQNSKERKKNTQI | NO: 104 |
| | | | | NSQRIPYRQGGQCSKVLSP | |
| HsmiPEP155 | miR155 | Homo sapiens | inflammation | MEMALMVAQTRKGKSVV | SEQ ID |
| | | | | | NO: 355 |
| AtmiPEP157c | miR157c | Arabidopsis thaliana | SPL gene family, | MMLHITHRFESDVGC | SEQ ID |
| (5.95 / 1776) | | | involved in | | NO: 375 |
| AtmiPEP157d | miR157d | Arabidopsis thaliana | development of the | MLYV | SEQ ID |
| (5.27 / 524) | | | stem and flowering | | NO: 376 |
| AtmiPEP160c | miR160c | Arabidopsis thaliana | ARF gene family, | MFMRRGLVYNNIYI | SEQ ID |
| (9.98 / 1790) | | | involved in | | NO: 377 |
| | | | germination, | | |
| | | | development and | | |
| | | | flowering | | |
| AtmiPEP164b | miR164b | Arabidopsis thaliana | NACgene family, | MMKVCDEQDGEAGHVHY | SEQ ID |
| (4.72 / 1949) | | | involved in root, foliar | | NO: 378 |
| | | | and floral | | |
| | | | development | | |
| AtmiPEP166c | miR166c | Arabidopsis thaliana | HD-ZIPIII gene | MKKRITRINLEEQIKKTLDDSRTRLHS | SEQ ID |
| (10.42 / 3407) | | | family, involved in | P | NO: 379 |
| AtmiPEP166d | miR166d | Arabidopsis thaliana | vascular, root, foliar | MKKIGSIDSF | SEQ ID |
| (8.35 / 1125) | | | and floral | | NO: 380 |
| | | | development and | | |
| | | | nodulation | | |
| AtmiPEP169a | miR169a | Arabidopsis thaliana | Gene family of | MTCRFK | SEQ ID |
| (9.5 / 784) | | | CCAAT-bing factor, | | NO: 381 |
| AtmiPEP169h1 | miR169h | Arabidopsis thaliana | involved in | MVT | |
| (5.28 / 349) | | | nodulation, drought | | |
| AtmiPEP169h2 | miR169h | Arabidopsis thaliana | resistance, resistance | MKNENLCGSQG | SEQ ID |
| | | | to nitrogen deficiency | | NO: 382 |
| AtmiPEP169n | miR169n | Arabidopsis thaliana | | MKCMMKKRGLTWRKASCLVAKDDL | SEQ ID |
| (8.96 /5315) | | | | PDLFRLHDSISNSCILDYYTF | NO: 383 |
| AtmiPEP170 | miR170 | Arabidopsis thaliana | GRAS gene family, | MFPRESL | SEQ ID |
| (5.75 / 879) | | | involved in floral, | | NO: 384 |
| | | | foliar, and root | | |
| | | | development, | | |
| | | | mycorrhization, | | |
| | | | nodulation | | |
| AtmiPEP396a | miR396a | Arabidopsis thaliana | Family of the GRF | MTLSVFFHSFLELQNFFRFFFFSFDISY | SEQ ID |
| (5.3 / 3636) | | | genes involved in root | A | NO: 385 |
| | | | development and | | |
| | | | cellular proliferation, | | |
| | | | mycorrhization | | |
| AtmiPEP399c | miR399c | Arabidopsis thaliana | PHO2 gene family, | MSLAKGELPCHCFRLNTVYNRFC | SEQ ID |
| (8.66/2703) | | | involved in | | NO: 386 |
| | | | phosphorus | | |
| | | | metabolism | | |

TABLE 2
List of the miORFs

| miPEP | Organism | Sequence of the miORF | SEQ ID |
|---|---|---|---|
| AtmiPEP156a1 | Arabidopsis thaliana | ATGTTCTGTTCAATTCAATGCGTCGCCAGACATCTGTTCCCTTTGC | SEQ ID NO: 105 |
| | | ATGTAAGAGAGATAAAGAAAGCGACAAGAGCCATAAAGAAAGG | |
| | | TAA | |
| AtmiPEP156a2 | Arabidopsis thaliana | ATGCGTCGCCAGACATCTGTTCCCTTTGCATGTAAGAGAGATAAA | SEQ ID NO: 106 |
| | | GAAAGCGACAAGAGCCATAAAGAAAGGTAA | |
| AtmiPEP156a3 | Arabidopsis thaliana | ATGGTTATGTTTTTTCTCGATTTAGACAAAAACCCTAGATTTGATC | SEQ ID NO: 107 |
| | | TTCTAAAGGGTCTCAAATGGAATCTCTTCTCTTCTCATATCTCTCC | |
| | | CTCTCTCCCTCCCTCTCTTTGA | |
| AtmiPEP156c1 | Arabidopsis thaliana | ATGAAGGACAACTTTCCTCTTCTCCTTCGGTTATAA | SEQ ID NO: 108 |
| AtmiPEP156c2 | Arabidopsis thaliana | ATGAGTGATGACTGA | SEQ ID NO: 109 |
| AtmiPEP156e1 | Arabidopsis thaliana | ATGATATATATAAATAAATATGGGTCGATATCGGCTGTGGAGGAC | SEQ ID NO: 110 |
| | | GACTAG | |
| AtmiPEP156f1 | Arabidopsis thaliana | ATGAGCCAAAGATAA | SEQ ID NO: 111 |
| AlmiPEP159a | Arabidopsis lyrata | ATGACGTGTCCTCTTCTCTCTCTCTCTTTCCTTCTCTCTAAGTATAT | SEQ ID NO: 112 |
| | | TTAG | |
| AtmiPEP159a1 | Arabidopsis thaliana | ATGACGTGGCCTCTTCTCTCTCTCTCTTTCCTTCTCTCTAAGTATGT | SEQ ID NO: 113 |
| | | TTAG | |
| CrmiPEP159a | Capsella rubella | ATGACGTGTACTCTCTCTGCTCTATCTCTCTCTCTAAATATGTTTA | SEQ ID NO: 114 |
| | | GGGTTAA | |
| AtmiPEP159b1 | Arabidopsis thaliana | ATGTTTTATCTTTCATAA | SEQ ID NO: 115 |
| AtmiPEP159b2 | Arabidopsis thaliana | ATGGTTAATACTAGTAGCTTTTTCATTTCAAGTTTTATCCTTCCAT | SEQ ID NO: 116 |
| | | TGGTTCTTTCTGAGTCAAATTGTCTCCTGTTTCGAACCATATATAA | |
| | | GTTTTCAATGGTTTTGTATTAA | |
| AtmiPEP160a1 | Arabidopsis thaliana | ATGTTTTGTTTGTTGATTCCCATCTTCTCTTTTGTCTTTTCACCAAA | SEQ ID NO: 117 |
| | | TCGTCATTTAAGGCTTCAAGAACAGTAA | |
| AtmiPEP160b1 | Arabidopsis thaliana | ATGTTTTCCCCTCAATGA | SEQ ID NO: 118 |
| AtmiPEP160b2 | Arabidopsis thaliana | ATGAAATACATACACATTTTGATTTTATTTAAATCAAGATCGACG | SEQ ID NO: 119 |
| | | TATAAGCTATCCACCAATCATATTTAA | |
| AtmiPEP161 | Arabidopsis thaliana | ATGAAAATTCCATTGTTTCTGCCGAAGCTTTGA | SEQ ID NO: 120 |
| AtmiPEP162a1 | Arabidopsis thaliana | ATGGTATCTGGTCAAGAAGATTCCTGGTTAAAACTTTCATCTCTCT | SEQ ID NO: 121 |
| | | GTTTCCTTTTTCTTTCTTTGTTGGATTCATTAATTTGA | |
| AtmiPEP162b1 | Arabidopsis thaliana | ATGTTTCTTTTAATCTTTTTGAGATTAATAATGATTTGTGTTTGTTC | SEQ ID NO: 122 |
| | | ATCAACCGATTTTCTCAGATCTGTCAATTATTTTTGTTTATTTATTT | |
| | | ATGATTTATGA | |
| AtmiPEP163-1 | Arabidopsis thaliana | ATGTCCACTACTCAAGAGCATAGGTCTTGA | SEQ ID NO: 123 |
| AtmiPEP163-2 | Arabidopsis thaliana | ATGATACTAAAGTGCTGGAGTTCCCGGTTCCTGAGAGTGAGTCCA | SEQ ID NO: 124 |
| | | TATCAAAATGCGCATTCGTTATCACTTGGTTGA | |
| AlmiPEP164a1 | Arabidopsis lyrata | ATGCCCTTAGCAGTTATTAGACAAGGGATTGTTTGGCCCTAG | SEQ ID NO: 125 |
| AlmiPEP164a2 | Arabidopsis lyrata | ATGCCATCATGGCATGACATGGTTCTTTTGCCTTACGTAAAACAC | SEQ ID NO: 126 |
| | | ACTCACGCCAACACACGCCACATAACATAA | |
| AlmiPEP164a3 | Arabidopsis lyrata | ATGACATGGTTCTTTTGCCTTACGTAA | SEQ ID NO: 127 |
| AtmiPEP164a1 | Arabidopsis thaliana | ATGCCATCATGGCATGGTATGGTTCTTTTGCCTTACGTAAAACAC | SEQ ID NO: 128 |
| | | ACTCACGCCAGCACACACACACACACACATAACATATACGGATG | |
| | | TGCGTGTGAGCTAGTCTTCCATTAA | |
| AtmiPEP164a2 | Arabidopsis thaliana | ATGGCATGGTATGGTTCTTTTGCCTTACGTAAAACACACTCACGC | SEQ ID NO: 129 |
| | | CAGCACACACACACACACACATAA | |
| AtmiPEP164a3 | Arabidopsis thaliana | ATGGTATGGTTCTTTTGCCTTACGTAA | SEQ ID NO: 130 |
| BrmiPEP164a1 | Brassica rapa | ATGATGATAATTTTGTGGAAATAA | SEQ ID NO: 131 |
| BrmiPEP164a2 | Brassica rapa | ATGCTTTGGGCCAAGCTAGTTTCTTTTAGCACTCTTCACTCACTAG | SEQ ID NO: 132 |
| | | TTTTTCTTCTCAGCCCTTCTTTTGCGTGA | |
| BrmiPEP164a3 | Brassica rapa | ATGCCATCATGGCATGGCATTGTCATTTTGCCTTTCGTAAAACAC | SEQ ID NO: 133 |
| | | ACTCACGCCAACATACATTATTCATATTCATGTGTATGTATATGA | |
| | | ATGCCATCATGGCATATGCCATCATGGCAT | |
| CpmiPEP164a1 | Carica papaya | ATGATTGCATGCCATCCCTACTTGCCTTTTCCCCTTTTCCTTTCTCT | SEQ ID NO: 134 |
| | | AACATTTTACTCAATCTTCTTCTCCCCCTCCCCCCCTTCCCCCTCTC | |
| | | TGCCATTATAA | |
| CpmiPEP164a2 | Carica papaya | ATGCCATCCCTACTTGCCTTTTCCCCTTTTCCTTTCTCTAACATTTT | SEQ ID NO: 135 |
| | | ACTCAATCTTCTTCTCCCCCTCCCCCCCTTCCCCCTCTCTGCCATTA | |
| | | TAACCATAATTAAACCTCTCTCCCTCTCTCTCCCTCTCTCTCTCTCT | |
| | | CTCTCTGGGTTCTCAGTATAA | |
| CrmiPEP164a1 | Capsella rubella | ATGGAATTAAAAGGTTTGAGAACTTGGCAGTTATTAGACAAGGTA | SEQ ID NO: 136 |
| | | TAG | |
| CrmiPEP164a2 | Capsella rubella | ATGCCATCATGGCATGGCATGGCATGTTTCTATTGCCTTACGTAA | SEQ ID NO: 137 |
| CrmiPEP164a3 | Capsella rubella | ATGGCATGGCATGTTTCTATTGCCTTACGTAAAACACACTCACGC | SEQ ID NO: 138 |
| | | CAACACATACTCACTATACATGTAAATAAGTATGTGCGCGTGTGA | |
| GrmiPEP164a1 | Gossypium raimondii | ATGATGAGATCAAGAATTTTAAAGTTTCAATATAGATTTGGCATG | SEQ ID NO: 139 |
| | | GGTATTGGCGGCAGAAAGCAATTAAAAAACCAGTTATGTCAAAT | |
| | | TCAAGGTCGTATCAGTTAA | |
| GrmiPEP164a2 | Gossypium raimondii | ATGTCAAATTCAAGGTCGTATCAGTTAAAATGA | SEQ ID NO: 140 |
| GrmiPEP164a3 | Gossypium raimondii | ATGAATGAAGATTTAGAAATTTCAACAAGGAAGAGGACCCCACA | SEQ ID NO: 141 |
| | | GCTTTGTTAA | |
| MtmiPEP164a1 | Medicago truncatula | ATGCCCAAATTTGATATTTTTTTTTATATATTTGTATAG | SEQ ID NO: 142 |
| MtmiPEP164a2 | Medicago truncatula | ATGTCATATATCTCTCTCTCTCCTAAGTTGCTACCTATAAATACTA | SEQ ID NO: 143 |
| | | AGCCTTTCCCTTGGTTGGTTCAATTCAACTTCTACTTCTCATCAAA | |
| | | CACAAAGTGCAATAAGCTTCATTTCCTGGGTGAGAAGCTCCTTGT | |
| | | TGGAGAAGCAGGGCACGTGCAAATCCTCTTTCTGATTCATTCTCT | |
| | | CATAATGCATATCAATATCTTTTGCACGTGCTCCCCTTCTCCAACT | |
| | | AGG | |
| OsmiPEP164a1 | Oryza saliva | ATGCAAACCCACTCCAACACTCCACAATCCACATACTCTCTCTCT | SEQ ID NO: 144 |
| | | CTCTCTCTCTCTGAGTAG | |
| OsmiPEP164a2 | Oryza saliva | ATGTGTGTGTGTGATATCAATATGCATTCGATGTTGATGCTACTGT | SEQ ID NO: 145 |
| | | AG | |
| AlmiPEP165a | Arabidopsis lyrata | ATGAGAATTAAGCTATTTCAGTTGAGGGGAATGTTGTCTGGATCG | SEQ ID NO: 146 |
| | | AGGATATTATACATATATACATGTGTATGTTGA | |
| AtmiPEP165a | Arabidopsis thaliana | ATGAGGGTTAAGCTATTTCAGTTGAGGGGAATGTTGTCTGGATCG | SEQ ID NO: 147 |
| | | AGGATATTATAG | |
| BemiPEP165a | Brassica carinata | ATGAGAATGAAGCTATTTCAGTTGAGGGGAATGTTGTCTGGATCG | SEQ ID NO: 148 |
| | | AGGATATTATATATACACAAATACGTATATATGTTAATACAAGTG | |
| | | TTTGATCATATATGTATATAG | |
| BjmiPEP165a | Brassica juncea | ATGAGAATGAAGCTATTTCAGTTGAGGGGAATGTTGTCTGGATCG | SEQ ID NO: 149 |
| | | AGGATATTATATATACACAAATATGTATATATATGTTAA | |
| BnmiPEP165a | Brassica napus | ATGAGAATGAAGCTATTTCAGTTGAGGGGAATGTTGTCTGGATCG | SEQ ID NO: 150 |
| | | AGGATATTATATATACACAAATACGTATATATGATAATACAAGTG | |
| | | TTTGATCATATATGTATATAG | |
| BomiPEP165a | Brassica oleracea | ATGAGAATGAAGCTATTTCAGTTGAGGGGAATGTTGTCTGGATCG | SEQ ID NO: 151 |
| | | AGGATATTATATATACACAAGTACGTATATATGTTAATACAAGTG | |
| | | TTTGATCATATATGTATATAG | |
| BrmiPEP165a | Brassica rapa | ATGAGAATGAAGCTATTTCAGTTGAGGGGAATGTTGTCTGGATCG | SEQ ID NO: 152 |
| | | AGGATATTATATATACACAAATATGTATATATATGTTAA | |
| AtmiPEP166a | Arabidopsis thaliana | ATGTTGGATCTCTTTCGATCTAACAATCGAATTGAACCTTCAGATT | SEQ ID NO: 153 |
| | | TCAGATTTGATTAG | |
| AtmiPEP166b | Arabidopsis thaliana | ATGAGAGATAGATAA | SEQ ID NO: 154 |
| AtmiPEP167a | Arabidopsis thaliana | ATGAACAGAAAAATCTCTCTTTCTCTTTCTTGA | SEQ ID NO: 155 |
| AtmiPEP167b1 | Arabidopsis thaliana | ATGATGGGTTGTTTTGTGGGATTTTAA | SEQ ID NO: 156 |
| AtmiPEP167b2 | Arabidopsis thaliana | ATGCAGGAGGAAACATATGAGGGGTGA | SEQ ID NO: 157 |
| AtmiPEP169c1 | Arabidopsis thaliana | ATGCCACATACAAACTTGAAAGATCTCTTCATCTTTTCTCCAAATG | SEQ ID NO: 158 |
| | | TTTTTTTTTCGTTTGCTATTTATCTCCACAATTCTTGGAACAAAAA | |
| | | CTACATTCACAAACGAGAGAATTTTCACAACACCTCTTTTGCTCTC | |
| | | ATTTTTTTTTTTTCGTCCATTATGAGTATTAATTATGGTTAG | |
| AtmiPEP169c2 | Arabidopsis thaliana | ATGTTTTTTTTTCGTTTGCTATTTATCTCCACAATTCTTGGAACAA | SEQ ID NO: 159 |
| | | AAACTACATTCACAAACGAGAGAATTTTCACAACACCTCTTTTGC | |
| | | TCTCATTTTTTTTTTTTCGTCCATTATGA | |
| AtmiPEP16911 | Arabidopsis thaliana | ATGAGACATAAAGAGAGTTAA | SEQ ID NO: 160 |
| AtmiPEP171a1 | Arabidopsis thaliana | ATGAACCTCCTCAAGAAGGAAAGACAGAGGAGGAGACAAAGAA | SEQ ID NO: 161 |
| | | GTATAGGTTCACATTGCATAGCCAGTTTAGTTTTGAAGGATGGAT | |
| | | ATATGAAAAAAATATGA | |
| AtmiPEP171b | Arabidopsis thaliana | ATGGTTCTCTCCGGTAAATTAACATTTTAG | SEQ ID NO: 162 |
| MtmiPEP171b1 | Medicago truncatula | ATGCTTCTTCATAGGCTCTCCAAATTTTGCAAAATTGAAAGAGAC | SEQ ID NO: 163 |
| | | ATAGTATATATATCTTAG | |
| MtmiPEP171b2 | Medicago truncatula | ATGAAGATTGAAGAGTAA | SEQ ID NO: 164 |
| ZmmiPEP171b | Zea mays | ATGCATCTGCCTTCAACTCCCTCTCGCCCCCCACCCCAACACACAT | SEQ ID NO: 165 |
| | | CTCTCTCTTTTCTAGGGAAGGAAATGACGAAGGGGACGACGACG | |
| | | GCATGCTTCGGCTAG | |
| AtmiPEP171c1 | Arabidopsis thaliana | ATGTTGTCTCTTTCTCATTTTCATATCTGCTAA | SEQ ID NO: 166 |
| MtmiPEP171e | Medicago truncatula | ATGATGGTGTTTGGGAAGCCGAAAAAAGCGATGTTGGTGAGGTT | SEQ ID NO: 167 |
| | | CAATCCGAAGACGGATTTACATGTATAG | |
| MtmiPEP171h | Medicago truncatula | ATGGCTTCAGCTGCAAAAGTATACATGGCGTGA | SEQ ID NO: 168 |
| AtmiPEP172a1 | Arabidopsis thaliana | ATGGCTTCCAAGATCTGGTAA | SEQ ID NO: 169 |
| AtmiPEP172a3 | Arabidopsis thaliana | ATGGTTAGGTTCCAACTAAGTATACGAGATTAA | SEQ ID NO: 170 |
| AtmiPEP172b1 | Arabidopsis thaliana | ATGTGTACGTACTATTATCTCATAAATAAATATTTTTAA | SEQ ID NO: 171 |
| AtmiPEP172c1 | Arabidopsis thaliana | ATGTTTCCAGCAAAATGGTGCCGTCTTGAGTCTTGA | SEQ ID NO: 172 |
| AtmiPEP172e1 | Arabidopsis thaliana | ATGGGATCTCTCTCTTTATTTAAAAGTCAATTAGAGATCTTGATGC | SEQ ID NO: 173 |
| | | TACTTCTGTCCCTTTCCAAGTGA | |
| AtmiPEP172e2 | Arabidopsis thaliana | ATGAGTGTATATATTCATGTACCTATCTCTCTCAATTGCTTCTCAC | SEQ ID NO: 174 |
| | | CAAAATCATCTTGCTGA | |
| AtmiPEP172e3 | Arabidopsis thaliana | ATGGGAGTTCCCAACTTTAGACCTCGAAACCGATAA | SEQ ID NO: 175 |
| AcmiPEP319a1 | Arabidopsis cebennensis | ATGAGATCTAGGGTTTCTTTCTTTTTCTTCAAAATCATGCTTTTTC | SEQ ID NO: 176 |
| | | GCTTGCTAGGTTATAGATCCATGTAA | |
| AcmiPEP319a2 | Arabidopsis cebennensis | ATGCATACATACATACATACCATCTCTAATATTTCATCAATCTTCT | SEQ ID NO: 177 |
| | | TTTGTTCCAAACGCCTTTCTCTCCATTTACATACATACGAATCATT | |
| | | GTTGTCATAGATCCGTTTAGAATTGCTTTAACTTTTAGATGA | |
| AhmiPEP319a | Arabidopsis halleri | ATGAGATCTAGGGTTTCTTTGTTTCTTTCGTTTTCTTCAAATTTTGC | SEQ ID NO: 178 |
| | | TGCATATTCTCCAAGATCATGA | |
| AlmiPEP319a | Arabidopsis lyrata | ATGCATACATACATACCATCATCATCTTTTCCCATCTCTAATATTT | SEQ ID NO: 179 |
| | | CATCAGTCTTCTTTTGTTACAAACGCTCTTTCTCGCCATATACATA | |
| | | CATAAGAATCATTGTTGTCATAGATCCGTTTAGAATTGCTTTAACT | |
| | | TTTAGATGA | |
| AtmiPEP319a1 | Arabidopsis thaliana | ATGAATATACATACATACCATCATCTTCTTTTCCCATCTCTAGTTT | SEQ ID NO: 180 |
| | | TTCATCAATCTTCTGATGTTCCAAACGCTCTATCTCTTCATATACA | |
| | | TACATACGAATATATTATTGTTGTCATAGATCCATTTAGAATCACT | |
| | | TTAGCTTTTAGATGA | |
| AtmiPEP319a2 | Arabidopsis thaliana | ATGTTCCAAACGCTCTATCTCTTCATATACATACATACGAATATAT | SEQ ID NO: 181 |
| | | TATTGTTGTCATAG | |
| BrmiPEP319a | Brassica rapa | ATGTTTAAGCTCTACTTCTCAGCAATTCTCTCCACCCAATACATGC | SEQ ID NO: 182 |
| | | ATACATACCATCATCGTATCGCTCTAATTTTTCTATCAATCTTGTA | |
| | | TCCTTCCACAAATTATCTTATGTCTCCCATTTTAAATCCTACATAG | |
| CpmiPEP319a | Carica papaya | ATGAAGATTAAATTAGGTTTTAGTCTTATTAAGATTATTATATTAC | SEQ ID NO: 183 |
| | | TAGACAAAAACAGTTAA | |
| CrmiPEP319a | Capsella rubella | ATGCATCCACATACATACATACATATACCATCATCTTCTTTTCTCA | SEQ ID NO: 184 |
| | | TCTCTAGTTTTTGTTTATAA | |
| EgmiPEP319a | Eucalyptus grandis | ATGAAGCATATTCAAAGGTGGAGATATGGGGAGACTTCCGGAAG | SEQ ID NO: 185 |
| | | GCAAGGGGATTGGAAAAGGCTCGAGATCAAAGTGCATAGCAACC | |
| | | CTTCGCTAAAGGTGAAAAAGAATACGAATAACTTCAGTAGCTCAC | |
| | | TTTAA | |
| GrmiPEP319a | Gossypium raimondii | ATGATCCATTTCAACCTGTCACAGTGGAGAGCAATTTGTATGGCT | SEQ ID NO: 186 |
| | | AATTTCCATCTCACCTATTCTTTTCTGTTTGGGGTTCTCTAG | |
| MtmiPEP319a | Medicago truncatula | ATGCATGTATATCTTGAATTGTTTATGGTAATAAAGGGGTTAGGA | SEQ ID NO: 187 |
| | | TTTCTCCTTTTGGTGAAGTGA | |
| OsmiPEP319a | Olyza sativa | ATGGAAATGATACAAAGGCCGTGTTTAATTTTAAAATTTTTTTTCA | SEQ ID NO: 188 |
| | | AACTTTCAACACTTTACATCCCATAA | |
| PpmiPEP319a | Physcomitrella patens | ATGTTCCACCGTCGGAGATCCTCGGTGCTGCTACCCCCGTTCGGC | SEQ ID NO: 189 |
| | | CAAACCCAACCCAACCCTAGGTGTCTGCCGGACCTCCGCTTCCCC | |
| | | TCCTGCTTCACCCCCTGCACCGCTTAA | |
| ThmiPEP319a1 | Thellungiella halophila | ATGACGATATGTAAAGTAAGCAAGGCATGTTTTTATGCAGGGAA | SEQ ID NO: 190 |
| | | GATTGAAAATTCAAGATTAATCAAGAAAATTGGAATACCAAAAA | |
| | | GAGAGGGAGCTCCCTTCAGTCCAATCAGAGAGAATCAATGA | |
| ThmiPEP319a2 | Thellungiella halophila | ATGGAGATTCAAATTAAAAAGAAAAACTTATATATAATGAATAC | SEQ ID NO: 191 |
| | | ACAAAAGCTACCTAATCTGTATATATATATATATAAATATGTCTT | |
| | | CATTAAATTAATGGTCGTGGAATAG | |
| AtmiPEP319b1 | Arabidopsis thaliana | ATGGTACCTCAAATTAATCTATGGTCATCTAGGGTTATCTTGAAG | SEQ ID NO: 192 |
| | | ATTAGAATTGATTCTAGCACGCACAGAGAGGAAGATCATTGCATC | |
| | | CAGAATCACAAACATG3GCCTATCTTTTATCTTTTCTTTTTGA | |
| AtmiPEP394a1 | Arabidopsis thaliana | ATGTCTCTCCAATTTTATGAGAGGGTTTCCTTCAAGAACACAGTA | SEQ ID NO: 193 |
| | | AAATAG | |
| AtmiPEP395c1 | Arabidopsis thaliana | ATGACAGAGCAAGAAGAAGAAAGTCAAATGTCCACATGA | SEQ ID NO: 194 |
| AtmiPEP395e1 | Arabidopsis thaliana | ATGTATCTACAATATATTGATAATGTAATATCTATATATTCAAAC | SEQ ID NO: 195 |
| | | AATCGTCGTGTTGGTCGGATGTTTTCTAGAGTTCCTCTGAGCACTT | |
| | | CATTGGAGATACAATTTTTTATAAAATAG | |
| AtmiPEP397b1 | Arabidopsis thaliana | ATGAGCAAGGAGATATTTTTTTCCCCTGGGTTTGAATGA | SEQ ID NO: 196 |
| AtmiPEP398c1 | Arabidopsis thaliana | ATGAGAACACACGAGCAATCAACGGCTATAACGACGCTACGTCA | SEQ ID NO: 197 |
| | | TTGTTACAGCTCTCGTTTCATGTGTTCTCAGGTCACCCCTGCTGAG | |
| | | CTCTTTCTCTACCGTCCATGTTTTATCAACGCCGTGGCCCGTG | |
| AtmiPEP399b | Arabidopsis thaliana | ATGAAGAGAAACATGTAA | SEQ ID NO: 198 |
| AtmiPEP399d1 | Arabidopsis thaliana | ATGCAATGTGAAATATGA | SEQ ID NO: 199 |
| AtmiPEP403 | Arabidopsis thaliana | ATGTTTTGTGCTTGA | SEQ ID NO: 200 |
| AtmiPEP447a1 | Arabidopsis thaliana | ATGGTCATGGCTCATCATTAG | SEQ ID NO: 201 |
| AtmiPEP447a2 | Arabidopsis thaliana | ATGATGAAACCTCGATGGAACTGCTCTCTTTATGGAATCACGGAA | SEQ ID NO: 202 |
| | | TGGACAAATAATCAAAATCAGAAATCGAAGCGAAAAGGGAGGA | |
| | | GAAAAACGCAGATTTGGAGGATTGGGGACAGATTAGATACTGTT | |
| | | GAATGCATCACTCTAATGCTATCAGCCTATTAA | |
| AtmiPEP447b1 | Arabidopsis thaliana | ATGCTGCTTATCATCGTGGAGTTGGTTCTGTAA | SEQ ID NO: 203 |
| AtmiPEP447b2 | Arabidopsis thaliana | ATGCTTTGTTTCAATTTCAGGTGCGTTAGAAGGTTTGCAGAGTAG | SEQ ID NO: 204 |
| AtmiPEP447c | Arabidopsis thaliana | ATGTACACCTACCAGCTTGATAACTCTTTTTCGTGGTTTCTGTGTA | SEQ ID NO: 205 |
| | | CTCGTTTCTGTTTGTACAGATACTTCTTGTTCAATTTCAGATGCTTT | |
| | | AGAAGGTTTTCGGAG | |
| dmmiPEP1a | Drosophila melanogaster | ATGTGGCGCGAAGTATGCGCACAAAAAAGTCAAACAAAAAGGCG | SEQ ID NO: 206 |
| | | CAATTTTATTACGGGCAACCAACGACGAAACAAAACAAAAGCCA | |
| | | ACCGAAAAGCAGAAACAAAGCAGCAAAAAGTTTATGAATTTTTT | |
| | | GTGCAGGCGCGTGAAAGATGCAAAACGAGAAAAAAACATGAAA | |
| | | AAAAAACATTAAAAAAAACAAAAAAAATCCAAAACAGATACCG | |
| | | AGCTGTATCCGAAAACGAGTGGGGAAAGGGGTTTCCCAGTCACA | |
| | | TATAA | |
| DmmiPEP1b | Drosophila melanogaster | ATGCGCACAAAAAAGTCAAACAAAAAGGCGCAATTTTATTACGG | SEQ ID NO: 207 |
| | | GCAACCAACGACGAAACAAAACAAAAGCCAACCGAAAAGCAGA | |
| | | AACAAAGCAGCAAAAAGTTTATGA | |
| DmmiPEP8 | Drosophila melanogaster | ATGGAGCCTGGCTTTGTTTTTGTTTTATTTCCAACCCACTTGAGCA | SEQ ID NO: 208 |
| | | CACAGCACACACAGAGAGAAAAATCAATACTCGTTATGGGATTA | |
| | | AATTTACAAAGCGCAAAGCAAAGCGACAAACAAAATTCAAAAGA | |
| | | AAGAAAAAAAAACACTCAAATAAACTCACAAAGAATTCCTTATC | |
| | | GCCAAGGGGGCCAATGTTCTAAGGTTCTTTCGCCTTGA | |
| HsmiPEP155 | Homo sapiens | TGGAGATGGCTCTAATGGTGGCACAAACCAGGAAGGGGAAATCT | SEQ ID NO: 356 |
| | | GTGGTTTAA | |
| AtmiPEP157c | Arabidopsis thaliana | ATGATGTTGCATATCACACATAGGTTTGAGAGTGATGTTGGTTGT | SEQ ID NO: 387 |
| | | TGA | |
| AtmiPEP 157d | Arabidopsis thaliana | ATGCTGTATGTATAG | SEQ ID NO: 388 |
| AtmiPEP160c | Arabidopsis thaliana | ATGTTCATGCGTAGAGGTTTGGTATACAACAATATATACATATAA | SEQ ID NO: 389 |
| AtmiPEP164b | Arabidopsis thaliana | ATGATGAAGGTGTGTGATGAGCAAGATGGAGAAGCAGGGCACGT | SEQ ID NO: 390 |
| | | GCATTAC | TAG |
| AtmiPEP166c | Arabidopsis thaliana | ATGAAGAAGAGAATCACTCGAATTAATTTGGAAGAACAAATTAA | SEQ ID NO: 391 |
| | | GAAAACCCTAGATGATTCTCGGACCAGGCTTCATTCCCCCTAA | |
| AtmiPEP166d | Arabidopsis thaliana | ATGAAGAAGATCGGTAGIATTGATTCATTTTAA | SEQ ID NO: 392 |
| AtmiPEP169a | Arabidopsis thaliana | ATGACTTGCCGATTTAAATGA | SEQ ID NO: 393 |
| AtmiPEP169h1 | Arabidopsis thaliana | ATGGTGACATGA | SEQ ID NO: 394 |
| AtmiPEP169h2 | Arabidopsis thaliana | ATGAAGAATGAGAACTTGTGTGGTAGCCAAGGATGA | SEQ ID NO: 395 |
| AtmiPEP169n | Arabidopsis thaliana | ATGAAGTGTATGATGAAGAAGAGAGGTCTAACATGGCGGAAAGC | SEQ ID NO: 396 |
| | | GTCATGTTTAGTAGCCAAGGATGACTTGCCTGATCTTTTTCGCCTC | |
| | | CACGATTCAATTTCAAATTCATGCATTTTGGATTATTATACCTTTT | |
| | | AA | |
| AtmiPEP170 | Arabidopsis thaliana | ATGTTTCCGAGAGAGTCCCTCTGA | SEQ ID NO: 397 |
| AtmiPEP396a | Arabidopsis thaliana | ATGACCCTCTCTGTATTCTTCCACAGCTTTCTTGAACTGCAAAACT | SEQ ID NO: 398 |
| | | TCTTCAGATTTTTTTTTTTTTCTTTTGATATCTCTTACGCATAA | |
| AtmiPEP399c | Arabidopsis thaliana | ATGTCACTTGCCAAAGGAGAGTTGCCCTGTCACTGCTTCCGCTTA | SEQ ID NO: 399 |
| | | AACACAGTCTATAACCGGTTCTGCTAA | |

TABLE 3
List of the primary transcripts (pri-miRNAs)

| miPEP | Organism | Sequence of the Pri-miRNA | SEQ ID |
|---|---|---|---|
| AtmiPEP156a1 | Arabidopsis thaliana | ATTCATTGTTCACTCTCAAATCTCAAGTTCATTGCCATTTTTAGGTCTCTC | SEQ ID NO: 209 |
| AtmiPEP156a2 | | TATAAATTCAAATGTTCTGTTCAATTCAATGCGTCGCCAGACATCTGTTC | |
| AtmiPEP156a3 | | CCTTTGCATGTAAGAGAGATAAAGAAAGCGACAAGAGCCATAAAGAAA | |
| | | GGTAAGACTCTTTGAAATAGAGAGAGATAAGGTTTTCTCTTATCTTCTTC | |
| | | TCATCAGATCTTTGTTTCTTTACCCTCTTTCTTTCTTTTTTTTGCTTTTTATG | |
| | | GTTATGTTTTTTCTCGATTTAGACAAAAACCCTAGATTTGATCTTCTAAAG | |
| | | GGTCTCAAATGGAATCTCTTCTCTTCTCATATCTCTCCCTCTCTCCCTCCC | |
| | | TCTCTTTGATTCTTTGTCTTCTCCAGTTAAAACTCAGATCTAACACAAAGC | |
| | | TTAAAAGATTCTCATCGTTTCTTGTTTTCTTTGTTTCATCTTGTAGATCTCT | |
| | | GAAGTTGGACTAATTGTGAATGAAAGAGTTGGGACAAGAGAAACGCAA | |
| | | AGAAACTGACAGAAGAGAGTGAGCACACAAAGGCAATTTGCATATCATT | |
| | | GCACTTGCTTCTCTTGCGTGCTCACTGCTCTTTCTGTCAGATTCCGGTGCT | |
| | | GATCTCTTT | |
| AtmiPEP156c1 | Arabidopsis thaliana | CTCTGCCTTTAGTTCTTTCTTTTTTGGTAATATATTTATTTTTCGTTACGAT | SEQ ID NO: 210 |
| AtmiPEP156c2 | | TTGGTCAAAACCCTAGATTTGTTTTCCAAAAGCATATCTGAAAATGAAGG | |
| | | ACAACTTTCCTCTTCTCCTTCGGTTATAAATATTCTCTCCGGTTTTGCTTGT | |
| | | TTAACCTAAAAGCCTCAGATCTAACTCCAACACCTTCAAAGTCTGCCTCC | |
| | | TTTCCAATCTTCTTTCTTCTGTTCGATCTCTAATCTCAGAATTTGTGTCGGT | |
| | | AAGGTAAAGGTGATAATGAGTGATGACTGATGAGGGAGTTTTGGGACAA | |
| | | ATTTTAAGAGAAACGCATAGAAACTGACAGAAGAGAGTGAGCACACAA | |
| | | AGGCACTTTGCATGTTCGATGCATTTGCTTCTCTTGCGTGCTCACTGCTCT | |
| | | ATCTGTCAGATTCCGGCT | |
| AtmiPEP156e1 | Arabidopsis thaliana | TCCCACATCCAAAGATAGAAAGATGTAAGGTCTAGAGTCTTGTTCTTAAT | SEQ ID NO: 211 |
| | | CCCCTAACAGAACAATGATATATATAAATAAATATGGGTCGATATCGGC | |
| | | TGTGGAGGACGACTAGCTACGGTTTCGAGCCTGGTCACATGCGTAGAGT | |
| | | GTGAAAGGTAATTAGGAGGTGACAGAAGAGAGTGAGCACACATGGTGG | |
| | | TTTCTTGCATGCTTTTTTGATTAGGGTTTCATGCTTGAAGCTATGTGTGCT | |
| | | TACTCTCTCTCTGTCACCCCT | |
| AtmiPEP156f1 | Arabidopsis thaliana | TCCCACAGCCAATGAGCCAAAGATAAAGAAACACCTATCCTATAATAAT | SEQ ID NO: 212 |
| | | TTAGAGCAATATACCTCCATAATGGAACATCTATATATATAAAGGTATCC | |
| | | GTATATCTCTATATATTATATTCATTGAGTTTAAAGTGGCTAGGGTTTATA | |
| | | GATGTATGTGATATTAAGAGATATGAAACATATTTGTCGACGGTTTGAGT | |
| | | GGTGAGGAATTGATGGTGACAGAAGAGAGTGAGCACACATGGTGGCTTT | |
| | | CTTGCATATTTGAAGGTTCCATGCTTGAAGCTATGTGTGCTCACTCTCTAT | |
| | | CCGTCACCCCCTTCTCTCCCTCTCCCTC | |
| AlmiPEP159a | Arabidopsis lyrata | AAAAAATGACGTGTCCTCTTCTCTCTCTCTCTTTCCTTCTCTCTAAGTATA | SEQ ID NO: 213 |
| | | TTTAGGGTTAATTATTAGGGTTCTTTATCTCTTTCTTCAGTCTTTGAAGTTT | |
| | | CTTCAATAGCTTTAATTGAAGTGATTTACCTCTCTGGGTGTTTTTAGTATA | |
| | | TATATCATGTACATGATCGAATTTCTTTCTATCCAAGTTCTCATCAAACCT | |
| | | TCTCATGTTTTGAAGAGTTAAAGGCTTTATAGTTTGCTTAGGTCAGATCC | |
| | | ATAACATACTGTATTTGACAAGTTTCTTTGTCTCACGATAGATCTTGGTCT | |
| | | GACCAAAATGATTTTCTCGAGAAAAAAAAAGATGGAAGTAGAGCTCCTT | |
| | | GAAGTTCAAACGAGAGTTGAGCAGGGTAAAGAAAAGCTGCTAAGCTATG | |
| | | GATCCCATAAGCCCTAATCCTTATAAAGAAAAAAAAGGATTTGGTTATAT | |
| | | GGCTTGCATATCTCAGGAGCTTTAACTTGCCCTTTAATGGCTTTTACTCTT | |
| | | CTTTGGATTGAAGGGAGCTCTACATCTTCTTTCACCTTCTCTATTTTTCTTT | |
| | | CTTTATTTTCTCCTCTACAGTAATTTATTTGGATT | |
| AtmiPEP159a1 | Arabidopsis thaliana | TTCCAAAACATGACGTGGCCTCTTCTCTCTCTCTCTTTCCTTCTCTCTAAG | SEQ ID NO: 214 |
| | | TATGTTTAGGGTTAATAATTAGGGTTCCTCCTCTCTTTTGTTCTGTCTTTAT | |
| | | ATCTCCTTCATAGCTCTAATGTAAGAGATTTACCTCTTTTGGTGTTTTTGT | |
| | | TAATCCACGTTCTCATCAAAACTTTCTCATTGTTTTATGAAGAGTTAAAG | |
| | | GTCTTTACAGTTTGCTTATGTCAGATCCATAATATATTTGACAAGATACTT | |
| | | TGTTTTTCGATAGATCTTGATCTGACGATGGAAGTAGAGCTCCTTAAAGT | |
| | | TCAAACATGAGTTGAGCAGGGTAAAGAAAAGCTGCTAAGCTATGGATCC | |
| | | CATAAGCCCTAATCCTTGTAAAGTAAAAAAGGATTTGGTTATATGGATTG | |
| | | CATATCTCAGGAGCTTTAACTTGCCCTTTAATGGCTTTTACTCTTCTTTGG | |
| | | ATTGAAGGGAGCTCTACATCTTCTTTCACCTTCTCTATTTTTTATTTTTCTT | |
| | | TATTTCTACTCAACAATTATTTATTCGGATTCATCTTTAATTTTCCGTTAT | |
| | | AATTTCTTTTTGGTAAGGATTATTCGCTATAATTTGAGAAT | |
| CrmiPEP159a | Capsella rubella | TTCCAACGAATGACGTGTACTCTCTCTGCTCTATCTCTCTCTCTAAATATG | SEQ ID NO: 215 |
| | | TTTAGGGTTAATTAGGGTTCTTCATCTGTCTCTCTCTCTCTCTCTCTTCAG | |
| | | AGTCTTTATAGCTTCTTCCAAGATTTTTAATTGAAAGTAATTTACCTCTTT | |
| | | TGGAGTTCTGTACATATAGAATATCAGGAGTCGTGTTTCTTTTTTATCAA | |
| | | GGTTCTCATCTAACCTTTATAGTATTTTCATTAGTTGATAAAGGTCTTCAT | |
| | | AGTTTGCTTAGATCAGATCTTGTCTTCGTCTTTTCGATAGATCTTGTTCTG | |
| | | TCCAATATACAGTGATTTTATTTCGAGAGCAAAAAAGATGAGAGGTAGA | |
| | | GCTCCTTGAAGTTCAAACGAGAGTTTAGCAGGGTAGAGAAAAGCTGCTA | |
| | | AGCTATGGATCCCATAAGCCCTAATCCTTGTTAATGATAAAGGATTTGGT | |
| | | TATATGGCTTGCATATCTCAGGAGCTTTAACTTGCCCTTTAATTGCTTTTA | |
| | | CTCTTCTTTGGATTGAAGGGAGCTCTACATCTTCTTTGACTTCTCTCTCTA | |
| | | TTAAGTCTTTCTTTATTTTCTTCTCTACAATAGTTGTTTTGGATCGGAAGA | |
| | | TCTTTAAGTTTCCCTTA | |
| AtmiPEP159b1 | Arabidopsis thaliana | TTTCACTTTTGTTCTCCTCCTCCCTTTTTTTCTTTTCAGGATTCTTCTTTTCT | SEQ ID NO: 216 |
| AtmiPEP159b2 | | ATGTTTTATCTTTCATAATAGATCTGATAATTTTGATTTTTCACTATATAT | |
| | | ATTATGGTTAATACTAGTAGCTTTTTCATTTCAAGTTTTATCCTTCCATTG | |
| | | GTTCTTTCTGAGTCAAATTGTCTCCTGTTTCGAACCATATATAAGTTTTCA | |
| | | ATGGTTTTGTATTAACTCAAGTATTCAACATTATGTCTCTCTTTTTCTTGC | |
| | | TTGGATCTCTAATGCTGTTCATATTTTAAAGCATAGGTTTAGGTTAGATG | |
| | | CATGTAACTGCCAATTAAAAGAAGGTCAAGAGTTTTTTGATTGTATGAAT | |
| | | ATATGAGTTAGTCAAAGCAGATCCACACGATTATATAGAAAAACAAAGG | |
| | | AAGAAGAAGAGGAAGAGCTCCTTGAAGTTCAATGGAGGGTTTAGCAGGG | |
| | | TGAAGTAAAGCTGCTAAGCTATGGATCCCATAAGCCTTATCAAATTCAAT | |
| | | ATAATTGATGATAAGGTTTTTTTTATGGATGCCATATCTCAGGAGCTTTC | |
| | | ACTTACCCCTTTAATGGCTTCACTCTTCTTTGGATTGAAGGGAGCTCTTCA | |
| | | TCTCTC | |
| AtmiPEP160a1 | Arabidopsis thaliana | CATCCCACCCTTAATTGTTTTATATAAACCATTTCTCCTCCTCTCTCCATC | SEQ ID NO: 217 |
| | | ACCTTCAATCTCTCTCGATCTCTCTCTGGATCCCCAATCTCACCTCCATGT | |
| | | TTTGTTTGTTGATTCCCATCTTCTCTTTTGTCTTTTCACCAAATCGTCATTT | |
| | | AAGGCTTCAAGAACAGTAACCCCAATTCCTCCACAAGAGGGAGAGAAAA | |
| | | CAAAAGATCTTCCAATTCCATTCTCGTACATGCAAATCACAATCCATGCC | |
| | | ATAGATTGTTTCTATTCCTCCTTATTTATTGCTTGTATCTGTTCATGCATG | |
| | | GACCAGGTGGAGAGAGCATTACTTAAAAATAGAATTAGCTATCTGTTTTA | |
| | | GGCGAATTAGTTTCCTTACATAACCATGTATATGTCATGACGCATATACA | |
| | | TATGTAGATGTATATGTATTATATATGTATGCCTGGCTCCCTGTATGCCAT | |
| | | ATGCTGAGCCCATCGAGTATCGATGACCTCCGTGGATGGCGTATGAGGA | |
| | | GCCATGCATAT | |
| AtmiPEP160b1 | Arabidopsis thaliana | ACTCATAACTCTCCCCAAATTCTTGACCAAAAATATCCGCCACTTTCTCTC | SEQ ID NO: 218 |
| AtmiPEP160b2 | | TGGTTCATGTTTTCCCCTCAATGAAATACATACACATTTTGATTTTATTTA | |
| | | AATCAAGATCGACGTATAAGCTATCCACCAATCATATTTAAGGGTTCCCG | |
| | | TATACATATATACTATATATATATATGGAATAATAGTCGTGCCTGGCTCC | |
| | | CTGTATGCCACAAGAAAACATCGATTTAGTTTCAAAATCGATCACTAGTG | |
| | | GCGTACAGAGTAGTCAAGCATGAC | |
| AtmiPEP161 | Arabidopsis thaliana | CTCTAACTCATCCTTCTCTTCTATGAAAATTCCATTGTTTCTGCCGAAGCT | SEQ ID NO: 219 |
| | | TTGATCAGTACTTCTCTTTTGCTTGATCTCGGTTTTTGACCAGTTTATTGC | |
| | | GTCGATCAATGCATTGAAAGTGACTACATCGGGGTTCCGATTTTTTTTGT | |
| | | TCTTCATATGATGAAGCGGAAACAGTAATCAACCCTGGTTTAGTCACTTT | |
| | | CACTGCATTAATCAATGCATTTGTAAAAAGAGGGAAAAGCA | |
| AtmiPEP162a1 | Arabidopsis thaliana | CTAGAAGAAAAAACCAGATCTATAAAGTTTGTTATTAAAAGATAGAGAG | SEQ ID NO: 220 |
| | | AGAGGAGGGATGTAGTAGGCCAATAGGCAAATCAGAGAATCACAAATG | |
| | | GTATCTGGTCAAGAAGATTCCTGGTTAAAACTTTCATCTCTCTGTTTCCTT | |
| | | TTTCTTTCTTTGTTGGATTCATTAATTTGACATATCTCTATCATCACACTG | |
| | | ATTCTCTTTCTCCCAGTTTGTCTGCAGATGCATGTGTGTAATCTAGGGTAT | |
| | | ATGTTTTTGTCCATTTGGTTTCATAAGGCAATAAAGATCCAGCTATTTACT | |
| | | ACTTGTGGTATAGATTTTGACTGTTGAATTTTCAGATCTGATGTGTTTCGT | |
| | | TTGATCCGATTCGGAAAATTTATGTTTCGTTGACATTTTGGAGTTTAGTTG | |
| | | GAAGAAGAGTGAGAGTCGCTGGAGGCAGCGGTTCATCGATCTCTTCCTG | |
| | | TGAACACATTAAAAATGTAAAAGCATGAATAGATCGATAAACCTCTGCA | |
| | | TCCAGCGTTTGCCTCTTGTATCTTTCTTATTGACTT | |
| AtmiPEP162b1 | Arabidopsis thaliana | CTGCATCTATCCACCTCTCTCTGTAAATTTATCTAAATGTTTCTTTTAATC | SEQ ID NO: 221 |
| | | TTTTTGAGATTAATAATGATTTGTGTTTGTTCATCAACCGATTTTCTCAGA | |
| | | TCTGTCAATTATTTTTGTTTATTTATTTATGATTTATGAATGAGGAAAGAG | |
| | | TGAAGTCGCTGGAGGCAGCGGTTCATCGATCAATTCCTGTGAATATTTAT | |
| | | TTTTGTTTACAAAAGCAAGAATCGATCGATAAACCTCTGCATCCAGCGCT | |
| | | GCTTGCTC | |
| AtmiPEP163- 1 | Arabidopsis thaliana | TATCACAGTTCTCATCAAATATTTGAAAGTATCAAACAAAAAAAGGAGA | SEQ ID NO: 222 |
| AtmiPEP163- 2 | | GTGAGAAAAATAAAGAGAGAGATAGAGAGAGATCATGTCCACTACTCA | |
| | | AGAGCATAGGTCTTGATTGGTGGAAGACAAGTACCTTAGATAAACCGAC | |
| | | CAAAACCCGGTGGATAAAATCGAGTTCCAACCTCTTCAACGACAACGAT | |
| | | TTCAACACTCTCTTCCAGGAACAACTTCCTCCAGGCAGATGATACTAAAG | |
| | | TGCTGGAGTTCCCGGTTCCTGAGAGTGAGTCCATATCAAAATGCGCATTC | |
| | | GTTATCACTTGGTTGAACCCATTTGGGGATTTAAATTTGGAGGTGAAATG | |
| | | GAACGCGTAATTGATGACTCCTACGTGGAACCTCTTCTTAGGAAGAGCAC | |
| | | GGTCGAAGAAGTAACTGCGCAGTGCTTAAATCGTAGATGCTAAAGTCGT | |
| | | TGAAGAGGACTTGGAACTTCGATATTATCCCCCGTGT | |
| AlmiPEP164a1 | Arabidopsis lyrata | AGTAGGGTTGGAAAATTTTTTTACATTTTTACTCTAAAATAGAATAGAGT | SEQ ID NO: 223 |
| AlmiPEP164a2 | | TGGAGATGCCCTTAGCAGTTATTAGACAAGGGATTGTTTGGCCCTAGCGA | |
| AlmiPEP164a3 | | TCCTCTCTTCACTCTCTCACTTTTGTAGTTCAACCCTTCTTTTGCGTGAGAT | |
| | | GCCATCATGGCATGACATGGTTCTTTTGCCTTACGTAAAACACACTCACG | |
| | | CCAACACACGCCACATAACATAAATAAATTATATATACATATACGTATGT | |
| | | GCGTGTGAGTCTTCCATTAATGCAATCTTTGGGCCTATATATATATACAA | |
| | | ACCTTCCATAACCAAAGTTATCATACTACAAAAGCTCTCTCGTACTTGGA | |
| | | AATGCGGGTGAGAATCTCCATGTTGGAGAAGCAGGGCACGTGCAAACCA | |
| | | ACAAACACGAAATCCGTCTCATGTGTTTTGCACGTACTCCCCTTCTCCAA | |
| | | CATGAGCTCCTGACCCATTG | |
| AtmiPEP164a1 | Arabidopsis thaliana | AGACAAGCCCCCACACTAAAAAAACAGTAATATGGAATAAAAAAAAGC | SEQ ID NO: 224 |
| AtmiPEP164a2 | | TTTCAAAACTTAGCAGTTATTAGACAAGGTATTGTTTGGCCCTAGCTAGC | |
| AtmiPEP164a3 | | GATCGTTTAGCTCTCTTCACTCTCTCACTTTTTTAGTTCAACCCTTCTTTTG | |
| | | CGTGAGATGCCATCATGGCATGGTATGGTTCTTTTGCCTTACGTAAAACA | |
| | | CACTCACGCCAGCACACACACACACACACATAACATATACGGATGTGCG | |
| | | TGTGAGCTAGTCTTCCATTAATGCAATCTTTGGGCCTATATATACAAACC | |
| | | TTTCCATAACCAAAGTTCTCATACTACAAACGCCCCTCATGTGCTTGGAA | |
| | | ATGCGGGTGAGAATCTCCATGTTGGAGAAGCAGGGCACGTGCAAACCAA | |
| | | CAAACACGAAATCCGTCTCATTTGCTTATTTGCACGTACTTAACTTCTCCA | |
| | | ACATGAGCTCTTCACCC | |
| BrmiPEP164a1 | Brassica rapa | AGACAACCCCACGTTTTAAAATAAGAAATGATGATAATTTTGTGGAAAT | SEQ ID NO: 225 |
| BrmiPEP164a2 | | AAAAGCTAGTATACTTTTGCAATAATTAGACAAGGTATTGATGCTTTGGG | |
| BrmiPEP164a3 | | CCAAGCTAGTTTCTTTTAGCACTCTTCACTCACTAGTTTTTCTTCTCAGCC | |
| | | CTTCTTTTGCGTGAAATGCCATCATGGCATGGCATTGTCATTTTGCCTTTC | |
| | | GTAAAACACACTCACGCCAACATACATTATTCATATTCATGTGTATGTAT | |
| | | ATGAATGTTCCATTAATGCAATCTTTGGGGCCTATATATACGAAGCTTAC | |
| | | ATCACCAAAGCTCTCATATTACAAAAGCTCACATATATACTTGGAAATGT | |
| | | AGGTGAGAACCTCCATGTTGGAGAAGCAGGGCACGTGCAAACCAAAAA | |
| | | ACATGAAATCTGTTTCATATGCTTTGCACGTGCTCCCCTCCTCCAACATG | |
| | | A | |
| CpmiPEP164a1 | Carica papaya | AGACAACACTCCTCTTTGTTCCCTTCCTCACGTATCCACTTTTGAAATTTG | SEQ ID NO: 226 |
| CpmiPEP164a2 | | TAATTTGTGTGCACCACCATGATTGCATGCCATCCCTACTTGCCTTTTCCC | |
| | | CTTTTCCTTTCTCTAACATTTTACTCAATCTTCTTCTCCCCCTCCCCCCCTT | |
| | | CCCCCTCTCTGCCATTATAACCATAATTAAACCTCTCTCCCTCTCTCTCCC | |
| | | TCTCTCTCTCTCTCTCTCTGGGTTCTCAGTATAAATGCAGCTCTGCTTATA | |
| | | CTTCCACACCTATATATATATACCTGACCCTTCTTCACCTCCTTCATCCAC | |
| | | CTCCTCCTTCTTCCCCAAAACTTTCTTAACTGTTCTCTGCATACATATATA | |
| | | TCCACATACATATATATATATATAGAGAGAGAGTGAGACAGAGAGGTTA | |
| | | CCGAGGCAATTGGGTGAGTAGCTCCCTGTTGGAGAAGCAGGGCACGTGC | |
| | | AAATTCTCCATGGCTTTCCCCTCTTTGCACGTGCTCCCCTTCTCCAACATG | |
| | | GGTTCC | |
| CrmiPEP164a1 | Capsella rubella | CGGCCACCCCCACATTTAACAAGAAAAAAACTGATGGAATTAAAAGGTT | SEQ ID NO: 227 |
| CrmiPEP164a2 | | TGAGAACTTGGCAGTTATTAGACAAGGTATAGTTTGGCCCTAGCTTCTTT | |
| CrmiPEP164a3 | | TAATTTAGCTCTCTCCACTCTCACACTTTTCAACTTTCACCCTTCTCTTGC | |
| | | GTGAGTCGCGAGATGCCATCATGGCATGGCATGGCATGTTTCTATTGCCT | |
| | | TACGTAAAACACACTCACGCCAACACATACTCACTATACATGTAAATAA | |
| | | GTATGTGCGCGTGTGAGTCTTCCATCCATCAATGCAATCTTTGGGGCTAT | |
| | | ATATATACAAACCTTTTCCATAACCAAAGCTCTCATATAAACTACAAAAG | |
| | | GCTCACTTOGGAAATGCOGGTGAGAATCTCCACGTTGGAGAAGCAGGGC | |
| | | ACGTGCAAACCAACAAACACGAAACCCTCCTCATGTGCTTTGCACGTACT | |
| | | CCCCTTCTCCAACATG | |
| GrmiPEP164a1 | Gossypium raimondii | GAAAACCCAAGTTCAGGCTAACAAGTTATCTGATGATGAGATCAAGAAT | SEQ ID NO: 228 |
| GrmiPEP164a2 | | TTTAAAGTTTCAATATAGATTTGGCATGGGTATTGGCGGCAGAAAGCAAT | |
| GrmiPEP164a3 | | TAAAAAACCAGTTATGTCAAATTCAAGGTCGTATCAGTTAAAATGAATG | |
| | | AAGATTTAGAAATTTCAACAAGGAAGAGGACCCCACAGCTTTGTTAAAT | |
| | | TAAGTGTGTGGTTTTTATAATTATCATCTCGAAAGTTTCATAATATCAATT | |
| | | AGATTAAAACATCTCTGAATTTCATAATTACAAACCAGATAGATAGATAC | |
| | | ATGAAAACTTAGACCCCAGAGATCTGTCTTTAAAGAATGCCCACTTCTAG | |
| | | ACTCAATCTCTATTACTCTCTTTTTTTCTCTCTCTCTCTCTTCGGAAAAACT | |
| | | TGTATATAAATAAATGACACTTTCTTTGCTTTCTGCACTCAACTCATGAAC | |
| | | TTGAAAAGCTTTACTTGGATGGGTTGGTTGGGGGTGAGTATCTCTTGTTG | |
| | | GAGAAGCAGGGCACGTGCAAGTTCCTATGTTTAAGTGAACTTTGCACGT | |
| | | GCTCCCCTTCTCCACCGTGAG | |
| MtmiPEP164a1 | Medicago truncatula | GAAGAGAAAAAACCTAGTGTAAAATTTGATATACTCTTTATGTATAGTAC | SEQ ID NO: 229 |
| MtmiPEP164a2 | | GAATGTTTTTTTAAAAATTATGTAAAAAATGATAAAATAATAACTAACTA | |
| | | AATTAACAGTAAAATTAGAAAAGTAAAATACTATGCCCAAATTTGATAT | |
| | | TTTTTTTTATATATTTGTATAGATTATTATTATTTGATATGTAAAGTCCAA | |
| | | TTAAAAATTTGTTTTAACTAAGATTTGAACTAGGTTTTCTTAAAAGACTC | |
| | | ATCTTTTACTTCAAATTTATTTATCATTTGAATTCAATCACTTTCTAATATT | |
| | | ATTATTATTATTTCCACCATACTCATTGCTTCTGCCACGTTACTTTAGTTA | |
| | | GATCTCTTATGTCATATATCTCTCTCTCTCCTAAGTTGCTACCTATAAATA | |
| | | CTAAGCCTTTCCCTTGGTTGGTTCAATTCAACTTCTACTTCTCATCAAACA | |
| | | CAAAGTGCAATAAGCTTCATTTCCTGGGTGAGAAGCTCCTTGTTGGAGAA | |
| | | GCAGGGCACGTGCAAATCCTCTTTCTGATTCATTCTCTCATAATGCATAT | |
| | | CAATATCTTTTGCACGTGCTCCCCTTCTCCAACTAGG | |
| osmiPEP164a1 | Oryza sativa | ATGCAAACCCACTCCAACACTCCACAATCCACATACTCTCTCTCTCTCTCT | SEQ ID NO: 230 |
| osmiPEP164a2 | | CTCTCTGAGTAGGAGTACATGTGTGTGTGTGATATCAATATGCATTCGAT | |
| | | GTTGATGCTACTGTAGCCATCTTGTGGCTATATAAACCCAGCAGGCAGCA | |
| | | GCACAGCTTAGCTAGAGAGCCATATTGCATGCACACTCGCTAATCTCTTT | |
| | | TCTCTACTCTACTTGCATTACACCACCTCTGCATTGCACTTCAGTTCATTC | |
| | | ATTCCACTGATGCATGGATCGATGTTGCTACCTTCTTCTCTTCTCCTCATG | |
| | | CATCCATGCATCGATCTCACCTAGCTTCTTCCTCATCCTCTCTCGATCGAT | |
| | | TACAAGAGAAAAGTGTTTGCTGTTCTTGCTATCGATCTACAGGTGAGTAG | |
| | | GTTCTTGTTGGAGAAGCAGGGTACGTGCAAAATGCACACCGGTTGGTCG | |
| | | AGCTAATTAACAAGCTCTGACGACCATGGTGATCGAATGCACGTGCTCCC | |
| | | CTTCTCCACCATGGCCT | |
| AlmiPEP165a | Arabidopsis lyrata | CTAGGGTTTAGGAATGACGACTTGTTTCTGTTGTGTCTTATTAAAAGCCC | SEQ ID NO: 231 |
| | | ATCTTCGTCTCCGCCACTCATCATTCCCTCATCATAACACCATCATCACCA | |
| | | TTCACCAACCTCTCTCTCTTTCTCTCTCTCCTCTCGATCTACAACAAAATG | |
| | | TGAATCTGCTAAGATCGATTATCATGAGAATTAAGCTATTTCAGTTGAGG | |
| | | GGAATGTTGTCTGGATCGAGGATATTATACATATATACATGTGTATGTTG | |
| | | ATACATGTGATCATAGAGAGTATCCTCGGACCAGGCTTCATCCCCCCCAA | |
| | | CATGTTATTGCCTCTGATCACCATATATATGTCGTTACATTTCATGGTTAA | |
| | | TTACTTGCACAAATCACAAAAGCTTGGTTTGTAACTTTCTATGACCTTTTT | |
| | | TAATGACTTTGAATCTTTCATGCATGACTTCTTAAGAGTAGATTTACACA | |
| | | TTTGCGGATCCGTTTATGCTTTTTGCTTTTGTTTCGTTTATATATAT | |
| AtmiPEP165a | Arabidopsis thaliana | CTAGGGTTTAGGAATGACGACCTGTTTCTGTTGTGTCTTATTAAAAGCCC | SEQ ID NO: 232 |
| | | ATCTTCGTCTCCGCCACTCATCATTCCCTCATCATAACACCATCATCACCA | |
| | | TTCACCAACCTCTCTCTCTCTCTCCTCTATCACTCTCTACAACAAAAATTT | |
| | | GTGAATCTGCTAAGATCGATTATCATGAGGGTTAAGCTATTTCAGTTGAG | |
| | | GGGAATGTTGTCTGGATCGAGGATATTATAGATATATACATGTGTATGTT | |
| | | AATGATTCAAGTGATCATAGAGAGTATCCTCGGACCAGGCTTCATCCCCC | |
| | | CCAACATGTTATTGCCTCTGATCACCATTTATTGTTACATTTTTTTTTGTTA | |
| | | ATTACTTGCGCAAATTACAAAAGCTTGGTTTTTGTGATGACTTTGAATCTT | |
| | | TCTTGCATGGCTTCTTAAGAGTAGATTTACGGATCCGTCTATGCTTTTTGC | |
| | | TTTTTGTTTCGTTTATTTGTATTTAAAC | |
| BemiPEP165a | Brassica carinata | GAGATCAATGAAATTATCCTGCCAAATAAAACGTGTGACGTTTATTCAAA | SEQ ID NO: 233 |
| | | AATATATGCATTAGATGCTTTGATATTAAAATATTTCCTTTTAAAAGCTA | |
| | | GCTAGGGTTTAGGAATGACGAGTTGTGTCTTATTAAAAGCCCTTCTTCTC | |
| | | CTCCGCCACTCATCATTCCCTCATCATAACACCATCATCACCATTCACCC | |
| | | ACCTCTCCTCTTTCTCTCTCTCTCTCTCTCTCTCTCTCTCTAGAACAACAAG | |
| | | TGAGAATCTGCTAAAATATTGTGACTATTATCATGAGAATGAAGCTATTT | |
| | | CAGTTGAGGGGAATGTTGTCTGGATCGAGGATATTATATATACACAAAT | |
| | | ACGTATATATGTTAATACAAGTGTTTGATCATATATGTATATAGATTATT | |
| | | CTCGGACCAGGCTTCATCCCCCCTAACATGTTATTGCCTCTGATCACCAG | |
| | | ATTCTATCAACTCTTCGCTTATTATTTTGTCACAAACAAGTAATAAGCTCA | |
| | | TAATTTTCTTTGAGTCTTTCAGCATCGTTTCATTATGTTTTTCGAATCCG | |
| BjmiPEP165a | Brassica juncea | GAGATCAATGAAATTATCCTGCCAAATAAAACGTGTGACGTTTATTCAAA | SEQ ID NO: 234 |
| | | AATATATGCATTAAATGCTTTGATATTAAAATATTTCCTTTTAAAAGCTA | |
| | | GCTAGGGTTTAGGAATGACGAGTTGTGTCTTATTAAAAGCCCTTCTTCTC | |
| | | CTCCGCCACTCATCATTCCCTCATCATAACACCATCATCACCATTCATCCA | |
| | | CCTCTTCTCTCCTCTCTCTCTCTCTCTCTCTCTCTCTCTCTCTCTCTCTCTCT | |
| | | AGAACAACAAGTGAGAATCTGCTAAAATATTGTGATTATTATCATGAGA | |
| | | ATGAAGCTATTTCAGTTGAGGGGAATGTTGTCTGGATCGAGGATATTATA | |
| | | TATACACAAATATGTATATATATGTTAATATCAGTGTTTGATCATATATA | |
| | | TGTATATAGATTATTCTCGGACCAGGCTTCATCCCCCCTAACATGTTATTG | |
| | | CCTCTGATCACCAGATTCTATCAACTCTTAGCTTATTATTTGTCACAAACA | |
| | | AGTAATAAGCTCAATAATGTCTTTGAGTCTTTCAGCATCGTTTCATATGTT | |
| | | TTCGAATCCG | |
| BnmiPEP165a | Brassica napus | GATATCAATGAAATTATCCTGCCAAATAAAACGTGTGACGTTTATTCAAA | SEQ ID NO: 235 |
| | | AATATATGCTTTAAATGCTTTCATATTAAAATATTTCCTTTTAAAAGCTAG | |
| | | CTAGGGTTTAGGAATGACGAGTTGTGTCTTATTAAAAGCCCTTCTTCTCC | |
| | | TCCGCCACTCATCATTCCCTCATCATAACACCATCATCACCATTCACCCA | |
| | | CCTCTCCTCTTTCTCTCTCTCTCTCTCTCTCTCTCTCTCTAGAACAACAAGT | |
| | | GAGAATCTGCTAAAATATTGTGACTATTATCATGAGAATGAAGCTATTTC | |
| | | AGTTGAGGGGAATGTTGTCTGGATCGAGGATATTATATATACACAAATA | |
| | | CGTATATATGATAATACAAGTGTTTGATCATATATGTATATAGATTATTC | |
| | | TCGGACCAGGCTTCATCCCCCCTAACATGTTATTGCCTCTGATCACCAGA | |
| | | TTCTATCAACTCTTCGCTTATTATTTGTCACAAACAAGTAATAAGCTCAAT | |
| | | AATGTCTTTGAGTCTTTCAGCATCGTTTCATATGTTTTCGAATCCG | |
| BomiPEP165a | Brassica oleracea | GGGATCAATGAAAATTATCCTGCCAAATAAAAACGTGTGACGTTTATCC | SEQ ID NO: 236 |
| | | AAAAATATATGCATTAAATGCTGTGATATGAAGTATTTCCTTTAAAAGCT | |
| | | AGCTAGGGTTTAGGAATTACGAGTTGTGTTTTATTAAAAGCCCTTCTTCT | |
| | | CCTCCGCCACTCATCATTCCCTCATCATAACACCATCATCACCATTCACCC | |
| | | ACCTCTCCTCTTTCTCTCTCTCTCTCTCTCTCTCTAGAACAACAAGTGAGA | |
| | | ATCTGCTAAAATATTGTGACTATTATCATGAGAATGAAGCTATTTCAGTT | |
| | | GAGGGGAATGTTGTCTGGATCGAGGATATTATATATACACAAGTACGTA | |
| | | TATATGTTAATACAAGTGTTTGATCATATATGTATATAGATTATTCTCGG | |
| | | ACCAGGCTTCATCCCCCCTAACATGTTATTGCCTCTGATCACCAGATTCT | |
| | | ATCAACTCTTCGCTTATTATTTGTCACAAACAAGTAGTAAGCTCAATAAT | |
| | | GTCTTTGAGCCTTTCAGCATCGTTTCATATGTTTTCGAATCCG | |
| BrmiPEP165a | Brassica rapa | GAGATCAATGAAATTATCCTGCCAAATAAAACGTGTGACGTTTATTCAAA | SEQ ID NO: 237 |
| | | AATATATGCATTAAATGCTTTGATATTAAAATATTTCCTTTTAAAAGCTA | |
| | | GCTAGGGTTTAGGAATGACGAGTTGTGTCTTATTAAAAGCCCTTCTTCTC | |
| | | CTCCGCCACTCATCATTCCCTCATCATAACACCATCATCACCATTCATCCA | |
| | | CCTCTTCTCTCCTCTCTCTCTCTCTCTCTCTCTCTCTCTCTCTCTCTCTCTCT | |
| | | AGAACAACAAGTGAGAATCTGCTAAAATATTGTGATTATTATCATGAGA | |
| | | ATGAAGCTATTTCAGTTGAGGGGAATGTTGTCTGGATCGAGGATATTATA | |
| | | TATACACAAATATGTATATATATGTTAATATCAGTGTTTGATCATATATA | |
| | | TGTATATAGATTATTCTCGGACCAGGCTTCATCCCCCCTAACATGTTATTG | |
| | | CCTCTGATCACCAGATTCTATCAACTCTTAGCTTATTATTTGTCACAAACA | |
| | | AGTAATAAGCTCAATAATGTCTTTGAGTCTTTCAGCATCGTTTCATATGTT | |
| | | TTCGAATCCG | |
| AtmiPEP166a | Arabidopsis thaliana | CATCATCACCACTCACTTATCTTCTTCTCCATCTCTCTCTCTGCTTCTCCCT | SEQ ID NO: 238 |
| | | TAATCTTAGCCGGGTCTCGTGGGGGACGAACATAGAAAGAGAGAGATAT | |
| | | AAAGATATATATTCAGAAACCCTAGATTCTATAATTTCGACTGAAAAGA | |
| | | AAAAGGGGCTTTCTCTTTTGAGGGGACTGTTGTCTGGCTCGAGGACTCTG | |
| | | GCTCGCTCTATTCATGTTGGATCTCTTTCGATCTAACAATCGAATTGAACC | |
| | | TTCAGATTTCAGATTTGATTAGGGTTTTAGCGTCTTCGGACCAGGCTTCAT | |
| | | TCCCCCCAATTGTTGCTCCCTGTTTACTCCATATTTCTTCCTTCTTTTCAAA | |
| | | TTAGGGTTTCAGATCCAGTGAATGAACCCTTGTTAAAGGTTTGATCTCTT | |
| | | ACCTTACTTT | |
| AtmiPEP166b | Arabidopsis thaliana | TCTCATCATTCTCTTCATCATCACCACATTCATCTCTCTCTCTCTCTCTCTC | SEQ ID NO: 239 |
| | | TCTCTCTCTTTCTCTTCCTTGATCTTAGCCGGATCTGTTGGGGGACGAACA | |
| | | CATGAGAGATAGATAAAATATAAGAAATTTCTCGAAAAAACCTAATAGA | |
| | | AAAAGGTCTGTTTCTTAAAGAAGAAGAAGAAGAGGATTTAAAGAGGGAT | |
| | | TTTTCTTTTGAGGGGACTGTTGTCTGGCTCGAGGACTCTTATTCTAATACA | |
| | | ATCTCATTTGAATACATTCAGATCTGATGATTGATTAGGGTTTTAGTGTC | |
| | | GTCGGACCAGGCTTCATTCCCCCCAA | |
| AtmiPEP167a | Arabidopsis thaliana | TGATGAACAGAAAAATCTCTCTTTCTCTTTCTTGATCTGCTACGGTGAAG | SEQ ID NO: 240 |
| | | TCTATGGTGCACCGGCATCTGATGAAGCTGCCAGCATGATCTAATTAGCT | |
| | | TTCTTTATCCTTTGTTGTGTTTCATGACGATGGTTAAGAGATCAGTCTCGA | |
| | | TTAGATCATGTTCGCAGTTTCACCCGTTGACTGTCGCACCC | |
| AtmiPEP167b1 | Arabidopsis thaliana | AACCACAAAGTACCGCTGCTATTTTCTTTTTACGTCTTTGTATTTGCATCG | SEQ ID NO: 241 |
| AtmiPEP167b2 | | TCTAAGAGAATGATGGGTTGTTTTGTGGGATTTTAATGCAGGAGGAAAC | |
| | | ATATGAGGGGTGATTAAGGCAAAAACCTTAAGATGTGGTCATTTAGATA | |
| | | CATGGAGTCAAACTAAGAATGGACCTTGGCGAAAGCTTCTTCACGGTCA | |
| | | AGATTTAAAATCAGGTACGACACTGTGTACGTGAGAGAGAGAGAGAGAG | |
| | | AGAGAAAGAGATTATAGAAAGAAAGAGATGTATCACAATAAAGGAGTA | |
| | | TATTTAGGGTCACAGGTGGTGGAGATATAGGTATGCAGGGCCAAGGCTC | |
| | | TAATCTCTTCATAGCCCTATTGATTTTGTCCCTCTCTCTCTCTCTTTCTTCC | |
| | | TCTCTTAGCTGTATGCATTATGATGCGTCTTTTAATTCACTGTTTCAGGCT | |
| | | TCTTTAATTCGTGGTGTCTCTCTCCTTTTTACCCAACCATCTCTTAAAATTT | |
| | | TTAACATCTGTTCCTCAAATCCTCTCTCATCTCTTTCTATAAGTATCTATA | |
| | | GCGCCTCTTAAACCACAAAGCATCACCTCTGTCTTCTCTCATCTCCTTTCT | |
| | | GTATTCTCTTTCATTGCCTTCACGTCTGTTGCAATTTCTCCACTTCTTGAG | |
| | | CTTCCGTTTTTTACAATTATTGATCCGTCAAATATGTGAGATTTGCACAAC | |
| | | TTGTTGCTCAGGTATTTTGAAGACAAGTCCACAAGGGAACAAGTGAAGC | |
| | | TGCCAGCATGATCTATCTTTGGTTAAGAGATGAATGTGGAAACATATTGC | |
| | | TTAAACCCAAGCTAGGTCATGCTCTGACAGCCTCACTCCTTCCT | |
| AtmiPEP169c1 | Arabidopsis thaliana | GAGCAAGACAATGCCACATACAAACTTGAAAGATCTCTTCATCTTTTCTC | SEQ ID NO: 242 |
| AtmiPEP169c2 | | CAAATGTTTTTTTTTCGTTTGCTATTTATCTCCACAATTCTTGGAACAAAA | |
| | | ACTACATTCACAAACGAGAGAATTTTCACAACACCTCTTTTGCTCTCATT | |
| | | TTTTTTTTTTCGTCCATTATGAGTATTAATTATGGTTAGGGAATCTTACAG | |
| | | AATGAAAATGAAGGTGTGAATGGATTGTCTCATCTAAAGCCTTGAATGT | |
| | | GGGAAAAAGGCCATTGTTGTTCAGCCAAGGATGACTTGCCGGTAGCTTG | |
| | | TATTATGATTACTCTATATTCGATTTATATTATGGAGATGATGGTTTATAT | |
| | | ATATTTACTTATCTACATAGTTTTAGTTGATTTTTTTTCGTACGTAATATA | |
| | | ATACGAAAAAGTATTTACTTATTTATATATGTGTGTTGGGGCAAGAAGTG | |
| | | TAACCAAGCTAGCCCGGCAAGTCATCTATGGCTATGCAACTGTCTCTTCC | |
| | | TCTCATTCTAGGCTTACGATGACACGTAAAAAATCCCAAATATCACTAAT | |
| | | ATGATATGAATATGGATGA | |
| AtmiPEP16911 | Arabidopsis thaliana | AGGCATGAGACATAAAGAGAGTTAAATATAATGAAGAAGAGAGGTCTA | SEQ ID NO: 243 |
| | | ATATGGCGAAAAGAGTCATGTTTAATAGCCAAGGATGACTTGCCTGATCT | |
| | | TTTTCACCTCCATGATTCAATTTTAAGTTCGTGGATTTTGGATTATTATGC | |
| | | GTTTAAAAGGTATAATAATTTGAGATCATGTTGAATCTTGCGGGTTAGGT | |
| | | TTCAGGCAGTCTCTTTGGCTATCTTGACATGCTTTCTTCATC | |
| AtmiPEP171a1 | Arabidopsis thaliana | GAATTTTGATTTATGAACCTCCTCAAGAAGGAAAGACAGAGGAGGAGAC | SEQ ID NO: 244 |
| | | AAAGAAGTATAGGTTCACATTGCATAGCCAGTTTAGTTTTGAAGGATGG | |
| | | ATATATGAAAAAAATATGAAGAGAGAGAAGAGAGAAGAAGAGGAGGAT | |
| | | TAAAGAGGGTGAGGCCAGCTTTTGTGCTTTGGTAGTAGATGAGGTTTAAA | |
| | | TGCTCCATACCTTCCATTTCCTTCTCTCTTACCCTAATTTAATTCTTCCTCT | |
| | | CCTTTATAACTCCCCACAGACATTCTCACTTCTCCTCCTCACACTTCACAT | |
| | | CAACACTTCTTTCTTGTTTTTTCATTTTACAATGTTTCCTTTGATATCCGCA | |
| | | CTTTAAGCATGAGAGAGTCCCTTTGATATTGGCCTGGTTCACTCAGATCT | |
| | | TACCTGACCACACACGTAGATATACATTATTCTCTCTAGATTATCTGATT | |
| | | GAGCCGCGCCAATATCTCAGTACTCTCTCGTCTCTATTTTGGACTTTGTGG | |
| | | TCTTGTAGATCGATTTGTATGTGTGTGTTGAAATGGAGACAAGTACTTGT | |
| | | AACTTCTTTGTTGTTATATTGTTTACCTATAGGCTGATGTCATAAACTCTT | |
| | | TTGATCTTGTTTCTAACTTCCAGATTCTTGAAAAATCAAGTCGTGTGTGTG | |
| | | TCTCCATGGAAGCCTTTTCCATTTCTTCCTTTCCA | |
| AtmiPEP171b | Arabidopsis thaliana | ACTCATAAACTTTGCTACTGGCCGCATTTCTATTTTCTCCTTCGATTCTTC | SEQ ID NO: 245 |
| | | TAATTCGTACTTTGGTTTCTGACGTCCCTAAAATTTTTAGACAGTAAGAG | |
| | | TTTCTCCAGGATCCGATGGTTCTCTCCGGTAAATTAACATTTTAGTGTCAA | |
| | | TAGTCATTTATACATATTTTTATTTCACTTTTTGTTTTGTTTATTGGTTTTC | |
| | | TGGAGCTAAGTGGAGATTATAGTCGAACAAGAGTGGTTTTATGCAAGGT | |
| | | AACGCGAGATATTAGTGCGGTTCAATCAAATAGTCGTCCTCTTAACTCAT | |
| | | GGAGAACGGTGTTGTTCGATTGAGCCGTGCCAATATCACGCGGTAAACC | |
| | | AAAAATGGCAAAGATAGTTATTATAACCTTAAAGGTATGTATCATTATCG | |
| | | TTTTATTGTTTCAATTTTGATTAATGGCTTTGATATTTCATTTTTTTTTT | |
| MtmiPEP171b1 | Medicago truncatula | ATTGGTCAAACATACATACAGTAGCACTAGCTGGTTTCATTATTCCACTA | SEQ ID NO: 246 |
| MtmiPEP171b2 | | TGCTTCTTCATAGGCTCTCCAAATTTTGCAAAATTGAAAGAGACATAGTA | |
| | | TATATATCTTAGCAAGGAGAAATTCAGGATATTGAGGATGAAGATTGAA | |
| | | GAGTAATCAGTGATGAAGAAAGCAAGCAAGGTATTGGCGCGCCTCAATT | |
| | | TGAATACATGGCTATAAAAATGCATCATATCAGCCATGTAGTTTGATTGA | |
| | | GCCGCGTCAATATCTTGTTTCCATCTCCAAATTTACCAATCTCATCAAATC | |
| | | AAATTAACACCACAATCAAGGCTTTCATTTAATGCAGTCAAAATAGGTTG | |
| | | ACCTTATCATCGAAGAAATTGTTTTCTCATTCCTATCGAAGTTGGACTTGC | |
| | | CGAAAATGCTCGAAAGCATGTGTTTTAGTTCGACAGGCGAAAAAGTTAC | |
| | | CGAAGGACAATTTGGTTGTGGTTCGGATAAGATCAAGCAACGGATATTTT | |
| | | CAAGACACGTTCGAAATTCAAATCAAATGGATAAGTATCGTTAGTTTACT | |
| | | GCAGTTATAGTTTTAAATTCAAATCTAGGCAGTTGTTTCTATTTGTATAAA | |
| | | TAGTAGTTTTTCCCTAGGGAAAAGGGGTCGCAATTCAATCATACAAAAA | |
| | | ACTTACAATCAAATTATCCGCATGGAAGAGAGAAACGAGTCACAAGTTG | |
| | | CAATGTATGAACATGTGTACCAATTTACATTCAATCAGTACAATTTAAGT | |
| | | TCATTTCCATAAAAAAAAA | |
| ZmmiPEP171b | Zea mays | ACCAGGGTTAGGTATCCATCCACACAGCAATGCATCTGCCTTCAACTCCC | SEQ ID NO: 247 |
| | | TCTCGCCCCCCACCCCAACACACATCTCTCTCTTTTCTAGGGAAGGAAAT | |
| | | GACGAAGGGGACGACGACGGCATGCTTCGGCTAGCTCGTTGGTGCTAGG | |
| | | ACAAGGGCGGAGGTATTGGCGCGCCTCAATCCGAAGGCGTGGCTGATAG | |
| | | ATTGGCGCGGCAGCCATGTTCTTGGATTGAGCCGCGTCAATATCTCCCCT | |
| | | TGCCTGTCCCGTACCTAGCTAGCTTGCTTGCCTCACTGATCGATGTCGTCC | |
| | | CTATTTCATGGAGAAGCTGATGATTGATTATTCTCACAAGCAAGAACTGT | |
| | | CTGATCTGTTGCCTGCATCGATCAGGATCTATATGCTGGAGAGTTCACAA | |
| | | GAACATGGACAGAACTCGCTTCAACAACCGATCAATCGATTGATTAGGT | |
| | | ATGTACCTACCTCATATGCCTCAGCTCTTCGTTATGGATTTCTTCAACCGA | |
| | | AGGGTCAGTAAGCTCTTGGTTCCATGCCACTGCGTGAACTAAGCGTTCAC | |
| | | AAAATCCGTTCCMCGGCATGAACCAAGCACTCAAAATCGCATGCAGCAT | |
| | | CTTTCGTTTCAAAAAAAAATTGACTTYTGAAAACAATAGATGAATCAGTT | |
| | | TCAAACATATATGATTATCCATTTTCTCAACCGGGAATTTATATCTCGTTG | |
| | | GGATGCAAAACCGTTCAGTAGTAAAACTACTCCACGAGTATAAACTGTTT | |
| | | CAGTTATTTTACTATTAATTAGTTACCCGTATGCTGTTATGGTTTCTATAT | |
| | | ATCTATAAGTAAACCTTACTTAAATAAGATAGTTATACAAAAAAAAAAA | |
| | | AAAAA | |
| AtmiPEP171c1 | Arabidopsis thaliana | CAAGAAAAAACATTGAAATAGCTCATGTTGTCTCTTTCTCATTTTCATAT | SEQ ID NO: 248 |
| | | CTGCTAAAAAAAGAACCGTGTTTTCTAAACTGGTTTAACGGTAAGTACCT | |
| | | GTCTCTAGTAACTTACCTATCAATTTGTTCCAATCATTTACTTGCTTTGAC | |
| | | TTATTTGGTTTCCTTTTGTTTTGTTTTTCTTTAATATGTGGATGGAGTTTGG | |
| | | TGTAATAAGCAACTGAAGAGTCGATGAGCGCACTATCGGACATCAAATA | |
| | | CGAGATATTGGTGCGGTTCAATCAGAAAACCGTACTCTTTTGTTTTAAAG | |
| | | ATCGGTTTATTTGATTGAGCCGTGCCAATATCACGCGTTTAAATAGTTTA | |
| | | AAGATTCTATGTTAGTTGATGTGATCAATCAAGGTATGAATCTATATCAA | |
| | | TTCTCTTATGCATAGTTTTATATTTACAGAGATGAGGTATTATCAATGTCT | |
| | | ATCGTCGAGGATCACGCTCTTACTTATGTTATATTTCTATATAATTTTATT | |
| | | AATTAGTTTTCTAAAAGAGAAGGACAATTTAAAATTATTTTAAAGAGTTT | |
| | | TTTTTAAGTAGTTTTGTTTTCATGTTTATCTTCTGCAGGCTCTGAAGTTAG | |
| | | GATAGTAACAAGAAAAAAGACAGAAAAAAAGAAGAAAATTCATATACA | |
| | | TTCGTGA | |
| MtmiPEP171e | Medicago truncatula | GAATAAGTGAATATTATCGATATTTATATCATATATCAACTTTTCTTCTGT | SEQ ID NO: 249 |
| | | GCTTGCTTGCAAATTTGCAATTAAGCTTTTTTGATCTTATGTAAGAGAAT | |
| | | ATTATTGATGATGGTGTTTGGGAAGCCGAAAAAAGCGATGTTGGTGAGG | |
| | | TTCAATCCGAAGACGGATTTACATGTATAGAGTTGTAAAATACGATCTCA | |
| | | GATTGAGCCGCGCCAATATCACTTT | |
| MtmiPEP171h | Medicago truncatula | CCACAAAACTATAACTAGCTAGAAGCTTTAATCGCCTTATTTATTATAAT | SEQ ID NO: 250 |
| | | AATAATAATAAATATGGCTTCAGCTGCAAAAGTATACATGGCGTGATATT | |
| | | GATCCGGCTCATCTATATCTTCAAGTTCAATCATCCATATTCATATCAATT | |
| | | TCAGACGAGCCGAATCAATATCACTCTTGTTTGCTTCATTGCATATTAATT | |
| | | ATATACTTCATTTATAAGTTATAGTTTGCCATATATATATTAGATTGATTC | |
| | | TGCAGAAGTAGACAGGAGTGGTGTTGTTTCTGCTCATCTTATTAAATAAT | |
| | | GAATGAATGAATGACATTTGCTTACTTATAAGACGAGCCGAATCAATATC | |
| | | ACTCCAGTACACCT | |
| AtmiPEP172a1 | Arabidopsis thaliana | CTCTCTCTCTCTCTCTCTCTCATCTGTGTTCTAGATCTCACCAGGTCTTTCT | SEQ ID NO: 251 |
| AtmiPEP172a3 | | CTGGTTAATATATGGCTTCCAAGATCTGGTAATATGTTATAAATACGTCA | |
| | | TACTTAAGCTTTTTTCAAATCAAAAATAGAAATTTGTGGGTTTGTCTCGTT | |
| | | TTACTATTTTAGCAGTATATATTAAGAAGTTCAGATGTTATTCGATCATCT | |
| | | GTTTTTGCTTCCCCTCTGCCATCTTTATCTTTTAGGGTTTCAATTCTTTTTC | |
| | | ACTTTTTTCTTCTGGTTTGGAGATGGTTAGGTTCCAACTAAGTATACGAG | |
| | | ATTAAATTTGACATCTTAGTTACTTCAAAATTCCTTCAATCAAAACAAGT | |
| | | CATCTCGACTATTCCGCCATGTTTGTATATACATATTTATATATTATATAT | |
| | | ATGAAGGTACGAGTTTCTAGTGTCTATAAATTAAGAAGGTTAAGTACCAT | |
| | | ATAGATGATATTTGTTAAGTAGTAAGTCACTCAAAGTTTGAGTTTGGGTT | |
| | | TGAGTTTGAGTTTGAGTTTGAGTTTGAGAGACAAAAGATTACTACAAGA | |
| | | AGATTGTTAAACAAAAATGGAAGACTAATTTCCGGAGCCACGGTCGTTG | |
| | | TTGGCTGCTGTGGCATCATCAAGATTCACATCTGTTGATGGACGGTGGTG | |
| | | ATTCACTCTCCACAAAGTTCTCTATGAAAATGAGAATCTTGATGATGCTG | |
| | | CATCGGC | |
| AtmiPEP172b1 | Arabidopsis thaliana | ACTTGCACCTCTCACTCCCTTTCTCTAACTAGTCTTGTGTGCACCCATTTA | SEQ ID NO: 252 |
| | | TGTGTACGTACTATTATCTCATAAATAAATATTTTTAAAATTAGATGCATT | |
| | | TATTGATATGAAAAAGTTACAAGATTAGTTTGTTGTGTGTGAGACTTTGG | |
| | | ATCGACAGATCGAAAAATTAACTAACCGGTCAGTATTGAATATCAACTA | |
| | | TTATATGCTCCATGCATTCGCTTATAGTTTCACACAATTTGTTTTCTTCAC | |
| | | GGTCTAAAATCAGAAGATTCCATATATTTTCTTATGACGTAAAAGGACCA | |
| | | CTTATAAGTTGACACGTCAGCCCTTGGATTCGTGAGGTTTTTCTCTCTACT | |
| | | TCACCTATCTACTTTTCCTCATATCCCACTGCTTTTCTCCTTCTTGTTCTTG | |
| | | TTTTTCTCGTTTTTTTCTTCTTCTTCTCCAAGAAAATAGAGATCGAAAAGA | |
| | | TTAGATCTATTTTGTGTAGCAAGAAATTATCATTTTCGTTTCTTCATTCAT | |
| | | ATATTGTTCTATTATGTTGTACAATAATAGATACTCGATCTCTTGTGCGTG | |
| | | CGTAAATTTTATACAAGTTGTCGGCGGATCCATGGAAGAAAGCTCATCTG | |
| | | TCGTTGTTTGTAGGCGCAGCACCATTAAGATTCACATGGAAATTGATAAA | |
| | | TACCCTAAATTAGGGTTTTGATATGTATATGAGAATCTTGATGATGCTGC | |
| | | ATCAAC | |
| AtmiPEP172c1 | Arabidopsis thaliana | TCACCAAATAGGCTCTTCTTTATCGCTTCATATATATAAAAGTCTACATCT | SEQ ID NO: 253 |
| | | ATCTCTTTCTAGGTCACTAGCTAGACTCTAGATTAAGGATTGAAATTAGG | |
| | | GTTTCATGTTTCCAGCAAAATGGTGCCGTCTTGAGTCTTGAAAAGATCCA | |
| | | AGACAAAACCAAATCACTACATACATCCCTATCATCAACCAGCTACTGTT | |
| | | CGCTGTTGGAGCATCATCAAGATTCACAAATCATCAAGTATTCGTGTAAA | |
| | | TAAACCCATTTATGATTAGATTTTTGATGTATGTATGAGAATCTTGATGA | |
| | | TGCTGCAGCTGCAATCAGTGGCT | |
| AtmiPEP172e1 | Arabidopsis thaliana | TGTCATATTGAGAACTCTTTAGCCTTTGGCTTCTGTTCCTGACACTTGTAT | SEQ ID NO: 254 |
| AtmiPEP172c2 | | AGTGAAGTGGGCTTGTGTTATATAGATGGGATCTCTCTCTTTATTTAAAA | |
| AtmiPEP172e3 | | GTCAATTAGAGATCTTGATGCTACTTCTGTCCCTTTCCAAGTGATTTTACG | |
| | | TCGACCAACTAGCTTTTTTCATATGAGTGTATATATTCATGTACCTATCTC | |
| | | TCTCAATTGCTTCTCACCAAAATCATCTTGCTGATTCATTTGCTGTCTGAA | |
| | | TCCTCTTGCTTTCCTCTTTGCTTTTTCATTTGTTGATTTAAACCATGGGAGT | |
| | | TCCCAACTTTAGACCTCGAAACCGATAAGGATCTTTCTCTGCGGTTGAAA | |
| | | TAGCTAGGTTCTCGATGAATAGGCTAGCCTTTGGTGGATGTTATCAGCCA | |
| | | GTAGTCGCAGATGCAGCACCATTAAGATTCACAAGAGATGTGGTTCCCTT | |
| | | TGCTTTCGCCTCTCGATCCGCAGAAAAGGGTTCCTTATCGAGTGGGAATC | |
| | | TTGATGATGCTGCATCAGCAAATAC | |
| AcmiPEP319a1 | Arabidopsis | TTGTATCCATAGTGTATTTCCTCGCATCTACCATCCATTTTCTACGCCTCT | SEQ ID NO: 255 |
| AcmiPEP319a2 | cebennensis | CTCTCTCTCTTTCTCCATCAAATCTTGTTTTGTTCAAACTCTCTCTCTCATC | |
| | | AATTCTCTCCATACAATACATGCATACATACATACATACCATCTCTAATA | |
| | | TTTCATCAATCTTCTTTTGTTCCAAACGCTCTTTCTCTCCATTTACATACAT | |
| | | ACGAATCATTGTTGTCATAGATCCGTTTAGAATTGCTTTAACTTTTAGATG | |
| | | AGATCTAGGGTTTCTTTCTTTTTCTTCAAAATCATGCTTTTTCGCTTGCTA | |
| | | GGTTATAGATCCATGTAAGTTTAGAGTAGATGTACACACACACGCTCGG | |
| | | ACACTTATTAAATACATGTTGATACACTTAATACTCGCTGTTTTGAATTG | |
| | | ATGTTGTAGGAATATATAAATGTAGAGAGAGCTTCCTTGAGTCCATTCAC | |
| | | AGGTCGGATATGATCCAATTAGCTTCCGACTCATTCATCCAAATACCGAG | |
| | | TCGCCAAAATTCAAATTAGACTCGTTAAATGAATGAATGATGCGGTAGA | |
| | | CAAATTGGATCATTGATTCTCTTTGATTGGACTGAAGGGAGCTCCCTCTC | |
| | | TCTTCTGTATTCC | |
| AhmiPEP319a | Arabidopsis halleri | TTGTATCCATAGTGTATTTCCTCGCATCTACCATCCATTTTCTACGCCTCA | SEQ ID NO: 256 |
| | | CTCTCTCTTTCTCCATCAAATCTTGTTTTGATCAAACTCTCTCTCTCTCTCT | |
| | | CTCATCAATTGTCTCCATACAATACATACATACCATCATCTTTCCCATCTC | |
| | | TAATATTTCATCAATCTTCTTTTGTTCAAACGCTCTTCCTCTCCATATACA | |
| | | TATACATACATACGAATCACATTGGTGTCATAGATCCGTTTAGAATTGCT | |
| | | TTAACTTTTAGATGAGATCTAGGGTTTCTTTGTTTCTTTCGTTTTCTTCAA | |
| | | ATTTTGCTGCATATTCTCCAAGATCATGATTTTTCGCTTGCTAGGTTATAG | |
| | | ATCCATGCAAATATAGAGTAGATTTACACACACACACGCTCGGACACTT | |
| | | ATTACATACATGTTGATACACTTAATACTCGCTGTTTTTAATTGATGTTGT | |
| | | AGGAATATATATATGTAGAGAGAGCTTCCTTGAGTCCATTCACAGGTCGT | |
| | | GATATGATCCAATTAGCTTCCGACTCATTCATCCAAATACCGAGTCGCCA | |
| | | AAATTCGAACTAGACTCGTTAAATGAATGAATGATGCGGTAGACAAATT | |
| | | GGATCATTGATTCTCTTTGATTGGACTGAAGGGAGCTCCCTCTCTCTTCTG | |
| | | TAT | |
| ALmiPEP319a | Arabidopsis lyrata | TTGTATCCATAGTGTATTTCCTCGCATCTACCATCCATTTTCTACGCCTCT | SEQ ID NO: 257 |
| | | CTCTTTCTCCATCAAATCTTGTTTTGTTCCAACTCTCTCTCTCATCAATTCA | |
| | | TTCCATACAATACATGCATACATACATACCATCATCATCTTTTCCCATCTC | |
| | | TAATATTTCATCAGTCTTCTTTTGTTACAAACGCTCTTTCTCGCCATATAC | |
| | | ATACATAAGAATCATTGTTGTCATAGATCCGTTTAGAATTGCTTTAACTTT | |
| | | TAGATGAGATCTAGGGTTTCTTTCTTTTTCTTCAATTTTTGCTGCATATTCT | |
| | | TCAAAATCATGATTTTTCGCTTGCTAGGTTATAGATCCATGCAAATATAG | |
| | | AGTAGATGTACACACATTCACGCTCGGACACTTATTACATACATGTTGAT | |
| | | ACACTTAATACTCGCTGTTTTGAATGGATGTTGTAGGAATATATATGTAG | |
| | | AGAGAGCTTCCTTGAGTCCATTCACAGGTCGTGATATGATCCAATTAGCT | |
| | | TCCGACTCATTCATCCAAATACCGAGTCGCCAAAATTCGAACTAGACTCG | |
| | | TTAAATGAATGAATGATGCGGTAGACAAATTGGATCATTGATTCTCTTTG | |
| | | ATTGGACTGAAGGGAGCTCCCTCT | |
| AtmiPEP319a1 | Arabidopsis thaliana | TTGTATCCGCAGTGTATTTCCTCGCATCTACCATCCCTTTTCTACGCCTCT | SEQ ID NO: 258 |
| AtmiPEP319a2 | | CTCCCTCTCTCTCTTTCTCCATCAAATCTTGTTTTGTTCAAACTCTCTCTCT | |
| | | CTCATCTATTCTCTCCATACAATACATGAATATACATACATACCATCATCT | |
| | | TCTTTTCCCATCTCTAGTTTTTCATCAATCTTCTGATGTTCCAAACGCTCT | |
| | | ATCTCTTCATATACATACATACGAATATATTATTGTTGTCATAGATCCATT | |
| | | TAGAATCACTTTAGCTTTTAGATGAGATCTAGGGTTTCTTTGTTTTCTTTC | |
| | | AAATTTTGTTGCATATTCTTCTAAATCATGGTTTTTCGCTTGCTAGGTTAT | |
| | | AGATCCATGCAAATATGGAGTAGATGTACAAACACACGCTCGGACGCAT | |
| | | ATTACACATGTTCATACACTTAATACTCGCTGTTTTGAATTGATGTTTTAG | |
| | | GAATATATATGTAGAGAGAGCTTCCTTGAGTCCATTCACAGGTCGTGATA | |
| | | TGATTCAATTAGCTTCCGACTCATTCATCCAAATACCGAGTCGCCAAAAT | |
| | | TCAAACTAGACTCGTTAAATGAATGAATGATGCGGTAGACAAATTGGAT | |
| | | CATTGATTCTCTTTGATTGGACTGAAGGGAGCTCCCTCT | |
| BrmiPEP319a | Brassica rapa | TTGTATCCATTGTGTATTTCCTTGCATCCATCAATAAATTTTATGTTACGC | SEQ ID NO: 259 |
| | | CTCTCTATTATTTCTCTCTACATCACACTGTCTTATGTTTAAGCTCTACTTC | |
| | | TCAGCAATTCTCTCCACCCAATACATGCATACATACCATCATCGTATCGC | |
| | | TCTAATTTTTCTATCAATCTTGTATCCTTCCACAAATTATCTTATGTCTCCC | |
| | | ATTTTAAATCCTACATAGATCCACACATACGAATTATTCTTGTCTGAAGA | |
| | | TCCATCCATTTACGATTGCTTTAACTTTTACATGAGATCTAGGGCTTCTTT | |
| | | ATTTTTCTTCAAATCTTGCTGCATATATTTCAAGATCATGCTTTTCGGCTT | |
| | | GCTAGGGTTCTAGATCCATGGATGTATAGCGTACATACATACACGCACTA | |
| | | ATTCATACCTGTAGTTTGTACGGAGAACATCATAAAATATCACTGTTTGG | |
| | | AATTAATCGTGTAGGAAATATAGATAGGTAGGGAGCTTCCTTTAGTCCAT | |
| | | TCACAGATCATGATATGATCCAATTAGCTTCCGACTCATTCATCCAAATA | |
| | | CCGAGTCACGAAAATTTCAACTTAGACTCGTTAAATGAATGAATGATGC | |
| | | GGTAAACAAATTGGATCATTGATTCTCTCTGATTGGACTGAAGGGAGCTC | |
| | | CCTC | |
| CpmiPEP319a | Carica papaya | CTATATCCCTGTCAAATAGTACTTGGTTTTGTTTTAGCCACAAATCTTCTG | SEQ ID NO: 260 |
| | | GTCTTGCAAAGTTCCTTCTGATCTCTCCCCCTTTCTCATTTTTTCCTCCTCT | |
| | | TATATATGGATCATCAATTTGCTGTACACACACACACATTTACTGTAGTG | |
| | | ATAATTAGCTAGCTAATTTGTTAGTATGTAAATTAGATCCCAAGTACCCT | |
| | | GTTATATTTTTTTAGGCTTATCCTATGCATACCTGATAGTACAAGAACTTA | |
| | | GTTTGTAATTAGGTACTTGGTAGTAGGGTTAGATTAATTACTGTCTTGAA | |
| | | AGAGAACTTATCCAACAAATAGAGCTATGAAGATTAAATTAGGTTTTAG | |
| | | TCTTATTAAGATTATTATATTACTAGACAAAAACAGTTAAATTTTTTTAAT | |
| | | TGGGTAATTAGGTACTTAGCAATAGGGTTAGATTAATTACTGTTTTGAAA | |
| | | GAGAACCTACCTACAAATAGAGTTGAAATGATTATGTTTTAGTCTTACTA | |
| | | AGATTGTCATATTTCTGGAAAAAAACAAATCTTGAAACAGATAATTCAG | |
| | | ATAGTCATGATCAATGGAAAAAACATCATGGGTGTGTGCTTAATTAAGCT | |
| | | AATATATATATATATGAAGATATAATGTTATGCACACTAGCTATGAATTT | |
| | | GTAAGAATAATGAAGGATAAAGATGATATATTTAGATGTTATAAGTGTA | |
| | | AGTAAGGTGGAATGGGTTGATGGGTAGTAGTAGTAGTAGTAGAGATGAT | |
| | | TGGTGGAGAGAGCTTCCTTCAGCCCACTCATGGATGGGTATGAAGGGGT | |
| | | AGAAGTAGCTGCCGACTCATTCATTCAGCCACTCAGTATGTAAACTCGTC | |
| | | CCACTGTTGACTGTATGAATGATGCGGGAGATATTTTTACATCCATCTTT | |
| | | CTCTGTGCTTGGACTGAAGGGAGCTCCTTCTT | |
| CrmiPEP319a | Capsella rubella | TTGTATCCATAGTGTATTTCCTCGCATCTACCATCTACTATTTTCTACGCC | SEQ ID NO: 261 |
| | | TCTCTCTCTTTATCCCTCTATCTCTTTCTTCATCAAATCTTCTTTTGTTCAA | |
| | | AGTCTCTCTCATCATTTTTCTCTATACACATACATGCATCCACATACATAC | |
| | | ATACATATACCATCATCTTCTTTTCTCATCTCTAGTTTTTGTTTATAAATTT | |
| | | TGTTCCAAGGATCTGTATCTCTCCAATAAAGATACATACAAATTATTGTT | |
| | | GTCATAGATCTATTAGAATTGCTTTAACTTTTATATGAGATCTAGGATTTC | |
| | | TTCCTTTTCTTTCAAAATTTGCTGCATATTTTTCAAAATCATGATTTATCG | |
| | | CTTGCTAGGTTCTAGATCCATGCAAATTTAGAGTATTTTTACACACACAC | |
| | | ACGCTTGGACACAAGTACATACATGTAGTTTTCTTTTATGTGGTGAAAAG | |
| | | TACATAACATGTAGTTTATAGTTACTAGTCGCTATATAATTTAAAATTGA | |
| | | TGTTATAGGAATATATGTACGGAGAGAGCTTCCTTGAGTCCATTCACAGG | |
| | | TCGTGATATGATCCAATTAGCTTCCGACTCATTCATCCAAATACCGAGTC | |
| | | TCACCAAAATTGGAACAAGACTTGTTAAATGAATGAATGATGCGGTAGA | |
| | | CAAATTGGATCATTGATTCTCTTTGATTGGACTGAAGGGAGCTCC | |
| EgmiPEP319a | Eucalyptus grandis | TCGTTTCCCATCCCCATTTCATAGAATAATGCCACCAAACAAAGAAGCAT | SEQ ID NO: 262 |
| | | TAGCTCAAAGACTAATTACCATCTGTTTTATTGATAGATACGTGCGAAAC | |
| | | GGTGATTGTTTTTTCCCAAATAAGAAACCAAAATGAAGCATATTCAAAG | |
| | | GTGGAGATATGGGGAGACTTCCGGAAGGCAAGGGGATTGGAAAAGGCT | |
| | | CGAGATCAAAGTGCATAGCAACCCTTCGCTAAAGGTGAAAAAGAATACG | |
| | | AATAACTTCAGTAGCTCACTTTAAATTCCGAAACATTAAACAAATCAAAT | |
| | | CTCCCTCGCCCTCCTTGCCTCCTCTCTTTACCTATATAAAGCCACCGCCCC | |
| | | TTCAATGAAATCCACGAGTGGAAGGTCACAGTATAGTAGGGTCCTGCAA | |
| | | AGGGAGAGCGAGAGCGGCTCCACTGTCTACCTATAAGCAGTTCCTTTCTT | |
| | | TTGTTTACATGTCTGTTGCACCTCACCGAGTTTTTCTATTCTCTTTCCTCTG | |
| | | GTTGGTTAGCAGATTTCTCAGGGGACTTCCCTCCTCCCTTGAGGATCCTTC | |
| | | TCTTGAAGCGATATGTCTCGAATGGGTAAGAGAGAGAGAGGAAGGGAGC | |
| | | TTTCTTCAGTCCACCCATGGGACGTGTTGGGTTTTAATTAGCTGCCGACTC | |
| | | ATTCATCCAAATACCGAGCGAGAGCAAGTAACAGAGCTCCGTAAATGAA | |
| | | TGGATGATGCGGGAGTCTTGTTGATTCCCAAGCTTTCCGTGATTGGACTG | |
| | | AAGGGAGCTCCCTCTATCT | |
| GrmiPEP319a | Gossypium raimondii | GCGTATCCTTCCACTTACGGATTCACCATAGCTGCTATAGCCCGAGTTTG | SEQ ID NO: 263 |
| | | CTTGTCATAATAGAAATAAACTAAGGGAGAAAAAAGCTCTCCACTCTCG | |
| | | CCTTTTTCTTCTTCGAGTCTCTCTCTTTGACTGGCCTTTGTGCAAAGATCTT | |
| | | CCTTTTTTAAACAGTCGCTTTCTTTACTCTCCCTTTCCCTTCTTTCTCTTAA | |
| | | TTGCTAAAGCAGCTTCCACTTCCACTCACTTTAACCCATGATCCATTTCAA | |
| | | CCTGTCACAGTGGAGAGCAATTTGTATGGCTAATTTCCATCTCACCTATT | |
| | | CTTTTCTGTTTGGGGTTCTCTAGTCTAGGGTTGCATGACATGAGAGACAT | |
| | | GGCTCTTTTTTTTTTTTTTCAGTTCACAGGTGTTTATATATGTTGTTGTGTT | |
| | | ATTTTGTCTTAAAGCTTTGTGATTGATGATCTGATAGGTAAGAGAGAGCT | |
| | | TTCTTCAGTCCACTCATGGGATGGGGATGGGGTTTAATTAGCTGCCGACT | |
| | | CATTCATCCAAATACTGTGTTACAAAACCCAGTAAATGAGTGAATGATGC | |
| | | GGGAGACAAATTGAATCCTAATCTTCCTGTGCTTGGACTGAAGGGAGCTC | |
| | | CCTCCC | |
| MtmiPEP319a | Medicago truncatula | ATTTATCCAATCATGTTGCTCTCACTTCTTTTCAGCAGTTCTGTTGATATC | SEQ ID NO: 264 |
| | | ACCCTTTTAGTTGAAGCTTTGGATTCATGACTTTATGAGATGGTAAATCTT | |
| | | TAAATTAAGTTTAAGTATGCACTTGATTTGTTTATATAATTTGTTTATTTA | |
| | | GATTTTAAACCCTAAGTTGACTTTTTTAATTTGATTTAAATTATGATATGA | |
| | | TTGTTATTTGGCTTACCATGGTCATAATTTAGGGTTTAGAAGATGCATGT | |
| | | ATATCTTGAATTGTTTATGGTAATAAAGGGGTTAGGATTTCTCCTTTTGGT | |
| | | GAAGTGAGAAAATCTCATAATTTTGTTCTGAAGGTAGTTTTTAAGATTTA | |
| | | GGGTTATGGGTTCTTTGTTTGAATGCTTTTTCAAGTCTTTTTCAATGATAT | |
| | | TTGCCTAGATCTGTTTTAATTTTGAATTAAAATTCTGGGTTTGGATTAATA | |
| | | TTATTGAAGATTATTATTAATTAATTTATTAGAAATAGATGAAGAGAGCT | |
| | | TCCTTCAGTCCACTCATGGAAGGGTAAGGGGTTTGAATTTACCTGCTGAC | |
| | | TCATTGATTCAAACACAATAGACAATTATGGGGTTATGCTATTGTGAATT | |
| | | GTGTGAATGATGCAGGAGGTGAATTTCTTCCTTTTCTTCTTTGCTTGGACT | |
| | | GAAGGGAGTCTCCCTTT | |
| OsmiPEP319a | Oryza sattva | TTATATCTGACGCGTTGTAATCCTGTTTAATTAGGGCTTTGCCCATTTCTT | SEQ ID NO: 265 |
| | | TTGACCCCTTCCGGACATTCGCTAGTTGGAACCTTGTTTTACTCCTAGCAG | |
| | | TGTACTGTGTAGTACTTATTACGAGCAAACGTAAAAATAAATAAATGGA | |
| | | AATGATACAAAGGCCGTGTTTAATTTTAAAATTTTTTTTCAAACTTTCAAC | |
| | | ACTTTACATCCCATAAAAAGCTTACTATACATACAAACTTCCAACCTTTT | |
| | | CGTCACATCGTATCTAATTTCAACTAAACTTTTAATTTTAGCGTGAACTA | |
| | | AACACAGCCGAAGCCCGGCCACATTCTCACTATTTTTATTCATTTTATCAT | |
| | | GCCTGTGATGTCACGCCTTGGCCCTATTTAATAGGCCTTCTCCATTTCTCT | |
| | | CCATATGATGTCTTCTCTTCTCTATCCCTCTTGCCATCTTCTATCTTCCCTC | |
| | | TTGCACCCATCTTTGTGATAACTTCTACTAGCTCCTCTCCTACTACCAGTC | |
| | | ATACCACTCTCACAAATCCTCCAAGATCCGCATGGGGAGAAGCTCCAAA | |
| | | AGTTTCGTGGTTAGTTTAATTTCATGCTTGTTTGCTGCCGTTTTTCATGTT | |
| | | GATCTGATCTTAATATATGTAGACTGCTGTTAACATATTCTTTTAATTTGA | |
| | | TGGAAGAAGCGATCGATGGATGGAAGAGAGCGATCCTTCAGTCCACTCA | |
| | | TGGGCGGTGCTAGGGTCGAATTAGCTGCCGACTCATTCACCCACAATGCC | |
| | | AAGCAAGAAACGCTTGAGATAGCGAAGCTTAGCAGATGAGTGAATGAA | |
| | | GCGGGAGAGTAACGTTCCGATCTCGCGCCGTCTTTGCTTGGACTGAAGGG | |
| | | TGCTCCCTCCT | |
| PpmiPEP319a | Physcomitrella patens | TGGGATCCACACGAGTAATTATTCCCTCTCTCATGCATCACAACTTGGAC | SEQ ID NO: 266 |
| | | TTGCCCAGCTTTTACCTCTCTCCATGTTCCACCGTCGGAGATCCTCGGTGC | |
| | | TGCTACCCCCGTTCGGCCAAACCCAACCCAACCCTAGGTGTCTGCCGGAC | |
| | | CTCCGCTTCCCCTCCTGCTTCACCCCCTGCACCGCTTAAATTTGCACCTCG | |
| | | TAGTATTTATCTATGCCCCATTTCAGATGACTTGCATGACACCCATCTGG | |
| | | ATTCGTCTCCAGGCGCCTTGTCGTATCTATTCGCTATCAGTCTTTCTTTCA | |
| | | GTCTGTCTTTCTGCCAAGCACACCGTGCTGGTGGTGGTGCTATCAGTCGA | |
| | | AGCAGTCGCCGAAGGGTCCTTGTATCCAGCCCTCACCGGAGACAGTTCG | |
| | | CTTCGGGGTGGGGAGAACTGTTGAGACCGTCGATGTTGCATGCAGCAGC | |
| | | ACTGGCGAAGGTGCTTCTGATACTGGGTATTCCAGCCTCGCCGCTCGGTG | |
| | | CACTGCAGGTACGTGGTTACATGACACTACTGTGTGGGTAGTCTGGGCAC | |
| | | AAGTAGATCGACATGCCAGATTTGGCCCGTATGCGTGTATGTGCGGTTAT | |
| | | GTTCCCGTTTCGATTGCGGATACCTTGATTGTGGAGCTCCGTTTCGGTCCA | |
| | | ATAGTGGCTGCGACGGAAGGTGGTCCCGCTGCCGAATCACACGTCCGGG | |
| | | TTGCTTATCGGGGCAGGGCCCCGATACGGTATCCGAACGTTTGTCCCGGG | |
| | | AACTGGTCGACCTTCCGCCCGGCGTCTCTTGGACTGAAGGGAGCTCCACT | |
| ThmiPEP319a1 | Thellungiella | TTTTATACAAATAATGTTCGATAACACTAAACCCTAGCCATCCAACTAAT | SEQ ID NO: 267 |
| ThmiPEP319a2 | halophila | AGACAAAACCCTACTTGTAATTTACAACCGCAAATTCCCAGAGAACAGA | |
| | | GTAACTACGAGAGAGAGATGGAGATTCAAATTAAAAAGAAAAACTTATA | |
| | | TATAATGAATACACAAAAGCTACCTAATCTGTATATATATATATATAAAT | |
| | | ATGTCTTCATTAAATTAATGGTCGTGGAATAGAAAAAGGAAAACCTAAT | |
| | | TTGATCGCTAGGGCTTATCAGAGTAAAGATGGTTAACCTTCAAAAGATG | |
| | | ACTAATTAACCGGGGAGATAATTAAAAGATTAAATACGCCAACAGAGAG | |
| | | TTAAGAGATACCAGATTTAAATTCCACAATTTGGTCATGTTCTTCTTCAC | |
| | | GTATTCATGACGATGTCTGAATTATAGAGAAACCCAAAATATAAAATGTT | |
| | | AATTTTACCAGACATTTACATACCAATAACTCTATGACGATATGTAAAGT | |
| | | AAGCAAGGCATGTTTTTATGCAGGGAAGATTGAAAATTCAAGATTAATC | |
| | | AAGAAAATTGGAATACCAAAAAGAGAGGGAGCTCCCTTCAGTCCAATCA | |
| | | GAGAGAATCAATGACCCAATTTGTCTACCGCATCATTCATTCATTTAACA | |
| | | AGTCTAGCTCGAATTCTTGGTGACTCGGTATTTGGATGAATGAGTCGGAA | |
| | | GCTAATTGGATCATATCACGACCTATGAATGGACTCAAGGAAGCTCTCTA | |
| | | CAAATGTATTCCTACCACATCAACCCAAATATAGTGATTACAGATGCTGT | |
| | | TCTCACTGTAGACTACATTTACGTTT | |
| AtmiPEP319b1 | Arabidopsis thaliana | AGACATCTCTTCTTCTCTCATCTCTCTTTTCTTCTCTCTTTTCCTCACATAA | SEQ ID NO: 268 |
| | | ACTCTCTTTTTTTACTATTAAATCCATATGGTACCTCAAATTAATCTATGG | |
| | | TCATCTAGGGTTATCTTGAAGATTAGAATTGATTCTAGCACGCACAGAGA | |
| | | GGAAGATCATTGCATCCAGAATCACAAACATGGCCTATCTTTTATCTTTT | |
| | | CTTTTTGATCTAAGTCACTGTTITATGCTATATATAGTATAATCAAATTCT | |
| | | TTACATGTGCTTGTATGTATGCGTATATATAGTAACGGAATTGTTAATAT | |
| | | GCTTATAGATGTTGAGTTGGTGGAGGAAGAGAGCTTTCTTCGGTCCACTC | |
| | | ATGGAGTAATATGTGAGATTTAATTGACTCTCGACTCATTCATCCAAATA | |
| | | CCAAATGAAAGAATTTGTTCTCATATGGTAAATGAATGAATGATGCGAG | |
| | | AGACAAATTGAGTCTTCACTTCTCTATGCTTGGACTGAAGGGAGCTCCCT | |
| AtmiPEP394a1 | Arabidopsis thaliana | TCTTATTCCATCACAATCATCTAGGGTTTTAAGCCAAGCTTATATAGCCC | SEQ ID NO: 269 |
| | | GTCATAAAGAGAACTCATCTGCCTCTCTCTCAATACCAATAAATATCACC | |
| | | ACCGTCCTTCTCTCCTATCACTATTCAATCTATCGCAAACTCCTTTATGTC | |
| | | TCTCCAATTTTATGAGAGGGTTTCCTTCAAGAACACAGTAAAATAGATTG | |
| | | GATCTTTAAACTTTTGTTCCTTTTCATGAGGGTTTGACAAAGATTTTCTTA | |
| | | CAGTCATCTTTGGCATTCTGTCCACCTCCTTCTATACATATATGCATGTGT | |
| | | ATATATATATGCGTTTCGTGTGAAAGAAGGAGGTGGGTATACTGCCAAT | |
| | | AGAGATCTGTTAG | |
| AtmiPEP395c1 | Arabidopsis thaliana | TTGTATCATGACAGAGCAAGAAGAAGAAAGTCAAATGTCCACATGAGTT | SEQ ID NO: 270 |
| | | CCCTTTAACGCTTCATTGTTGAATACTCAAAGCCACATTGGTTTGTATATA | |
| | | ACACTGAAGTGTTTGGGGGGACTCTTGGTGTCAT | |
| AtmiPEP395e1 | Arabidopsis thaliana | TTTCAAACCCTAACACTCTTATAAACCGATTCGCCAAAATGTATCTACAA | SEQ ID NO: 271 |
| | | TATATTGATAATGTAATATCTATATATTCAAACAATCGTCGTGTTGGTCG | |
| | | GATGTTTTCTAGAGTTCCTCTGAGCACTTCATTGGAGATACAATTTTTTAT | |
| | | AAAATAGTTTTCTACTGAAGTGTTTGGGGGAACTCCCGGGCTGATTCGGT | |
| | | ATTTTAAATTCAGTAGACTAGCTAGCTG | |
| AtmiPEP397b1 | Arabidopsis thaliana | TGGTAATAGAAATGAGCAAGGAGATATTTTTTTCCCCTGGGTTTGAATGA | SEQ ID NO: 272 |
| | | ACATCATTGAGTGCATCGTTGATGTAATTTTACTTATTTTATTCCATTGTT | |
| | | GAATTAATTAAAGAAGTATATATCAGCGTTGCATTCAATTATGTTTTTCT | |
| | | AATTTTCAGGAAATACAAAAAAAATGAAAAAAAAAAATCACTTAAAAG | |
| | | ACCTTGAGAGTTCTTTTGACT | |
| AtmiPEP398c1 | Arabidopsis thaliana | GGATATCGAAACTCAAACTGTAACAGTCCTTTTATTACTGGTTTAGAAGA | SEQ ID NO: 273 |
| | | TAGATAAATATTGTTAAGGTAGTGGATCTCGACAGGGTTGATATGAGAA | |
| | | CACACGAGCAATCAACGGCTATAACGACGCTACGTCATTGTTACAGCTCT | |
| | | CGTTTCATGTGTTCTCAGGTCACCCCTGCTGAGCTCTTTCTCTACCGTCCA | |
| | | TGTTTTATCAACGCCGTGGCCCGTG | |
| AtmiPEP399b | Arabidopsis thaliana | TCTTATAGAGATGAAGAGAAACATGTAAACTCACTAGTTTTAGGGCGCCT | SEQ ID NO: 274 |
| | | CTCCATTGGCAGGTCCTTTACTTCCAAATATACACATACATATATGAATA | |
| | | TCGAAAATTTCCGATGATCGATTTATAAATGACCTGCCAAAGGAGAGTTG | |
| | | CCCTGAAACTGGTTC | |
| AtmiPEP399d1 | Arabidopsis thaliana | CAATAACTCAAAATGCAATGTGAAATATGAAGAATATATTAAATAGTAG | SEQ ID NO: 275 |
| | | TGAAGATGCATGTTTATGAAGACAGAGAGATAATGTATGGTTGGATTAC | |
| | | TGGGCGAATACTCCTATGGCAGATCGCATTGGCTAGATATGCAAGTAAA | |
| | | ATGCTTCTCTGCCAAAGGAGATTTGCCCCGCAATTCATCC | |
| AtmiPEP403 | Arabidopsis thaliana | ATTTAGGTCTCTCTTCTTCTTCTTCTTTTTCTTCTTGAGCGCCGGCGAAAA | SEQ ID NO: 276 |
| | | AAGTCTCTGTGAGAAAAAGATACGACGATTGTCATTAGAAGAGTCGTAT | |
| | | TACATGTTTTGTGCTTGAATCTAATTCAACAGGCTTTATGTAAGAGATTCT | |
| | | TTAACAATTCCTATAATCTTTGTTGTTGGATTAGATTCACGCACAAACTC | |
| | | GTAATCTGTCTTTTCGATTTTTACCAGATCTGTC | |
| AtmiPEP447a1 | Arabidopsis thaliana | AATTATATCCATGGTCATGGCTCATCATTAGTCGCACTGCTCTCCTTTTCT | SEQ ID NO: 277 |
| AtmiPEP447a2 | | CAAAGTTTAAATTCGACATTTGGTAAAATGATGAAACCTCGATGGAACT | |
| | | GCTCTCTTTATGGAATCACGGAATGGACAAATAATCAAAATCAGAAATC | |
| | | GAAGCGAAAAGGGAGGAGAAAAACGCAGATTTGGAGGATTGGGGACAG | |
| | | ATTAGATACTGTTGAATGCATCACTCTAATGCTATCAGCCTATTAATAGC | |
| | | GTCCTATATTTTCGAAGACTTTTAATGTTTAGGGTTATGGATTTTTCGAGC | |
| | | GAAGCATGGAGAGATGTTGAATTGGATACTATAGGATTTGGTACAACAC | |
| | | ATACATATGTTCTGCTTCTGCAAAACTAACATATCAAGTTCAGAGAAACC | |
| | | AGTAAGTCGTTGAATATTTTATTATCCATTCAACGCTTTCTTCTTTTGGAT | |
| | | CATGTCTTGTTTGCTTGACCACTTCTTCTTGCTTAAGAGGATGGACAATAT | |
| | | ATAAAAACTGGAGCCTTCTTTTTCTATGAATGCTTATCATCGCGGAGTTG | |
| | | ATCTGTTCAATTCACCTGCCATTGGATGCTTTTTTTATATATACTTCACTG | |
| | | TTCAATTTCAGATGCTTTAGAAGGTTTGCGGAGTAGCTAGAGAATCTGGT | |
| | | ATCTTCAGTTCTTCAATTTCAGCTACTTGGTATCAGCTTCGTCATTGTATA | |
| | | TCAACACATTCTTAATATATAATACTACTTTTTCATCCATTAAACCCCTTA | |
| | | CAATGTCGAGTAAACGAAGCATCTGTCCCCTGGTATTGTCTTCGAGCTTG | |
| | | GTGTTTTTTTCTAGCCAACTCCAAGTTCTCGAGTTGATCATTGTTTGTATT | |
| | | CTTGAGACATTATTTGGGGACGAGATGTTTTGTTGACTCGATATAAGAAG | |
| | | GGGCTTTATGGAAGAAATTGTAGTATTATATATCGAGAGTG | |
| AtmiPEP447b1 | Arabidopsis thaliana | CTATAAATGCTGCTTATCATCGTGGAGTTGGTTCTGTAAACATTTGAAAA | SEQ ID NO: 278 |
| AtmiPEP447b2 | | TTCTGAACAGTTTCACCTGCCATTGGATGCTTTGTTTCAATTTCAGGTGCG | |
| | | TTAGAAGGTTTGCAGAGTAGCTAGAGAATCTCGTATCTTCACTTTCTGCT | |
| | | ACTTGGTATCAGCTTCGTCACTTTATATCAACACATTCTTAATATACAATA | |
| | | CTACTTTTTCATCCATTAATCCCCTTACAATGTCGAGTAAACGAAGCATC | |
| | | TGTCCCCTGGTATTGTCTTCGAGCTTGGTGTGTTTTTCTAGCCAGCCCCAA | |
| | | GTTCTCGAGTTGATCATTGTTTGTATTCTGACACATTATTTGGGGACGAG | |
| | | ATGTTTTGTTGACTCGATATAAGAAGGGGCTTTATGGAAGAAATTGTAGT | |
| | | ATTATATATTGAGAATG | |
| AtmiPEP447c | Arabidopsis thaliana | TAGTATAACCGCTGATGTACACCTACCAGCTTGATAACTCTTTTTCGTGG | SEQ ID NO: 279 |
| | | TTTCTGTGTACTCGTTTCTGTTTGTACAGATACTTCTTGTTCAATTTCAGA | |
| | | TGCTTTAGAAGGTTTTCGGAGTGGCTAGAGATCTGTTATCTGTATGAACA | |
| | | GCTACTTGGTATCAGCTTCGTCATTTTATCAACACATTCTTAATATACAAT | |
| | | ACTTCTTTTTCATGCATTAAGCCCCTTACAATGTCGAGTAAACAAAGCAT | |
| | | GTGTCCGCTAATATTGTCTTCGAGCTTGGTATTTTTGTATTCTGATACGGT | |
| | | ATTTGGGGACGACATCTTTTGTTGACTCGATATAAGAAGGGGGTTTGTGG | |
| | | AAGAAATTGTAGTATTATATATCAAGAATG | |
| DmmiPEP1a | Drosophila | TTTGTGGAACACATTCGACCCACTGAAAAATTGATATAATTTAATGAAAG | SEQ ID NO: 280 |
| DmmiPEP1b | melanogaster | TGCATAAAAATGGTGGACAGTGCATTAAACTGAGCATTGAACACAAAGG | |
| | | CCGCTCAGCAAATTGCTAATTAAAATTCACGATTGCCATTTCACCTGACA | |
| | | CGTTGACGATTTTCATTACAATTCGATTATGTTTCGTTGCAGGGAATTTTA | |
| | | AATGTTAATTGCCAAGAATGTTTCAACAAATTCATTTCTCATTAATGTGT | |
| | | CTTTTCATTTAATTTTATGTTGTATGAGCTGCACGAGAAATGAGTTGTACT | |
| | | TTTAGTTCGACGGCAGAGTCATGAATGTTCGGCAAAGAATGTAATAATA | |
| | | ACTATCCTCTTTAGACAAATATAGATACAAATCTATCAGATTCTAAAAGT | |
| | | AGAATAATCAATTAATCAGAAAGCTAAAAATAAATAGGCATATTTATAT | |
| | | TTTAATGCGGATTTTTGAAGTTCAACGGGAGAAATGAATCCTTTTTACCA | |
| | | GCCACAGGCGCAATTTGCAACAGAAAGTGTAGCAGAAGTACTCCTCGAA | |
| | | TATTTCCCTGCTCCAGGAGTCATCCATGTGGTTTCGAGGCACACATTTGA | |
| | | CAAACTCATGCCCCGCTATTTGTTGTAAAAACACAATCGCACACATGGCC | |
| | | GCATTTCGGCGACTTCCAGAGAGCGGTACACTTAAGGCGGCCTGGGAAA | |
| | | CGCCTGCAATCTGCTGGTCGCGAACTGCAGATTGCATCCATGTGCCAGGC | |
| | | GACCATGCGACCATGTGACCATGTGCCCGCCCGACGCCTCGCAGCCCAC | |
| | | ATCCTGCCCATCGAGGGCACAACTCAGCGTGGGTATTGCCGCTCCGGCTG | |
| | | CTTCAAGTAGGTAAAAACCGAGAAGATTGAGGATGAATGTATGAGTATG | |
| | | AGAAAATACTCGGCGGAACATATGCTGCCGGGCTTGACCTGACCCTGCC | |
| | | TCATGTGTGGGTCTCCGATTTAATTTTAGGCACCTATATAAACGCGTGTTT | |
| | | ACACTGCAGCCAGAACACAGTCGCCGTCTTCAGTTCGCGCCGTCAACTCC | |
| | | TCGATCGATCGATCGCATCGTCTCGGATCGAATAGAGCTGGGCTTCTGCT | |
| | | CCGGAGCTACATCGCCGTACTTGTCGGACGAGTGTGGTGATGAAAAGTC | |
| | | GCTTAGTCCGGGATTCCTGCCAGATCTCTAAGGGATGAGCTGGCATCCCA | |
| | | GGCTGGCCATGTGGCGCGAAGTATGCGCACAAAAAAGTCAAACAAAAA | |
| | | GGCGCAATTTTATTACGGGCAACCAACGACGAAACAAAACAAAAGCCAA | |
| | | CCGAAAAGCAGAAACAAAGCAGCAAAAAGTTTATGAATTTTTTGTGCAG | |
| | | GCGCGTGAAAGATGCAAAACGAGAAAAAAACATGAAAAAAAAACATTA | |
| | | AAAAAAACAAAAAAAATCCAAAACAGATACCGAGCTGTATCCGAAAAC | |
| | | GAGTGGGGAAAGGGGTTTCCCAGTCACATATAAACACACTTCAGTGCGC | |
| | | TTAAAAATTGCTTTATTGCAGTTGGACTATAAAAACGCACGGCAGCGAA | |
| | | CACCGCACAACAAAAAGGACGAGCAGAAGTGGGCAAATAAAACGAAAG | |
| | | CTCTTAAACGAAAAACAGGAAAATTTGCATGCCACAAAAATAAGCATAA | |
| | | GGATTTGCCGCGCACAAAGTAGAAGCAAAAAGGAATTGCCCAAATGCAG | |
| | | CCACAAAAGACTGTGGCAAATGTTTTGCAGCTTGCCCCTTTTTCCCTGCA | |
| | | ATTACCGTCAGTCGTTGTCATTATTCAGCAGATTATATGGTTTTGCTTATT | |
| | | CCGGACCACCATCATCATCATCATTATCATCATCTTCGGTAAGTTAGACA | |
| | | ATCCCATAAAAAACTGTCCAAGTGAGTAGTGCCACCAAAAGTTAGCCGC | |
| | | GTTGTGGAAAATCCAAAACAAAGACCATCCCATATTCAGCCTTTGAGAG | |
| | | TTCCATGCTTCCTTGCATTCAATAGTTATATTCAAGCATATGGAATGTAA | |
| | | AGAAGTATGGAGCGAAATCTGGCGAGACATCGGAGTTGAAACTAAAACT | |
| | | GAAATTTGATTGAAACAGAAGTAGAACCGTAATGAAATGAATGAAATAT | |
| | | TAACCCGTTTCTACAATCCCTGAATAAAATTATTAATTAATTATAGAGCG | |
| | | GGCTAATTTTACAATATATATTGATTTTTTTTTGAAG | |
| DmmiPEP8 | Drosophila | ATTCTTTTTTGGTGCTCGATCGTGACGGTTTGCTCGCGCTCTCCGCTGCGC | SEQ ID NO: 281 |
| | melanogaster | CGCTCTTTCCGTTGCATATGTGTGCGGGCGTTATTGTGCATGTTTCCGGTG | |
| | | GCCGAAAAAAAATAGTAAAATAAAATATAGAAAACAGAAACCAAGAAT | |
| | | AATAACAGCCATACGATAAACAGTGTGCCAATGTGTGTGTCTGTGTGTGT | |
| | | GTGCATCTCGCGTAACAACAATAATTGCATTTATCGGATGGCGCCAGCTT | |
| | | CAATTTAATTATAAATAACATGTTCAACTTTTTATACTATTTTCCCTGCGT | |
| | | CAAAGTGGGCGTTGCAACTGCCCCCGGAAAATCACGCGCCCCGGTTCAA | |
| | | AGTTAAAGTTTGCTGGGTAACGCACACACACACACACACAATCACTCAC | |
| | | ACGCGGTCACACGCACATTTCAATAAACTAATG¹GAGCCTGGCTTTGTTTT | |
| | | TGTTTTATTTCCAACCCACTTGAGCACACAGCACACACAGAGAGAAAAA | |
| | | TCAATACTCGTTATGGGATTAAATTTACAAAGCGCAAAGCAAAGCGACA | |
| | | AACAAAATTCAAAAGAAAGAAAAAAAAACACTCAAATAAACTCACAAA | |
| | | GAATTCCTTATCGCCAAGGGGGCCAATGTTCTAAGGTTCTTTCGCCTTGA¹ | |
| | | GAACTTTGAGCTTCCTCTGGCAAAGGAGATTATAATGTACAAATAATGTT | |
| | | GCAATAACCAGTTGAAACCAATGGAATACCGAATCTTGCTAATTAGCAA | |
| | | GGACATCTGTTCACATCTTACCGGGCAGCATTAGATCCTTTTTATAACTCT | |
| | | AATACTGTCAGGTAAAGATGTCGTCCGTGTCCTTAACCTTCAGTACCACC | |
| | | AACAGCAGCAGCAGCACCAAAAAAAAAAAAAAAAAAATGCGTAAAAAT | |
| | | CCAAACAAATCATAAAAGTCGAAGGA | |
| HsmiPEP155 | Homo sapiens | GCCGAGCCCGGGCCCAGCGCCGCCTGCAGCCTCGGGAAGGGAGCGGATA | SEQ ID NO: 357 |
| | | GCGGAGCCCCGAGCCGCCCGCAGAGCAAGCGCGGGGAACCAAGGAGAC | |
| | | GCTCCTGGCACTGCAGATAACTTGTCTGCATTTCAAGAACAACCTACCAG | |
| | | AGACCTTACCTGTCACCTTGGCTCTCCCACCCAATGGAGATGGCTCTAAT | |
| | | GGTGGCACAAACCAGGAAGGGGAAATCTGTGGTTTAAATTCTTTATGCCT | |
| | | CATCCTCTGAGTGCTGAAGGCTTGCTGTAGGCTGTATGCTGTTAATGCTA | |
| | | ATCGTGATAGGGGTTTTTGCCTCCAACTGACTCCTACATATTAGCATTAA | |
| | | CAGTGTATGATGCCTGTTACTAGCATTCACATGGAACAAATTGCTGCCGT | |
| | | GGGAGGATGACAAAGAAGCATGAGTCACCCTGCTGGATAAACTTAGACT | |
| | | TCAGGCTTTATCATTTTTCAATCTGTTAATCATAATCTGGTCACTGGGATG | |
| | | TTCAACCTTAAACTAAGTTTTGAAAGTAAGGTTATTTAAAAGATTTATCA | |
| | | GTAGTATCCTAAATGCAAACATTTTCATTTAAATGTCAAGCCCATGTTTG | |
| | | TTTTTATCATTAACAGAAAATATATTCATGTCATTCTTAATTGCAGGTTTT | |
| | | GGCTTGTTCATTATAATGTTCATAAACACCTTTGATTCAACTGTTAGAAA | |
| | | TGTGGGCTAAACACAAATTTCTATAATATTTTTGTAGTTAAAAATTAGAA | |
| | | GGACTACTAACCTCCAGTTATATCATGGATTGTCTGGCAACGTTTTTTAA | |
| | | AAGATTTAGAAACTGGTACTTTCCCCCAGGTAACGATTTTCTGTTCAGGC | |
| | | AACTTCAGTTTAAAATTAATACTTTTATTTGACTCTTAAAGGGAAACTGA | |
| | | AAGGCTATGAAGCTGAATTTTTTTAATGAAATATTTTTAACAGTTAGCAG | |
| | | GGTAAATAACATCTGACAGCTAATGAGATATTTTTTCCATACAAGATAAA | |
| | | AAGATTTAATCAAAAAATTTCATATTTGAAATGAAGTCCCAAATCTAGGT | |
| | | TCAAGTTCAATAGCTTAGCCACATAATACGGTTGTGCGAGCAGAGAATCT | |
| | | ACCTTTCCACTTCTAAGCCTGTTTCTTCCTCCATATGGGGATAATACTTTA | |
| | | CAAGGTTGTTGTGAGGCTTAGATGAGATAGAGAATTATTCCATAAGATA | |
| | | ATCAAGTGCTACATTAATGTTATAGTTAGATTAATCCAAGAACTAGTCAC | |
| | | CCTACTTTATTAGAGAAGAGAAAAGCTAATGATTTGATTTGCAGAATATT | |
| | | TAAGGTTTGGATTTCTATGCAGTTTTTCTAAATAACCATCACTTACAAAT | |
| | | ATGTAACCAAACGTAATTGTTAGTATATTTAATGTAAACTTGTTTTAACA | |
| | | ACTCTTCTCAACATTTTGTCCAGGTTATTCACTGTAACCAAATAAATCTCA | |
| | | TGAGTCTTTAGTTGATTTAAAATAAAAAAAAAAAAAAAAAAAAAAAAAA | |
| | | AA | |
| AtmiPEP157c | Arabidopsis thaliana | CTTTGTCACTTCATACACTCCCTATTGTCTATATATATATATATACTTACA | SEQ ID NO: 400 |
| | | CATATTCAAACATTATAATACTTAATTACACATACATACTTTATGATGTT | |
| | | GCATATCACACATAGGTTTGAGAGTGATGTTGGTTGTTGACAGAAGATA | |
| | | GAGAGCACTAAGGATGACATGCAAGTACATACATATATATCATCACACC | |
| | | GCATGTGGATGATAAAATATGTATAACAAATTCAAAGAAAGAGAGGGAG | |
| | | AGAAAGAGAGAGAACCTGCATCTCTACTCTTTTGTGCTCTCTATACTTCT | |
| | | GTCACCACCTTTATCTCTTCTTCTCTCTAACCT | |
| AtmiPEP157d | Arabidopsis thaliana | ATTTACTCTTCACCGCCCTCTCTCTATATATAGTCTCTATCCTCACATATT | SEQ ID NO: 401 |
| | | ATATATCAAACCGCAAGAATGCTGTATGTATAGTGGAGGGTGATAGTGT | |
| | | GGTTGCTGACAGAAGATAGAGAGCACTAAGGATGCTATGCAAAACAGAC | |
| | | ACAGATATGTGTTTCTAATTGTATTTCATACTTTAACCTCAAAGTTGATAT | |
| | | AAAAAAAGAAAGAAAGATAGAAGAGCTAGAAGACTATCTGCATCTCTAT | |
| | | TCCTATGTGCTCTCTATGCTTCTGTCATCACCTTTCTTTCTCTATTTCTCTC | |
| | | TAC | |
| AtmiPEP160c | Arabidopsis thaliana | AACCAAAACTCTTCAACATTTCTCTCTGACTACTTCATTTCCTCTTCCCAA | SEQ ID NO: 402 |
| | | CAGTTAAAAAAAGTTCTGATTCGATTCAAGCCAAGATCCACGTATAAAG | |
| | | ATATGTTCATGCGTAGAGGTTTGGTATACAACAATATATACATATAATAG | |
| | | TTTGTCGTTATGCCTGGCTCCCTGTATGCCACGAGTGGATACCGATTTTG | |
| | | GTTTTAAAATCGGCTGCCGGTGGCGTACAAGGAGTCAAGCATGAC | |
| AtmiPEP164b | Arabidopsis thaliana | ATACATTCTCTCTTTCTCTCTCTCTCTCTCTCTCATCCCGGCCCAGTTATGT | SEQ ID NO: 403 |
| | | GGTCGGAGAGAATGATGAAGGTGTGTGATGAGCAAGATGGAGAAGCAG | |
| | | GGCACGTGCATTACTAGCTCATATATACACTCTCACCACAAATGCGTGTA | |
| | | TATATGCGGAATTTTGTGATATAGATGTGTGTGTGTGTTGAGTGTGATGA | |
| | | TATGGATGAGTTAGTTCTTCATGTGCCCATCTTCACCATC | |
| AtmiPEP166c | Arabidopsis thaliana | TCACACATACCTTTCTTTCTCTTCTTCTTCTTACGAAAAGTTTCATCACAT | SEQ ID NO: 404 |
| | | TCACATTATCTTTAACTTTGGTCTCTITTCTTTTTTGTCTCTTTTCTCTTCTT | |
| | | GATAACGTGGTTCTAGTCTTGATTAATTCATTGTTGTGCGATTTAGTGTTG | |
| | | AGAGGATTGTTGTCTGGCTCGAGGTCATGAAGAAGAGAATCACTCGAAT | |
| | | TAATTTGGAAGAACAAATTAAGAAAACCCTAGATGATTCTCGGACCAGG | |
| | | CTTCATTCCCCCTAACCTACTTATCGC | |
| AtmiPEP166d | Arabidopsis thaliana | ATTTAGCTTCTTCTTCTTCTTCTTCTTCTGTCTACTTACATAAAGTTATCCTt | SEQ ID NO: 405 |
| | | GCTTTGGTTTAGGGTTGAGAGGAATATTGTCTGGCTCGAGGTCATGAAGA | |
| | | AGATCGGTAGtATTGATTCATTTTAAAGAGTGAAATCCCTAAATGATTCTC | |
| | | GGACCAGGCTTCATTCCCCCCAACC | |
| AtmiPEP169a | Arabidopsis thaliana | TAGTATTCATAAGCACCAAAACAAATATGTAGAGATCTCCTCTTCCATTC | SEQ ID NO: 406 |
| | | TCTATTGTTACTTTCGAGAAGAAACATACAAAACACATACATTTTTCTTTT | |
| | | GTTTGTGGTTTTCATATATACAAGTGGGTATAGCTAGTGAAACGCGAATG | |
| | | TGACGAAAGTAGTGTGCAGCCAAGGATGACTTGCCGATTTAAATGATCTT | |
| | | TCTTTATACTCTATTAAGACAATTTAGTTTCAAACTTTTTTTTTTTTTTTTT | |
| | | TTTGAAGGATTCAGGAAGAAATTAGGATATATTATTCCGTATAAAATACA | |
| | | AGATATATAAAACCAAAAAGAAAAAGTAACATGATCGGCAAGTTGTCCT | |
| | | TGGCTACACGTTACTTTGTGTCGC | |
| AtmiPEP169h1 | Arabidopsis thaliana | ACTCATCAACAACCTCTTCATAAATACATAAATCATATAAGAGAAAATG | SEQ ID NO: 407 |
| AtmiPEP169h2 | | GTGACATGAAGAATGAGAACTTGTGTGG | |
| AtmiPEP169n | Arabidopsis thaliana | AGGCAAAAACATATAGAGAGTAATGAAGTGTATGATGAAGAAGAGAGG | SEQ ID NO: 408 |
| | | TCTAACATGGCGGAAAGCGTCATGTTTAGTAGCCAAGGATGACTTGCCTG | |
| | | ATCTTTTTCGCCTCCACGATTCAATTTCAAATTCATGCATTTTGGATTATT | |
| | | ATACCTTTTAAAGTATAATAGGTCAAATATCATGTTGAATCTTGCGGGTT | |
| | | AGGTTTCAGGCAGTCTCTTTGGCTATCTTGACATGCTTTTTCCATCCAT | |
| AtmiPEP170 | Arabidopsis thaliana | ATTCACTCCCTTCTTCTTCTTAATCTCCTTACAGTTACAGACATTCTCTCA | SEQ ID NO: 409 |
| | | CTTGCGTTCTTGTTTCTTTTACAAAACAGATACACTATGTTTCCGAGAGA | |
| | | GTCCCTCTGATATTGGCCTGGTTCACTCAGATTCTCTTTTACTAACTCATC | |
| | | TGATTGAGCCGTGTCAATATCTCAGTCCTCTCTCG | |
| AtmiPEP396a | Arabidopsis thaliana | TCTCACAACTTCAACTTCCCTCTTTCTCTATATTACGCTTTTGCCCCTCACT | SEQ ID NO: 410 |
| | | CCCTCTTTCCACAATTAGGGTTTCGTCTGCTCTACATGACCCTCTCTGTAT | |
| | | TCTTCCACAGCTTTCTTGAACTGCAAAACTTCTTCAGATTTTTTTTTTTTTC | |
| | | TTTTGATATCTCTTACGCATAAAATAGTGATTTTCTTCATATCTCTGCTCG | |
| | | ATTGATTTGCGGTTCAATAAAGCTGTGGGAAGATACAGAC | |
| AtmiPEP399c | Arabidopsis thaliana | GAATAACCAACCAGCCTTCTCTCAAAGCAAACCAAAAAGAAAAACCAAC | SEQ ID NO: 411 |
| | | ATTGAAAGAGGAAGTTACGATAAGCGGAGCAGTAATAGGGCATCTTTCT | |
| | | ATTGGCAGGCGACTTGGCTATTTGTATCTTTTGTGTTCTTGACTATTGGCT | |
| | | ATGTCACTTGCCAAAGGAGAGTTGCCCTGTCACTGCTTCCGCTTAAACAC | |
| | | AGTCTATAACCGGTTCTGCTAATATCAATCCTTCTTTTGGACATGTCCAA | |
| | | AGCCGAGATTGATTGATAGAGAATTGGTCTCTCTGGCTACAAAACTAGTG | |
| | | CGGTTCTCTCGATTTAAGTTTTAATAGCATTCACTTTGCACATTGCATCTT | |
| | | TCACATCAAATTTCCATTTCATCAACCATCTAAACCTCTTTGTTAGCTTTG | |
| | | ATATAAGCAACGATCTAAAGTCTAAAAACCATTAATCCTCTGAAAAAAA | |
| | | AGACAATTTCGATGGTTCTATTATGTTTCTCCAATGCAGAAATTGTATCG | |
| | | TCTGAATTATAGTAGATTTTTTCTAGACTAAAGTGTAAACCAAGACGAAT | |
| | | CTGCACTAACAAGACACACCAATAGACTTTACAGAGAAAGGTTACGAGT | |
| | | TTTGAAAATATTAACGGACCATAGTCATCGCG | |

TABLE 4
List of the microRNAs (miRNAs)

| miPEP | Organism | Sequence of the miRNA | SEQ ID |
|---|---|---|---|
| AtmiPEP156a1 | Arabidopsis thaliana | ugacagaagagagugagcac | SEQ ID NO: 282 |
| AtmiPEP156a2 | | | |
| AtmiPEP156a3 | | | |
| AtmiPEP156c1 | Arabidopsis thaliana | ugacagaagagagugagcac | SEQ ID NO: 283 |
| AtmiPEP156c2 | | | |
| AtmiPEP156e1 | Arabidopsis thaliana | ugacagaagagagugagcac | SEQ ID NO: 284 |
| AtmiPEP156f1 | Arabidopsis thaliana | ugacagaagagagugagcac | SEQ ID NO: 285 |
| AlmiPEP159a | Arabidopsis lyrata | uuuggauugaagggagcucua | SEQ ID NO: 286 |
| AtmiPEP159a1 | Arabidopsis thaliana | uuuggauugaagggagcucua | SEQ ID NO: 287 |
| CrmiPEP159a | Capsella rubella | uuuggauugaagggagcucua | SEQ ID NO: 288 |
| AtmiPEP159b1 | Arabidopsis thaliana | uuuggauugaagggagcucuu | SEQ ID NO: 289 |
| AtmiPEP159b2 | | | |
| AtmiPEP160a1 | Arabidopsis thaliana | ugccuggcucccuguaugcca | SEQ ID NO: 290 |
| AtmiPEP160b1 | Arabidopsis thaliana | ugccuggcucccuguaugcca | SEQ ID NO: 291 |
| AtmiPEP160b2 | | | |
| AtmiPEP161 | Arabidopsis thaliana | ucaaugcauugaaagugacua | SEQ ID NO: 292 |
| AtmiPEP162a1 | Arabidopsis thaliana | ucgauaaaccucugcauccag | SEQ ID NO: 293 |
| AtmiPEP162b1 | Arabidopsis thaliana | ucgauaaaccucugcauccag | SEQ ID NO: 294 |
| AtmiPEP163-1 | Arabidopsis thaliana | uugaagaggacuuggaacuucgau | SEQ ID NO: 295 |
| AtmiPEP163-2 | | | |
| AlmiPEP164a1 | Arabidopsis lyrata | uggagaagcagggcacgugca | SEQ ID NO: 296 |
| AlmiPEP164a2 | | | |
| AlmiPEP164a3 | | | |
| AtmiPEP164a1 | Arabidopsis thaliana | uggagaagcagggcacgugca | SEQ ID NO: 297 |
| AtmiPEP164a2 | | | |
| AtmiPEP164a3 | | | |
| BrmiPEP164a1 | Brassica rapa | uggagaagcagggcacgugca | SEQ ID NO: 298 |
| BrmiPEP164a2 | | | |
| BrmiPEP164a3 | | | |
| CpmiPEP164a1 | Carica papaya | uggagaagcagggcacgugca | SEQ ID NO: 299 |
| CpmiPEP164a2 | | | |
| CrmiPEP164a1 | Capsella rubella | uggagaagcagggcacgugca | SEQ ID NO: 300 |
| CrmiPEP164a2 | | | |
| CrmiPEP164a3 | | | |
| GrmiPEP164a1 | Gossypium raimondii | uggagaagcagggcacgugca | SEQ ID NO: 301 |
| GrmiPEP164a2 | | | |
| GrmiPEP164a3 | | | |
| MtmiPEP164a1 | Aledicago truncatula | uggagaagcagggcacgugca | SEQ ID NO: 302 |
| MtmiPEP164a2 | | | |
| OsmiPEP164a1 | Oryza sativa | uggagaagcaggguacgugca | SEQ ID NO: 303 |
| OsmiPEP164a2 | | | |
| AlmiPEP165a | Arabidopsis lyrata | ucggaccaggcuucauccccc | SEQ ID NO: 304 |
| AtmiPEP165a | Arabidopsis thaliana | ucggaccaggcuucauccccc | SEQ ID NO: 305 |
| BcmiPEP165a | Brassica carinata | ucggaccaggcuucauccccc | SEQ ID NO: 306 |
| BjmiPEP165a | Brassica juncea | ucggaccaggcuucauccccc | SEQ ID NO: 307 |
| BnmiPEP165a | Brassica napus | ucggaccaggcuucauccccc | SEQ ID NO: 308 |
| BomiPEP165a | Brassica oleracea | ucggaccaggcuucauccccc | SEQ ID NO: 309 |
| BrmiPEP165a | Brassica rapa | ucggaccaggcuucauccccc | SEQ ID NO: 310 |
| AtmiPEP166a | Arabidopsis thaliana | ucggaccaggcuucauucccc | SEQ ID NO: 311 |
| AtmiPEP166b | Arabidopsis thaliana | ucggaccaggcuucauucccc | SEQ ID NO: 312 |
| AtmiPEP167a | Arabidopsis thaliana | ugaagcugccagcaugaucua | SEQ ID NO: 313 |
| AtmiPEP167b1 | Arabidopsis thaliana | ugaagcugccagcaugaucua | SEQ ID NO: 314 |
| AtmiPEP167b2 | | | |
| AtmiPEP169c1 | Arabidopsis thaliana | cagccaaggaugacuugccgg | SEQ ID NO: 315 |
| AtmiPEP169c2 | | | |
| AtmiPEP16911 | Arabidopsis thaliana | uagccaaggaugacuugccug | SEQ ID NO: 316 |
| AtmiPEP171a1 | Arabidopsis thaliana | ugauugagccgcgccaauauc | SEQ ID NO: 317 |
| AtmiPEP171b | Arabidopsis thaliana | uugagccgugccaauaucacg | SEQ ID NO: 318 |
| MtmiPEP171b1 | Medicago truncatula | ugauugagccgcgucaauauc | SEQ ID NO: 319 |
| MtmiPEP171b2 | | | |
| ZmmiPEP171b | Zea mays | ggauugagccgcgucaauauc | SEQ ID NO: 320 |
| AtmiPEP171c1 | Arabidopsis thaliana | uugagccgugccaauaucacg | SEQ ID NO: 321 |
| MtmiPEP171e | Medicago truncatula | agauugagccgcgccaauauc | SEQ ID NO: 322 |
| MtmiPEP171h | Medicago truncatula | cgagccgaaucaauaucacuc | SEQ ID NO: 323 |
| AtmiPEP172a1 | Arabidopsis thaliana | agaaucuugaugaugcugcau | SEQ ID NO: 324 |
| AtmiPEP172a3 | | | |
| AtmiPEP172b1 | Arabidopsis thaliana | gcagcaccauuaagauucac | SEQ ID NO: 325 |
| AtmiPEP172c1 | Arabidopsis thaliana | agaaucuugaugaugcugcag | SEQ ID NO: 326 |
| AtmiPEP172e1 | Arabidopsis thaliana | ggaaucuugaugaugcugcau | SEQ ID NO: 327 |
| AtmiPEP172e2 | | | |
| AtmiPEP172e3 | | | |
| AcmiPEP319a1 | Arabidopsis cebennensis | uuggacugaagggagcucccu | SEQ ID NO: 328 |
| AcmiPEP319a2 | | | |
| AhmiPEP319a | Arabidopsis halleri | uuggacugaagggagcucccu | SEQ ID NO: 329 |
| AlmiPEP319a | Arabidopsis lyrata | uuggacugaagggagcucccu | SEQ ID NO: 330 |
| AtmiPEP319a1 | Arabidopsis thaliana | uuggacugaagggagcucccu | SEQ ID NO: 331 |
| AtmiPEP319a2 | | | |
| BrmiPEP319a | Brassica rapa | uuggacugaagggagcucccu | SEQ ID NO: 332 |
| CpmiPEP319a | Carica papaya | uuggacugaagggagcuccuu | SEQ ID NO: 333 |
| CrmiPEP319a | Capsella rubella | uuggacugaagggagcucc | SEQ ID NO: 334 |
| EgmiPEP319a | Eucalyptus grandis | uuggacugaagggagcucccu | SEQ ID NO: 335 |
| GrmiPEP319a | Gossypium raimondii | uuggacugaagggagcucccu | SEQ ID NO: 336 |
| MtmiPEP319a | Medicago truncatula | uuggacugaagggagucucccu | SEQ ID NO: 337 |
| OsmiPEP319a | Oryza sativa | uuggacugaagggugcucccu | SEQ ID NO: 338 |
| PpmiPEP319a | Physcomitrella patens | cuuggacugaagggagcucc | SEQ ID NO: 339 |
| ThmiPEP319a1 | Thellungiella halophila | uggacucaaggaagcucucu | SEQ ID NO: 340 |
| ThmiPEP319a2 | | | |
| AtmiPEP319b1 | Arabidopsis thaliana | uuggacugaagggagcucccu | SEQ ID NO: 341 |
| AtmiPEP394a1 | Arabidopsis thaliana | uuggcauucuguccaccucc | SEQ ID NO: 342 |
| AtmiPEP395c1 | Arabidopsis thaliana | cugaaguguuuggggggacuc | SEQ ID NO: 343 |
| AtmiPEP395e1 | Arabidopsis thaliana | cugaaguguuugggggaacuc | SEQ ID NO: 344 |
| AtmiPEP397b1 | Arabidopsis thaliana | ucauugagugcaucguugaug | SEQ ID NO: 345 |
| AtmiPEP398c1 | Arabidopsis thaliana | uguguucucaggucaccccug | SEQ ID NO: 346 |
| AtmiPEP399b | Arabidopsis thaliana | ugccaaaggagaguugcccug | SEQ ID NO: 347 |
| AtmiPEP399d1 | Arabidopsis thaliana | ugccaaaggagauuugccccg | SEQ ID NO: 348 |
| AtmiPEP403 | Arabidopsis thaliana | uuagauucacgcacaaacucg | SEQ ID NO: 349 |
| AtmiPEP447a1 | Arabidopsis thaliana | uuggggacgagauguuuuguug | SEQ ID NO: 350 |
| AtmiPEP447a2 | Arabidopsis thaliana | uuggggacgagauguuuuguug | |
| AtmiPEP447b1 | Arabidopsis thaliana | uuggggacgagauguuuuguug | SEQ ID NO: 351 |
| AtmiPEP447b2 | Arabidopsis thaliana | uuggggacgagauguuuuguug | |
| AtmiPEP447c | Arabidopsis thaliana | ccccuuacaaugucgaguaaa | SEQ ID NO: 352 |
| DmmiPEP1a | Drosophila melanogaster | uggaauguaaagaaguauggag | SEQ ID NO: 353 |
| DmmiPEP1b | | | |
| DmmiPEP8 | Drosophila melanogaster | uaauacugucagguaaagauguc | SEQ ID NO: 354 |
| HsmiPEP155 | Homo sapiens | uuaaugcuaaucgugauaggggu | SEQ ID NO: 358 |
| AtmiPEP157c | Arabidopsis thaliana | uugacagaagauagagagcac | SEQ ID NO: 412 |
| AtmiPEP157d | Arabidopsis thaliana | ugacagaagauagagagcac | SEQ ID NO: 413 |
| AtmiPEP160c | Arabidopsis thaliana | ugccuggcucccuguaugcca | SEQ ID NO: 414 |
| AtmiPEP164b | Arabidopsis thaliana | uggagaagcagggcacgugca | SEQ ID NO: 415 |
| AtmiPEP166c | Arabidopsis thaliana | ucggaccaggcuucauucccc | SEQ ID NO: 416 |
| AtmiPEP166d | Arabidopsis thaliana | ucggaccaggcuucauucccc | SEQ ID NO: 417 |
| AtmiPEP169a | Arabidopsis thaliana | cagccaaggaugacuugccga | SEQ ID NO: 418 |
| AtmiPEP169h | Arabidopsis thaliana | uagccaaggaugacuugccug | SEQ ID NO: 419 |
| AtmiPEP169n | Arabidopsis thaliana | uagccaaggaugacuugccug | SEQ ID NO: 420 |
| AtmiPEP170 | Arabidopsis thaliana | ugauugagccgugucaauauc | SEQ ID NO: 421 |
| AtmiPEP396a | Arabidopsis thaliana | uuccacagcuuucuugaacug | SEQ ID NO: 422 |
| AtmiPEP399c | Arabidopsis thaliana | ugccaaaggagaguugcccug | SEQ ID NO: 423 |

TABLE 5
List of the control Pre-miRNAs

| Pre- | | | SEQ |
|---|---|---|---|
| miRNA | Organism | Sequence of the Pre-miRNA | ID |
| Pre- | Medicago | TTAGGGTTTTCAGCTCATGGTAATA | SEQ |
| miR169 | truncatula | AAAATGTCATCTAATGTCTTGCATG | ID |
| | | TGGGAATGAGGTCATATATGCAGCC | NO: |
| | | AAGGATGACTTGCCGGCGAGCCTCT | 359 |
| | | TTCGATACTTTTATGACATAATTAA | |
| | | TCATGTGGATAGCCAAGGTACTAAA | |
| | | CTCACTTTGCACTAAAACAAATATT | |
| | | TTTGCTTTAGTGCAAACTTAGTTTA | |
| | | GGCGCTTCGCAACGGCTAGTCAAAT | |
| | | GTCCTAGTTCCAATGTGATTGGTTG | |
| | | TCCGGCAAGTCGTCTCTGGCTACGT | |
| | | AAAGGCCTCCTTTTTTCATGCTAGA | |
| | | TTTTTGATGATTTGATATAGCCACA | |
| | | CATATTTTGGAA | |
| Pre- | Medicago | AAGAGGCAGAGAGAGTAATGCAGCC | SEQ |
| miR169a | truncatula | AAGGATGACTTGCCGACAACATTGG | ID |
| | | CGAATGTTCATGTGATTTCTGCCTC | NO: |
| | | ATTGTGCCGGCAAGTTGTCCTTGGC | 360 |
| | | TATGTTAGTCTCTCATCTTCT | |
| Pre- | Medicago | TGAATTCCCCTCCGCTTTTTGATGT | SEQ |
| miR171a | truncatula | TGGCTTGTCTCAATCAAATCAAAGT | ID |
| MI | | TCTTGAAATTTGAGTTCTTTAGTCT | NO: |
| 0001753 | | GATTGAGTCGTGCCAATATCATATT | 361 |
| | | AAGCGATAAAAGTC | |
| Pre- | Medicago | CCACAAAACTATAACTAGCTAGAAG | SEQ |
| miR171h | truncatula | CTTTAATCGCCTTATTTATTATAAT | ID |
| | | AATAATAATAAATATGGCTTCAGCT | NO: |
| | | GCAAAAGTATACATGGCGTGATATT | 362 |
| | | GATCCGGCTCATCTATATCTTCAAG | |
| | | TTCAATCATCCATATTCATATCAAT | |
| | | TTCAGACGAGCCGAATCAATATCAC | |
| | | TCTTGTTTGCTTCATTGCATATTAA | |
| | | TTATATACTTCATTTATAAGTTATA | |
| | | GTTTGCCATATATATATTAGATTGA | |
| | | TTCTGCAGAAGTAGACAGGAGTGGT | |
| | | GTTGTTTCTGCTCATCTTATTAAAT | |
| | | AATGAATGAATGAATGACATTTGCT | |
| | | TACTTATAAGACGAGCCGAATCAAT | |
| | | ATCACTCCAGTACACCT | |
| Pre- | Medicago | AACTGCAACTTGAGGAGGCATCCAA | SEQ |
| miR393a | truncatula | AGGGATCGCATTGATCCTATAATAT | ID |
| | | TTCAACTTTAGTCACTTTAATTTTC | NO: |
| | | TCTCATATAATACTTAATTGGGATC | 363 |
| | | ATGCCATCCCTTTGGATTTCTCCTT | |
| | | TAGTAGCTAC | |
| Pre- | Medicago | AGGCATCCAAAGGGATCGCATTGAT | SEQ |
| miR393b | truncatula | CCCAAATCTAATTAAGTCCCTAGCT | ID |
| | | CATTAATTAACAACTTAATTTCCTT | NO: |
| | | AATATCTCATAATATTTGGGATCAT | |
| | | GCTATCCCTTTGGATTCAT | 364 |
| Pre- | Medicago | TGCTTTTCCACAGCTTTCTTGAACT | SEQ |
| miR396a | truncatula | TCTTTCGTATCTTAAATCTGTTTTC | ID |
| | | AAGATTAAAAGTCCTAGAAGCTCAA | NO: |
| | | GAAAGCTGTGGGAGAATA | 365 |
| Pre- | Medicago | TATTCTTCCACAGCTTTCTTGAACT | SEQ |
| miR396b | truncatula | GCATCCAAATTGAGTTCCTTTGCAT | ID |
| | | TGCCATGGCCATTGTTTTGCGGTTC | NO: |
| | | AATAAAGCTGTGGGAAGATA | 366 |

TABLE 6
List of the control miRNAs

| | | Sequence of the | |
|---|---|---|---|
| miRNA | Organism | miRNA | SEQ ID |
| miR169 | Medicago | CAGCCAAGGAUGACUU | SEQ ID NO: 367 |
| | truncatula | GCCGG | |
| miR169a | Medicago | CAGCCAAGGAUGACUU | SEQ ID NO: 368 |
| | truncatula | GCCGA | |
| miR171a | Medicago | UGAUUGAGUCGUGCCA | SEQ ID NO: 369 |
| | truncatula | AUAUC | |
| miR171h | Medicago | GAGCCGAAUCAAUAUC | SEQ ID NO: 370 |
| | truncatula | ACUC | |
| miR393a | Medicago | UCCAAAGGGAUCGCAU | SEQ ID NO: 371 |
| | truncatula | UGAUC | |
| miR393b | Medicago | UCCAAAGGGAUCGCAU | SEQ ID NO: 372 |
| | truncatula | UGAUC | |
| miR396a | Medicago | UUCCACAGCUUUCUUG | SEQ ID NO: 373 |
| | truncatula | AACUU | |
| miR396b | Medicago | UUCCACAGCUUUCUUG | SEQ ID NO: 374 |
| | truncatula | AACUG | |

TABLE 7
Polymorphism of the DNA sequence of
the different regions of pri-miR171b

| | Size | # SNPs | # mutations | % SNP | # haplotypes |
|---|---|---|---|---|---|
| pri-mir171b | 1127 | 91 | 100 | 8.07 | 161 |
| 5′ pri-miR171b | 129 | 4 | 4 | 3.1 | 5 |
| miPEP171b | 62 | 2 | 2 | 3.22 | 3 |
| Pre-miR171b | 118 | 1 | 1 | 0.85 | 2 |
| miR171b + | 42 | 0 | 0 | 0 | 1 |
| miR171b* | | | | | |
| 3′ pri-miR171b | 259 | 39 | 42 | 15.06 | 89 |

### EXAMPLES

### A: Analysis of the miPEPS in Plants

### Example 1—Characterization in the Model Plant Medicago truncatula

a) Identification and Characterization of MtmiPEP171b1 (miPEP171b1 Identified in Medicago truncatula)

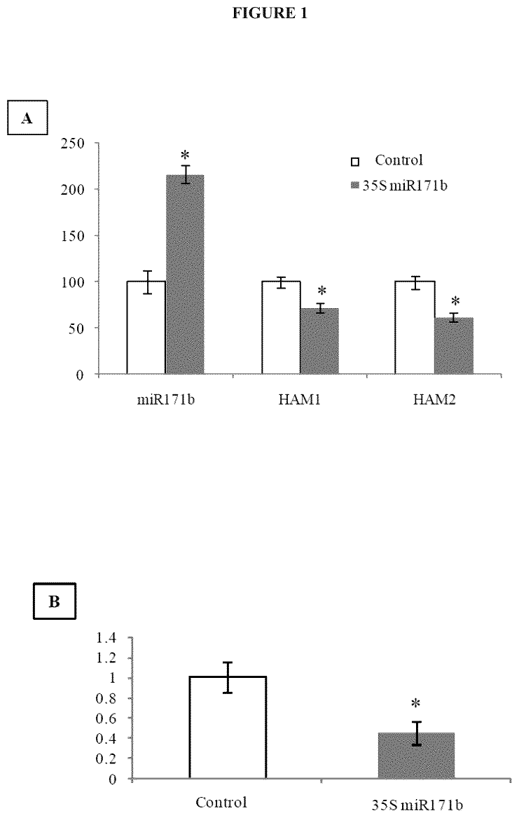

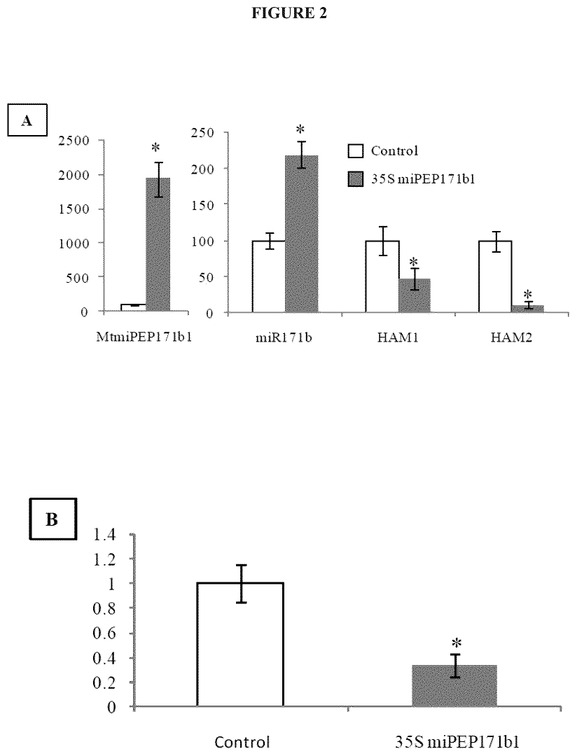

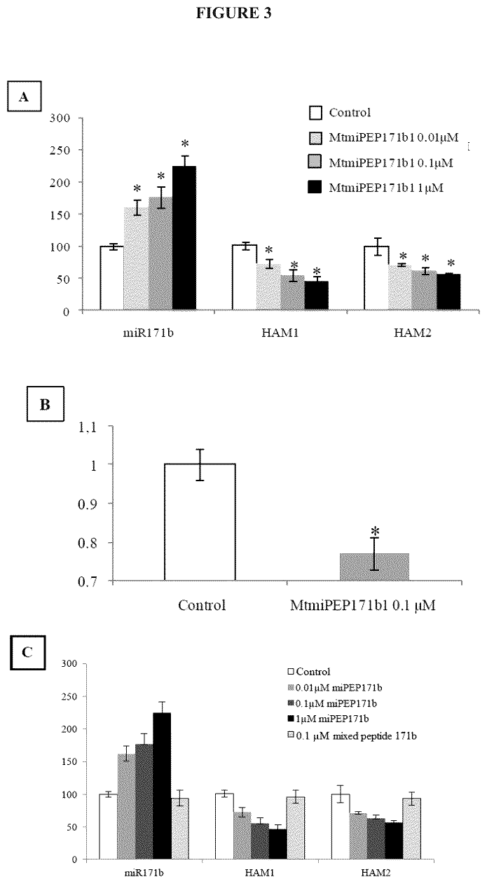

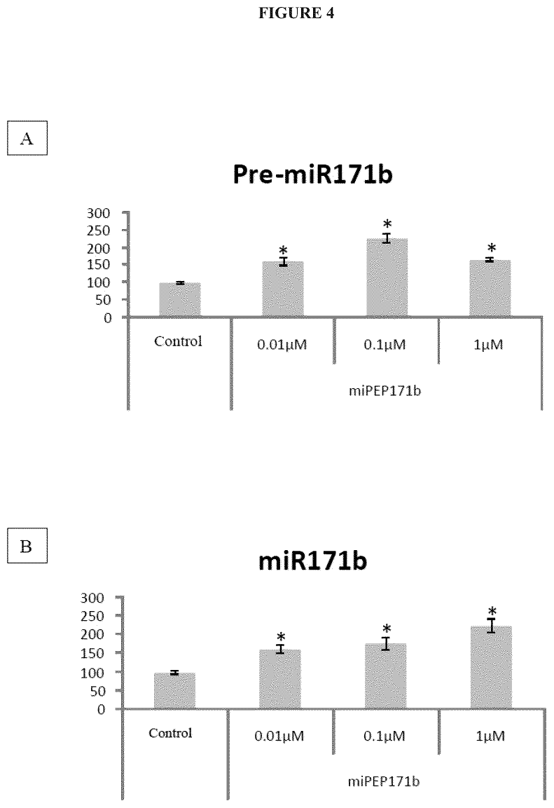

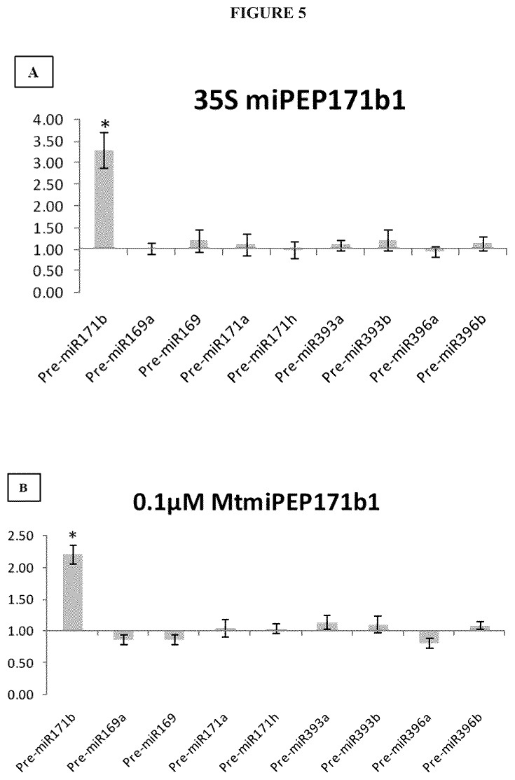

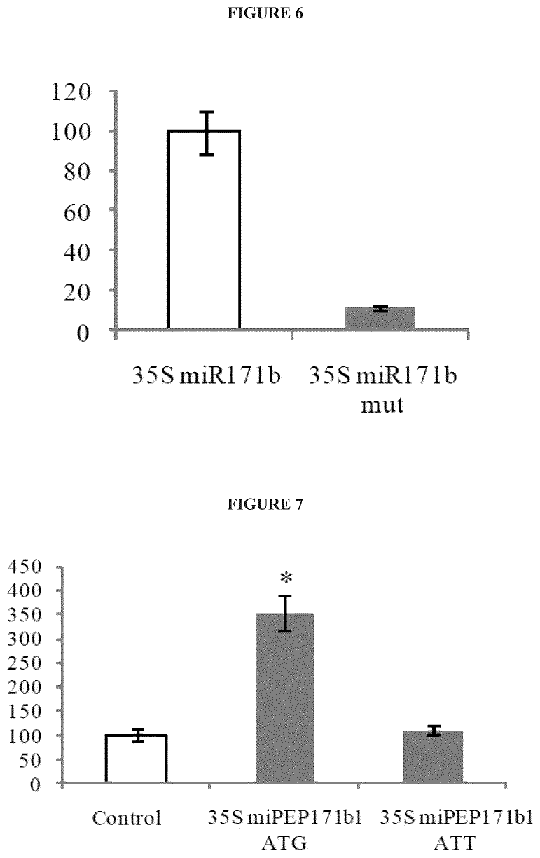

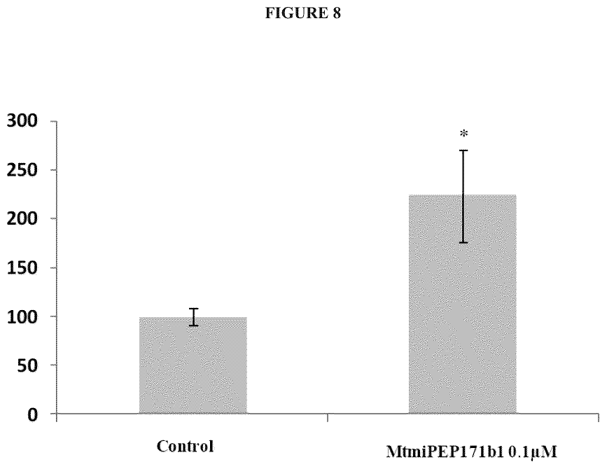

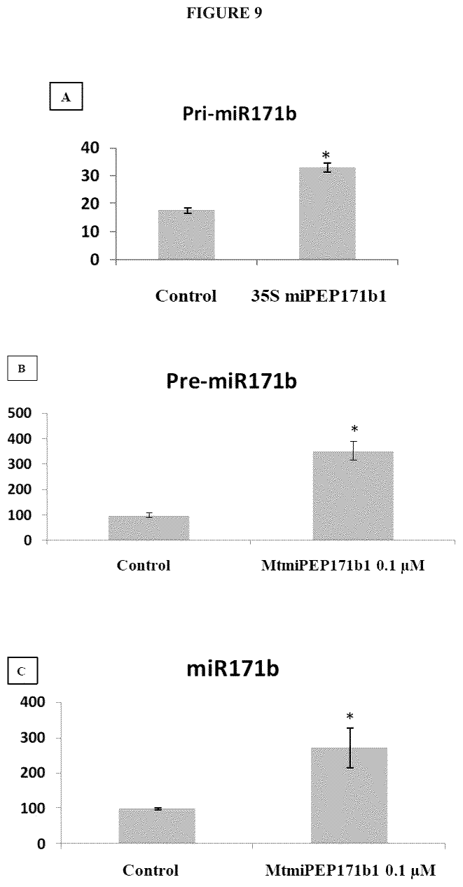

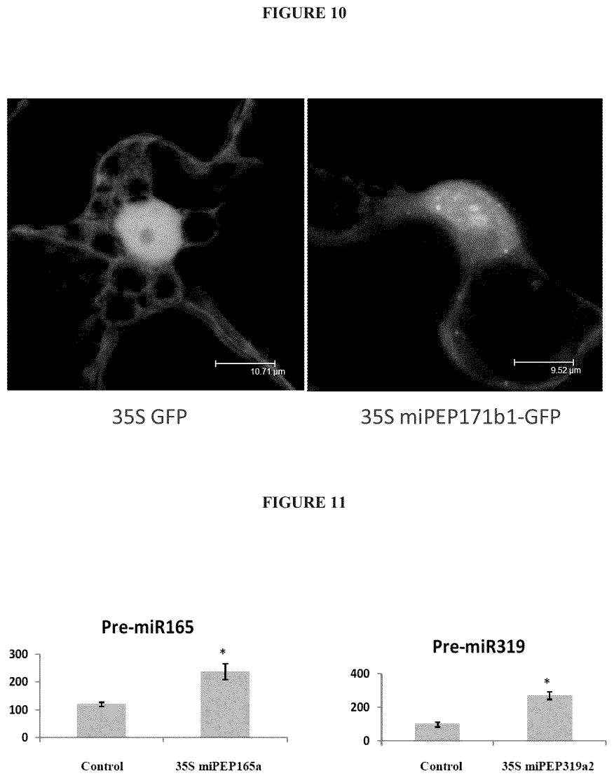

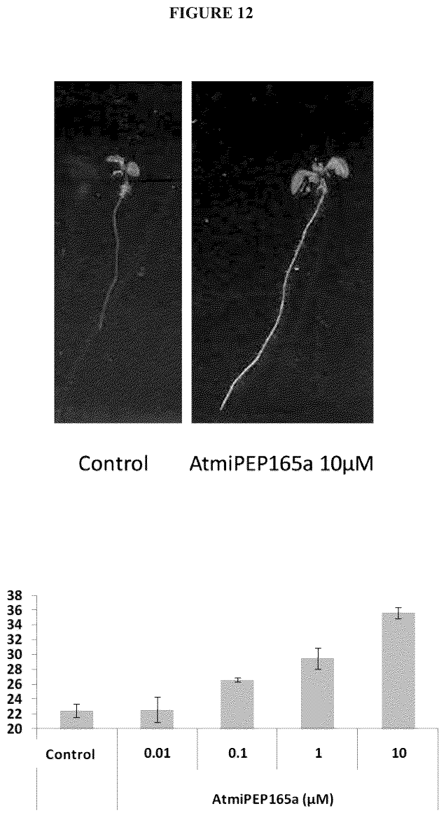

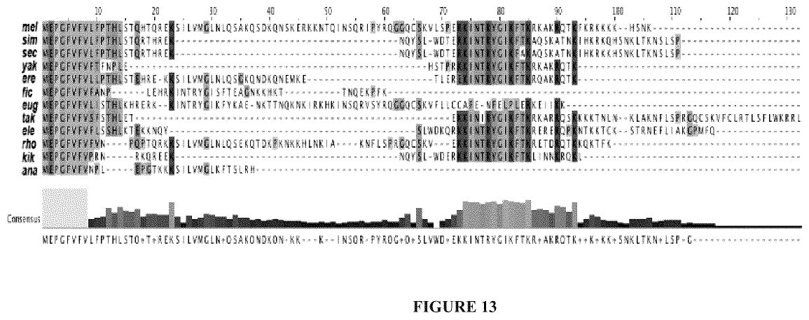

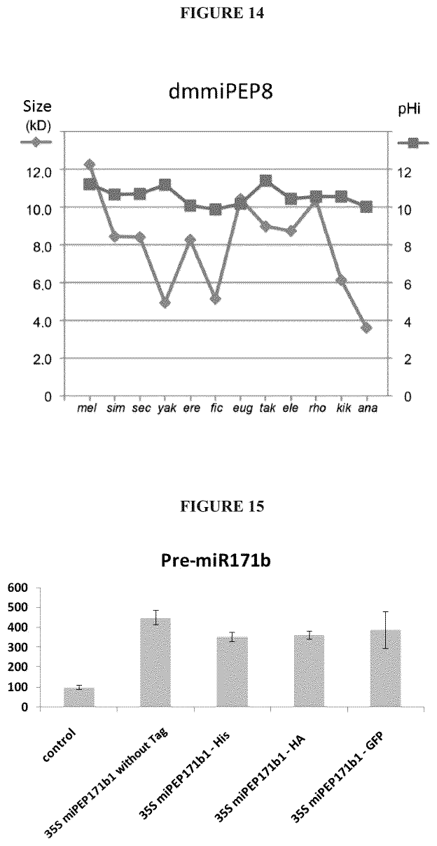

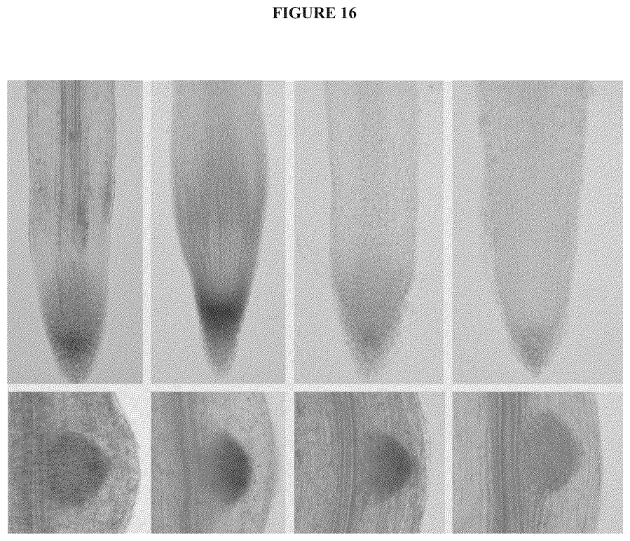

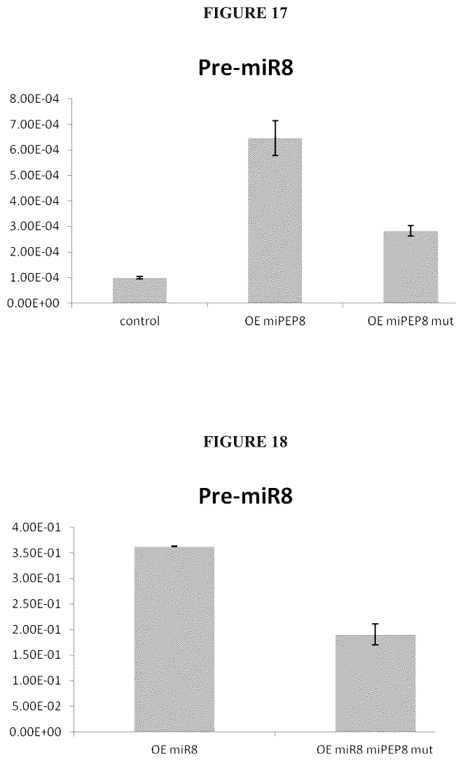

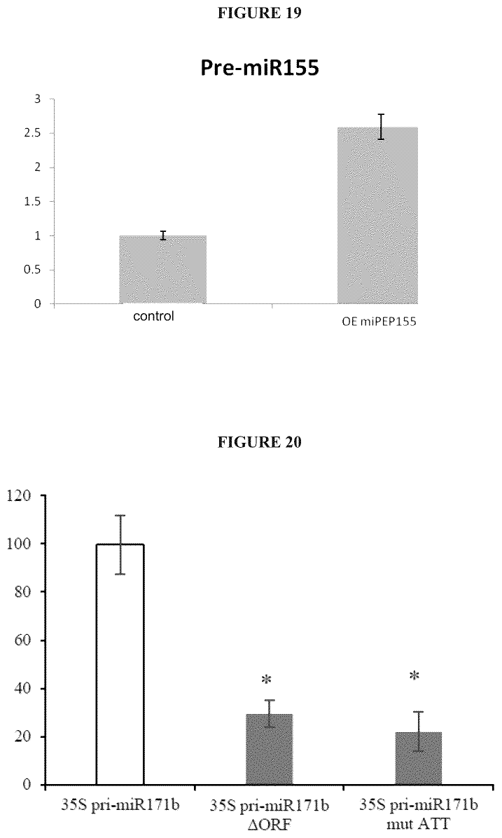

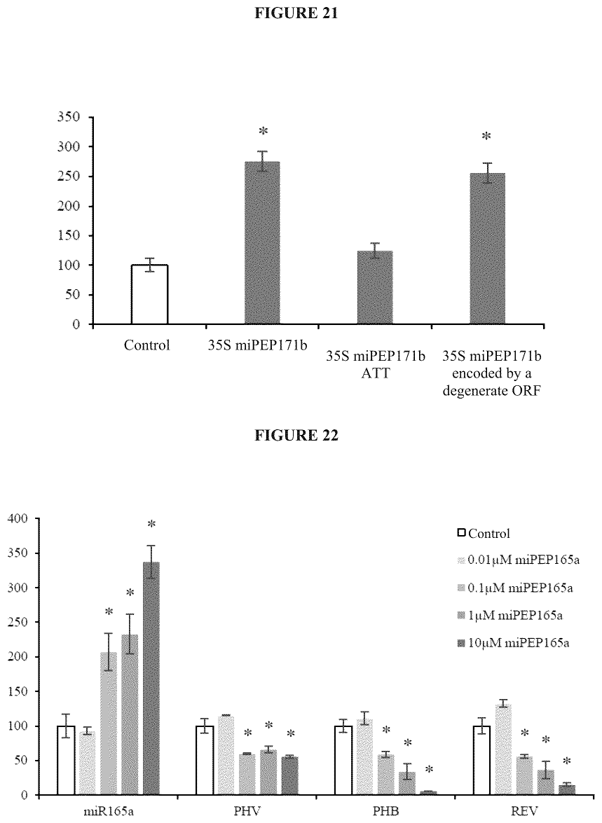

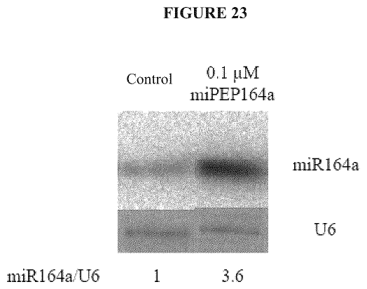

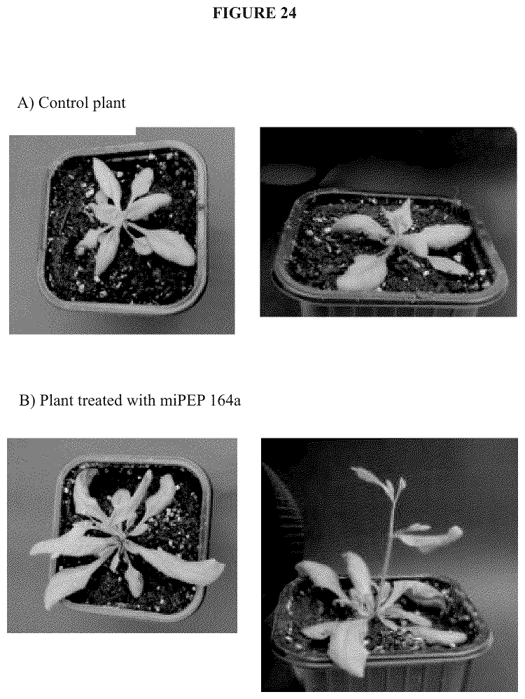

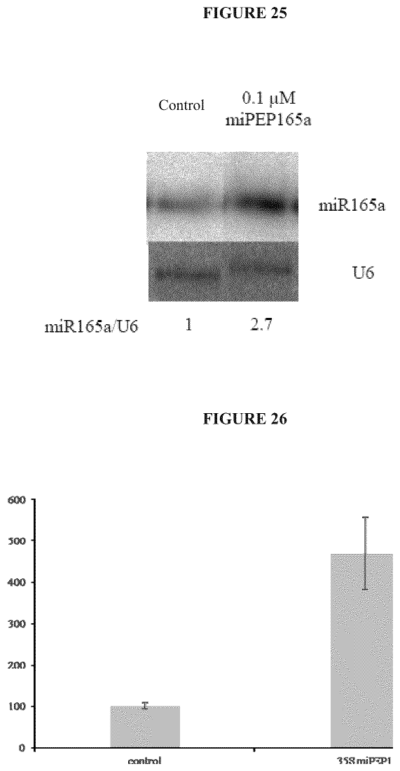

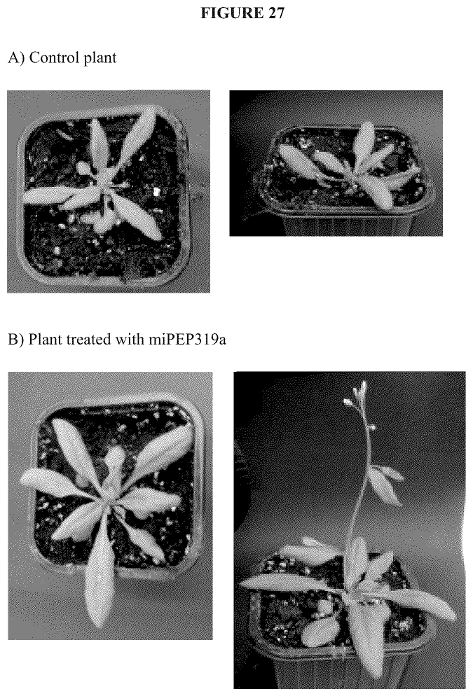

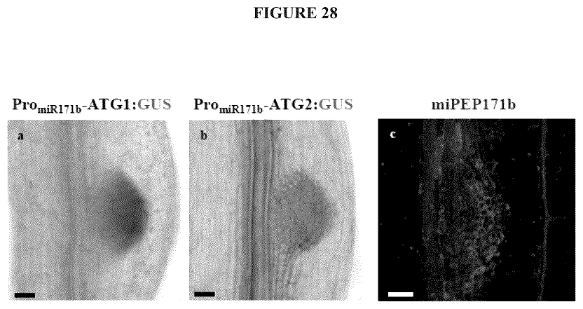

This microRNA is expressed in the meristematic region of the roots. The overexpression of this microRNA in particular leads to a reduction in the expression of the genes HAM1 (Accession No. MtGI9-TC114268) and HAM2 (Accession No. MtGI9-TC120850) (FIG. 1A), as well as to a reduction in the number of lateral roots (FIG. 1B).

The sequence of the primary transcript of MtmiR171b was determined using the RACE-PCR technique. Analysis of the sequence of the primary transcript made it possible to identify the presence of several completely unexpected small open reading frames (sORFs). These sORFs were called miORFs for “microRNA ORFs”. These miORFs potentially encode short peptides, from about 4 to 100 amino acids. No significant homology relating to these miORFS was found in the databases.

The overexpression of the first miORF, called MtmiORF171b, leads to an increase in the accumulation of MtmiR171b and a reduction in the expression of the HAM1 and HAM2 genes (see FIG. 2A), as well as to a reduction in the number of lateral roots (FIG. 2B), as was already observed in the overexpression of MtmiR171b.

In order to determine whether MtmiORF171b leads to the real production of a peptide and whether the regulatory function observed above is indeed carried by said peptide, a synthetic peptide, the sequence of which is identical to that potentially encoded by MtmiORF171b, was applied on the roots of Medicago truncatula. Application of this peptide leads to the phenotype already observed above in the overexpression of MtmiORF171b, i.e. it leads to an increase in the accumulation of MtmiR171b and a reduction in the expression of the HAM1 and HAM2 genes (see FIG. 3A), as well as a reduction in the number of lateral roots (FIG. 3B).

The results of these experiments demonstrate that MtmiORF171b encodes a peptide capable of modulating the accumulation of MtmiR171b, and the expression of the target genes of MtmiR171b: HAM1 and HAM2. Said peptide has been called MtmiPEP171b1 (“miPEP” corresponding to microRNA encoded PEPtide).

Moreover, MtPEP171b1 leads to an increase in the accumulation of MtmiR171b (FIG. 4A) and of pre-MtmiR171b (FIG. 4B).

b) Specificity of miPEP171b1

The expression of different microRNA precursors of Medicago truncatula (MtmiR171b SEQ ID NO: 319, MtmiR169 SEQ ID NO: 367, MtmiR169a SEQ ID NO: 368, MtmiR171a SEQ ID NO: 369, MtmiR171h SEQ ID NO: 370, MtmiR393a SEQ ID NO: 371, MtmiR393b SEQ ID NO: 372, MtmiR396a SEQ ID NO: 373 and MtmiR396b SEQ ID NO: 374) was determined and compared between control plants and plants in which MtmiORF171b encoding MtmiPEP171b1 was overexpressed (FIG. 5A), or between control plants and plants grown on culture medium containing MtmiPEP171b1 (FIG. 5B).

The results obtained indicate that MtmiPEP171b1 only leads to an increase in the accumulation of MtmiR171b and not of the other miRNAs, which indicates that a miPEP only has an effect on the microRNA from which it is derived.

c) Localization of miPEP171b1

Moreover, the immunolocalization of miPEP171b1 in the roots of M. truncatula reveals the presence of miPEP171b1 in the lateral root initiation sites, showing a co-localization between the microRNA and the corresponding miPEP (FIG. 28).

### Example 2—Characterization in the Model Plant of Tobacco

### a) Conservation of the Mechanism in Tobacco

In order to determine whether the mechanism of regulation of the microRNAs is conserved in other plant species, the regulation of MtmiR171b by MtmiPEP171b1 was tested in a different cellular model. For this, MtmiR171b and MtmiPEP171b1 were introduced into tobacco leaves.

The accumulation of MtmiR171b was measured in tobacco leaves transformed in order to express MtmiR171b of Medicago truncatula starting from wild-type pri-miRNA capable of producing MtmiPEP171b1, or starting from a mutated version of pri-miRNA incapable of producing MtmiPEP171b1 (in which the start codon ATG of MtmiORF171b has been replaced with ATT) (FIG. 6 and FIG. 20). Absence of translation of MtmiPEP171b1 leads to a marked decrease in the accumulation of MtmiR171b.

The accumulation of pre-MtmiR171b was measured in tobacco leaves transformed in order to express MtmiR171b of Medicago truncatula alone (control), or additionally expressing the wild-type MtmiORF171b of Medicago truncatula (35SmiPEP171b1 ATG), or a mutated version of MtmiORF171b in which the start codon ATG has been replaced with ATT (35SmiPEP171b1 ATT) (FIG. 7 and FIG. 21). The expression of MtmiORF171b leads to an increase in the accumulation of pre-miR171b, and this accumulation of pre-miR171b depends on the translation of MtmiORF171b to micropeptide.

Moreover, in the tobacco leaves transformed in order to express MtmiR171b of Medicago truncatula, untreated or treated by spraying with MtmiPEP171b1 (0.1 μM) for the first time 12 h before sampling and then a second time 30 minutes before sampling, it was observed that MtmiPEP171b1 may be used directly in peptide form by foliar sprayings (FIG. 8). Moreover, it was observed in tobacco (as in Medicago truncatula) that the MtmiPEP171b1 leads to an increase in the accumulation of MtmiR171b (FIG. 9A) and of pre-MtmiR171b (FIG. 9B), but reduces the accumulation of pri-MtmiR171b (FIG. 9C).

Taken together, these results indicate that the mechanism of regulation of the microRNAs and of their target genes under the control of miPEPs is conserved between the species.

### b) Intracellular Localization of MtmiPEP171b1

Tobacco leaves were transformed in order to overexpress MtmiPEP171b1 of Medicago truncatula fused with a fluorescent protein (GFP) (FIG. 10). The results obtained indicate that the miPEP is localized in small nuclear bodies.

c) Identification of miPEPS from Databases

Genomic databases of plants were searched for the presence of open reading frames within primary transcripts of 70 miRNAs, and 101 miORFs capable of encoding a miPEP were identified.

At present, AtmiPEP165a and AtmiPEP319a2, identified in Arabidopsis thaliana, have already been characterized. The experiments conducted in the model plant of tobacco made it possible to demonstrate that the overexpression of AtmiORF165a or of AtmiORF319a leads to an increase in the accumulation of AtmiR165a or of AtmiR319a respectively (FIG. 11).

miR165a regulates transcription factors such as Revoluta, Phavoluta and Phabulosa. miR319 regulates genes of the TCP family.

### Example 3—Characterization in the Arabidopsis thaliana Model Plant

### Example 3A—AtmiPEP165a

Regarding AtmiPEP165a, it has been demonstrated in vivo in Arabidopsis thaliana that treatment with AtmiPEP165a leads to a phenotype with greatly accelerated root growth (FIG. 12).

Moreover, treatment of plants with higher and higher concentrations of miPEP165a shows a dose-dependent effect on the accumulation of miR165a and the negative regulation of its target genes (PHAVOLUTA: PHV, PHABOLUSA: PHB and REVOLUTA: REV) as a function of the amount of miPEP165A (see FIG. 22).

### Example 3B—AtmiPEP164a

Regarding AtmiPEP164a, this was synthesized and was used for investigating an increase in the accumulation of miR164a in roots of A. thaliana treated with the synthetic peptide.

Northern blot analyses indicate that treatment of the plant with the peptide miPEP164a leads to an increase in the accumulation of miR164a (FIG. 23).

It has also been demonstrated in vivo in Arabidopsis thaliana that treatment of the plant with AtmiPEP164a increases plant growth significantly (FIG. 24).

### Example 3C—AtmiPEP165a

Regarding AtmiPEP165a, this was synthesized and was used for investigating an increase in the accumulation of miR165a in roots of A. thaliana treated with the synthetic peptide.

Northern blot analyses indicate that treatment of the plant with the peptide miPEP165a leads to an increase in the accumulation of miR165a (FIG. 25).

### Example 4C—AtmiPEP319a1

Regarding AtmiPEP319a1, this was also synthesized and was used for investigating an increase in the accumulation of miR319a in roots of A. thaliana treated with the synthetic peptide.

Analyses by qRT-PCR show that the overexpression of AtmiPEP319a1 leads to an increase in the accumulation of miR319a (FIG. 26).

It was also demonstrated in vivo in Arabidopsis thaliana that treatment of the plant with AtmiPEP319a1 increases plant growth significantly (FIG. 27).

### Material and Methods

### Biological Material

The surface of seeds of M. truncatula was sterilized and they were left to germinate on agar plates for 5 days at 4° C. in the dark. The young shoots were then grown on 12-cm square plates filled with Fahraeus medium without nitrogen and containing 7.5 UM phosphate (Lauressergues et al., Plant J., 72(3): 512-22, 2012). The lateral roots were counted every day. In pots, the plants were watered every other day with modified Long Ashton medium with low phosphorus content (Balzergue et al., Journal of Experimental Botany, (62)1049-1060, 2011).

The peptides were synthesized by Eurogentec or Smartox-Biotech. MtmiPEP171b1 was resuspended in a solution of 40% water/50% acetonitrile/10% acetic acid (v/v/v), and the other peptides were resuspended in water.

The leaves were watered by spraying with the peptides using peptide solutions at different concentrations (0.01, 0.1, 1 μM), firstly 12 h before sampling and then a second time 30 min before sampling.

Reverse Transcription of the microRNAs

The RNA was extracted using the reagent Tri-Reagent (MRC) according to the manufacturer's instructions, except for precipitation of the RNA, which was carried out with 3 volumes of ethanol. The reverse transcription of the RNA was carried out using the specific stem-loop primer RTprimer171b in combination with hexamers for performing the reverse transcription of RNA of high molecular weight.

In brief, 1 μg of RNA was added to the stem-loop primer MIR171b (0.2 μM), the hexamer (500 ng), the buffer RT (1×), the enzyme SuperScript Reverse transcriptase (SSIII) (one unit), the dNTPs (0.2 mM each), DTT (0.8 mM) in a final reaction mixture of 25 μl. In order to carry out the reverse transcription, a reaction of pulsed reverse transcription was performed (40 repetitions of the following cycle: 16° C. for 2 minutes, 42° C. for one minute and 50° C. for one second, followed by a final inactivation of the reverse transcription at 85° C. for 5 minutes).

Analyses by Quantitative RT-PCR (qRT-PCR)

The total RNA was extracted from roots of M. truncatula or from tobacco leaves using the extraction kit RNeasy Plant Mini Kit (Qiagen). The reverse transcription was performed using the reverse transcriptase SuperScript II (Invitrogen) starting from 500 ng of total RNA. Three repetitions (n=3) were carried out, each with two technical repetitions. Each experiment was repeated from two to three times. The amplifications by qPCR were carried out using a LightCycler 480 System thermocycler (Roche Diagnostics) by the method described in Lauressergues et al. (Plant J., 72(3): 512-22, 2012).

### Statistical Analyses

The mean values of the relative expression of the genes or of the production of lateral roots were analysed using the Student test or the Kruskal-Wallis test. The error bars represent the SEM (Standard Error of the Mean). The asterisks indicate a significant difference (p<0.05).

### Plasmid Constructs

The DNA fragments of interest were amplified with Pfu polymerase (Promega). The DNA fragments were cloned using the XhoI and NotI enzymes into a pPEX-DsRED plasmid for an overexpression under the control of the constitutive strong promoter 35S, and using the KpnI-NcoI enzymes into a pPEX GUS plasmid for the reporter genes, by the method described in Combier et al. (Genes & Dev, 22: 1549-1559, 2008).

For the miPEPs 165a and 319a, the corresponding miORFs were cloned into pBIN19 by the method described in Combier et al. (Genes & Dev, 22: 1549-1559, 2008).

### Transformation of the Plants

The composite plants having roots transformed with Agrobacterium rhizogenes were obtained by the method described in Boisson-Dernier et al. (Mol Plant-Microbe Interact, 18: 1269-1276, 2005). The transformed roots were verified and selected by observations of DsRED with a binocular fluorescence magnifier. The control roots correspond to roots transformed with A. rhizogenes not containing the pPEX-DsRED vector. Transformation of the tobacco leaves was carried out by the method described in Combier et al. (Genes & Dev, 22: 1549-1559, 2008).

### Northern Blot

Northern blot analysis was carried out according to the protocol described in Lauressergues et al. Plant J, 72(3): 512-22, 2012.

The biological samples were homogenized in a buffer containing 0.1 M of NaCl, 2% of SDS, 50 mM of Tris-HCl (pH 9), 10 mM of EDTA (pH 8) and 20 mM of mercaptoethanol, and the RNA was extracted twice with a phenol/chloroform mixture and was precipitated with ethanol.

The RNA was loaded on PAGE 15% gel and transferred to a nylon membrane (HybondNX, Amersham). RNA was hybridized with a radioactive oligonucleotide probe labelled at its end, in order to detect the RNA U6 or for miR164a.

The hybridizations were carried out at 55° C. The hybridization signals were quantified using a phosphorimager (Fuji) and normalized with the signal of the specific probe of RNA U6.

### Histochemical Labelling

Labelling with GUS was carried out by the method described in Combier et al., (Genes & Dev, 22: 1549-1559, 2008). The samples were observed with a microscope (axiozoom).

### Immunolocalization

Roots or plantlets of tissues of Medicago were fixed for 2 hours in 4% formol (v/v) with 50 mM of phosphate buffer (pH 7.2), and then embedded in agarose LMP 5% in water (with a low melting point). Thin sections (100 μm) were obtained and were placed in Pbi (phosphate buffer for immunology) on Teflon-coated slides, blocked in Pbi, 2% Tween and 1% of bovine serum albumin for 2 hours (PbiT-BSA), then labelled overnight (12 h) at 4° C. with the primary antibody diluted in BSA-PbiT. The sections were washed with PBiT and incubated at ambient temperature for 2 h with a secondary antibody diluted in PbiT-BSA. The slides were then washed in Pbi for 30 min and mounted in Citifluor (mounting medium). The primary antibodies and the dilutions were as follows: 1716a (1:500, v/v). The secondary antibody was a goat anti-rabbit IgG antibody coupled to the Alexa Fluor 633 fluorescent probe (Molecular Probes), and was used at a dilution of 1:1000 (v/v).

### B: Analysis of the miPEPs in Animals

### Example 4—Identification of Candidate miPEPs in Drosophila

A first study carried out by RACE-PCR in the model animal Drosophila melanogaster shows the existence of miRNAs that are expressed during embryogenesis, miR1 and miR8.

As in the plants, miORFs were identified in each of the two miRNAs studied. For example, miR8, known for its role in the regulation of growth in insects, has a miORF potentially encoding miPEP8.

Regarding DmmiR1 (identified in Drosophila melanogaster), it has two DmmiORFs potentially encoding DmmiPEP1a and DmmiPEP1b.

A phylogenetic analysis shows evolutionary conservation of the presence of the miORFs among the dozen Drosophila species analysed, i.e. since their divergence more than 60 million years ago (FIG. 13).

Moreover, the miPEPs identified in Drosophila have several similarities with the plant miPEPs. If their primary sequence and therefore their size evolve rapidly between species, a reduced size (from 32 to 104 AA) is found, as well as strong conservation for a basic overall charge (pHi from 9.5 to 12) (FIG. 14).

Taken together, these results therefore indicate the existence of regulatory miPEPs, encoded by the primary transcript of the microRNAs, over a broad spectrum of eukaryotic species. These discovered peptides represent an as yet unexplored reservoir of natural molecules that may regulate a variety of fundamental biological functions, both in plants and in animals.

Cells of Drosophila melanogaster

S2 cells are cultured in a T75 flask in 12 mL of Schneider's medium (GIBCO), containing 1% of penicillin 100 U/mL and streptavidin 100 mg/mL (Sigma) and 10% of decomplemented foetal calf serum (30 min at 56° C.).

The transient transfections are carried out using the FuGENE® HD transfection kit (Roche), according to the recommendations. Conventionally, 1.5 million S2 cells, previously seeded in 6-well plates (3 ml of medium per well), are transfected with 250 ng of total plasmid DNA. The DNA is brought into contact with the Fugene (3 μl) in 100 μl of OPTIMEM (GIBCO). After 20 minutes, the transfection reagent formed is brought into contact with the cells in the culture medium. The RNA of the cells is extracted 66 h after transfection.

### C: Characterization of miPEPs in Humans

### Example 5—Characterization of HsmiPEP155

The DNA fragments of interest (HsmiPEP155 and the mutated miPEP) were synthesized or amplified by PCR using specific primers, and then cloned using the enzymes XhoI and NotI into a pUAS plasmid permitting their overexpression by means of the GAL4 transcription factor, the expression of which is controlled by a constitutive strong promoter.

The different constructs were produced either by PCR amplification on genomic DNA of HeLa cells, or by RT-PCR on total RNAs of L428 human cells. The amplified PCR fragments are digested with the HindIII/EcoRI restriction enzymes and then cloned into the vector pcDNA3.1. The DH5a strain of Escherichia coli is electroporated and then cultured on a solid medium (2YT+agar+ampicillin). The plasmid DNA from different clones is then prepared and sequenced for verification. The constructs are then prepared using the QIAfilter Plasmid Midi kit (QIAGEN) and stored at −20° C.

The Hela cells (established tumour line, ATCC CCL-2.2) are cultured in a 6-well plate in complete medium [(DMEM (1×)+Glutamax+4.5 g/L glucose without pyruvate+1× penicillin/streptomycin+1 mM Na-pyruvate+10% calf serum] and placed in an incubator at 37° C. and 5% CO₂.

The cells are transfected when they are at 50% confluence. At the start of the experiment, the complete medium containing the antibiotics is replaced with complete medium without antibiotics.

For each well, a mix A [250 μl of Optimem (+Glutamax) (Gibco)+2 μg of DNA] and a mix B [250 μl of Optimem+4 μl of Lipofectamine 2000 (Invitrogen)] is prepared, and left for 5 min at ambient temperature. Then mix B is mixed dropwise into mix A, and left to incubate for 25 min at ambient temperature. The mixture is then deposited dropwise into the well. 4-5 hours later, the medium is changed and replaced with complete medium with antibiotics. 48 hours after transfection, the cells are stopped. The medium is aspirated and discarded; the cells are rinsed with PBS 1×. It is then possible to store the cells at −20° C. or extract the total RNAs directly.

For each well, the RNAs are extracted by depositing 1 ml of Tri-Reagent (Euromedex) on the cells. The Tri-reagent is aspirated and returned several times so that the cells are lysed correctly, and then it is transferred into a 1.5-ml tube. 0.2 ml of water-saturated chloroform is added. It is mixed by vortexing, then left for 2 to 3 minutes at ambient temperature. It is centrifuged for 5 minutes at 15300 rpm and at 4° C. The aqueous phase is precipitated from 0.5 ml of isopropanol after incubation for 10 minutes at ambient temperature and centrifugation for 15 minutes at 15300 rpm and at 4° C. The supernatant is discarded and the pellet is rinsed with 1 ml of 70% ethanol, with centrifugation for 5 minutes at 15300 rpm at 4° C. The supernatant is again discarded and the pellet is dried for a few minutes in the air.

For best-possible removal of the genomic DNA potentially remaining, the RNAs are treated with DNase. For this, the pellet is resuspended in 170 μl of ultra-pure water, 20 μl of DNase buffer 10× and 10 μl of RQ1 RNase-free DNase and held at 37° C. for 30 minutes. Then 20 μl of SDS10% and 5 μl of proteinase K (20 mg/ml) are added over 20 minutes at 37° C.

A last phenol extraction is carried out with 225 μl of a phenol/H₂O/chloroform mixture, and centrifuged for 5 minutes at 15300 rpm at 4° C.

The aqueous phase is then precipitated from 20 μl of 3M sodium acetate and 600 μl of 100% ethanol for 20 minutes at −80° C. Then it is centrifuged for 15 min at 4° C. at 15300 rpm. The supernatant is discarded. The pellet is rinsed in 1 ml of 70% ethanol, centrifuged for 5 min at 15300 rpm at 4° C., the supernatant is discarded again and the pellet is left to dry for some minutes in the air.

The pellet is then taken up in 15-20 μl of ultra-pure water and the RNAs are assayed.

10-15 μg of total RNAs is then analysed by Northern blot on 15% acrylamide gel [solution of acrylamide/40% bis-acrylamide, ratio 19:1], 7M urea in TBE 1×. Migration is carried out at 400V, in TBE1× as migration buffer, after preheating the gel. The RNAs are then electro-transferred onto a Biodyne Plus 0.45 μm nylon membrane, for 2 hours, at 1V and 4° C. in a transfer tank. At the end of transfer, the membrane is irradiated with UV at 0.124 J/cm². The membrane is then pre-hybridized in a buffer 5×SSPE, 1×Denhardt's, 1% SDS and 150 μg/ml of yeast tRNA, for 1 hour at 50° C. in a hybridization oven. Then the nucleotide probe is added, labelled at 5′ with γ-³²P-ATP (0.5 to 1.106 cpm/ml of hybridization buffer) and is hybridized overnight at 50° C. The membrane is then washed twice in 0.1×SSPE/0.1% SDS at ambient temperature and exposed in an autoradiography cassette containing a BioMax HE screen (Kodak) and a BioMax MS film (Kodak), in order to detect a microRNA, for 24-48 hours, at −80° C.

1. A process comprising:
a) identifying an open reading frame (ORF) from 12 to 303 nucleotides in length contained in the 5′ or 3′ portion of the primary transcript sequence of a microRNA (miRNA);
b1) exogenously contacting a first plant cell expressing the miRNA with a peptide encoded by the ORF or a nucleotide encoding the peptide; and
b2) comparing between:
i) a phenotype in the first plant cell expressing the miRNA in the presence of the peptide encoded by the ORF, the peptide being present in the first plant cell independently of transcription of the primary transcript of the miRNA, and
ii) the phenotype in a second plant cell of the same type as the first plant cell expressing the miRNA that has not been exogenously contacted with the peptide encoded by the ORF or the nucleotide encoding the peptide,
wherein a change of the phenotype in the presence of the peptide relative to the phenotype in the absence of the peptide indicates that the ORF encodes a miPEP that modulates the amount of the miRNA; and
c) synthesizing (i) the miPEP that modulates the amount of the miRNA, or (ii) a nucleic acid sequence encoding the miPEP that modulates the amount of the miRNA and that does not comprise mature miRNA.

2. The process of claim 1, wherein the nucleic acid sequence encoding the miPEP that modulates the amount of the miRNA and that does not comprise the mature miRNA is operably linked to a heterologous promoter.

3. The process of claim 1, wherein the synthesis in step c) is chemical synthesis or synthesis in a cell that transgenically expresses the miPEP that modulates the amount of the miRNA or the nucleic acid sequence encoding the miPEP that modulates the amount of the miRNA and that does not comprise the mature miRNA.

4. The process of claim 3, further comprising the step d) of isolating the miPEP from the cell that transgenically expresses the miPEP.

5. The process of claim 4, further comprising the step e) of formulating the miPEP isolated in step d) for administration to a plant or part thereof.

6. A process comprising synthesizing (i) a miPEP that modulates the amount of a microRNA (miRNA), or (ii) a nucleic acid sequence encoding the miPEP that modulates the amount of the miRNA and that does not comprise mature miRNA, wherein said miPEP is encoded by an open reading frame (ORF) from 12 to 303 nucleotides in length contained in the 5′ or 3′ portion of the primary transcript sequence of the miRNA, and wherein said miPEP was identified by:
a) exogenously contacting a first plant cell expressing the miRNA with a peptide encoded by the ORF or a nucleotide encoding the peptide; and
b) comparing between:
i) a phenotype in the first plant cell expressing the miRNA in the presence of the peptide encoded by the ORF, the peptide having been present in the first plant cell independently of transcription of the primary transcript of the miRNA, and
ii) the phenotype in a second plant cell of the same type as the first plant cell expressing the miRNA that had not been exogenously contacted with the peptide encoded by the ORF or the nucleotide encoding the peptide,
wherein a change of the phenotype in the presence of the peptide relative to the phenotype in the absence of the peptide indicated that the ORF encodes the miPEP that modulates the amount of the miRNA.

7. The process of claim 4, wherein the nucleic acid sequence encoding the miPEP that modulates the amount of the miRNA and that does not comprise the mature miRNA is operably linked to a heterologous promoter.

8. The process of claim 4, wherein the synthesizing is chemical synthesis or synthesis in a cell that transgenically expresses the miPEP that modulates the amount of the miRNA or the nucleic acid sequence encoding the miPEP that modulates the amount of the miRNA and that does not comprise the mature miRNA.

9. The process of claim 8, further comprising the step d) of isolating the miPEP from the cell that transgenically expresses the miPEP.

10. The process of claim 9, further comprising the step e) of formulating the miPEP isolated in step d) for administration to a plant or part thereof.
